(19) United States
(12) Patent Application Publication
(10) Pub. No.: US 2005/0026902 A1
(43) Pub. Date: Feb. 3, 2005

(54) Methods and compositions for the treatment or prevention of human immunodeficiency virus and related conditions using cyclooxygenase-2 selective inhibitors and antiviral agents

(76) Inventor: Timothy Maziasz, Northbrook, IL (US)

Correspondence Address: PHARMACIA CORPORATION;GLOBAL PATENT DEPARTMENT, POST OFFICE BOX 1027, ST. LOUIS, MO, 63006, US

(21) Appl. No.: 10/769,485

(22) Filed: Jan. 30, 2004

Related U.S. Application Data
(60) Provisional application No. 60/443,910, filed on Jan. 31, 2003.

(51) Int. Cl. A61K 31/55; A61K 31/54

(52) U.S. Cl. 514/217; 514/226.5

(57) ABSTRACT

The present invention provides compositions and methods for the treatment of human immunodeficiency virus (HIV) infection as well as HIV associated diseases and related disorders. More particularly, the invention provides a combination therapy for the treatment of HIV infection as well as HIV associated diseases and related disorders comprising the administration to a subject of an anti-human immunodeficiency virus agent in combination with a cyclooxygenase-2 selective inhibitor or an isomer or a pharmaceutically acceptable salt, ester, or prodrug thereof.

### CROSS-REFERENCE TO RELATED APPLICATIONS

This application claims the benefit of U.S. Provisional Application No. 60/443,910, filed Jan. 31, 2003, which is hereby incorporated by reference in its entirety.

### FIELD OF THE INVENTION

The present invention provides methods and compositions related to the treatment or prevention of human immunodeficiency virus (HIV) as well as related conditions. More particularly, the invention is directed toward a combination therapy for the treatment or prevention of HIV infection and related conditions comprising the administration to a subject of an anti-human immunodeficiency virus agent in combination with a cyclooxygenase-2 selective inhibitor.

### BACKGROUND OF THE INVENTION

The continued spread of the HIV epidemic in both the industrialized countries and the developing world provides compelling evidence that there is a continuing need for better anti-HIV treatments and for better anti-HIV drugs. Although HIV transmission is in theory largely preventable, in practice, without the development of better anti-HIV treatments and better anti-HIV drugs, HIV will continue to infect millions throughout the world. While programs to reduce transmission of HIV have achieved some success in both developed and developing countries, it is unlikely that widespread application of these programs will be able to achieve a sustained decrease in HIV transmission. Similarly, although the advent of highly effective antiretroviral therapy has resulted in significant increases in survival for HIV-infected individuals, the impact of combination antiretroviral therapy will be largely confined to the industrialized world, which constitutes only a small portion of the worldwide HIV-infected population.

Despite the urgent need for new anti-HIV drugs, which are both safe and effective, progress toward achieving this goal has been frustratingly slow. Agents currently used to treat HIV infection attempt to block replication of the HIV virus by blocking HIV reverse transcriptase or by blocking HIV protease. Three categories of anti-retroviral agents in clinical use are nucleoside analogs (such as AZT), protease inhibitors (such as nelfinavir), and non-nucleoside reverse transcriptase inhibitors (NNRTI), such as nevirapine. When any one of these agents is taken exclusively, however, only limited success has been achieved. Recently, the development of potent combination anti-retroviral regimens (cocktails) has improved prognosis for persons with HIV. The most commonly used combinations include two nucleoside analogs with or without a protease inhibitor. But, there is a continuing need for better anti-HIV treatments as well as better anti-HIV drugs.

Recent studies indicate that HIV infection may involve an inflammatory component. COX-2 is not normally expressed in lymph nodes or lymphocytes. International patent WO 02/07721 discloses, however, that mice infected by the immunodeficiency disorder MAIDs contained high levels of COX-2 within their lymph nodes. Moreover, is has also been disclosed that reducing certain inflammatory reactions by treatment of HIV patients with aspirin may beneficially affect the pathogenesis of the disease, improve some immune functions, slow the replication of HIV by reducing the levels of certain chemical messengers that may trigger the growth of the virus, and act as an immunostimulant by generating antigen-specific immune responses (James, J S, (1990) “Aspirin and AIDS” AIDS Treatment News Archive 109: 1-11).

Generally speaking, traditional NSAIDs, such as aspirin, are active in reducing the prostaglandin-induced pain and swelling associated with the inflammation process. But the use of high doses of most common NSAIDs can produce severe side effects, including life-threatening ulcers that limit their therapeutic potential. One reason proposed for the severe side effects associated with traditional NSAIDs is their non-selective inhibition of both of the cyclooxygenase enzymes (COX), commonly known as COX-1 and COX-2. COX-1 is constitutively expressed and mediates a number of physiological functions, such as kidney and gastrointestinal function. COX-2, contrastingly, is induced in response to an inflammation mediated event. While conventional NSAIDs block both forms of the enzyme, a new class of NSAID, selective cyclooxygenase-2 inhibitors, provide a viable target of inhibition that more effectively reduces inflammation and produces fewer and less drastic side effects.

Compounds that selectively inhibit cyclooxygenase-2 have been described in U.S. Pat. Nos. 5,380,738; 5,344,991; 5,393,790; 5,434,178; 5,474,995; 5,510,368 and WO documents WO96/06840, WO96/03388, WO96/03387, WO96/19469, WO96/25405, WO95/15316, WO94/15932, WO94/27980, WO95/00501, WO94/13635, WO94/20480, and WO94/26731. [Pyrazol-1-yl]benzenesulfonamides have been described as inhibitors of cyclooxygenase-2 and have shown promise in the treatment of inflammation, arthritis, and pain, with minimal side effects in pre-clinical and clinical trials.

Improved treatments for HIV infection are desperately needed to slow the continuing spread of this deadly disease. The current invention addresses this problem by providing a combination therapy comprising an anti-human immunodeficiency virus agent along with a cyclooxygenase-2 selective inhibitor.

### SUMMARY OF THE INVENTION

Among the several aspects of the invention is provided a method and a composition for the treatment of human immunodeficiency virus (HIV) as well as HIV associated diseases and related disorders in a subject. The method comprises administering to the subject a cyclooxygenase inhibitor or a pharmaceutically acceptable salt, isomer, ester or prodrug thereof and an anti-human immunodeficiency virus agent.

In one embodiment, the cyclooxygenase-2 selective inhibitor is a member of the chromene class of compounds. For example, the chromene compound may be a compound of the formula: 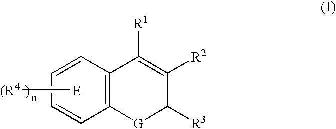
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;
or R⁴ together with the carbon atoms to which it is attached and the remainder of ring E forms a naphthyl radical.

In another embodiment, the cyclooxygenase-2 selective inhibitor or pharmaceutically acceptable salt, isomer, ester or prodrug thereof comprises a compound having the formula 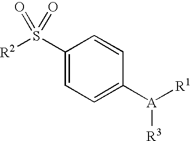
wherein
A is selected from the group consisting of partially unsaturated or unsaturated heterocyclyl and partially unsaturated or unsaturated carbocyclic rings;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl or amino; and
R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

In still a further embodiment, the anti-human immunodeficiency virus agent is selected from the group consisting of viral cellular entry inhibitors, viral replication inhibitors, viral assembly inhibitors, integrase inhibitors, human immune enhancing agents, virucidal agents, and antimitotic agents.

Other aspects and embodiments of the invention are more thoroughly detailed below.

Abbreviations and Definitions

The term “prevention” includes any of the following: (1) substantially preventing the onset of a clinically evident human immunodeficiency virus infection in a subject; (2) preventing the onset of a preclinically evident stage of a human immunodeficiency virus infection in a subject; or (3) substantially preventing human immunodeficiency virus colonization in a subject. This definition includes prophylactic treatment.

The term “inhibition” as used herein means a decrease in the severity of a human immunodeficiency virus infection as compared to that which would occur in the absence of the application of the present invention. This decrease in severity may result from a reduction in viral number, a reduction in viral replication, a reduction in the subject's cell growth infected with the virus, a reduction in cellular replication in the subject, a reduction in cellular mitosis in a subject, a reduction in viral colonization or any combination thereof.

The term “reduced cell growth” is intended to include any reduction in cell growth including the complete cessation of cell growth causing, e.g., apoptosis, in one or more human immunodeficiency virus-infected cells.

The phrase “human immunodeficiency virus (HIV) infection” means any presence of HIV in a subject, irrespective of the stage of infection or degree of colonization.

The phrase “human immunodeficiency virus (HIV) associated disease or related disorder” encompasses any kind of disease or related disorder caused by or resulting from HIV infection.

The phrase “therapeutically-effective” is intended to qualify the amount of each agent (i.e. cyclooxygenase-2 selective inhibitor or anti-HIV agent) which will achieve the goal of improvement in disorder severity and the frequency of incidence over no treatment or treatment of each agent by itself.

The term “subject” for purposes of treatment or prevention includes any species that is susceptible to HIV infection. In one embodiment, the subject is a human.

The term “cyclooxygenase-2 selective inhibitor” denotes a compound able to inhibit cyclooxygenase-2 without significant inhibition of cyclooxygenase-1. Preferably, it includes compounds that have a cyclooxygenase-2 IC₅₀ of less than about 0.2 micro molar, and also have a selectivity ratio of cyclooxygenase-2 inhibition over cyclooxygenase-1 inhibition of at least 50, and more preferably of at least 100. Even more preferably, the compounds have a cyclooxygenase-1 IC₅₀ of greater than about I micro molar, and more preferably of greater than 10 micro molar. Inhibitors of the cyclooxygenase pathway in the metabolism of arachidonic acid used in the present method may inhibit enzyme activity through a variety of mechanisms. By the way of example, and without limitation, the inhibitors used in the methods described herein may block the enzyme activity directly by acting as a substrate for the enzyme.

The term “hydrido” denotes a single hydrogen atom (H). This hydrido radical may be attached, for example, to an oxygen atom to form a hydroxyl radical or two hydrido radicals may be attached to a carbon atom to form a methylene (—CH2-) radical.

Where used, either alone or within other terms such as “haloalkyl”, “alkylsulfonyl”, “alkoxyalkyl” and “hydroxyalkyl”, the term “alkyl” embraces linear, cyclic or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkenyl” embraces linear or branched radicals having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl.

The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The terms “alkenyl”, “lower alkenyl”, embrace radicals having “cis” and “trans” orientations, or alternatively, “E” and “Z” orientations. The term “cycloalkyl” embraces saturated carbocyclic radicals having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The term “cycloalkenyl” embraces partially unsaturated carbocyclic radicals having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl.

The term “halo” means halogens such as fluorine, chlorine, bromine or iodine.

The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The terms “alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “heterocyclyl” embraces saturated, partially unsaturated and unsaturated heteroatom-containing ring-shaped radicals, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The term “heteroaryl” embraces unsaturated heterocyclyl radicals. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1 ,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also embraces radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino.

The term “alkylthio” embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “alkylthioalkyl” embraces radicals containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” embraces radicals containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “sulfonyl”, whether used alone or linked to other terms such as alkylsulfonyl, denotes respectively divalent radicals —SO2-. “Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” denote NH2O2S—.

The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, and trifluoroacetyl.

The term “carbonyl”, whether used alone or with other terms, such as “alkoxycarbonyl”, denotes —(C═O)—.

The term “aroyl” embraces aryl radicals with a carbonyl radical as defined above. Examples of aroyl include benzoyl, naphthoyl, and the like and the aryl in said aroyl may be additionally substituted.

The terms “carboxy” or “carboxyl”, whether used alone or with other terms, such as “carboxyalkyl”, denotes —CO2H.

The term “carboxyalkyl” embraces alkyl radicals substituted with a carboxy radical. More preferred are “lower carboxyalkyl” which embrace lower alkyl radicals as defined above, and may be additionally substituted on the alkyl radical with halo. Examples of such lower carboxyalkyl radicals include carboxymethyl, carboxyethyl and carboxypropyl.

The term “alkoxycarbonyl” means a radical containing an alkoxy radical, as defined above, attached via an oxygen atom to a carbonyl radical. More preferred are “lower alkoxycarbonyl” radicals with alkyl porions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “aralkyl” embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, haloalkyl and haloalkoxy. The terms benzyl and phenylmethyl are interchangeable.

The term “heterocyclylalkyl” embraces saturated and partially unsaturated heterocyclyl-substituted alkyl radicals, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, haloalkyl and haloalkoxy.

The term “aralkoxy” embraces aralkyl radicals attached through an oxygen atom to other radicals.

The term “aralkoxyalkyl” embraces aralkoxy radicals attached through an oxygen atom to an alkyl radical.

The term “aralkylthio” embraces aralkyl radicals attached to a sulfur atom.

The term “aralkylthioalkyl” embraces aralkylthio radicals attached through a sulfur atom to an alkyl radical.

The term “aminoalkyl” embraces alkyl radicals substituted with one or more amino radicals. More preferred are “lower aminoalkyl” radicals. Examples of such radicals include aminomethyl, aminoethyl, and the like.

The term “alkylamino” denotes amino groups that have been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like.

The term “arylamino” denotes amino groups, which have been substituted with one or two aryl radicals, such as N-phenylamino. The “arylamino” radicals may be further substituted on the aryl ring portion of the radical.

The term “aralkylamino” embraces aralkyl radicals attached through an amino nitrogen atom to other radicals. The terms “N-arylaminoalkyl” and “N-aryl-N-alkyl-aminoalkyl” denote amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aminocarbonyl” denotes an amide group of the formula —C(═O)NH2.

The term “alkylaminocarbonyl” denotes an aminocarbonyl group that has been substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above.

The term “alkylaminoalkyl” embraces radicals having one or more alkyl radicals attached to an aminoalkyl radical.

The term “aryloxyalkyl” embraces radicals having an aryl radical attached to an alkyl radical through a divalent oxygen atom.

The term “arylthioalkyl” embraces radicals having an aryl radical attached to an alkyl radical through a divalent sulfur atom.

### DESCRIPTION OF THE PREFERRED EMBODIMENTS

The present invention provides a combination therapy comprising the administration to a subject of a therapeutically effective amount of a COX-2 selective inhibitor in combination with a therapeutically effective amount of an anti-human immunodeficiency virus agent. The combination therapy is used to treat human immunodeficiency virus (HIV) as well as conditions resulting from HIV infection. When administered as part of a combination therapy, the COX-2 selective inhibitor together with the anti-human immunodeficiency virus agent provide enhanced treatment options as compared to administration of either the anti-human immunodeficiency virus agent or the COX-2 selective inhibitor alone.

### Cyclooxygenase-2 Selective Inhibitors

Any cyclooxygenase-2 selective inhibitor or prodrug or pharmaceutically acceptable salt thereof may be employed in the composition of the current invention. In one embodiment, the cyclooxygenase-2 selective inhibitor can be, for example, the cyclooxygenase-2 selective inhibitor meloxicam, Formula B-1 (CAS registry number 71125-38-7) or a pharmaceutically acceptable salt, ester, isomer or prodrug thereof.

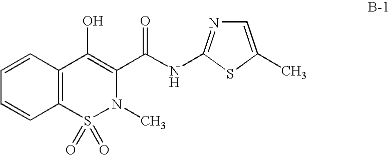

In yet another embodiment, the cyclooxygenase-2 selective inhibitor is the cyclooxygenase-2 selective inhibitor, 6-[[5-(4-chlorobenzoyl)-1,4-dimethyl-1H-pyrrol-2-yl]methyl]-3(2H)-pyridazinone, Formula B-2 (CAS registry number 179382-91-3) or a pharmaceutically acceptable salt, ester, isomer or prodrug thereof.

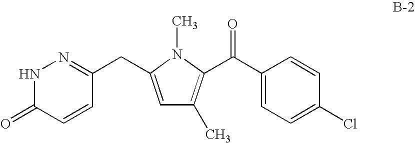

In a preferred embodiment the cyclooxygenase-2 selective inhibitor is preferably of the chromene structural class that is a substituted benzopyran or a substituted benzopyran analog, and even more preferably selected from the group consisting of substituted benzothiopyrans, dihydroquinolines, or dihydronaphthalenes having the general Formula I shown below and possessing, by way of example and not limitation, the structures disclosed in Table 1, including the diastereomers, enantiomers, racemates, tautomers, salts, esters, amides and prodrugs thereof. Furthermore, benzopyran cyclooxygenase-2 selective inhibitors useful in the practice of the present methods are described in U.S. Pat. Nos. 6,034,256 and 6,077,850 herein incorporated by reference in their entirety.

In one embodiment, the cyclooxygenase-2 selective inhibitor is a chromene compound represented by Formula I: 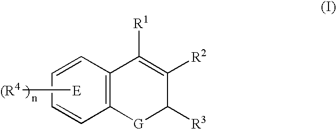
or an isomer, a pharmaceutically acceptable salt, ester, or prodrug thereof;
wherein n is an integer which is 0, 1, 2, 3 or 4;
wherein G is O, S or NR^{a};
wherein R^{a} is alkyl;
wherein R¹ is selected from the group consisting of H and aryl;
wherein R² is selected from the group consisting of carboxyl, aminocarbonyl, alkylsulfonylaminocarbonyl and alkoxycarbonyl;
wherein R³ is selected from the group consisting of haloalkyl, alkyl, aralkyl, cycloalkyl and aryl optionally substituted with one or more radicals selected from alkylthio, nitro and alkylsulfonyl; and
wherein each R⁴is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;
or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof wherein:
n is an integer which is 0, 1, 2, 3 or 4;
G is O, S or NR^{a};
R¹ is H;
R^{a} is alkyl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, beterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

In a further embodiment, the cyclooxygenase-2 selective inhibitor may also be a compound of Formula (I), or an isomer, a pharmaceutically acceptable salt, ester, or prodrug thereof; wherein:
n is an integer which is 0, 1, 2, 3 or 4;
G is oxygen or sulfur;
R¹ is H;
R² is carboxyl, lower alkyl, lower aralkyl or lower alkoxycarbonyl;
R³ is lower haloalkyl, lower cycloalkyl or phenyl; and
each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or
wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof; wherein:
R² is carboxyl;
R³ is lower haloalkyl; and
each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

The cyclooxygenase-2 selective inhibitor may also be a compound of Formula (I) or an isomer, a pharmaceutically acceptable salt, ester, or prodrug thereof; wherein:
n is an integer which is 0, 1, 2, 3 or 4;
R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and
each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof; wherein:
n is an integer which is 0, 1, 2, 3 or 4;
R³ is trifluoromethyl or pentafluoroethyl; and
each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

In yet another embodiment, the cyclooxygenase-2 selective inhibitor used in connection with the method(s) of the present invention can also be a compound having the structure of Formula (I) or an isomer, a pharmaceutically acceptable salt, ester, or prodrug thereof:

wherein:

n=4;
G is O or S;
R¹ is H;
R² is CO₂H;
R³ is lower haloalkyl;
a first R⁴ corresponding to R⁹ is hydrido or halo;
a second R⁴ corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6-membered nitrogen-containing heterocyclosulfonyl;
a third R⁴ corresponding to R¹¹ is H, lower alkyl, halo, lower alkoxy, or aryl; and
a fourth R⁴ corresponding to R¹² is H, halo, lower alkyl, lower alkoxy, and aryl;
wherein Formula (I) is represented by Formula (Ia): 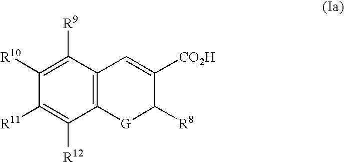
   or an isomer, a pharmaceutically acceptable salt, ester, or prodrug thereof.

The cyclooxygenase-2 selective inhibitor used in connection with the method(s) of the present invention can also be a compound of having the structure of Formula (Ia) or an isomer, a pharmaceutically acceptable salt, ester, or prodrug thereof; wherein:
R⁸ is trifluoromethyl or pentafluoroethyl;
R⁹ is H, chloro, or fluoro;
R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;
R¹¹ is H, methyl, ethyl, isopropyl, tert-butyl, chloro, methoxy, diethylamino, or phenyl; and
R¹² is H, chloro, bromo, fluoro, methyl, ethyl, tert-butyl, methoxy, or phenyl.

Examples of exemplary chromene cyclooxygenase-2 selective inhibitors are depicted in Table 1 below.

TABLE 1
Examples of Chromene Cyclooxygenase-2
Selective Inhibitors as Embodiments

| Compound | |
|---|---|
| Number | Structural Formula |
| B-3 | 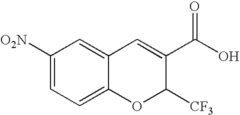 |
| | 6-Nitro-2-trifluoromethyl-2H-1- benzopyran-3-carboxylic acid |
| B-4 | 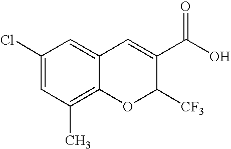 |
| | 6-Chloro-8-methyl-2-trifluoromethyl- 2H-1-benzopyran-3-carboxylic acid |
| B-5 | 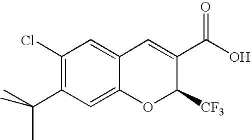 |
| | ((S)-6-Chloro-7-(1,1-dimethylethyl)-2-(trifluoro- methyl-2H-1-benzopyran-3-carboxylic acid |
| B-6 | 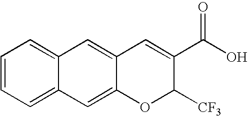 |
| | 2-Trifluoromethyl-2H-naphtho[2,3-b]pyran-3-carboxylic acid |
| B-7 | 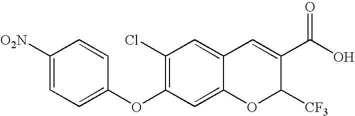 |
| | 6-Chloro-7-(4-nitrophenoxy)-2-(trifluoromethyl)-2H-1- benzopyran-3-carboxylic acid |
| B-8 | 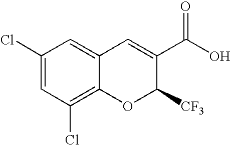 |
| | ((S)-6,8-Dichloro-2-(trifluoromethyl)- 2H-1-benzopyran-3-carboxylic acid |
| B-9 | 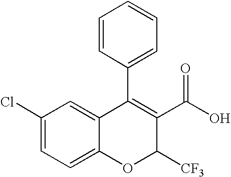 |
| | 6-Chloro-2-(trifluoromethyl)-4-phenyl-2H- 1-benzopyran-3-carboxyiic acid |
| B-10 | 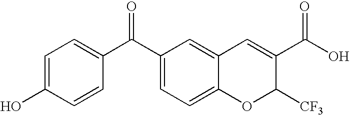 |
| | 6-(4-Hydroxybenzoyl)-2-(trifluoromethyl)- 2H-1-benzopyran-3-carboxylic acid |
| B-11 | 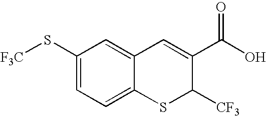 |
| | 2-(Trifluoromethyl)-6-[(trifluoromethyl)thio]- 2H-1-benzothiopyran-3-carboxylic acid |
| B-12 | 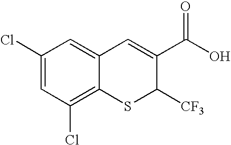 |
| | 6,8-Dichloro-2-trifiuoromethyl-2H-1- benzothiopyran-3-carboxylic acid |
| B-13 | 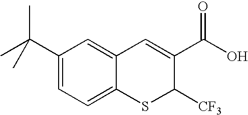 |
| | 6-(1,1-Dimethylethyl)-2-(trifluoromethyl)- 2H-1-benzothiopyran-3-carboxylic acid |
| B-14 | 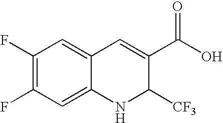 |
| | 6,7-Difluoro-1,2-dihydro-2-(trifluoro methyl)-3-quinolinecarboxylic acid |
| B-15 | 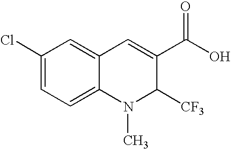 |
| | 6-Chloro-1,2-dihydro-1-methyl-2-(trifluoro methyl)-3-quinolinecarboxylic acid |
| B-16 | 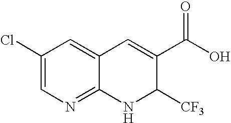 |
| | 6-Chloro-2-(trifluoromethyl)-1,2-dihydro [1,8]naphthyridine-3-carboxylic acid |
| B-17 | 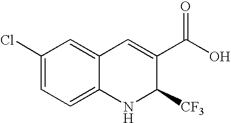 |
| | ((S)-6-Chloro-1,2-dihydro-2-(trifluoro methyl)-3-quinolinecarboxylic acid |

In a further preferred embodiment, the cyclooxygenase inhibitor is selected from the class of tricyclic cyclooxygenase-2 selective inhibitors represented by the general structure of Formula II: 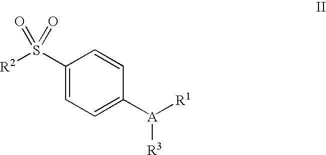
wherein A is selected from the group consisting of partially unsaturated or unsaturated heterocyclyl and partially unsaturated or unsaturated carbocyclic rings;
wherein R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
wherein R² is selected from the group consisting of methyl or amino; and
wherein R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N- arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically acceptable salt thereof.

In yet another embodiment, the cyclooxygenase-2 selective inhibitor is a compound of forumula II, wherein the compound is other than rofecoxib or celecoxib.

Yet another embodiment provides cyclooxygenase-2 selective inhibitors corresponding to formula II wherein A is a ring substituent selected from thienyl, oxazolyl, furyl, pyrrolyl, thiazolyl, imidazolyl, isothiazolyl, isoxazolyl, pyrazolyl, cyclopentenyl, phenyl, and pyridyl; wherein A is optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl, and lower hydroxyalkyl.

Another embodiment provides cyclooxygenase-2 selective inhibitors corresponding to formula II wherein A is a ring substituent selected from pyrrolyl, thiazolyl, imidazolyl, isothiazolyl, isoxazolyl, cyclopentenyl, phenyl, and pyridyl; wherein A is optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl, and lower hydroxyalkyl.

Yet another embodiment provides cyclooxygenase-2 selective inhibitors corresponding to formula II wherein A is a ring substituent selected from thienyl, oxazolyl, furyl, pyrrolyl, thiazolyl, imidazolyl, isothiazolyl, isoxazolyl, pyrazolyl, cyclopentenyl, phenyl, and pyridyl; wherein A is optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl, and lower hydroxyalkyl; provided that when A is pyrazolyl, R³ is other than trifluoromethyl, and provided that when A is furanone, R³ is other than hydrido.

In still another embodiment, the cycloxygenase-2 selective inhibitor is a compound of formula II, provided that when A is pyrazolyl, R³ is other than trifluoromethyl, and provided that when A is furanone, R³ is other than hydrido.

In a still more preferred embodiment of the invention the cyclooxygenase-2 selective inhibitor represented by the above Formula II is selected from the group of compounds, illustrated in Table 2, consisting of celecoxib (B-18; U.S. Pat. No. 5,466,823; CAS No. 169590-42-5), valdecoxib (B-19; U.S. Pat. No. 5,633,272; CAS No. 181695-72-7), deracoxib (B-20; U.S. Pat. No. 5,521,207; CAS No. 169590-41-4), rofecoxib (B-21; CAS No. 162011-90-7), etoricoxib (MK-663; B-22; PCT publication WO 98/03484), JTE-522 (B-23), or an isomer, ester, a pharmaceutically acceptable salt, ester, isomer or prodrug thereof.

TABLE 2
Examples of Tricyclic Cyclooxygenase-2
Selective Inhibitors as Embodiments

| Compound | |
|---|---|
| Number | Structural Formula |
| B-18 | 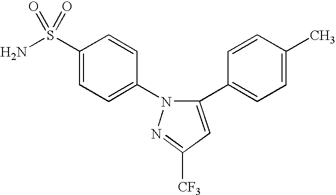 |
| B-19 | 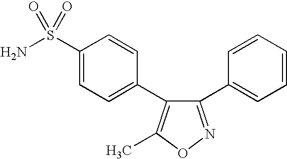 |
| B-20 | 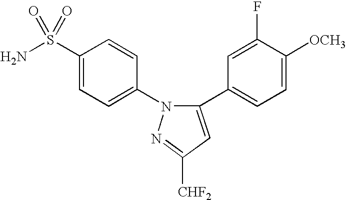 |
| B-21 | 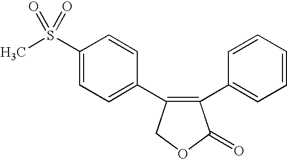 |
| B-22 | 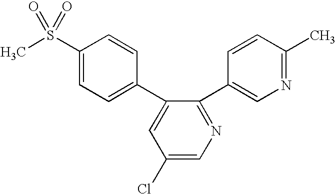 |
| B-23 | 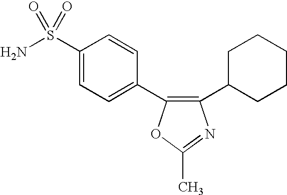 |

In an even more preferred embodiment, the cyclooxygenase-2 selective inhibitor is selected from the group consisting of celecoxib, rofecoxib and etoricoxib.

In another highly preferred embodiment of the invention, parecoxib (B-24, U.S. Pat. No. 5,932,598, CAS No. 198470-84-7), which is a therapeutically effective prodrug of the tricyclic cyclooxygenase-2 selective inhibitor valdecoxib, B-19, may be advantageously employed as a source of a cyclooxygenase inhibitor (U.S. Pat. No. 5,932,598, herein incorporated by reference).

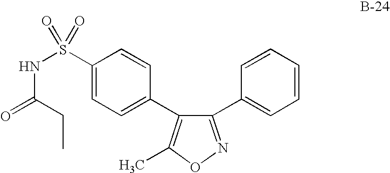

A preferred form of parecoxib is sodium parecoxib.

In another preferred embodiment of the invention, the compound having the formula B-25 that has been previously described in International Publication number WO 00/24719 (which is herein incorporated by reference), is another tricyclic cyclooxygenase-2 selective inhibitor which may be advantageously employed.

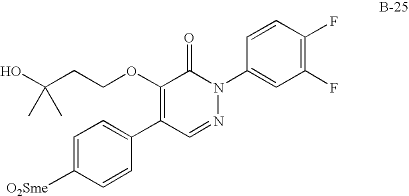

Another preferred cyclooxygenase-2 selective inhibitor that is useful in connection with the method(s) of the present invention is N-(2-cyclohexyloxynitrophenyl)-methane sulfonamide (NS-398) having a structure shown below as B-26.

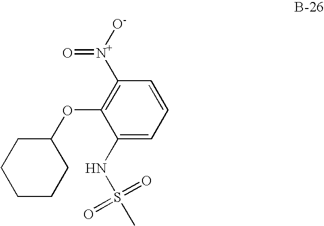

In yet a further preferred embodiment of the invention, the cyclooxygenase inhibitor used in connection with the method(s) of the present invention can be selected from the class of phenylacetic acid derivative cyclooxygenase-2 selective inhibitors represented by the general structure of Formula (III): 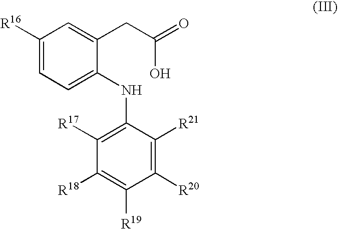
or an isomer, a pharmaceutically acceptable salt, ester, or prodrug thereof;
wherein
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

A particularly preferred phenylacetic acid derivative cyclooxygenase-2 selective inhibitor used in connection with the method(s) of the present invention is a compound that has the designation of COX 189 (B-211) and that has the structure shown in Formula (III) or an isomer, a pharmaceutically acceptable salt, ester, or prodrug thereof, wherein:
R¹⁶ is ethyl;
R¹⁷ and R¹⁹ are chloro;
R¹⁸ and R²⁰ are hydrogen; and
and R²¹ is methyl.

In yet another embodiment, the cyclooxygenase-2 selective inhibitor is represented by Formula (IV): 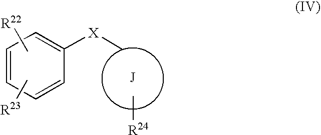
or an isomer, a pharmaceutically acceptable salt, an ester, or a prodrug thereof,
wherein:
X is O or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄.

According to another embodiment, the cyclooxygenase-2 selective inhibitors used in the present method(s) have the structural Formula (V): 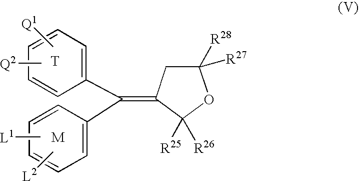
or an isomer, a pharmaceutically acceptable salt, an ester, or a prodrug thereof,
wherein: T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and
at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,
Q¹ and Q² are methylenedioxy; or
L¹ and L² are methylenedioxy; and
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²⁵ and R²⁶ are O; or,
R²⁷ and R²⁸ are O; or,
R²⁵, R²⁶, together with the carbon atom to which they are attached, form a saturated hydrocarbon ring having from 3 to 7 carbon atoms; or,
R²⁷, R²⁸, together with the carbon atom to which they are attached, form a saturated hydrocarbon ring having from 3 to 7 carbon atoms.

In a particularly preferred embodiment, the compounds N-(2-cyclohexyloxynitrophenyl)methane sulfonamide, and (E)-4-[(4-methylphenyl)(tetrahydro-2-oxo-3-furanylidene) methyl]benzenesulfonamide having the structure of Formula (V) are employed as cyclooxygenase-2 selective inhibitors.

Exemplary compounds that are useful for the cyclooxygenase-2 selective inhibitor in connection with the method(s) of the present invention, the structures for which are set forth in Table 3 below, include, but are not limited to:
6-chloro-2-trifluoromethyl-2H-1-benzopyran-3-carboxylic acid (B-27);
6-chloro-7-methyl-2-trifluoromethyl-2H-1-benzopyran-3-carboxylic acid (B-28);
8-(1-methylethyl)-2-trifluoromethyl-2H-1-benzopyran-3-carboxylic acid (B-29);
6-chloro-8-(1-methylethyl)-2-trifluoromethyl-2H-1-benzopyran-3-carboxylic acid (B-30);
2-trifluoromethyl-3H-naphtho[2,1-b]pyran-3-carboxylic acid (B-31);
7-(1,1-dimethylethyl)-2-trifluoromethyl-2H-1-benzopyran-3-carboxylic acid (B-32);
6-bromo-2-trifluoromethyl-2H-1-benzopyran-3-carboxylic acid (B-33);
8-chloro-2-trifluoromethyl-2H-1-benzopyran-3-carboxylic acid (B-34);
6-trifluoromethoxy-2-trifluoromethyl-2H-1-benzopyran-3-carboxylic acid (B-35);
5,7-dichloro-2-trifluoromethyl-2H-1-benzopyran-3-carboxylic acid (B-36);
8-phenyl-2-trifluoromethyl-2H-1-benzopyran-3-carboxylic acid (B-37);
7,8-dimethyl-2-trifluoromethyl-2H-1-benzopyran-3-carboxylic acid (B-38);
6,8-bis(dimethylethyl)-2-trifluoromethyl-2H-1-benzopyran-3-carboxylic acid (B-39);
7-(1-methylethyl)-2-trifluoromethyl-2H-1-benzopyran-3-carboxylic acid (B-40);
7-phenyl-2-trifluoromethyl-2H-1-benzopyran-3-carboxylic acid (B-41);
6-chloro-7-ethyl-2-trifluoromethyl-2H-1-benzopyran-3-carboxylic acid (B-42);
6-chloro-8-ethyl-2-trifluoromethyl-2H-1-benzopyran-3-carboxylic acid (B-43);
6-chloro-7-phenyl-2-trifluoromethyl-2H-1-benzopyran-3-carboxylic acid (B-44);
6,7-dichloro-2-trifluoromethyl-2H-1-benzopyran-3-carboxylic acid (B-45);
6,8-dichloro-2-trifluoromethyl-2H-1-benzopyran-3-carboxylic acid (B-46);
6-chloro-8-methyl-2-trifluoromethyl-2H-1-benzopyran-3-carboxylic acid (B-47);
8-chloro-6-methyl-2-trifluoromethyl-2H-1-benzopyran-3-carboxylic acid (B-48)
8-chloro-6-methoxy-2-trifluoromethyl-2H-1-benzopyran-3-carboxylic acid (B-49);
6-bromo-8-chloro-2-trifluoromethyl-2H-1-benzopyran-3-carboxylic acid (B-50);
8-bromo-6-fluoro-2-trifluoromethyl-2H-1-benzopyran-3-carboxylic acid (B-51);
8-bromo-6-methyl-2-trifluoromethyl-2H-1-benzopyran-3-carboxylic acid (B-52);
8-bromo-5-fluoro-2-trifluoromethyl-2H-1-benzopyran-3-carboxylic acid (B-53);
6-chloro-8-fluoro-2-trifluoromethyl-2H-1-benzopyran-3-carboxylic acid (B-54);
6-bromo-8-methoxy-2-trifluoromethyl-2H-1-benzopyran-3-carboxylic acid (B-55);
6-[[(phenylmethyl)amino]sulfonyl]-2-trifluoromethyl-2H-1-benzopyran-3-carboxylic acid (B-56);
6-[(dimethylamino)sulfonyl]-2-trifluoromethyl-2H-1-benzopyran-3-carboxylic acid (B-57);
6-[(methylamino)sulfonyl]-2-trifluoromethyl-2H-1-benzopyran-3-carboxylic acid (B-58);
6-[(4-morpholino)sulfonyl]-2-trifluoromethyl-2H-1-benzopyran-3-carboxylic acid (B-59);
6-[(1,1-dimethylethyl)aminosulfonyl]-2-trifluoromethyl-2H-1-benzopyran-3-carboxylic acid (B-60);
6-[(2-methylpropyl)aminosulfonyl]-2-trifluoromethyl-2H-1-benzopyran-3-carboxylic acid (B-61);
6-methylsulfonyl-2-trifluoromethyl-2H-1-benzopyran-3-carboxylic acid (B-62);
8-chloro-6-[[(phenylmethyl)amino]sulfonyl]-2-trifluoromethyl-2H-1-benzopyran-3-carboxylic acid (B-63);
6-phenylacetyl-2-trifluoromethyl-2H-1-benzopyran-3-carboxylic acid (B-64);
6,8-dibromo-2-trifluoromethyl-2H-1-benzopyran-3-carboxylic acid (B-65);
8-chloro-5,6-dimethyl-2-trifluoromethyl-2H-1-benzopyran-3-carboxylic acid (B-66);
6,8-dichloro-(S)-2-trifluoromethyl-2H-1-benzopyran-3-carboxylic acid (B-67);
6-benzylsulfonyl-2-trifluoromethyl-2H-1-benzopyran-3-carboxylic acid (B-68);
6-[[N-(2-furylmethyl)amino]sulfonyl]-2-trifluoromethyl-2H-1-benzopyran-3-carboxylic acid (B-69);
6-[[N-(2-phenylethyl)amino]sulfonyl]-2-trifluoromethyl-2H-1-benzopyran-3-carboxylic acid (B-70);
6-iodo-2-trifluoromethyl-2H-1-benzopyran-3-carboxylic. acid (B-71);
7-(1,1,1-dimethylethyl)-2-pentafluoroethyl-2H-1-benzopyran-3-carboxylic acid (B-72);
6-chloro-2-trifluoromethyl-2H-1-benzothiopyran-3-carboxylic acid (B-73);
3-[(3-Chloro-phenyl)-(4-methanesulfonyl-phenyl)-methylene]-dihydro-furan-2-one or BMS-347070 (B-74);
8-acetyl-3-(4-fluorophenyl)-2-(4-methylsulfonyl)phenyl-imidazo(1,2-a)pyridine (B-75);
5,5-dimethyl-4-(4-methylsulfonyl)phenyl-3-phenyl-2-(5H)-furanone (B-76);
5-(4-fluorophenyl)-1-[4-(methylsulfonyl)phenyl]-3-(trifluoromethyl)pyrazole (B-77);
4-(4-fluorophenyl)-5-[4-(methylsulfonyl)phenyl]-1-phenyl-3-(trifluoromethyl)pyrazole (B-78);
4-(5-(4-chlorophenyl)-3-(4-methoxyphenyl)-1H-pyrazol-1-yl)benzenesulfonamide (B-79);
4-(3,5-bis(4-methylphenyl)-1H-pyrazol-1-yl)benzenesulfonamide (B-80);
4-(5-(4-chlorophenyl)-3-phenyl-1H-pyrazol-1-yl)benzenesulfonamide (B-81);
4-(3,5-bis(4-methoxyphenyl)-1H-pyrazol-1-yl)benzenesulfonamide (B-82);
4-(5-(4-chlorophenyl)-3-(4-methylphenyl)-1H-pyrazol-1-yl)benzenesulfonamide (B-83);
4-(5-(4-chlorophenyl)-3-(4-nitrophenyl)-1H-pyrazol-1-yl)benzenesulfonamide (B-84);
4-(5-(4-chlorophenyl)-3-(5-chloro-2-thienyl)-1H-pyrazol-1-yl)benzenesulfonamide (B-85);
4-(4-chloro-3,5-diphenyl-1H-pyrazol-1-yl)benzenesulfonamide (B-86);
4-[5-(4-chlorophenyl)-3-(trifluoromethyl)-1H-pyrazol-1-yl]benzenesulfonamide (B-87);
4-[5-phenyl-3-(trifluoromethyl)-1H-pyrazol-1-yl]benzenesulfonamide (B-88);
4-[5-(4-fluorophenyl)-3-(trifluoromethyl)-1H-pyrazol-1-yl]benzenesulfonamide (B-89);
4-[5-(4-methoxyphenyl)-3-(trifluoromethyl)-1H-pyrazol-1-yl]benzenesulfonamide (B-90);
4-[5-(4-chlorophenyl)-3-(difluoromethyl)-1H-pyrazol-1-yl]benzenesulfonamide (B-91);
4-[5-(4-methylphenyl)-3-(trifluoromethyl)-1H-pyrazol-1-yl]benzenesulfonamide (B-92);
4-[4-chloro-5-(4-chlorophenyl)-3-(trifluoromethyl)-1H-pyrazol-1-yl]benzenesulfonamide (B-93);
4-[3-(difluoromethyl)-5-(4-methylphenyl)-1H-pyrazol-1-yl]benzenesulfonamide (B-94);
4-[3-(difluoromethyl)-5-phenyl-1H-pyrazol-1-yl]benzenesulfonamide (B-95);
4-[3-(difluoromethyl)-5-(4-methoxyphenyl)-1H-pyrazol-1-yl]benzenesulfonamide (B-96);
4-[3-cyano-5-(4-fluorophenyl)-1H-pyrazol-1-yl]benzenesulfonamide (B-97);
4-[3-(difluoromethyl)-5-(3-fluoro-4-methoxyphenyl)-1H-pyrazol-1-yl]benzenesulfonamide (B-98);
4-[5-(3-fluoro-4-methoxyphenyl)-3-(trifluoromethyl)-1H-pyrazol-1-yl]benzenesulfonamide (B-99);
4-[4-chloro-5-phenyl-1H-pyrazol-1-yl]benzenesulfonamide (B-100);
4-[5-(4-chlorophenyl)-3-(hydroxymethyl)-1H-pyrazol-1-yl]benzenesulfonamide (B-101);
4-[5-(4-(N,N-dimethylamino)phenyl)-3-(trifluoromethyl)-1H-pyrazol-1-yl]benzenesulfonamide (B-102);
5-(4-fluorophenyl)-6-[4-(methylsulfonyl)phenyl]spiro[2.4]hept-5-ene (B-103);
4-[6-(4-fluorophenyl)spiro[2.4]hept-5-en-5-yl]benzenesulfonamide (B-104);
6-(4-fluorophenyl)-7-[4-(methylsulfonyl)phenyl]spiro[3.4]oct-6-ene (B-105);
5-(3-chloro-4-methoxyphenyl)-6-[4-(methylsulfonyl)phenyl]spiro[2.4]hept-5-ene (B-106);
4-[6-(3-chloro-4-methoxyphenyl)spiro[2.4]hept-5-en-5-yl]benzenesulfonamide (B-107);
5-(3,5-dichloro-4-methoxyphenyl)-6-[4-(methylsulfonyl)phenyl]spiro[2.4]hept-5-ene (B-108);
5-(3-chloro-4-fluorophenyl)-6-[4-(methylsulfonyl)phenyl]spiro[2.4]hept-5-ene (B-109);
4-[6-(3,4-dichlorophenyl)spiro[2.4]hept-5-en-5-yl]benzenesulfonamide (B-110);
2-(3-chloro-4-fluorophenyl)-4-(4-fluorophenyl)-5-(4-methylsulfonylphenyl)thiazole (B-111);
2-(2-chlorophenyl)-4-(4-fluorophenyl)-5-(4-methylsulfonylphenyl)thiazole (B-112);
5-(4-fluorophenyl)-4-(4-methylsulfonylphenyl)-2-methylthiazole (B-113);
4-(4-fluorophenyl)-5-(4-methylsulfonylphenyl)-2-trifluoromethylthiazole (B-114);
4-(4-fluorophenyl)-5-(4-methylsulfonylphenyl)-2-(2-thienyl)thiazole (B-115);
4-(4-fluorophenyl)-5-(4-methylsulfonylphenyl)-2-benzylaminothiazole (B-116);
4-(4-fluorophenyl)-5-(4-methylsulfonylphenyl)-2-(1-propylamino)thiazole (B-117);
2-[(3,5-dichlorophenoxy)methyl)-4-(4-fluorophenyl)-5-[4-(methylsulfonyl)phenyl]thiazole (B-118);
5-(4-fluorophenyl)-4-(4-methylsulfonylphenyl)-2-trifluoromethylthiazole (B-119);
1-methylsulfonyl-4-[1,1-dimethyl-4-(4-fluorophenyl)cyclopenta-2,4-dien-3-yl]benzene (B-120);
4-[4-(4-fluorophenyl)-1,1-dimethylcyclopenta-2,4-dien-3-yl]benzenesulfonamide (B-121);
5-(4-fluorophenyl)-6-[4-(methylsulfonyl)phenyl]spiro[2.4]hepta-4,6-diene (B-122);
4-[6-(4-fluorophenyl)spiro[2.4]hepta-4,6-dien-5-yl]benzenesulfonamide (B-123);
6-(4-fluorophenyl)-2-methoxy-5-[4-(methylsulfonyl)phenyl]-pyridine-3-carbonitrile (B-124);
2-bromo-6-(4-fluorophenyl)-5-[4-(methylsulfonyl)phenyl]-pyridine-3-carbonitrile (B-125);
6-(4-fluorophenyl)-5-[4-(methylsulfonyl)phenyl]-2-phenyl-pyridine-3-carbonitrile (B-126);
4-[2-(4-methylpyridin-2-yl)-4-(trifluoromethyl)-1H-imidazol-1-yl]benzenesulfonamide (B-127);
4-[2-(5-methylpyridin-3-yl)-4-(trifluoromethyl)-1H-imidazol-1-yl]benzenesulfonamide (B-128);
4-[2-(2-methylpyridin-3-yl)-4-(trifluoromethyl)-1H-imidazol-1-yl]benzenesulfonamide (B-129);
3-[1-[4-(methylsulfonyl)phenyl]-4-(trifluoromethyl)-1H-imidazol-2-yl]pyridine (B-130);
2-[1-[4-(methylsulfonyl)phenyl-4-(trifluoromethyl)-1H-imidazol-2-yl]pyridine (B-131);
2-methyl-4-[1-[4-(methylsulfonyl)phenyl-4-(trifluoromethyl)-1H-imidazol-2-yl]pyridine (B-132);
2-methyl-6-[1-[4-(methylsulfonyl)phenyl-4-(trifluoromethyl)-1H-imidazol-2-yl]pyridine (B-133);
4-[2-(6-methylpyridin-3-yl)-4-(trifluoromethyl)-1H-imidazol-1-yl]benzenesulfonamide (B-134);
2-(3,4-difluorophenyl)-1-[4-(methylsulfonyl)phenyl]-4-(trifluoromethyl)-1H-imidazole (B-135);
4-[2-(4-methylphenyl)-4-(trifluoromethyl)-1H-imidazol-1-yl]benzenesulfonamide (B-136);
2-(4-chlorophenyl)-1-[4-(methylsulfonyl)phenyl]-4-methyl-1H-imidazole (B-137);
2-(4-chlorophenyl)-1-[4-(methylsulfonyl)phenyl]-4-phenyl-1H-imidazole (B-138);
2-(4-chlorophenyl)-4-(4-fluorophenyl)-1-[4-(methylsulfonyl)phenyl]-1H-imidazole (B-139);
2-(3-fluoro-4-methoxyphenyl)-1-[4-(methylsulfonyl)phenyl-4-(trifluoromethyl)-1H-imidazole (B-140);
1-[4-(methylsulfonyl)phenyl]-2-phenyl-4-trifluoromethyl-1H-imidazole (B-141);
2-(4-methylphenyl)-1-[4-(methylsulfonyl)phenyl]-4-trifluoromethyl-1H-imidazole (B-142);
4-[2-(3-chloro-4-methylphenyl)-4-(trifluoromethyl)-1H-imidazol-1-yl]benzenesulfonamide (B-143);
2-(3-fluoro-5-methylphenyl)-1-[4-(methylsulfonyl)phenyl]-4-(trifluoromethyl)-1H-imidazole (B-144);
4-[2-(3-fluoro-5-methylphenyl)-4-(trifluoromethyl)-1H-imidazol-1-yl]benzenesulfonamide (B-145);
2-(3-methylphenyl)-1-[4-(methylsulfonyl)phenyl]-4-trifluoromethyl-1H-imidazole (B-146);
4-[2-(3-methylphenyl)-4-trifluoromethyl-1H-imidazol-1-yl]benzenesulfonamide (B-147);
1-[4-(methylsulfonyl)phenyl]-2-(3-chlorophenyl)-4-trifluoromethyl-1H-imidazole (B-148);
4-[2-(3-chlorophenyl)-4-trifluoromethyl-1H-imidazol-1-yl]benzenesulfonamide (B-149);
4-[2-phenyl-4-trifluoromethyl-1H-imidazol-1-yl]benzenesulfonamide (B-150);
4-[2-(4-methoxy-3-chlorophenyl)-4-trifluoromethyl-1H-imidazol-1-yl]benzenesulfonamide (B-151);
1-allyl-4-(4-fluorophenyl)-3-[4-(methylsulfonyl)phenyl]-5-(trifluoromethyl)-1H-pyrazole (B-152);
4-[1-ethyl-4-(4-fluorophenyl)-5-(trifluoromethyl)-1H-pyrazol-3-yl]benzenesulfonamide (B-153);
N-phenyl-[4-(4-fluorophenyl)-3-[4-(methylsulfonyl)phenyl]-5-(trifluoromethyl)-1H-pyrazol-1-yl]acetamide (B-154);
ethyl [4-(4-fluorophenyl)-3-[4-(methylsulfonyl)phenyl]-5-(trifluoromethyl)-1H-pyrazol-1-yl]acetate (B-155);
4-(4-fluorophenyl)-3-[4-(methylsulfonyl)phenyl]-1-(2-phenylethyl)-1H-pyrazole (B-156);
4-(4-fluorophenyl)-3-[4-(methylsulfonyl)phenyl]-1-(2-phenylethyl)-5-(trifluoromethyl)pyrazole (B-157);
1-ethyl-4-(4-fluorophenyl)-3-[4-(methylsulfonyl)phenyl]-5-(trifluoromethyl)-1H-pyrazole (B-158);
5-(4-fluorophenyl)-4-(4-methylsulfonylphenyl)-2-trifluoromethyl-1H-imidazole (B-159);
4-[4-(methylsulfonyl)phenyl]-5-(2-thiophenyl)-2-(trifluoromethyl)-1H-imidazole (B-160);
5-(4-fluorophenyl)-2-methoxy-4-[4-(methylsulfonyl)phenyl]-6-(trifluoromethyl)pyridine (B-161);
2-ethoxy-5-(4-fluorophenyl)-4-[4-(methylsulfonyl)phenyl]-6-(trifluoromethyl)pyridine (B-162);
5-(4-fluorophenyl)-4-[4-(methylsulfonyl)phenyl]-2-(2-propynyloxy)-6-(trifluoromethyl)pyridine (B-163);
2-bromo-5-(4-fluorophenyl)-4-[4-(methylsulfonyl)phenyl]-6-(trifluoromethyl)pyridine (B-164);
4-[2-(3-chloro-4-methoxyphenyl)-4,5-difluorophenyl]benzenesulfonamide (B-165);
1-(4-fluorophenyl)-2-[4-(methylsulfonyl)phenyl]benzene (B-166);
5-difluoromethyl-4-(4-methylsulfonylphenyl)-3-phenylisoxazole (B-167);
4-[3-ethyl-5-phenylisoxazol-4-yl]benzenesulfonamide (B-168);
4-[5-difluoromethyl-3-phenylisoxazol-4-yl]benzenesulfonamide (B-169);
4-[5-hydroxymethyl-3-phenylisoxazol-4-yl]benzenesulfonamide (B-170);
4-[5-methyl-3-phenyl-isoxazol-4-yl]benzenesulfonamide (B-171);
1-[2-(4-fluorophenyl)cyclopenten-1-yl]-4-(methylsulfonyl)benzene (B-172);
1-[2-(4-fluoro-2-methylphenyl)cyclopenten-1-yl]-4-(methylsulfonyl)benzene (B-173);
1-[2-(4-chlorophenyl)cyclopenten-1-yl]-4-(methylsulfonyl)benzene (B-174);
1-[2-(2,4-dichlorophenyl)cyclopenten-1-yl]-4-(methylsulfonyl)benzene (B-175);
1-[2-(4-trifluoromethylphenyl)cyclopenten-1-yl]-4-(methylsulfonyl)benzene (B-176);
1-[2-(4-methylthiophenyl)cyclopenten-1-yl]-4-(methylsulfonyl)benzene (B-177);
1-[2-(4-fluorophenyl)-4,4-dimethylcyclopenten-1-yl]-4-(methylsulfonyl)benzene (B-178);
4-[2-(4-fluorophenyl)-4,4-dimethylcyclopenten-1-yl]benzenesulfonamide (B-179);
1-[2-(4-chlorophenyl)-4,4-dimethylcyclopenten-1-yl]-4-(methylsulfonyl)benzene (B-180);
4-[2-(4-chlorophenyl)-4,4-dimethylcyclopenten-1-yl]benzenesulfonamide (B-181);
4-[2-(4-fluorophenyl)cyclopenten-1-yl]benzenesulfonamide (B-182);
4-[2-(4-chlorophenyl)cyclopenten-1-yl]benzenesulfonamide (B-183);
1-[2-(4-methoxyphenyl)cyclopenten-1-yl]-4-(methylsulfonyl)benzene (B-184);
1-[2-(2,3-difluorophenyl)cyclopenten-1-yl]-4-(methylsulfonyl)benzene (B-185);
4-[2-(3-fluoro-4-methoxyphenyl)cyclopenten-1-yl]benzenesulfonamide (B-186);
1-[2-(3-chloro-4-methoxyphenyl)cyclopenten-1-yl]-4-(methylsulfonyl)benzene (B-187);
4-[2-(3-chloro-4-fluorophenyl)cyclopenten-1-yl]benzenesulfonamide (B-188);
4-[2-(2-methylpyridin-5-yl)cyclopenten-1-yl]benzenesulfonamide (B-189);
ethyl 2-[4-(4-fluorophenyl)-5-[4-(methylsulfonyl) phenyl]oxazol-2-yl]-2-benzyl-acetate (B-190);
2-[4-(4-fluorophenyl)-5-[4-(methylsulfonyl)phenyl]oxazol-2-yl]acetic acid (B-191);
2-(tert-butyl)-4-(4-fluorophenyl)-5-[4-(methylsulfonyl)phenyl]oxazole (B-192);
4-(4-fluorophenyl)-5-[4-(methylsulfonyl)phenyl]-2-phenyloxazole (B-193);
4-(4-fluorophenyl)-2-methyl-5-[4-(methylsulfonyl)phenyl]oxazole (B-194);
4-[5-(3-fluoro-4-methoxyphenyl)-2-trifluoromethyl-4-oxazolyl]benzenesulfonamide (B-195);
6-chloro-7-(1,1-dimethylethyl)-2-trifluoromethyl-2H-1-benzopyran-3-carboxylic acid (B-196);
6-chloro-8-methyl-2-trifluoromethyl-2H-1-benzopyran-3-carboxylic acid (B-197);
5,5-dimethyl-3-(3-fluorophenyl)-4-methylsulfonyl-2(5H)-furanone (B-198);
6-chloro-2-trifluoromethyl-2H-1-benzothiopyran-3-carboxylic acid (B-199);
4-[5-(4-chlorophenyl)-3-(trifluoromethyl)-1H-pyrazol-1-yl]benzenesulfonamide (B-200);
4-[5-(4-methylphenyl)-3-(trifluoromethyl)-1H-pyrazol-1-yl]benzenesulfonamide (B-201);
4-[5-(3-fluoro-4-methoxyphenyl)-3-(difluoromethyl)-1H-pyrazol-1-yl]benzenesulfonamide (B-202);
3-[1-[4-(methylsulfonyl)phenyl]-4-trifluoromethyl-1H-imidazol-2-yl]pyridine (B-203);
2-methyl-5-[1-[4-(methylsulfonyl)phenyl]-4-trifluoromethyl-1H-imidazol-2-yl]pyridine (B-204);
4-[2-(5-methylpyridin-3-yl)-4-(trifluoromethyl)-1H-imidazol-1-yl]benzenesulfonamide (B-205);
4-[5-methyl-3-phenylisoxazol-4-yl]benzenesulfonamide (B-206);
4-[5-hydroxymethyl-3-phenylisoxazol-4-yl]benzenesulfonamide (B-207);
[2-trifluoromethyl-5-(3,4-difluorophenyl)-4-oxazolyl]benzenesulfonamide (B-208);
4-[2-methyl-4-phenyl-5-oxazolyl]benzenesulfonamide (B-209);
4-[5-(2-fluoro-4-methoxyphenyl)-2-trifluoromethyl-4-oxazolyl]benzenesulfonamide (B-210);
[2-(2-chloro-6-fluoro-phenylamino)-5-methyl-phenyl]-acetic acid or COX 189 (B-211);
N-(4-Nitro-2-phenoxy-phenyl)-methanesulfonamide or nimesulide (B-212);
N-[6-(2,4-difluoro-phenoxy)-1-oxo-indan-5-yl]-methanesulfonamide or flosulide (B-213);
N-[6-(2,4-Difluoro-phenylsulfanyl)-1-oxo-1H-inden-5-yl]-methanesulfonamide, soldium salt or L-745337 (B-214);
N-[5-(4-fluoro-phenylsulfanyl)-thiophen-2-yl]-methanesulfonamide or RWJ-63556 (B-215);
3-(3,4-Difluoro-phenoxy)-4-(4-methanesulfonyl-phenyl)-5-methyl-5-(2,2,2-trifluoro-ethyl)-5H-furan-2-one or L-784512 or L-784512 (B-216);
(5Z)-2-amino-5-[[3,5-bis(1,1-dimethylethyl)-4-hydroxyphenyl]methylene]-4(5H)-thiazolone or darbufelone (B-217);
CS-502 (B-218);
LAS-34475 (B-219);
LAS-34555 (B-220);
S-33516 (B-221);
SD-8381 (B-222);
L-783003 (B-223);
N-[3-(formylamino)-4-oxo-6-phenoxy-4H-1-benzopyran-7-yl]-methanesulfonamide or T-614 (B-224);
D-1367 (B-225);
L-748731 (B-226);
(6aR,10aR)-3-(1,1-dimethylheptyl)-6a,7,10,10a-tetrahydro-1-hydroxy-6,6-dimethyl-6H-dibenzo[b,d]pyran-9-carboxylic acid or CT3 (B-227);
CGP-28238 (B-228);
4-[[3,5-bis(1,1-dimethylethyl)-4-hydroxyphenyl]methylene]dihydro-2-methyl-2H-1,2-oxazin-3(4H)-one or BF-389 (B-229);
GR-253035 (B-230);
6-dioxo-9H-purin-8-yl-cinnamic acid (B-231);
S-2474 (B-232);
4-[4-(methyl)-sulfonyl)phenyl]-3-phenyl-2(5H)-furanone;
4-(5-methyl-3-phenyl-4-isoxazolyl);
2-(6-methylpyrid-3-yl)-3-(4-methylsulfonylphenyl)-5-chloropyridine;
4-[5-(4-methylphenyl)-3-(trifluoromethyl)-1H-pyrazol-1-yl];
N-[[4-(5-methyl-3-phenyl-4-isoxazolyl)phenyl]sulfonyl];
4-[5-(3-fluoro-4-methoxyphenyl)-3-difluoromethyl)-1H-pyrazol-1-yl]benzenesulfonamide;
(S)-6,8-dichloro-2-(trifluoromethyl)-2H-1-benzopyran-3-carboxylic acid;
2-(3,4-difluorophenyl)-4-(3-hydroxy-3-methylbutoxy)-5-[4-(methylsulfonyl)phenyl]-3(2H)-pyridzainone;
2-trifluoromethyl-3H-naptho[2,1-b]pyran-3-carboxylic acid;
6-chloro-7-(1,1-dimethylethyl)-2-trifluoromethyl-2H-1-benzopyran-3-carboxylic acid;
[2-(2,4-dichloro-6-ethyl-3,5-dimethyl-phenylamino)-5-propyl-phenyl]-acetic acid;

or an isomer, a pharmaceutically acceptable salt, ester or prodrug thereof.

TABLE 3
Examples of Cyclooxygenase-2 Selective Inhibitors as Embodiments

| Com- | |
|---|---|
| pound | |
| Number | Structural Formula |
| B-26 | 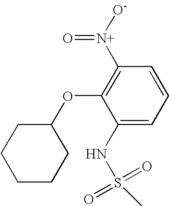 |
| | N-(2-cyclohexyloxynitrophenyl) methane |
| | sulfonamide or NS-398; |
| B-27 | 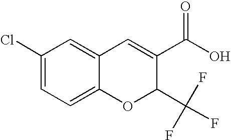 |
| | 6-chloro-2-trifluoromethyl-2H-1-benzopyran-3-carboxylic acid; |
| B-28 | 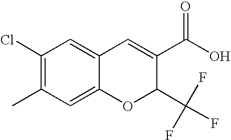 |
| | 6-chloro-7-methyl-2-trifluoromethyl-2H-1-benzopyran-3- |
| | carboxylic acid; |
| B-29 | 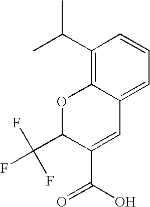 |
| | 8-(1-methylethyl)-2-trifluoromethyl-2H-1-benzopyran-3- |
| | carboxylic acid; |
| B-30 | 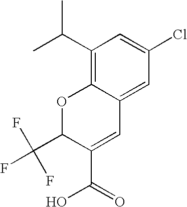 |
| | 6-chloro-8-(1-methylethyl)-2-trifluoromethyl- |
| | 2H-1-benzopyran-3-carboxylic acid; |
| B-31 | 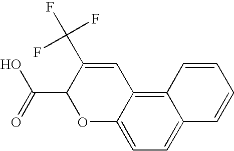 |
| | 2-trifluoromethyl-3H-naphtho[2,1-b]pyran-3-carboxylic acid; |
| B-32 | 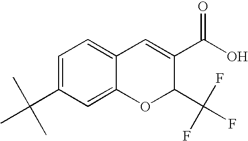 |
| | 7-(1,1-dimethylethyl)-2-trifluoromethyl-2H-1-benzopyran-3- |
| | carboxylic acid; |
| B-33 | 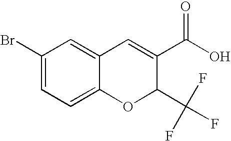 |
| | 6-bromo-2-trifluoromethyl-2H-1-benzopyran-3-carboxylic acid; |
| B-34 | 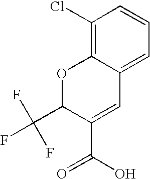 |
| | 8-chloro-2-trifluoromethyl-2H-1-benzopyran-3-carboxylic acid; |
| B-35 | 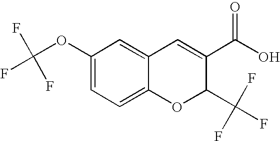 |
| | 6-trifluoromethoxy-2-trifluoromethyl-2H-1-benzopyran-3- |
| | carboxylic acid; |
| B-36 | 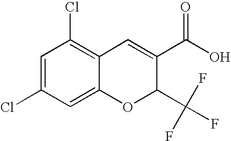 |
| | 5,7-dichloro-2-trifluoromethyl-2H-1-benzopyran-3- |
| | carboxylic acid; |
| B-37 | 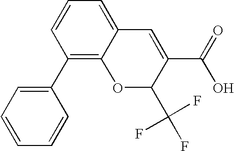 |
| | 8-phenyl-2-trifluoromethyl-2H-1-benzopyran-3-carboxylic acid; |
| B-38 | 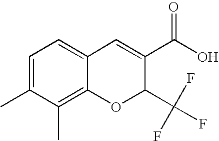 |
| | 7,8-dimethyl-2-trifluoromethyl-2H-1-benzopyran-3- |
| | carboxylic acid; |
| B-39 | 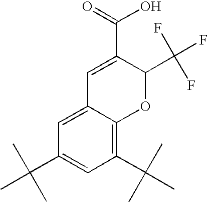 |
| | 6,8-bis(dimethylethyl)-2-trifluoromethyl-2H-1-benzopyran-3- |
| | carboxylic acid; |
| B-40 | 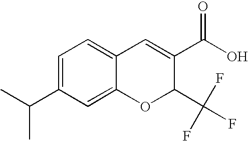 |
| | 7-(1-methylethyl)-2-trifluoromethyl-2H-1-benzopyran-3- |
| | carboxylic acid; |
| B-41 | 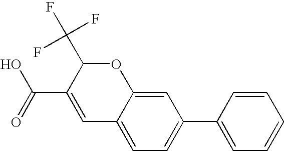 |
| | 7-phenyl-2-trifluoromethyl-2H-1-benzopyran-3-carboxylic acid; |
| B-42 | 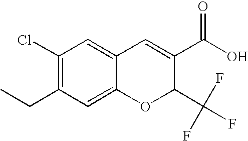 |
| | 6-chloro-7-ethyl-2-trifluoromethyl-2H-1-benzopyran-3- |
| | carboxylic acid; |
| B-43 | 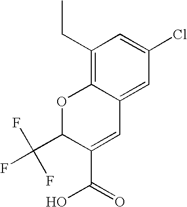 |
| | 6-chloro-8-ethyl-2-trifluoromethyl-2H-1-benzopyran-3- |
| | carboxylic acid; |
| B-44 | 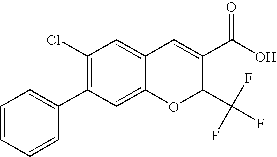 |
| | 6-chloro-7-phenyl-2-trifluoromethyl-2H-1-benzopyran-3- |
| | carboxylic acid; |
| B-45 | 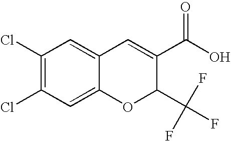 |
| | 6,7-dichloro-2-trifluoromethyl-2H-1-benzopyran-3- |
| | carboxylic acid; |
| B-46 | 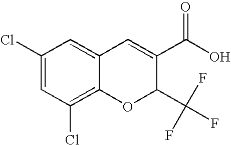 |
| | 6,8-dichloro-2-trifluoromethyl-2H-1-benzopyran-3- |
| | carboxylic acid; |
| B-47 | 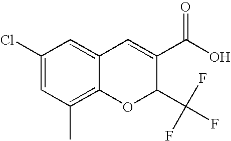 |
| | 6-chloro-8-methyl-2-trifluoromethyl-2H-1-benzopyran-3- |
| | carboxylic acid; |
| B-48 | 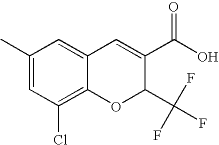 |
| | 8-chloro-6-methyl-2-trifluoromethyl-2H-1-benzopyran-3- |
| | carboxylic acid; |
| B-49 | 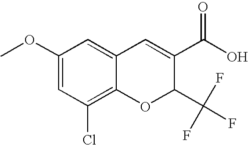 |
| | 8-chloro-6-methoxy-2-trifluoromethyl-2H-1-benzopyran-3- |
| | carboxylic acid; |
| B-50 | 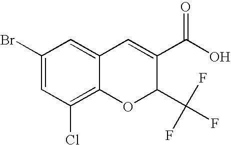 |
| | 6-bromo-8-chloro-2-trifluoromethyl-2H-1-benzopyran-3- |
| | carboxylic acid; |
| B-51 | 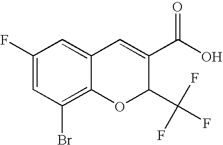 |
| | 8-bromo-6-fluoro-2-trifluoromethyl-2H-1-benzopyran-3- |
| | carboxylic acid; |
| B-52 | 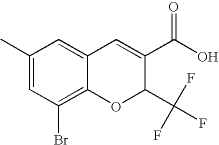 |
| | 8-bromo-6-methyl-2-trifluoromethyl-2H-1-benzopyran-3- |
| | carboxylic acid; |
| B-53 | 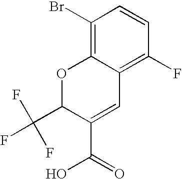 |
| | 8-bromo-5-fluoro-2-trifluoromethyl-2H-1-benzopyran-3- |
| | carboxylic acid; |
| B-54 | 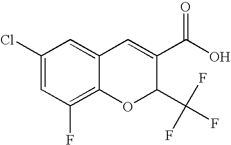 |
| | 6-chloro-8-fluoro-2-trifluoromethyl-2H-1-benzopyran-3- |
| | carboxylic acid; |
| B-55 | 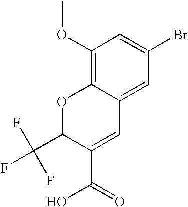 |
| | 6-bromo-8-methoxy-2-trifluoromethyl-2H-1-benzopyran-3- |
| | carboxylic acid; |
| B-56 | 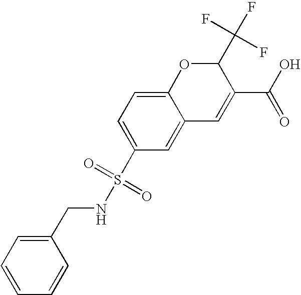 |
| | 6-[[(phenylmethyl)amino]sulfonyl]-2-trifluoromethyl-2H-1- |
| | benzopyran-3-carboxylic acid; |
| B-57 | 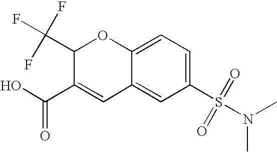 |
| | 6-[(dimethylamino)sulfonyl]-2-trifluoromethyl-2H-1- |
| | benzopyran-3-carboxylic acid; |
| B-58 | 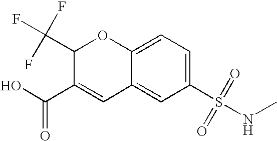 |
| | 6-[(methylamino)sulfonyl]-2-trifluoromethyl-2H-1-benzopyran- |
| | 3-carboxylic acid; |
| B-59 | 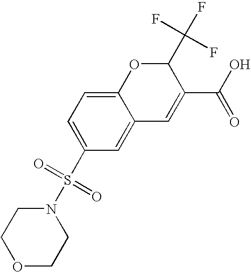 |
| | 6-[(4-morpholino)sulfonyl]-2-trifluoromethyl-2H-1-benzopyran- |
| | 3-carboxylic acid; |
| B-60 | 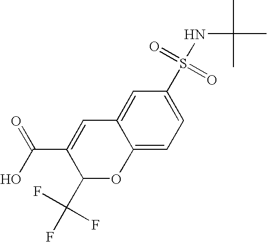 |
| | 6-[(1,1-dimethylethyl)aminosulfonyl]-2-trifluoromethyl- |
| | 2H-1-benzopyran-3-carboxylic acid; |
| B-61 | 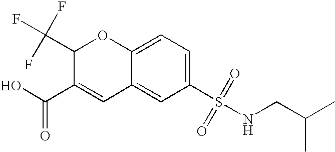 |
| | 6-[(2-methylpropyl)aminosulfonyl]-2-trifluoromethyl-2H-1- |
| | benzopyran-3-carbaxylic acid; |
| B-62 | 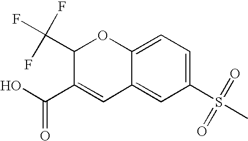 |
| | 6-methylsulfonyl-2-trifluoromethyl-2H-1-benzopyran-3- |
| | carboxylic acid; |
| B-63 | 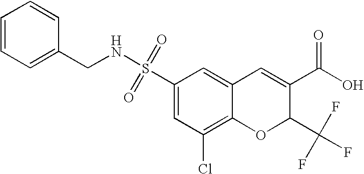 |
| | 8-chloro-6-[[(phenylmethyl)amino]sulfonyl]-2-trifluoromethyl- |
| | 2H-1-benzopyran-3-carboxylic acid; |
| B-64 | 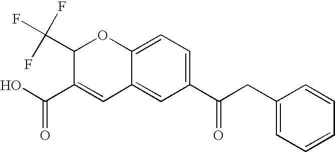 |
| | 6-phenylacetyl-2-trifluoromethyl-2H-1-benzopyran-3- |
| | carboxylic acid; |
| B-65 | 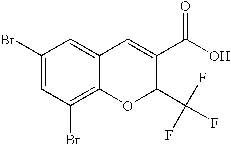 |
| | 6,8-dibromo-2-trifluoromethyl-2H-1-benzopyran-3- |
| | carboxylic acid; |
| B-66 | 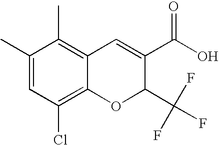 |
| | 8-chloro-5,6-dimethyl-2-trifluoromethyl-2H-1-benzopyran-3- |
| | carboxylic acid; |
| B-67 | 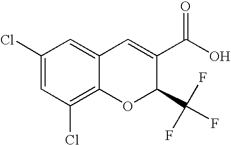 |
| | 6,8-dichloro-(S)-2-trifluoromethyl-2H-1-benzopyran-3- |
| | carboxylic acid; |
| B-68 | 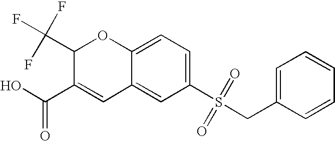 |
| | 6-benzylsulfonyl-2-trifluoromethyl-2H-1-benzopyran-3- |
| | carboxylic acid; |
| B-69 | 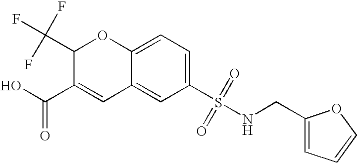 |
| | 6-[[N-(2-furylmethyl)amino]sulfonyl]-2-trifluoromethyl- |
| | 2H-1-benzopyran-3-carboxylic acid; |
| B-70 | 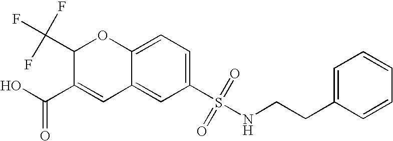 |
| | 6-[[N-(2-phenylethyl)amino]sulfonyl]-2-trifluoromethyl-2H-1- |
| | benzopyran-3-carboxylic acid; |
| B-71 | 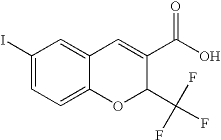 |
| | 6-iodo-2-trifluoromethyl-2H-1-benzopyran-3-carboxylic acid; |
| B-72 | 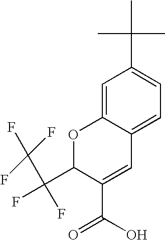 |
| | 7-(1,1-dimethylethyl)-2-pentafluoroethyl-2H- |
| | 1-benzopyran-3-carboxylic acid; |
| B-73 | 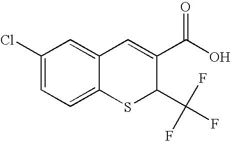 |
| | 6-chloro-2-trifluoromethyl-2H-1-benzothiopyran-3- |
| | carboxylic acid; |
| B-74 | 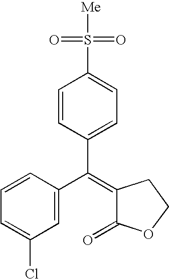 |
| | 3-[(3-chloro-phenyl)-(4-methanesulfonyl-phenyl)-methylene]- |
| | dihydro-furan-2-one or BMS-347070; |
| B-75 | 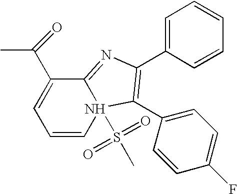 |
| | 8-acetyl-3-(4-fluorophenyl)-2-(4-methylsulfonyl)phenyl- |
| | imidazo(1,2-a)pyridine; |
| B-76 | 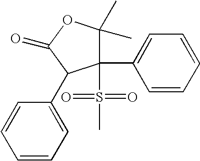 |
| | 5,5-dimethyl-4-(4-methylsulfonyl)phenyl-3-phenyl-2- |
| | (5H)-furanone; |
| B-77 | 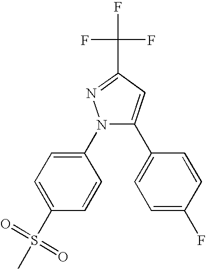 |
| | 5-(4-fluorophenyl)-1-[4-(methylsulfonyl)phenyl]-3- |
| | (trifluoromethyl)pyrazole; |
| B-78 | 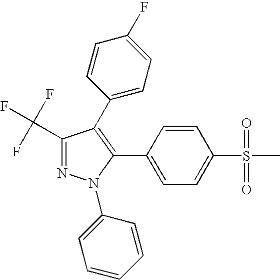 |
| | 4-(4-fluorophenyl)-5-[4-(methylsulfonyl)phenyl]- |
| | 1-phenyl-3-(trifluoromethyl)pyrazole; |
| B-79 | 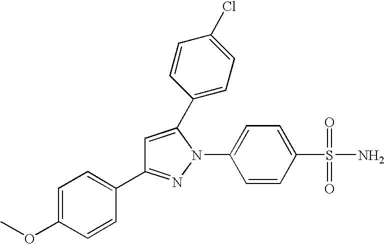 |
| | 4-(5-(4-chlorophenyl)-3-(4-methoxyphenyl)-1H-pyrazol-1-yl) |
| | benzenesulfonamide; |
| B-80 | 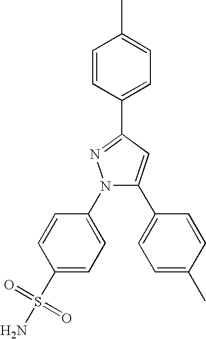 |
| | 4-(3,5-bis(4-methylphenyl)-1H-pyrazol-1- |
| | yl)benzenesulfonamide; |
| B-81 | 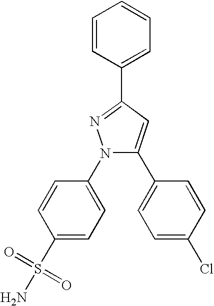 |
| | 4-(5-(4-chlorophenyl)-3-phenyl-1H-pyrazol-1-yl)- |
| | benzenesulfonamide; |
| B-82 | 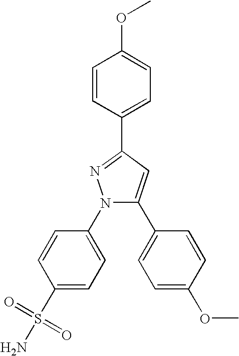 |
| | 4-(3,5-bis(4-methoxyphenyl)-1H-pyrazol-1-yl)- |
| | benzenesulfonamide; |
| B-83 | 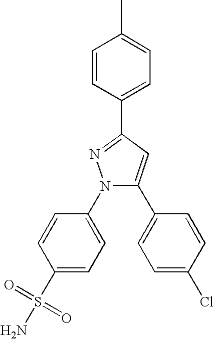 |
| | 4-(5-(4-chlorophenyl)-3-(4-methylphenyl)-1H-pyrazol-1-yl)- |
| | benzenesulfonamide; |
| B-84 | 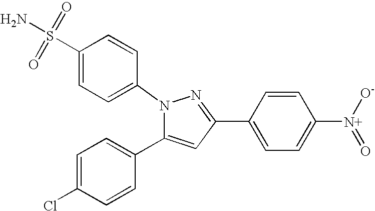 |
| | 4-(5-(4-chlorophenyl)-3-(4-nitrophenyl)-1H-pyrazol-1-yl)- |
| | benzenesulfonamide; |
| B-85 | 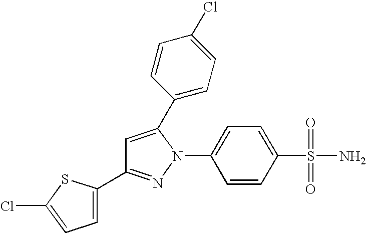 |
| | 4-(5-(4-chlorophenyl)-3-(5-chloro-2-thienyl)-1H-pyrazol-1-yl)- |
| | benzenesulfonamide; |
| B-86 | 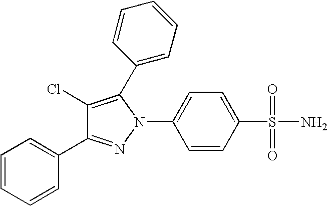 |
| | 4-(4-chloro-3,5-diphenyl-1H-pyrazol-1-yl)benzenesulfonamide; |
| B-87 | 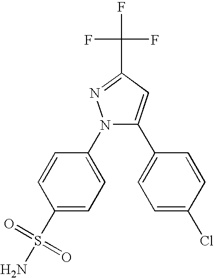 |
| | 4-[5-(4-chlorophenyl)-3-(trifluoromethyl)-1H-pyrazol-1- |
| | yl]benzenesulfonamide; |
| B-88 | 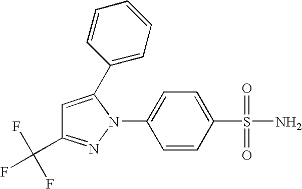 |
| | 4-[5-phenyl-3-(trifluoromethyl)-1H-pyrazol-1-yl]- |
| | benzenesulfonamide; |
| B-89 | 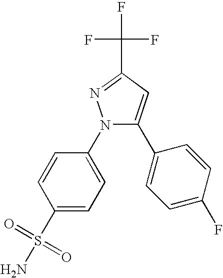 |
| | 4-[5-(4-fluorophenyl)-3-(trifluoromethyl)-1H-pyrazol-1-yl]- |
| | benzenesulfonamide; |
| B-90 | 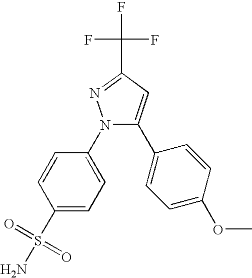 |
| | 4[5-(4-methoxyphenyl)-3-(trifluoromethyl)-1H-pyrazol-1- |
| | yl]benzenesulfonamide; |
| B-91 | 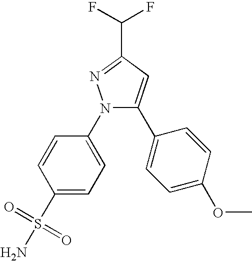 |
| | 4-[5-(4-chlorophenyl)-3-(difluoromethyl)-1H-pyrazol-1- |
| | yl]benzenesulfonamide; |
| B-92 | 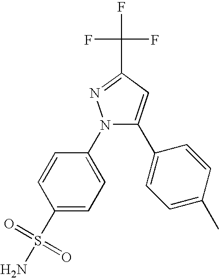 |
| | 4-[5-(4-methylphenyl)-3-(trifluoromethyl)-1H-pyrazol-1- |
| | yl]benzenesulfonamide; |
| B-93 | 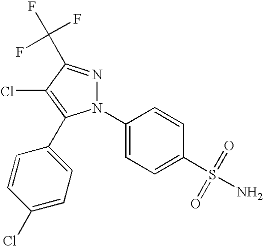 |
| | 4-[4-chloro-5-(4-chlorophenyl)-3-(trifluoromethyl)-1H-pyrazol- |
| | 1-yl] benzenesulfonamide; |
| B-94 | 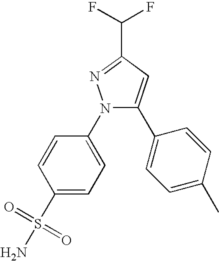 |
| | 4-[3-(difluoromethyl)-5-(4-methylphenyl)-1H-pyrazol-1- |
| | yl]benzenesulfonamide; |
| B-95 | 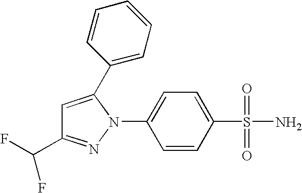 |
| | 4-[3-(difluoromethyl)-5-phenyl-1H-pyrazol-1-yl]- |
| | benzenesulfonamide; |
| B-96 | 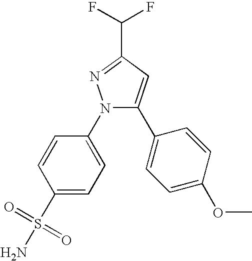 |
| | 4-[3-(difluoromethyl)-5-(4-methoxyphenyl)-1H-pyrazol-1- |
| | yl]benzenesulfonamide; |
| B-97 | 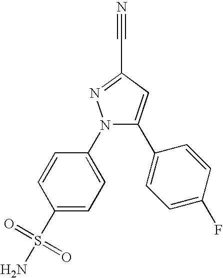 |
| | 4-[3-cyano-5-(4-fluorophenyl)-1H-pyrazol-1- |
| | yl]benzenesulfonamide; |
| B-98 | 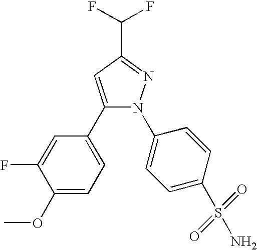 |
| | 4-[3-(difluoromethyl)-5-(3-fluoro-4-methoxyphenyl)-1H- |
| | pyrazol-1-yl] benzenesulfonamide; |
| B-99 | 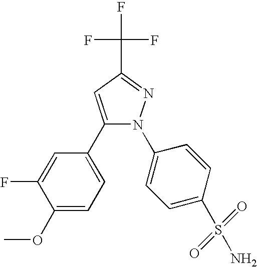 |
| | 4-[5-(3-fluoro-4-methoxyphenyl)-3-(trifluoromethyl)-1H- |
| | pyrazol-1-yl] benzenesulfonamide; |
| B-100 | 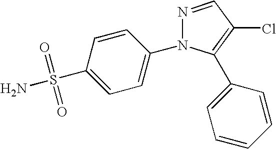 |
| | 4-[4-chloro-5-phenyl-1H-pyrazol-1-yl]benzenesulfonamide; |
| B-101 | 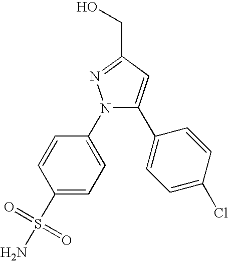 |
| | 4-[5-(4-chlorophenyl)-3-(hydroxymethyl)-1H-pyrazol-1- |
| | yl]benzenesulfonamide; |
| B-102 | 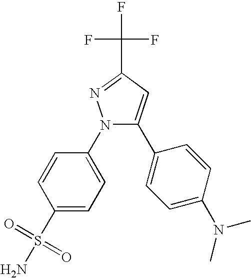 |
| | 4-[5-(4-(N,N-dimethylamino)phenyl)-3-(trifluoromethyl)- |
| | 1H-pyrazol-1-yl]benzenesulfonamide; |
| B-103 | 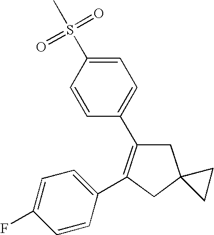 |
| | 5-(4-fluorophenyl)-6-[4-(methylsulfonyl)phenyl]spiro[2.4]hept- |
| | 5-ene; |
| B-104 | 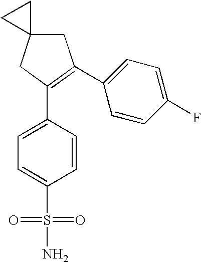 |
| | 4-[6-(4-fluorophenyl)spiro[2.4]hept-5-en-5- |
| | yl]benzenesulfonamide; |
| B-105 | 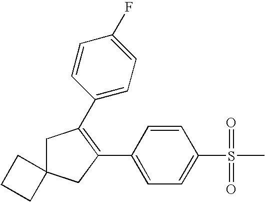 |
| | 6-(4-fluorophenyl)-7-[4-methylsulfonyl)phenyl]spiro[3.4]oct- |
| | 6-ene; |
| B-106 | 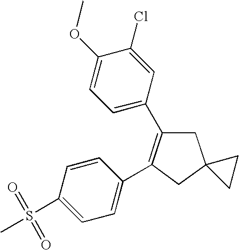 |
| | 5-(3-chloro-4-methoxyphenyl)-6-[4-(methylsulfonyl)- |
| | phenyl]spiro[2.4]hept-5-ene; |
| B-107 | 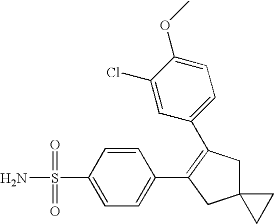 |
| | 4-[6-(3-chloro-4-methoxyphenyl)spiro[2.4]hept-5-en-5- |
| | yl]benzenesulfonamide; |
| B-108 | 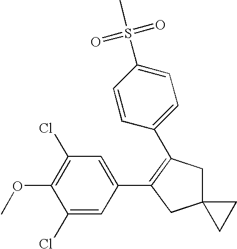 |
| | 5-(3,5-dichloro-4-methoxyphenyl)-6-[4-(methylsulfonyl)phenyl] |
| | spiro[2.4]hept-5-ene; |
| B-109 | 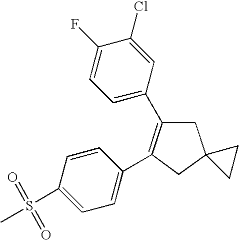 |
| | 5-(3-chloro-4-fluorophenyl)-6-[4-(methylsulfonyl)phenyl]- |
| | spiro[2.4]hept-5-ene; |
| B-110 | 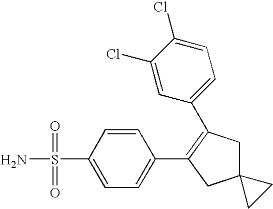 |
| | 4-[6-(3,4-dichlorophenyl)spiro[2.4]hept-5-en-5- |
| | yl]benzenesulfonamide; |
| B-111 | 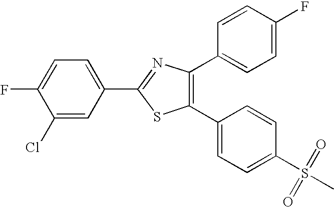 |
| | 2-(3-chloro-4-fluorophenyl)-4-(4-fluorophenyl)-5-(4- |
| | methylsulfonylphenyl)thiazole; |
| B-112 | 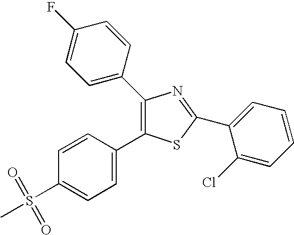 |
| | 2-(2-chlorophenyl)-4-(4-fluorophenyl)-5-(4- |
| | methylsulfonylphenyl)thiazole; |
| B-113 | 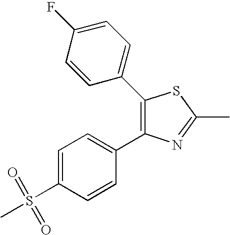 |
| | 5-(4-fluorophenyl)-4-(4-methylsulfonylphenyl)-2- |
| | methylthiazole; |
| B-114 | 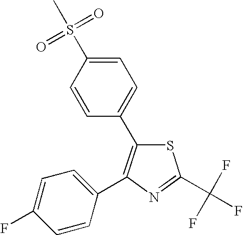 |
| | 4-(4-fluorophenyl)-5-(4-methylsulfonylphenyl)-2- |
| | trifluoromethylthiazole; |
| B-115 | 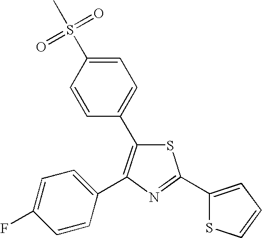 |
| | 4-(4-fluorophenyl)-5-(4-methylsulfonylphenyl)-2-(2- |
| | thienyl)thiazole; |
| B-116 | 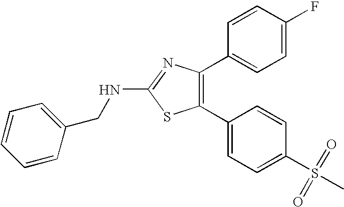 |
| | 4-(4-fluorophenyl)-5-(4-methylsulfonylphenyl)-2- |
| | benzylaminothiazole; |
| B-117 | 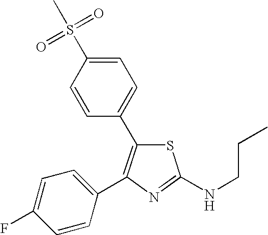 |
| | 4-(4-fluorophenyl)-5-(4-methylsulfonylphenyl)-2- |
| | (1-propylamino)thiazole; |
| B-118 | 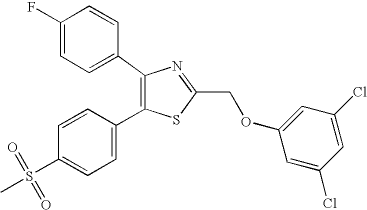 |
| | 2-((3,5-dichlorophenoxy)methyl)-4-(4-fluorophenyl)-5-[4- |
| | (methylsulfonyl)phenyl]thiazole; |
| B-119 | 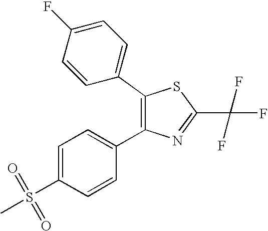 |
| | 5-(4-fluorophenyl)-4-(4-methylsulfonylphenyl)-2- |
| | trifluoromethylthiazole; |
| B-120 | 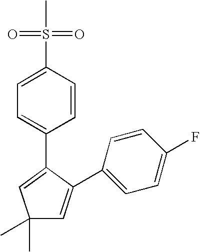 |
| | 1-methylsulfonyl-4-[1,1-dimethyl-4-(4-fluorophenyl) |
| | cyclopenta-2,4-dien-3-yl]benzene; |
| B-121 | 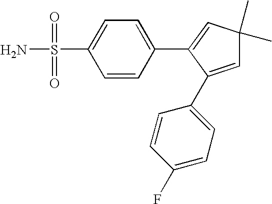 |
| | 4-[4-(4-fluorophenyl)-1,1-dimethylcyclopenta-2,4-dien-3-yl] |
| | benzenesulfonamide; |
| B-122 | 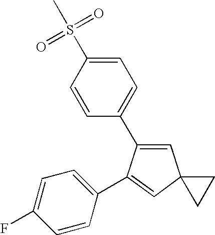 |
| | 5-(4-fluorophenyl)-6-[4-(methylsulfonyl)phenyl]spiro[2.4]- |
| | hepta-4,6-diene; |
| B-123 | 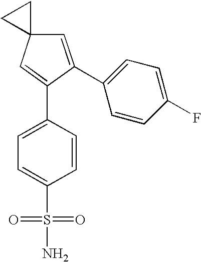 |
| | 4-[6-(4-fluorophenyl)spiro[2.4]hepta-4,6-dien-5- |
| | yl]benzenesulfonamide; |
| B-124 | 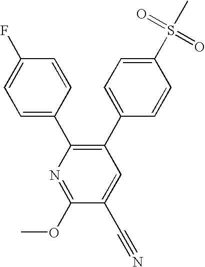 |
| | 6-(4-fluorophenyl)-2-methoxy-5-[4-(methylsulfonyl)phenyl]- |
| | pyridine-3-carbonitrile; |
| B-125 | 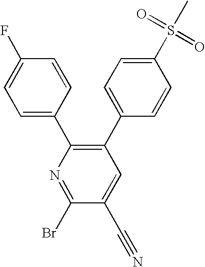 |
| | 2-bromo-6-(4-fluorophenyl)-5-[4-(methylsulfonyl)phenyl]- |
| | pyridine-3-carbonitrile; |
| B-126 | 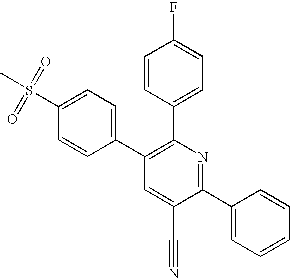 |
| | 6-(4-fluorophenyl)-5-[4-(methylsulfonyl)phenyl]-2-phenyl- |
| | pyridine-3-carbonitrile; |
| B-127 | 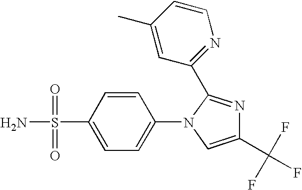 |
| | 4-[2-(4-methylpyridin-2-yl)-4-(trifluoromethyl)-1H-imidazol- |
| | 1-yl] benzenesulfonamide; |
| B-128 | 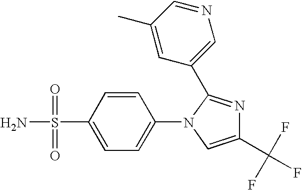 |
| | 4-[2-(5-methylpyridin-3-yl)-4-(trifluoromethyl)-1H-imidazol- |
| | 1-yl] benzenesulfonamide; |
| B-129 | 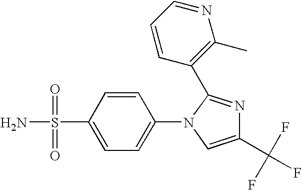 |
| | 4-[2-(2-methylpyridin-3-yl)-4-(trifluoromethyl)-1H-imidazol- |
| | 1-yl] benzenesulfonamide; |
| B-130 | 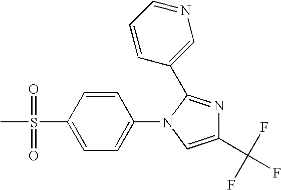 |
| | 3-[1-[4-(methylsulfonyl)phenyl]-4-(trifluoromethyl)-1H- |
| | imidazol-2-yl]pyridine; |
| B-131 | 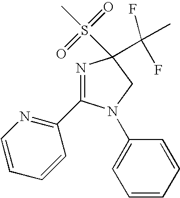 |
| | 2-[1-[4-(methylsulfonyl)phenyl-4-(trifluoromethyl)]-1H- |
| | imidazol-2-yl]pyridine; |
| B-132 | 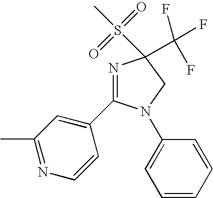 |
| | 2-methyl-4-[1-[4-(methylsulfonyl)phenyl-4-(trifluoromethyl)]- |
| | 1H-imidazol-2-yl]pyridine; |
| B-133 | 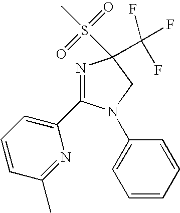 |
| | 2-methyl-6-[1-[4-(methylsulfonyl)phenyl-4-(trifluoromethyl)]- |
| | 1H-imidazol-2-yl]pyridine; |
| B-134 | 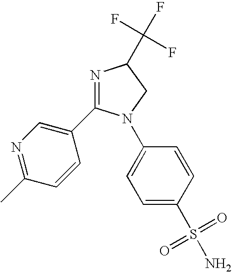 |
| | 4-[2-(6-methylpyridin-3-yl)-4-(trifluoromethyl)-1H-imidazol-1- |
| | yl]benzenesulfonamide; |
| B-135 | 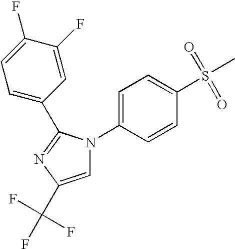 |
| | 2-(3,4-difluorophenyl)-1-[4-(methylsulfonyl)phenyl]- |
| | 4-(trifluoromethyl)-1H-imidazole; |
| B-136 | 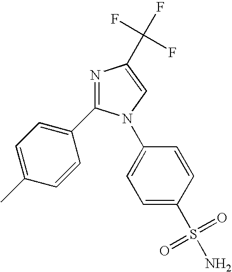 |
| | 4-[2-(4-methylphenyl)-4-(trifluoromethyl)-1H-imidazol-1- |
| | yl]benzenesulfonamide; |
| B-137 | 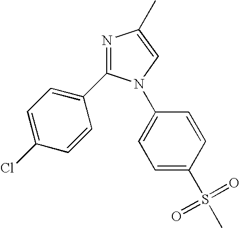 |
| | 2-(4-chlorophenyl)-1-[4-(methylsulfonyl)phenyl]-4-methyl- |
| | 1H-imidazole; |
| B-138 | 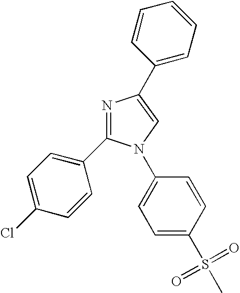 |
| | 2-(4-chlorophenyl)-1-[4-(methylsulfonyl)phenyl]-4-phenyl- |
| | 1H-imidazole; |
| B-139 | 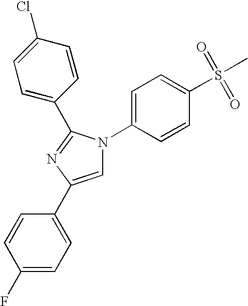 |
| | 2-(4-chlorophenyl)-4-(4-fluorophenyl)-1-[4-(methylsulfonyl)- |
| | phenyl]-1H-imidazole; |
| B-140 | 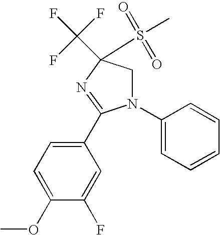 |
| | 2-(3-fluoro-4-methoxyphenyl)-1-[4-(methylsulfonyl)phenyl- |
| | 4-(trifluoromethyl)]-1H-imidazole; |
| B-141 | 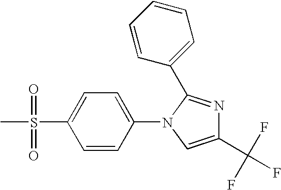 |
| | 1-[4-(methylsulfonyl)phenyl]-2-phenyl-4-trifluoromethyl-1H- |
| | imidazole; |
| B-142 | 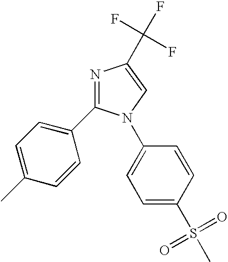 |
| | 2-(4-methylphenyl)-1-[4-(methylsulfonyl)phenyl]-4- |
| | trifluoromethyl-1H-imidazole; |
| B-143 | 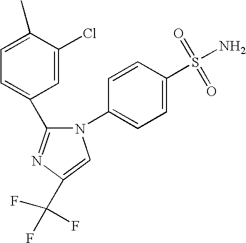 |
| | 4-[2-(3-chloro-4-methylphenyl)-4-(trifluoromethyl)- |
| | 1H-imidazol-1-yl]benzenesulfonamide; |
| B-144 | 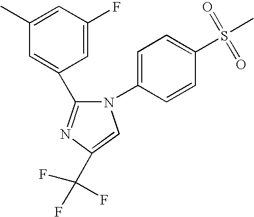 |
| | 2-(3-fluoro-5-methylphenyl)-1-[4-(methylsulfonyl)phenyl]- |
| | 4-(trifluoromethyl)-1H-imidazole; |
| B-145 | 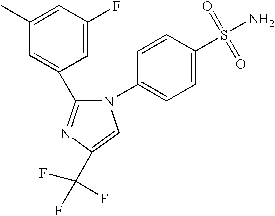 |
| | 4-[2-(3-fluoro-5-methylphenyl)-4-(trifluoromethyl-1H- |
| | 1H-imidazole-1-yl]benzenesulfonamide; |
| B-146 | 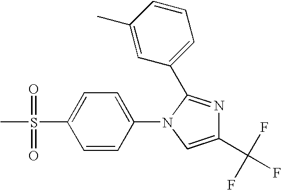 |
| | 2-(3-methylphenyl)-1-[4-(methylsulfonyl)phenyl]-4- |
| | trifluoromethyl-1H-imidazole; |
| B-147 | 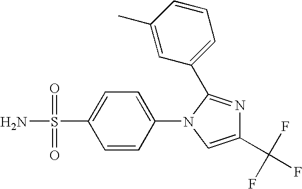 |
| | 4-[2-(3-methylphenyl)-4-trifluoromethyl-1H-imidazol-1- |
| | yl]benzenesulfonamide; |
| B-148 | 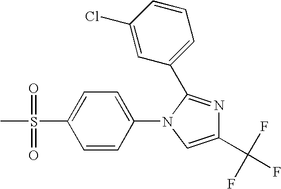 |
| | 1-[4-(methylsulfonyl)phenyl]-2-(3-chlorophenyl)-4- |
| | trifluoromethyl-1H-imidazole |
| B-149 | 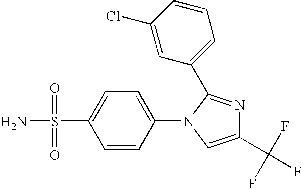 |
| | 4-[2-(3-chlorophenyl)-4-trifluoromethyl-1H-imidazol-1- |
| | yl]benzenesulfonamide; |
| B-150 | 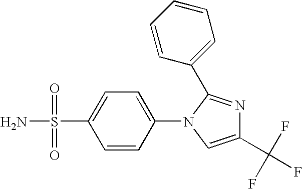 |
| | 4-[2-phenyl-4-trifluoromethyl-1H-imidazol-1- |
| | yl]benzenesulfonamide; |
| B-151 | 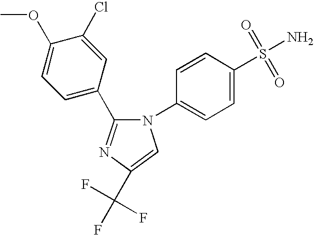 |
| | 4-[2-(4-methoxy-3-chlorophenyl)-4-trifluoromethyl-1H- |
| | imidazol-1-yl]benzenesulfonamide; |
| B-152 | 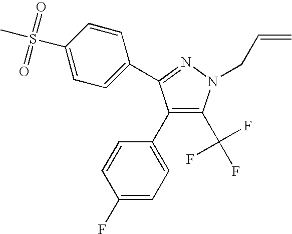 |
| | 1-allyl-4-(4-fluorophenyl)-3-[4-(methylsulfonyl)phenyl]- |
| | 5-(trifluoromethyl)-1H-pyrazole; |
| B-153 | 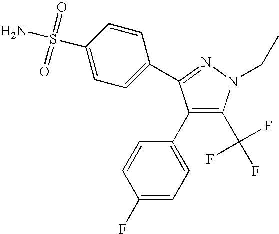 |
| | 4-[1-ethyl-4-(4-fluorophenyl)-5-(trifluoromethyl)-1H-pyrazol- |
| | 3-yl] benzenesulfonamide; |
| B-154 | 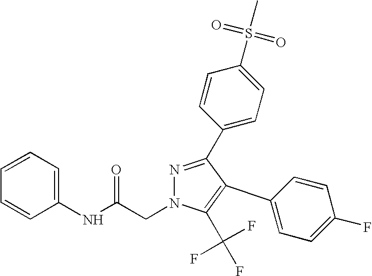 |
| | N-phenyl-[4-(4-fluorophenyl)-3-[4-(methylsulfonyl)phenyl]- |
| | 5-(trifluoromethyl)-1H-pyrazol-1-yl]acetamide; |
| B-155 | 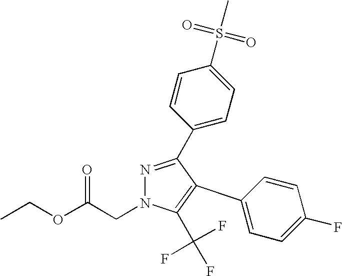 |
| | ethyl[4-(4-fluorophenyl)-3-[4-(methylsulfonyl)phenyl]- |
| | 5-(trifluoromethyl)-1H-pyrazol-1-yl]acetate; |
| B-156 | 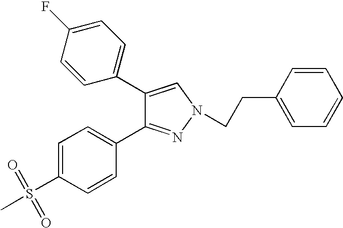 |
| | 4-(4-fluorophenyl)-3-[4-(methylsulfonyl)phenyl]-1- |
| | (2-phenylethyl)-1H-pyrazole; |
| B-157 | 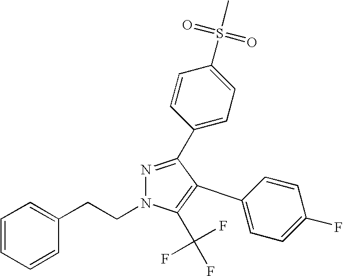 |
| | 4-(4-fluorophenyl)-3-[4-(methylsulfonyl)phenyl]-1- |
| | (2-phenylethyl)-5-(trifluoromethyl)pyrazole; |
| B-158 | 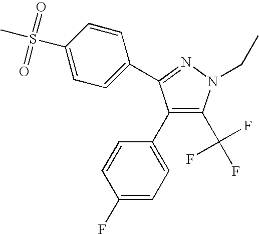 |
| | 1-ethyl-4-(4-fluorophenyl)-3-[4-methylsulfonyl)phenyl]-5- |
| | (trifluoromethyl)-1H-pyrazole; |
| B-159 | 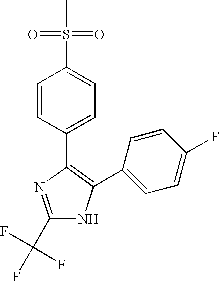 |
| | 5-(4-fluorophenyl)-4-(4-methylsulfonylphenyl)-2- |
| | trifluoromethyl-1H-imidazole; |
| B-160 | 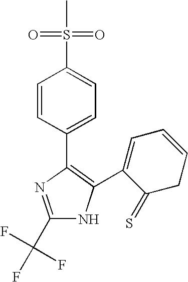 |
| | 4-[4-(methylsulfonyl)phenyl]-5-(2-thiophenyl)-2- |
| | (trifluoromethyl)-1H-imidazole; |
| B-161 | 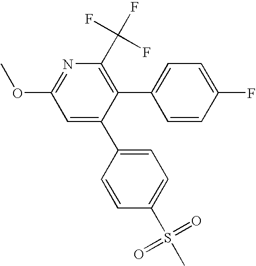 |
| | 5-(4-fluorophenyl)-2-methoxy-4-[4-(methylsulfonyl)phenyl]- |
| | 6-(trifluoromethyl)pyridine; |
| B-162 | 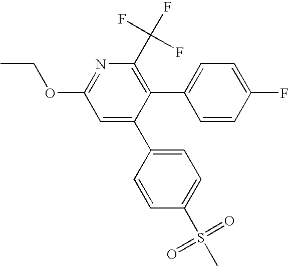 |
| | 2-ethoxy-5-(4-fluorophenyl)-4-[4-(methylsulfonyl)phenyl]- |
| | 6-(trifluoromethyl)pyridine; |
| B-163 | 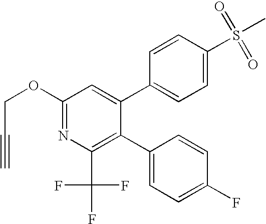 |
| | 5-(4-fluorophenyl)-4-[4-(methylsulfonyl)phenyl]-2- |
| | (2-propynyloxy)-6-(trifluoromethyl)pyridine; |
| B-164 | 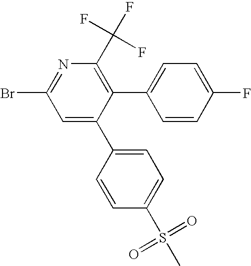 |
| | 2-bromo-5-(4-fluorophenyl)-4-[4-(methylsulfonyl)phenyl]- |
| | 6-(trifluoromethyl)pyridine; |
| B-165 | 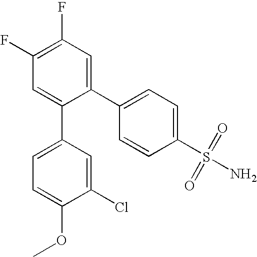 |
| | 4-[2-(3-chloro-4-methoxyphenyl)-4,5-difluorophenyl]- |
| | benzenesulfonamide; |
| B-166 | 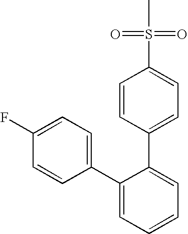 |
| | 1-(4-fluorophenyl)-2-[4-methylsulfonyl)phenyl]benzene; |
| B-167 | 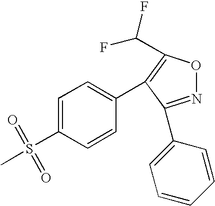 |
| | 5-difluoromethyl-4-(4-methylsulfonylphenyl)-3- |
| | phenylisoxazole; |
| B-168 | 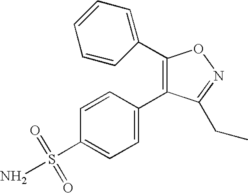 |
| | 4-[3-ethyl-5-phenylisoxazol-4-yl]benzenesulfonamide; |
| B-169 | 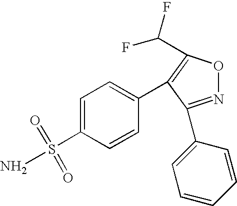 |
| | 4-[5-difluoromethyl-3-phenylisoxazol-4-yl]benzenesulfonamide; |
| B-170 | 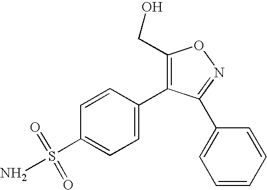 |
| | 4-[5-hydroxymethyl-3-phenylisoxazol-4-yl]benzenesulfonamide; |
| B-171 | 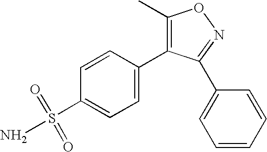 |
| | 4-[5-methyl-3-phenyl-isoxazol-4-yl]benzenesulfonamide; |
| B-172 | 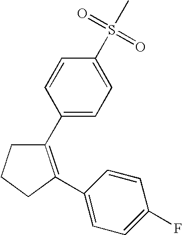 |
| | 1-[2-(4-fluorophenyl)cyclopenten-1-yl]-4- |
| | (methylsulfonyl)benzene; |
| B-173 | 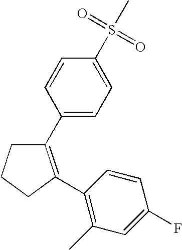 |
| | 1-[2-(4-fluoro-2-methylphenyl)cyclopenten-1-yl]-4- |
| | (methylsulfonyl)benzene; |
| B-174 | 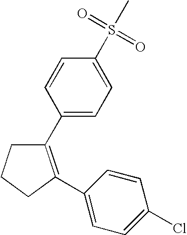 |
| | 1-[2-(4-chlorophenyl)cyclopenten-1-yl]-4- |
| | (methylsulfonyl)benzene; |
| B-175 | 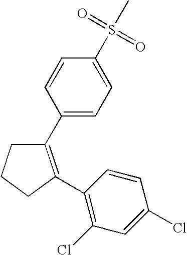 |
| | 1-[2-(2,4-dichlorophenyl)cyclopenten-1-yl]-4- |
| | (methylsulfonyl)benzene; |
| B-176 | 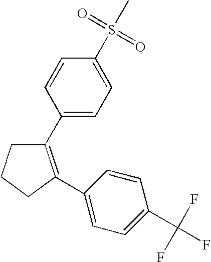 |
| | 1-[2-(4-trifloromethylphenyl)cyclopenten-1-yl]-4- |
| | (methylsulfonyl)benzene; |
| B-177 | 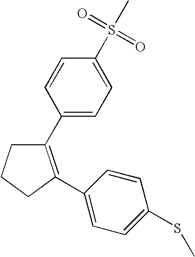 |
| | 1-[2-(4-methylthiophenyl)cyclopenten-1-yl]-4- |
| | (methylsulfonyl)benzene; |
| B-178 | 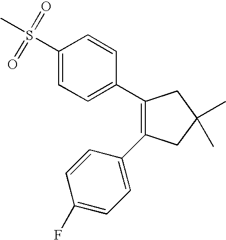 |
| | 1-[2-(4-fluorophenyl)-4,4-dimethylcyclopenten-1-yl]-4- |
| | (methylsulfonyl)benzene; |
| B-179 | 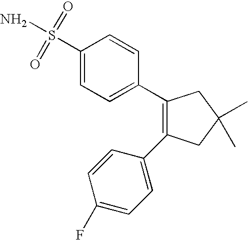 |
| | 4-[2-(4-fluorophenyl)-4,4-dimethylcyclopenten-1- |
| | yl]benzenesulfonamide; |
| B-180 | 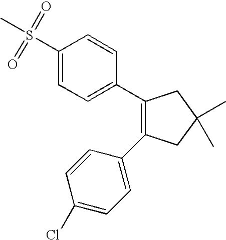 |
| | 1-[2-(3-chlorophenyl)-4,4-dimethylcyclopenten-1-yl]-4- |
| | (methylsulfonyl)benzene; |
| B-181 | 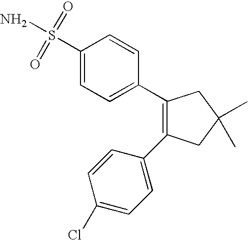 |
| | 4-[2-(4-chlorophenyl)-4,4-dimethylcyclopenten-1- |
| | yl]benzenesulfonamide; |
| B-182 | 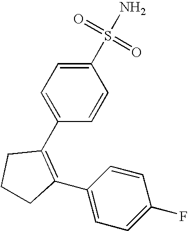 |
| | 4-[2-(4-fluorophenyl)cyclopenten-1-yl]benzenesulfonamide; |
| B-183 | 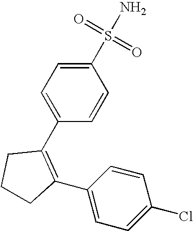 |
| | 4-[2-(4-chlorophenyl)cyclopenten-1-yl]benzenesulfonamide; |
| B-184 | 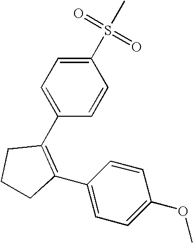 |
| | 1-[2-(4-methoxyphenyl)cyclopenten-1-yl]-4- |
| | (methylsulfonyl)benzene; |
| B-185 | 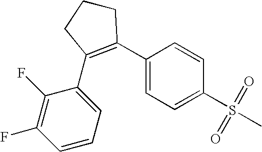 |
| | 1-[2-(2,3-difluorophenyl)cyclopenten-1-yl]-4- |
| | (methylsulfonyl)benzene; |
| B-186 | 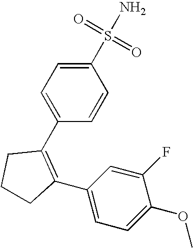 |
| | 4-[2-(3-fluoro-4-methoxyphenyl)cyclopenten-1- |
| | yl]benzenesulfonamide; |
| B-187 | 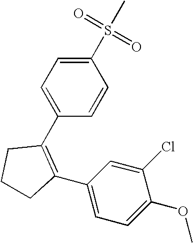 |
| | 1-[2-(3-chloro-4-methoxyphenyl)cyclopenten-1-yl]-4- |
| | (methylsulfonyl)benzene; |
| B-188 | 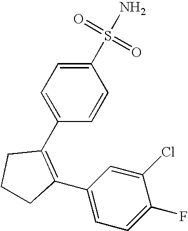 |
| | 4-[2-(3-chloro-4-fluorophenyl)cyclopenten-1- |
| | yl]benzenesulfonamide; |
| B-189 | 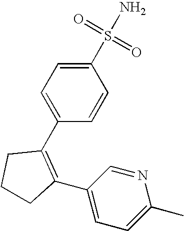 |
| | 4-[2-(2-methylpyridin-5-yl)cyclopenten-1- |
| | yl]benzenesulfonamide; |
| B-190 | 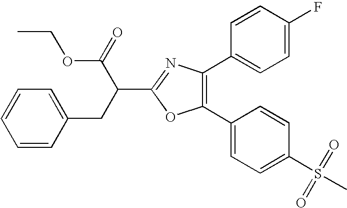 |
| | ethyl 2-[4-(4-fluorophenyl)-5-[4-(methylsulfonyl)- |
| | phenyl]oxazol-2-yl]-2-benzyl-acetate; |
| B-191 | 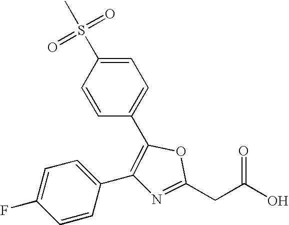 |
| | 2-[4-(4-fluorophenyl)-5-[4-(methylsulfonyl)phenyl]oxazol-2- |
| | yl]acetic acid; |
| B-192 | 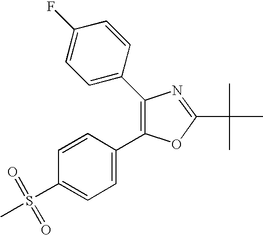 |
| | 2-(tert-butyl)-4-(4-fluorophenyl)-5-[4-(methylsulfonyl)- |
| | phenyl]oxazole; |
| B-193 | 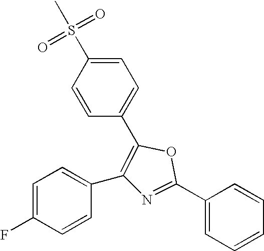 |
| | 4-(4-fluorophenyl)-5-[4-(methylsulfonyl)phenyl]-2- |
| | phenyloxazole; |
| B-194 | 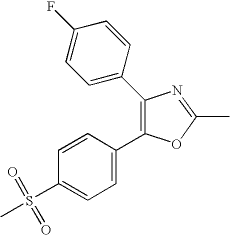 |
| | 4-(4-fluorophenyl)-2-methyl-5-[4-(methylsulfonyl)- |
| | phenyl]oxazole; |
| B-195 | 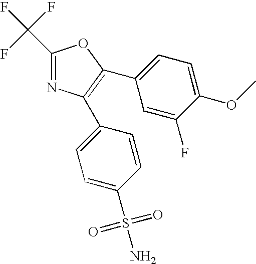 |
| | 4-[5-(3-fluoro-4-methoxyphenyl)-2-trifluoromethyl- |
| | 4-oxazolyl]benzenesulfonamide; |
| B-196 | 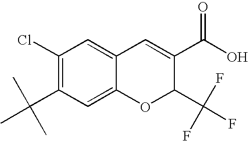 |
| | 6-chloro-7-(1,1-dimethylethyl)-2-trifluoromethyl-2H- |
| | 1-benzopyran-3-carboxylic acid; |
| B-197 | 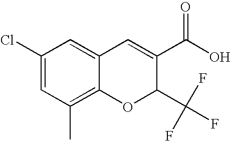 |
| | 6-chloro-8-methyl-2-trifluoromethyl-2H-1-benzopyran-3- |
| | carboxylic acid; |
| B-198 | 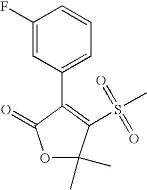 |
| | 5,5-dimethyl-3-(3-fluorophenyl)-4-methylsulfonyl- |
| | 2(5H)-furanone; |
| B-199 | 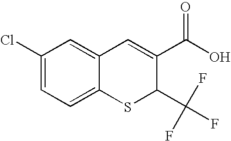 |
| | 6-chloro-2-trifluoromethyl-2H-1-benzothiopyran-3- |
| | carboxylic acid; |
| B-200 | 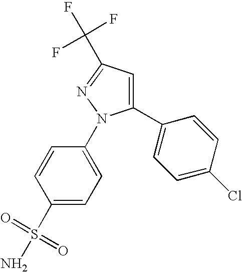 |
| | 4-[5-(4-chlorophenyl)-3-(trifluoromethyl)-1H-pyrazol-1- |
| | yl]benzenesulfonamide; |
| B-201 | 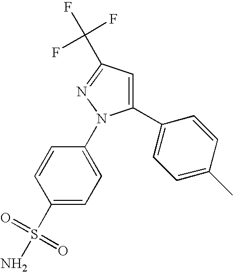 |
| | 4-[5-(4-methylphenyl)-3-(trifluoromethyl)-1H-pyrazol-1- |
| | yl]benzenesulfonamide; |
| B-202 | 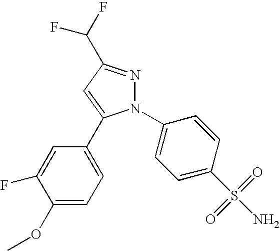 |
| | 4-[5-(3-fluoro-4-methoxyphenyl)-3-(difluoromethyl)-1H- |
| | pyrazol-1-yl]benzenesulfonamide; |
| B-203 | 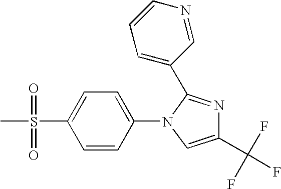 |
| | 3-[1-[4-(methylsulfonyl)phenyl]-4-trifluoromethyl-1H-imidazol- |
| | 2-yl]pyridine; |
| B-204 | 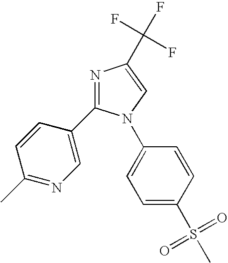 |
| | 2-methyl-5-[1-[4-(methylsulfonyl)phenyl]-4-trifluoromethyl- |
| | 1H-imidazol-2-yl]pyridine; |
| B-205 | 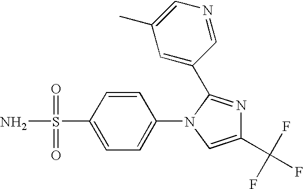 |
| | 4-[2-(5-methylpyridin-3-yl)-4-(trifluoromethyl)- |
| | 1H-imidazol-1-yl]benzenesulfonamide; |
| B-206 | 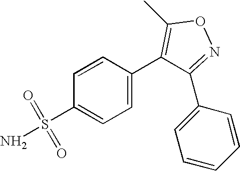 |
| | 4-[5-methyl-3-phenylisoxazole-4-yl]benzenesulfonamide; |
| B-207 | 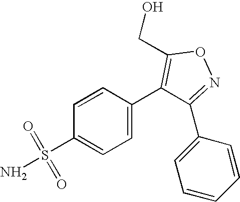 |
| | 4-[5-hydroxymethyl-3-phenylisoxazol-4-yl]benzenesulfonamide; |
| B-208 | 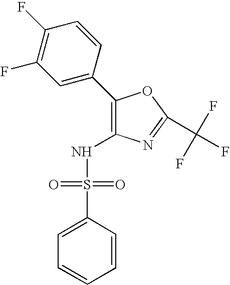 |
| | [2-trifluoromethyl-5-(3,4-difluorophenyl)-4-oxazolyl]- |
| | benzenesulfonamide; |
| B-209 | 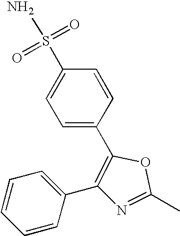 |
| | 4-[2-methyl-4-phenyl-5-oxazolyl]benzenesulfonamide; |
| B-210 | 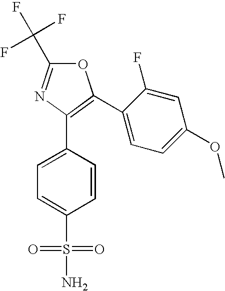 |
| | 4-[5-(2-fluoro-4-methoxyphenyl)-2-trifluoromethyl-4- |
| | oxazolyl]benzenesulfonamide; |
| B-211 | 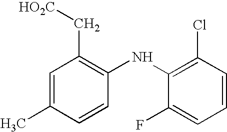 |
| B-212 | 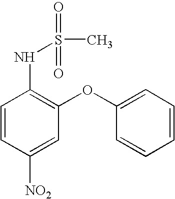 |
| | N-(4-nitro-2-phenoxy-phenyl)-methanesulfonamide or |
| | Nimesulide |
| B-213 | 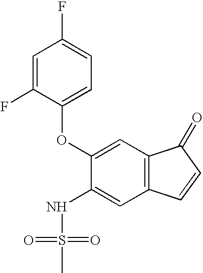 |
| | N-[6-(2,4-difluoro-phenoxy)-1-oxo-inden-5-yl]- |
| | methanesulfonamide or Flosulide |
| B-214 | 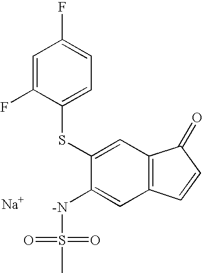 |
| | N-[6-(2,4-difluoro-phenylsulfanyl)-1-oxo-1H-inden-5-yl]- |
| | methanesulfonamide, soldium salt, or L-745337 |
| B-215 | 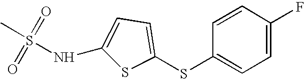 |
| | N-[5-(4-fluoro-phenylsulfanyl)-thiophen-2-yl]- |
| | methanesulfonamide or RWJ-63556 |
| B-216 | 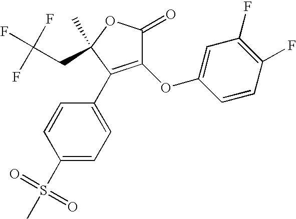 |
| | 3-(3,4-difluoro-phenoxy)-4-(4-methanesulfonyl-phenyl)-5- |
| | methyl-5-(2,2,2-trifluoro-ethyl)-5H-furan-2-one or L-784512 |
| B-217 | 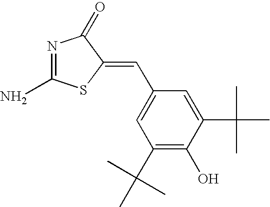 |
| | (5Z)-2-amino-5-[[3,5-bis(1,1-dimethylethyl)-4-hydroxyphenyl]- |
| | methylene]-4(5H)-thiazolone or Darbufelone |
| B-218 | CS-502 |
| B-219 | LAS-34475 |
| B-220 | LAS-34555 |
| B-221 | S-33516 |
| B-222 | SD-8381 |
| B-223 | L-783003 |
| B-224 | 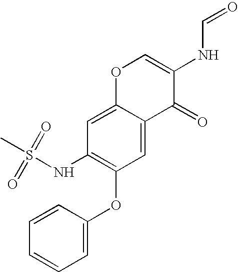 |
| | N-[3-(formylamino)-4-oxo-6-phenoxy-4H-1-benzopyran-7-yl]- |
| | methanesulfonamide or T614 |
| B-225 | D-1367 |
| B-226 | L-748731 |
| B-227 | 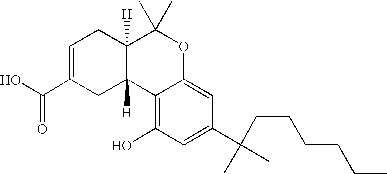 |
| | (6aR,10aR)-3-(1,1-dimethylheptyl)-6a,7,10,10a-tetrahydro-1- |
| | hydroxy-6,6-dimethyl-6H-dibenzo[b,d]pyran-9-carboxylic |
| | acid or CT3 |
| B-228 | CGP-28238 |
| B-229 | 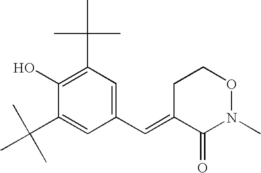 |
| | 4-[[3,5-bis(1,1-dimethylethyl)-4-hydroxyphenyl]methylene] |
| | dihydro-2-methyl-2H-1,2-oxazin-3(4H)-one or BF-389 |
| B-230 | GR-253035 |
| B-231 | 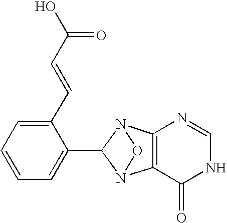 |
| | 2-(6-dioxo-9H-purin-8-yl)cinnamic acid |
| B-232 | S-2474 |
| B-233 | 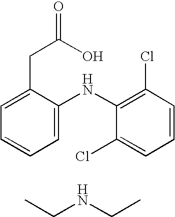 |

In yet another embodiment, the cyclooxygenase-2 selective inhibitor is other than celecoxib, rofecoxib, meloxicam, or nimesulide.

The cyclooxygenase-2 selective inhibitors utilized in the present invention may be in the form of free bases or pharmaceutically acceptable acid addition salts thereof. The term “pharmaceutically-acceptable salts” embraces salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, β-hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound of any Formula set forth herein.

The cyclooxygenase-2 selective inhibitors useful in the practice of the present invention can be formulated into pharmaceutical compositions and administered by any means that will deliver a therapeutically effective dose. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage of the cyclooxygenase-2 selective inhibitor will vary depending upon the patient and the particular mode of administration. In general, the pharmaceutical compositions may contain a cyclooxygenase-2 selective inhibitor in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight and most preferably from about 1 to 20 mg/kg body weight, may be appropriate. The daily dose can be administered in one to four doses per day.

In one embodiment, when the cyclooxygenase-2 selective inhibitor comprises rofecoxib, it is preferred that the amount used is within a range of from about 0.15 to about 1.0 mg/day.kg, and even more preferably from about 0.18 to about 0.4 mg/day·kg.

In still another embodiment, when the cyclooxygenase-2 selective inhibitor comprises etoricoxib, it is preferred that the amount used is within a range of from about 0.5 to about 5 mg/day.kg, and even more preferably from about 0.8 to about 4 mg/day·kg.

Further, when the cyclooxygenase-2 selective inhibitor comprises celecoxib, it is preferred that the amount used is within a range of from about 1 to about 20 mg/day.kg, even more preferably from about 1.4 to about 8.6 mg/day·kg, and yet more preferably from about 2 to about 3 mg/day·kg.

When the cyclooxygenase-2 selective inhibitor comprises valdecoxib, it is preferred that the amount used is within a range of from about 0.1 to about 5 mg/day.kg, and even more preferably from about 0.8 to about 4 mg/day·kg.

In a further embodiment, when the cyclooxygenase-2 selective inhibitor comprises parecoxib, it is preferred that the amount used is within a range of from about 0.1 to about 5 mg/day·kg, and even more preferably from about 1 to about 3 mg/day·kg.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

### Anti-Human Immunodeficiency Virus Agents

In addition to a cyclooxygenase-2 selective inhibitor, the composition of the invention also comprises an anti-human immunodeficiency virus agent. Any anti-human immunodeficiency virus agent can be used in the current invention to the extent that the agent is capable of achieving viral inhibition. In general terms, such viral inhibition is any decrease in the severity of an HIV infection as compared to that which would occur in the absence of the administration of the composition to the subject. This decrease in severity may result from a number of different factors including: a reduction in viral number, a reduction in viral replication, a reduction in the subject's cell growth infected with the virus, a reduction in cellular replication in the subject, a reduction in cellular mitosis in a subject, a reduction in viral colonization or any combination thereof. Generally speaking, the anti-human immunodeficiency virus agents typically fall into one of two categories: agents that inhibit HIV infection by substantially inhibiting the HIV virus directly, or agents that inhibit HIV infection by causing the human to substantially inhibit the HIV infection. Suitable anti-human immunodeficiency virus agents typically include viral cellular entry inhibitors, viral replication inhibitors, viral assembly inhibitors, integrase inhibitors, human immune enhancing agents, virucidal agents, and antimitotic agents.

One aspect of the invention encompasses anti-human immunodeficiency virus agents that are viral cellular entry inhibitors. Viral cellular entry inhibitors typically disrupt viral association with the subject's cell membrane thereby substantially inhibiting entry or release of the virus into the subject's cell. In one embodiment, the viral cellular entry inhibitor is enfuvirtide (Fuzeon®) or hydroxyurea (Droxia®). In another embodiment, the viral cellular inhibitor is a virion receptor/coreceptor-binding antagonist. Generally speaking, these agents bind to either the subject's gp120 or CD4 receptor and prevent binding of the virus to the host cell surface. Any agent capable of disrupting HIV association with the subject's cell membrane may be employed. By way of example, suitable virion receptor/coreceptor-binding antagonists are shown in Table A.

TABLE A

| Compound | |
|---|---|
| No. | Compound |
| A1 | N2-acetyl-D-arginyl-D-arginyl-D-arginyl-D-arginyl-D-arginyl-D-arginyl- |
| | D-arginyl-D-arginyl-D-argininamide, nonaacetate |
| A2 | 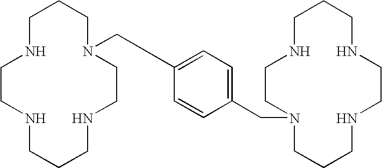 |
| | 1,1′-[1,4-Phenylenebis(methylene)bis[1,4,8,1 |
| | 1-tetraazacyclotetradecane]octohydrobromide |
| | dihydrate |
| A3 | 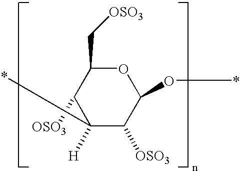 |
| | Curdlan Sulfate |
| A4 | Cyanovirin-N |
| A5 | 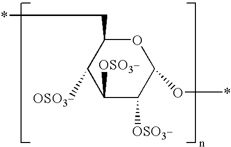 |
| | Dextran sulfate (α-1,6-Linked glucopyranose Units) |
| A6 | Macrophage Inflammation Protein-1 α (Human) |
| A7 | Macrophage Inflammation Protein-1 β (Human) |
| A8 | 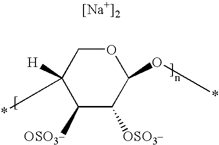 |
| A9 | 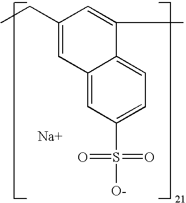 |
| | Naphthalene 2-sulphonate polymer |
| A10 | Rantes |
| A11 | 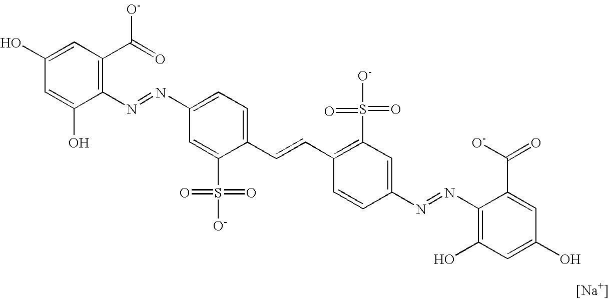 |
| | 4,4′-Bis(2-carboxy-4,6-dihydroxyphenylazo)stilbene-2,2′- |
| | disulfonic acid tetrasodium sal |
| A12 | SPC3 |

In another embodiment, the viral cellular entry inhibitor is an uncoating inhibitor. While these agents allow a degree of viral penetration into a subject's cell membrane, they typically prevent the release of viral nucleic acids into the subject's cytoplasm; thus, rendering the virus unable to replicate. Typically, any agent that prevents HIV uncoating may be employed in the current invention. Examples of suitable uncoating inhibitors are shown in Table BB.

TABLE BB

| Compound | |
|---|---|
| No. | Compound |
| BB1 | N2-acetyl-D-arginyl-D-arginyl-D-arginyl-D-arginyl-D-arginyl-D- |
| | arginyl-D-arginyl-D-arginyl-D-argininamide, nonaacetate |
| BB2 | 1,1-[1,4-phenylenebis(methylene)]bis[1,4,8,11,tetraazacyclotetradecane] |
| | octohydrobromide dihydrate (see Compound A2 for structure) |
| BB3 | 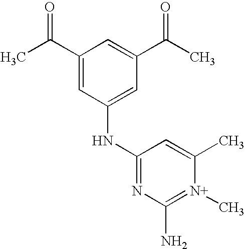 |
| | 4-(5-acetyl-3-((2-amino-1,6-dimethylpyrimidin-4-yl) |
| | amino)phenyl)ethan-1-o |
| BB4 | 5,5′-(1,1′-dihydroxy-8,8′-dimethoxy-6,6′-dimethyl[2,2′-binaphthalene]- |
| | 4,4′-diyl)bis[1,2,3,4-tetrahydro-1,3-dimethyl-6,8- |
| | isoquinolinediol],(1R,3R,5S,1′R,3′R,5′S) (see Compound D36 for |
| | structure) |
| BBS | 5,5′-(1,1′-dihydroxy-8,8′-dimethoxy-6,6′-dimethyl[2,2′-binaphthalene]- |
| | 4,4′-diyl)bis[1,2,3,4-tetrahydro-1,3-dimethyl-6,8- |
| | isoquinolinediol],(1R,3R,5S,1′R,3′R,5′S) (see Compound D37 for |
| | structure) |
| BB6 | 5,5′-(1,1′-dihydroxy-8,8′-dimethoxy-6,6′-dimethyl[2,2′-binaphthalene]- |
| | 4,4′-diyl)[3,4-dihydro-8-methoxy-1,3-dimethyl-6- |
| | isoquinolinol],[1,2,3,4-tetrahydro-1,3-dimethyl-6,8- |
| | isoquinolinediol],(3R,5S,1′R,3′R,5′S) (see Compound D38 for structure) |
| BB7 | Ac-YTSLIHSLIEESQNQQEKNEQELLELDKWASLWNWF-NH₂ |
| BB8 | [Tyr(5,12),Lys(7)-polyphemusin II |

Yet another aspect of the invention embraces anti-human immunodeficiency virus agents that are viral replication inhibitors. Generally speaking, viral replication inhibitors substantially inhibit the synthesis of viral nucleic acid from which new virus particles are produced. In one embodiment, the viral replication inhibitor inhibits the viral enzyme reverse transcriptase. The virus employs reverse transcriptase in order to transcribe its own RNA into viral DNA. In the absence of viral DNA, the virus is unable to replicate. Any agent capable of inhibiting reverse transcriptase may be utilized in the present invention. In one alternative of this embodiment, the reverse transcriptase inhibitor is a nucleoside analog. By way of example, suitable nucleoside analogs for use in the current invention are shown in Table C.

TABLE C

| Compound | |
|---|---|
| No. | Compound |
| C1 | 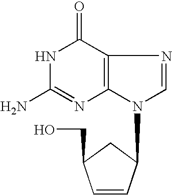 |
| | (−)-cis-2-amino-1,9-dihydro-9-[4-hydroxymethyl)-2- |
| | cyclopenten-1-yl)-6H-purin-6-one |
| C2 | 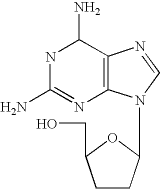 |
| | 2,6-diamino-2′,3′-dideoxypurine-9-ribofuranoside |
| C3 | 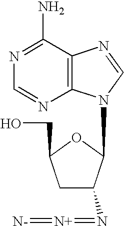 |
| | 9-(2-azido-2,3-dideoxy-β-D-erythro-pentofuranosyl)adenine |
| C4 | 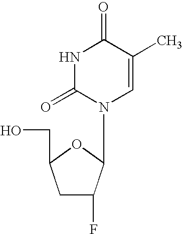 |
| | 1-(2′-fluoro-2′,3′-dideoxy-B-D-erythro-pentofuranosyl)thymine |
| C5 | 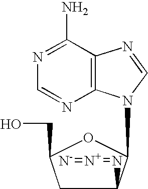 |
| | 9-(2-azido-2,3-dideoxy-β-D-threo-pentofuranosyl)adenine |
| C6 | 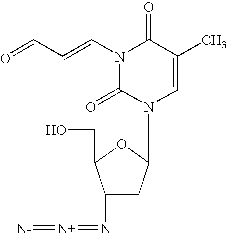 |
| | 3-(3-oxo-1-propenyl)-3′-azido-3′-deoxythymidine |
| C7 | 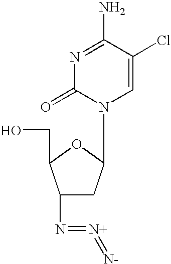 |
| | 3-azido-2′,3′-dideoxy-5-chlorocytidine |
| C8 | 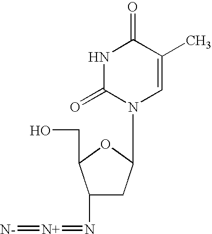 |
| | 3′-azido-3′-deoxy-6-azathymidine |
| C9 | 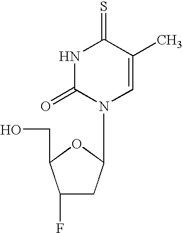 |
| | 2′,3′-dideoxy-3′-fluoro-4-thiothymidine |
| C10 | 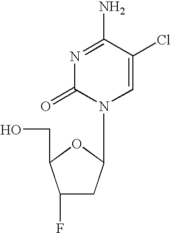 |
| | 2′,3′-dideoxy-3′-fluoro-5-chlorocytidine |
| C11 | 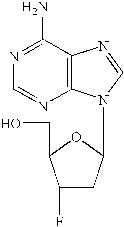 |
| | 9-(3′-fluoro-2′,3′-dideoxy-B-D-erythropentafuranosyl)adenine |
| C12 | 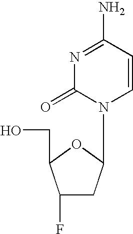 |
| | 3′-fluoro-2′,3′-dideoxycytidine |
| C13 | 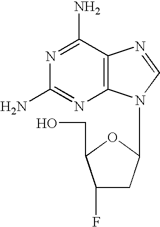 |
| | 2,6-diaminopurine-3′-fluoro-2′,3′-dideoxyriboside |
| C14 | 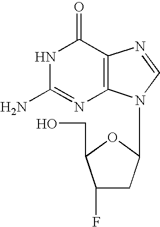 |
| | 3′-fluoro-2′,3′-dideoxyguanosine |
| C15 | 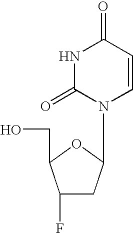 |
| | 3′-fluoro-2′,3′-dideoxyuridine |
| C16 | 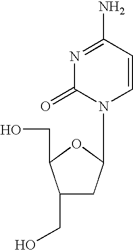 |
| | 1-[2′,3′-dideoxy-3′-C-(hydroxymethyl)-.beta.-D-erythro- |
| | pentofuranosyl]cytosine |
| C17 | 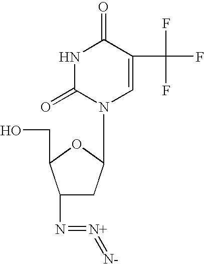 |
| | 3′-azido-2′,3′-dideoxy-5-trifluoromethyluridine |
| C18 | 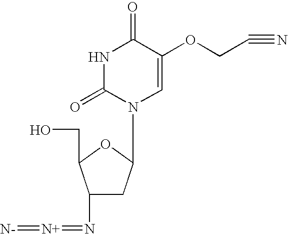 |
| | 3′-azido-2′,3′-dideoxy-5-[(cyanomethyl)oxy]uridine |
| C19 | 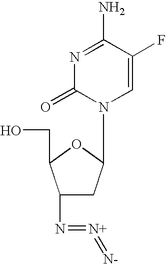 |
| | 3′-azido-2′,3′-dideoxy-5-fluorocytidine |
| C20 | 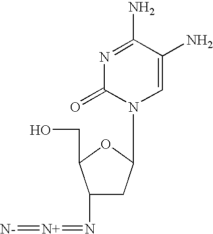 |
| | 3′-azido-2′,3′-dideoxy-5-methylcytidine |
| C21 | 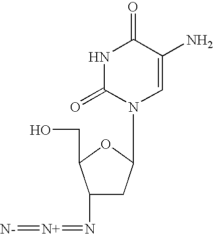 |
| | 3′-azido-2′,3′-dideoxy-5-aminouridine |
| C22 | 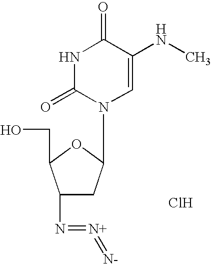 |
| | 3′-azido-2′,3′-dideoxy-5-methyaminouridine |
| C23 | 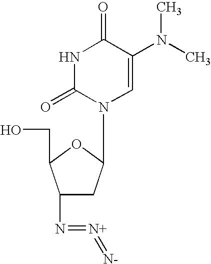 |
| | 3′-azido-2′,3′-dideoxy-5-dimethylaminouridine |
| C24 | 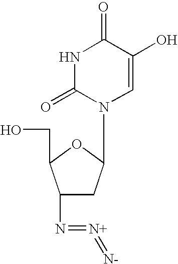 |
| | 3′-azido-2′,3′-dideoxy-5-hydroxyuridine |
| C25 | 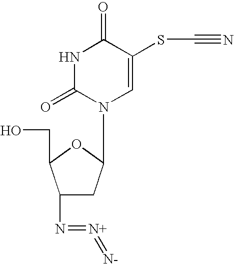 |
| | 3′-azido-2′,3′-dideoxy-5-thiocyanatouridine |
| C26 | 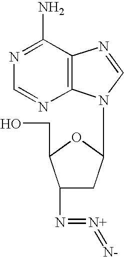 |
| | 9-(3′-azido-2′,3′-dideoxy-B-D-erythropentafuranosyl)adenine |
| C27 | 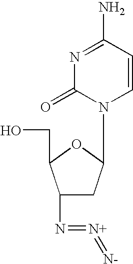 |
| | 3′-azido-2′,3′-dideoxycytidine |
| C28 | 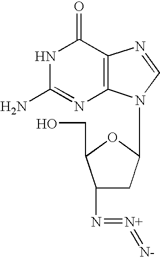 |
| | 3′-azido-2′,3′-dideoxyguanosine |
| C29 | 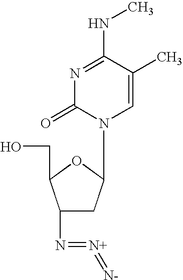 |
| | 3′-azido-2′,3′-dideoxy-N4-5-dimethylcytidine |
| C30 | 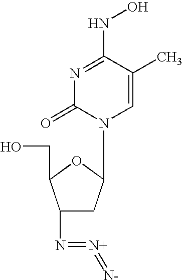 |
| | 3′-azido-2′,3′-dideoxy-N4-OH-5-methylcytidine |
| C31 | 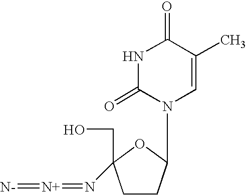 |
| | 4′-azido-3′-deoxythymidine |
| C32 | 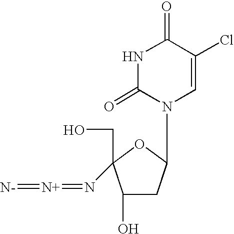 |
| | 4′-azido-5-chloro-2′-deoxyuridine |
| C33 | 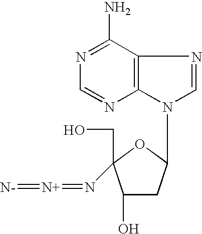 |
| | 4′-azido-2′-deoxyadenosine |
| C34 | 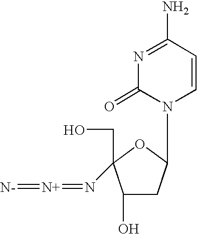 |
| | 4′-azido-2′-deoxycytidine |
| C35 | 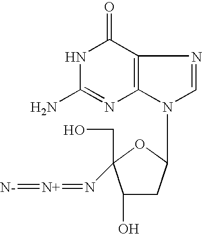 |
| | 4′-azido-2′-deoxyguanosine |
| C36 | 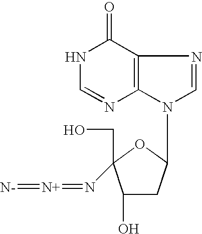 |
| | 4′-azido-2′-deoxyinosine |
| C37 | 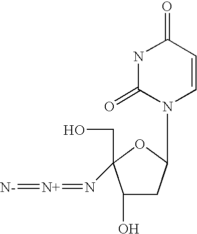 |
| | 4′-azido-2′-deoxyuridine |
| C38 | 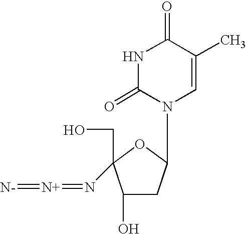 |
| | 1-(4-azido-2-deoxy-.beta.-D-erythro-pentofuranosyl)- |
| | 5-methyl-2,4-dioxopyrimidine |
| C39 | 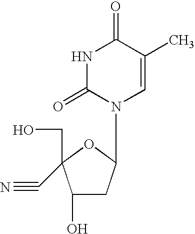 |
| | 4′-cyanothymidine |
| C40 | 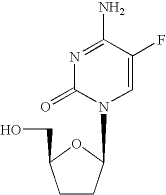 |
| | 5-fluoro-2′,3′-dideoxycytidine |
| C41 | 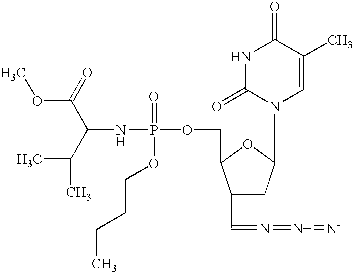 |
| | 3′-azido-3′-deoxythymidine-5′-(butylmethoxyvalinyl)phosphate |
| C42 | 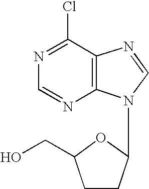 |
| | 6-chloro-9-(2,3-dideoxy-.beta.-D-g Iyceropentofuranosyl)-9H-purin |
| C43 | 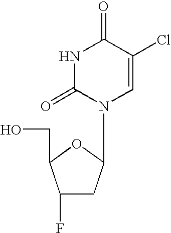 |
| | 2′,3′-dideoxy-3′-fluoro-5-chlorouridine |
| C44 | 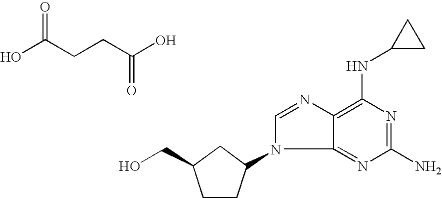 |
| | butanedioic acid, compd. with (1S-cis)-4-[2-amino-6-(cyclopropylamin |
| | 9H-purine-9-yl]-2-cyclopentene-1-methanol (1:1) |
| C45 | 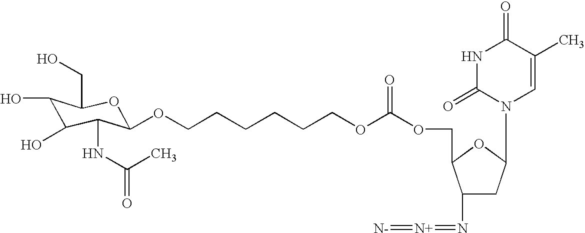 |
| | 5′-alkylglycoside carbonate of 3′-azido-3′-deoxythymidine |
| C46 | 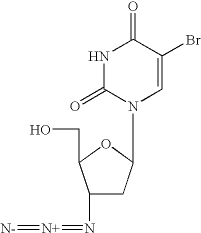 |
| | 3′-azido-2′,3′-dideoxy-5-bromouridine |
| C47 | 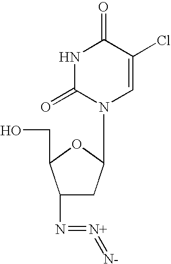 |
| | 3′-azido-5-chloro-2′,3′-dideoxyuridine |
| C48 | 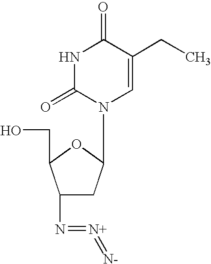 |
| | 3′-azido-2′,3′-dideoxy-5-ethyluridine |
| C49 | 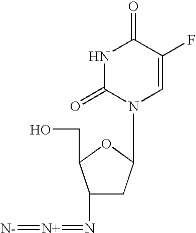 |
| | 3′-azido-2′,3′-dideoxy-5-fluorouridine |
| C50 | 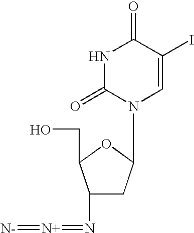 |
| | 3′-azido-2′,3′-dideoxy-5-iodouridine |
| C51 | 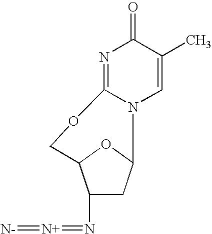 |
| | 2,5′-anhydro-3′-azido-3′-deoxythymidine |
| C52 | 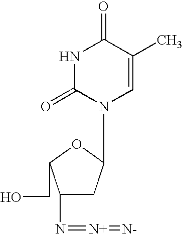 |
| | 1-(2,3-dideoxy-3-azido-α-L-threo-pentofuranosyl)thymine |
| C53 | 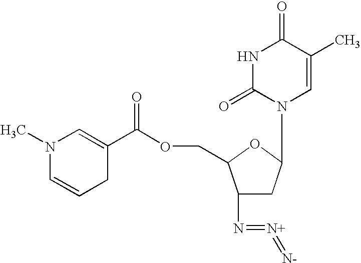 |
| | 5′-[(1,4-dihydro-1-methyl-3-pyridinylcarbonyl)oxy]- |
| | 3′-azido-2′3′-deoxythymi |
| C54 | 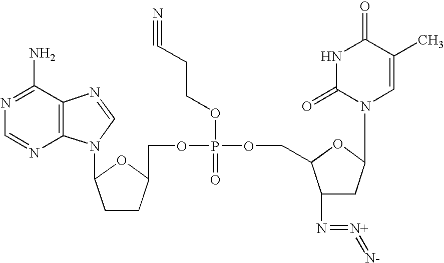 |
| | 3′-azido-3′-deoxythymidilyl-(5′,5′)-2′,3′- |
| | dideoxy-5′-adenylic acid, 2-cyanoethyl ester |
| C55 | 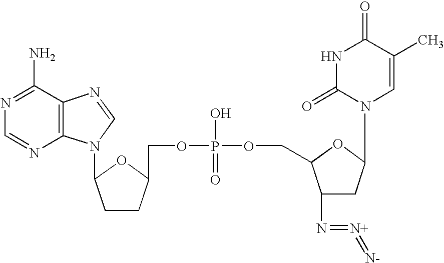 |
| | 3-azido-3′-deoxythymidilyl-(5′,5′)-2′,3′-dideoxy-5′-adenylic acid |
| C56 | 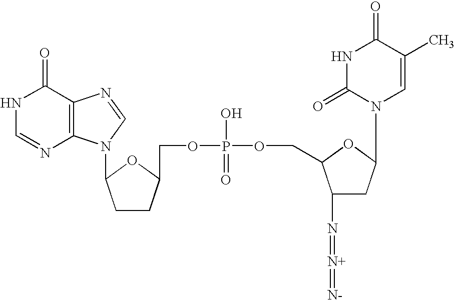 |
| | 3′-azido-3′-deoxythymidilyl-(5′,5′)-2′,3′-dideoxy-5′inosinic acid |
| C57 | 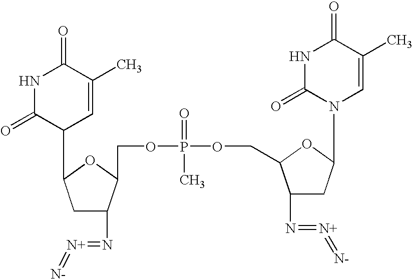 |
| | O,O′-bis(3′-azido-3′-deoxythymidin-5′-yl)methylphosphonate |
| C58 | 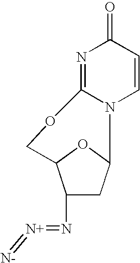 |
| | 2,5′-anhydro-3′-azido-2′,3′-dideoxyuridine |
| C59 | 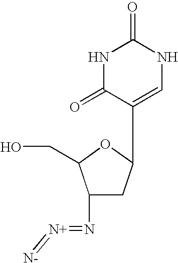 |
| | 2,4(1H,3H)-pyrimidinedione,5-(3-azido-2,3- |
| | dideoxy-.beta.-D-erythro-pentofuranosyl) |
| C60 | 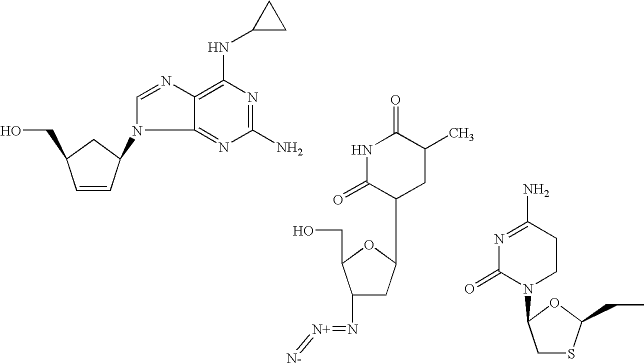 |
| | (1S,4R)-4-[2-amino-6-(cyclopropylamino)-9H-purin-9-yl]-2-cyclopentene-1-methan |
| | .beta.-L-(−)-2′3′-dideoxy-3′-thiacytidine & 3′-azido-3′-deoxythymidine |
| C61 | 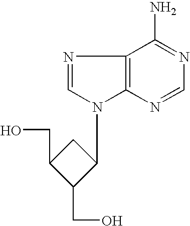 |
| | (+−)-9-[(1.beta.-2.alpha.-3.beta.)-2,3-bis(hydroxymethyl)- |
| | 1-cyclobutyl]adenine |
| C62 | 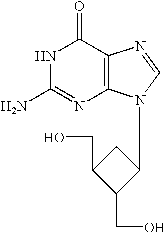 |
| | 9-[1.beta.-2.alpha.-3.beta.]-2,3-bis(hydroxymethyl)- |
| | 1-cyclobutyl]guanine |
| C63 | 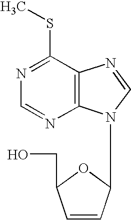 |
| | 9-(2,3-dideoxy-.beta.-D-ribofuranosyl)-6-(methylthio)purine |
| C64 | 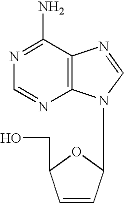 |
| | 2,3-dideoxydidehydroadenosine |
| C65 | 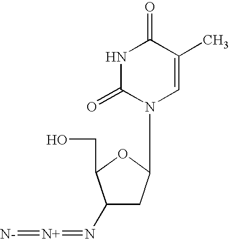 |
| | 3′-azido-3′-deoxythymidine |
| C66 | 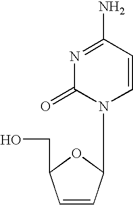 |
| | 2′,3′-dideoxydidehydrocytidine |
| C67 | 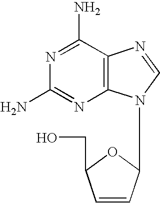 |
| | 2,6-diaminopurine-2′,3′-dideoxydidehydrorboside |
| C68 | 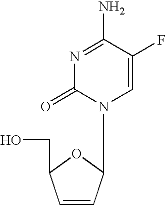 |
| | β-D-2′,3′-didehydro-2′,3′-dideoxy-5-fluorocytidine |
| C69 | 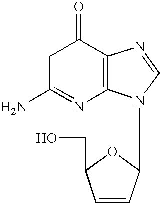 |
| | 2′,3′-didehydro-2′,3′-dideoxyguanosine |
| C70 | 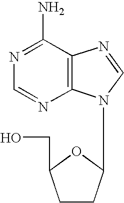 |
| | 2′,3′-dideoxyadenosine |
| C71 | 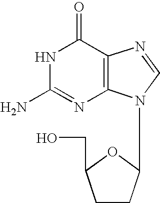 |
| | 2′,3′-dideoxyguanosine |
| C72 | 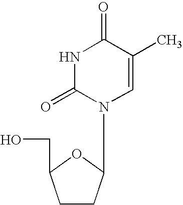 |
| | 3′-deoxythymidine |
| C73 | 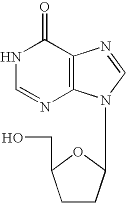 |
| | 2′,3′-dideoxyinosine |
| C74 | 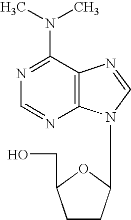 |
| | 6-dimethylaminopurine-2′,3′-dideoxyriboside |
| C75 | 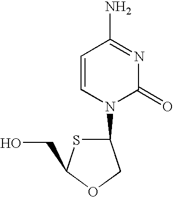 |
| | (−)-2′-deoxy-3′-oxa-4′-thiocytidine |
| C76 | 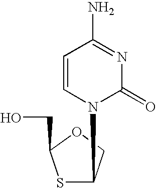 |
| | (+)-2′-deoxy-3′-oxa-4′-thiocytidine |
| C77 | 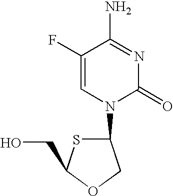 |
| | (−)-2′-deoxy-3′-oxa-4′-thio-5-fluorocytidine |
| C78 | 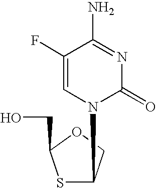 |
| | (+)-2′-deoxy-3′-oxa-4′-thio-5-fluorocytidine |
| C79 | 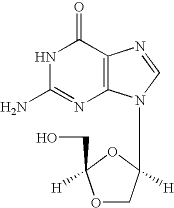 |
| | (−)-(2R,4R)-9-[2-(hydroxymethyl)-1,3- |
| | dioxolan-4-yl]guanine |
| C80 | 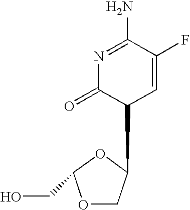 |
| | (+)-(2S,4R)-1-[2-(hydroxymethyl)-1,3-dioxolan- |
| | 4-yl]-5-fluorocytosine |
| C81 | 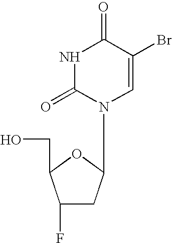 |
| | 2′,3′-dideoxy-3′-fluoro-5-bromouridine |
| C82 | 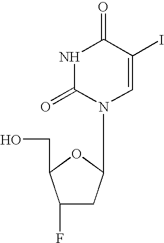 |
| | 3′-fluoro-2′,3′-dideoxy-5-iodouridine |
| C83 | 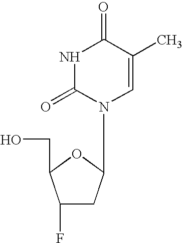 |
| | 3′-fluoro-3′-deoxythymidine |
| C84 | 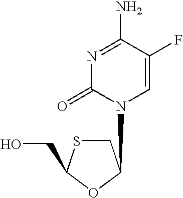 |
| | (−)-(2R,5S)-5-fluoro-1-[2-(hydroxymethyl)-1,3-oxathiolan-5-yl]cytos |
| C85 | 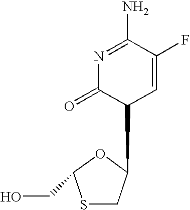 |
| | (+)-(2R,5R)-5-fluoro-1-[2-(hydroxymethyl)-1,3- |
| | oxathiolan-5-yl]cytosine |
| C86 | 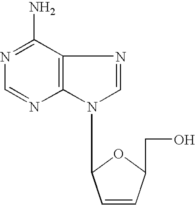 |
| | .beta.-L-2′,3′-didehydro-2′,3′-dideoxyadenosine |
| C87 | 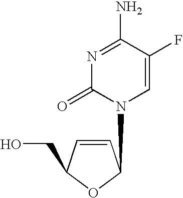 |
| | 2′,3′-dideoxy-2′,3′-didehydro-.beta.-L-5-5-fluorocytidine |
| C88 | 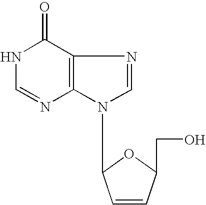 |
| | .beta.-L-2′,3′-didehydro-2′,3′-dideoxyinosine |
| C89 | 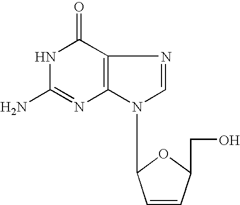 |
| | .beta.-L-2′,3′-didehydro-2′,3′-dideoxyguanosine |
| C90 | 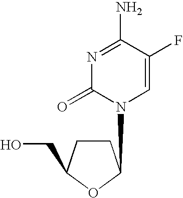 |
| | 2(1H)-pyrimidinone,4-amino-5-fluoro-1-[(2S,5R)-tetrahydro-5- |
| | (hydroxymethyl)-2-furanyl]- |
| C91 | 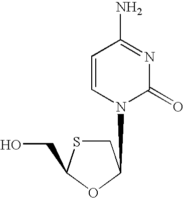 |
| | cis-1-[2′-hydroxymethyl-5′-(1,3-oxathiolanyl)]cytosine |
| C92 | 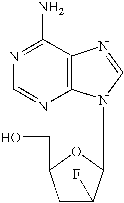 |
| | 9-(2″-fluoro-2′,3′-dideoxy-B-D-threopentafuranosyl)adenine |
| C93 | 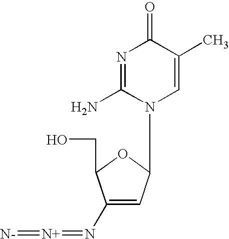 |
| | 5-methyl-3′-azido-2′3′-dideoxyisocytidine |
| C94 | 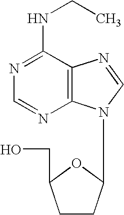 |
| | N-ethyl-2′,3′-dideoxyadenosine |
| C95 | 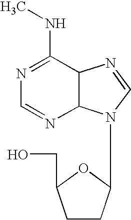 |
| | 6-methyl-2′,3′-dideoxyadenosine |
| C96 | 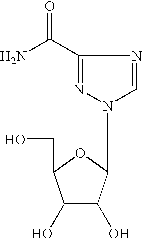 |
| | 1-.beta.-D-ribofuranosyl-1,2,4-triazolo-3-carboxamide |
| C97 | 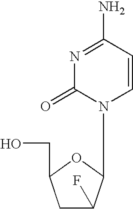 |
| | 1-(2′,3′-dideoxy-2′-fluoro-.beta.-D-threo-pentofuranosyl)cytosine |
| C98 | 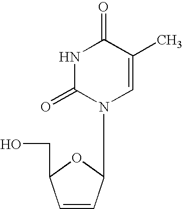 |
| | thymidine,2′,3′-didehydro-,3′-deoxy |
| C99 | 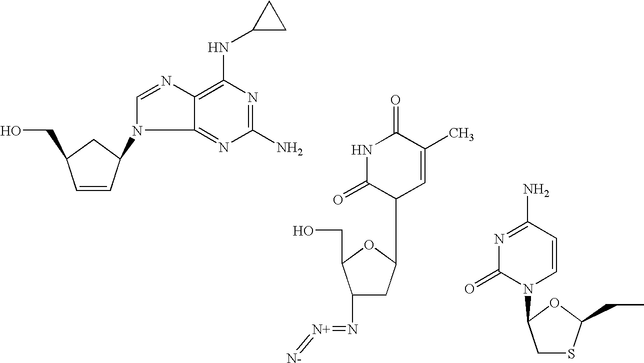 |
| | (1S,4R)-4-[2-amino-6-(cyclopropylamino)-9H-purin-9-yl]-2-cyclopentene-1-methan |
| | .beta.-L-(−)-2′,3′-dideoxy-3-thiacytidine & 3′-azido-3′-deoxythymidine |
| C100 | 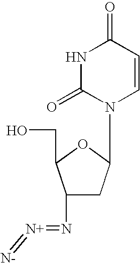 |
| | 3′-azido-2′,3′-dideoxyuridine |
| C101 | 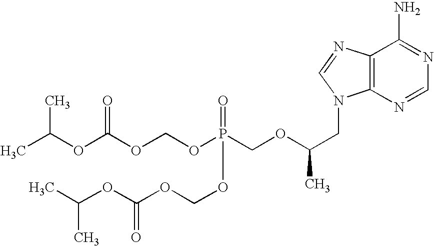 |
| | 9-[(R)-2-[[bis[[(isopropoxycarbonyl)oxy]methoxy]phosphinyl] |
| | methoxy]propyl]adenine |
| C102 | 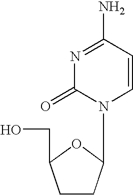 |
| | 2′,3′-dideoxycytidine |

In an alternative of this embodiment, the reverse transcriptase inhibitor is a non-nucleoside reverse transcriptase inhibitor. Examples of suitable non-nucleoside reverse transcriptase inhibitors for use in the current invention are shown in Table D.

TABLE D

| Compound | |
|---|---|
| No. | Compound |
| D1 | 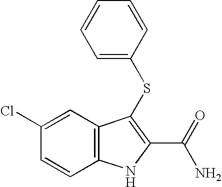 |
| | 6-chloro-3-(phenylthio)-2-indolecarboxamide |
| D2 | 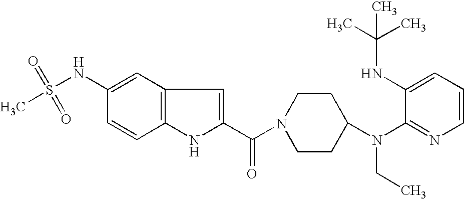 |
| | 1-[(5-methanesulfonamidoindol-2-yl)carbonyl]-4-[N-ethyl-N-[3- |
| | [(1,1-dimethylethyl)amino]-2-pyridinyl]amino]piperidine |
| D3 | 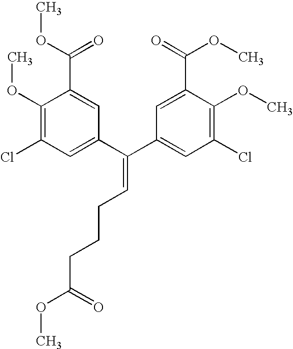 |
| | methyl-3′,3″-dichloro-4′,4″-dimethoxy-5′,5″-bis(methoxycarbonyl)-6,6-diphenylhexenoate |
| D4 | 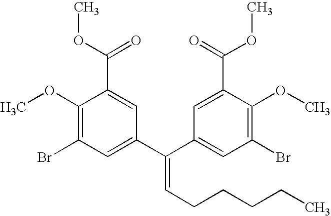 |
| | Methyl-3-bromo-5-(1-(5-bromo-4-methoxy-3-(methoxycarbonyl) phenyl)hept-1-enyl)-2-methoxybenzoate |
| D5 | 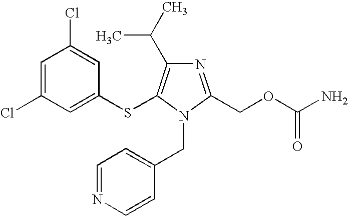 |
| | 5-(3,5-dichlorophenyl)thio-4-isopropyl-1-(4-pyridyl)methyl-1H-imidazol-2-ylmethylcarbamate |
| D6 | 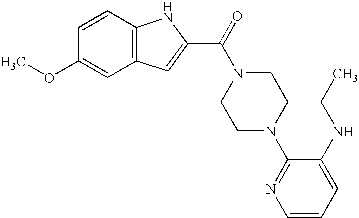 |
| | 1-[(5-methoxyindol-2-yl)carbonyl]-4-[3-(ethylamino)-2-pyridyl]piperazine |
| D7 | 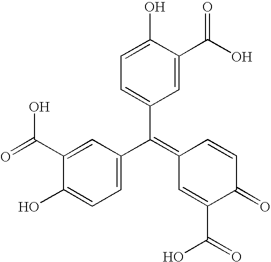 |
| | Aurintricarboxylic acid |
| D8 | 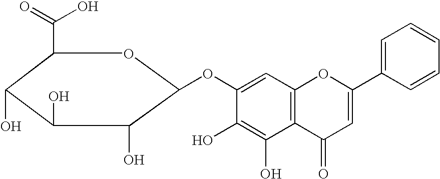 |
| | 5,6,7-trihydroxyflavone-7-O-β-D-glucopyranosideuronic acid |
| D9 | 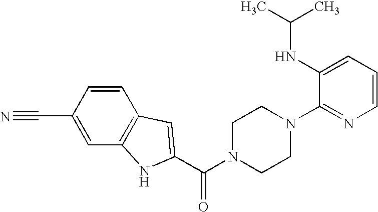 |
| | 1-[(6-cyano-2-indolyl)carbonyl]-4-[3-(isopropylamino)-2-pyridinyl]piperazine |
| D10 | 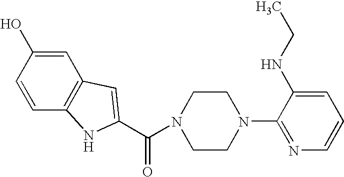 |
| | 1-[3-(ethylamino)-2-pyridinyl]-4-[(5-hydroxy-2-indolyl)carbonyl]piperazine |
| D11 | 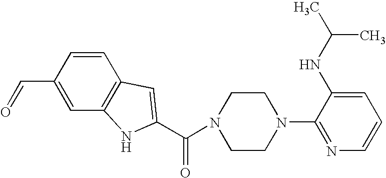 |
| | 1-[(6-formyl-2-indolyl)carbonyl]-4-[3-(isopropylamino)-2-pyridinyl]piperazine |
| D12 | 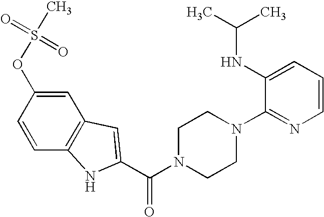 |
| | 1-[[5-(methylsulfonyloxy)-2-indolyl]carbonyl]-4-[3-(isopropylamino |
| | 2-pyridinyl]piperazine |
| D13 | 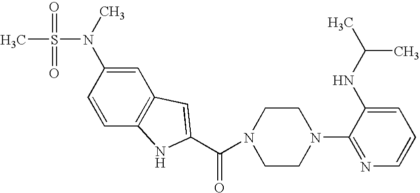 |
| | 1-[5-[[N-(methyl)methylsulfonylamino]-2-indolyl]carbonyl]-4-[3- |
| | (isopropylamino)-2-pyridinyl]piperazine |
| D14 | 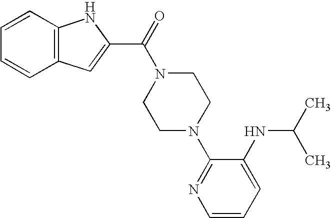 |
| | 1-(indolyl-2-carbonyl)-4-[3-[(1-methylethyl)amino]pyridyl]piperazine |
| D15 | 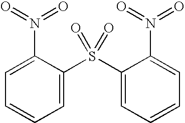 |
| | bis(2-nitrophenyl)sulfone |
| D16 | 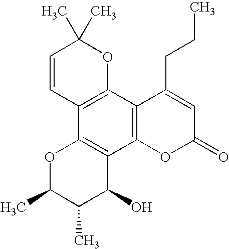 |
| | Calanolide A |
| D17 | 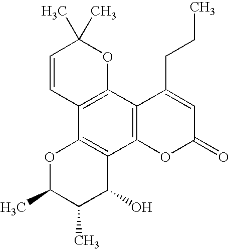 |
| | Calanolide B |
| D18 | 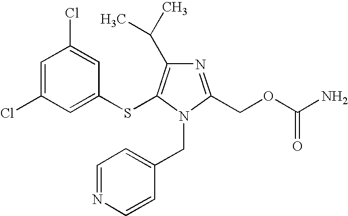 |
| | 5-(3,5-dichlorophenyl)thio-4-isopropyl-1-(4-pyridyl)methyl-1H-imidazol-2-ylmethylcarbamate |
| D19 | 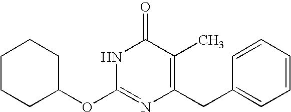 |
| | 6-benzyl-5-methyl-2-(cyclohexyloxy)pyrimidin-2-one |
| D20 | 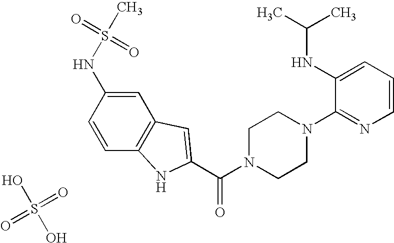 |
| | 1-(5-methanesulphonamido)-1H-indol-2-yl-carbonyl)-4-[3-(isopropylamino)-2-pyridinyl]piperazine |
| D21 | 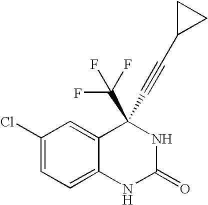 |
| | 2(1H)-quinazolinone,6-chloro-4-(cyclopropylethynyl)-3,4-dihydro-4-(trifluoromethyl)-,(4S)- |
| D22 | 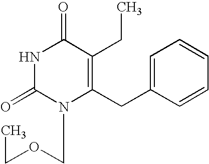 |
| | 6-benzyl-1-(ethoxymethyl)-5-ethyluracil |
| D23 | 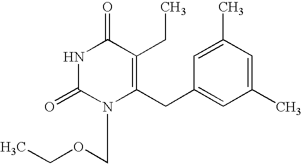 |
| | 5-ethyl-1-ethoxymethyl-6-(3,5-dimethylbenzyl)uracil |
| D24 | 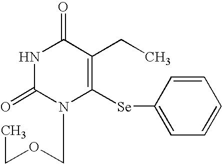 |
| | 5-ethyl-1-(ethoxymethyl)-6-(phenylselenenyl)uracil |
| D25 | 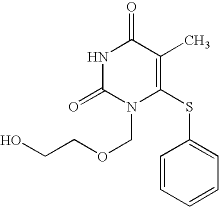 |
| | 1-[(2-hydroxyethoxy)methyl]-6-(phenylthio)thymine |
| D26 | 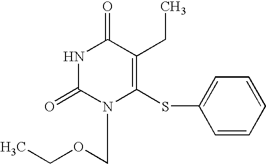 |
| | 1-[(ethoxy)methyl]-6-phenylthio)-5-ethyluracil |
| D27 | 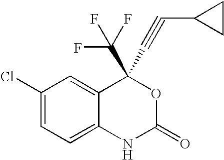 |
| | (−)-6-chloro-4-cyclopropylethynyl-4-trifluoromethyl-1,4-dihydro-2H-3,1-benzoxazin-one |
| D28 | 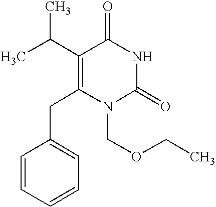 |
| | 2,4(1H,3H)-pyrimidinedione,1-(ethoxymethyl)-5-(1-methylethyl)-6-(phenylmethyl)- |
| D29 | 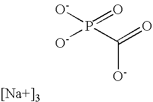 |
| | Phosphonoformic acid trisodium salt |
| D30 | 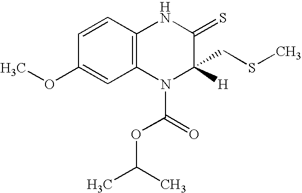 |
| | (S)-7-methoxy-3,4-dihydro-2-[(methylthio)methyl]-3-thioxo- |
| | 2(1H)-quinoxalinecarboxylic acid, isopropyl ester |
| D31 | 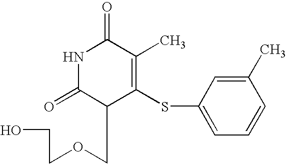 |
| | 1[(2-hydroxyethoxy)methyl]-6-(3-methylphenyl)thio)thymine |
| D32 | 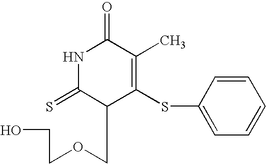 |
| | 1-[(2-hydroxyethoxy)methyl]-6-(phenylthio)-2-thiothymine |
| D33 | 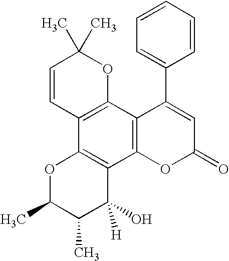 |
| | inophyllum B |
| D34 | 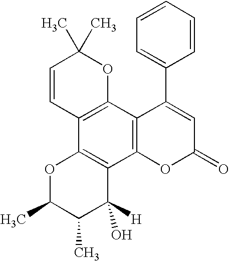 |
| | inophyllum P |
| D35 | 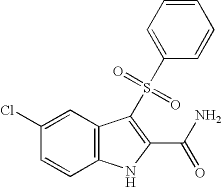 |
| | 5-chloro-3-(phenylsulfonyl)indole-2-carboxamide |
| D36 | 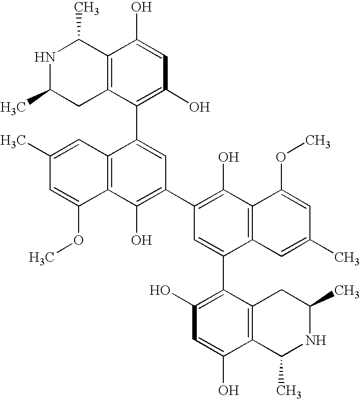 |
| | 5,5′-(1,1′-dihydroxy-8,8′-dimethoxy-6,6′-dimethyl[2,2′-binaphthalene]- |
| | 4,4′-diyl)bis[1,2,3,4,-tetrahydro-1,3-dimethyl-6,8-isoquinolinediol] |
| D37 | 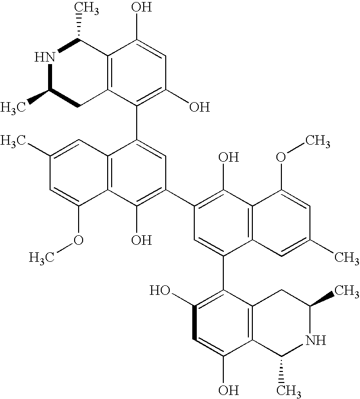 |
| | 5,5′-(1,1′-dihydroxy-8,8′-dimethoxy-6,6′-dimethyl[2,2′-binaphthalene]- |
| | 4,4′-diyl)bis[1,2,3,4,-tetrahydro-1,3-dimethyl-6,8-isoquinolinediol] |
| D38 | 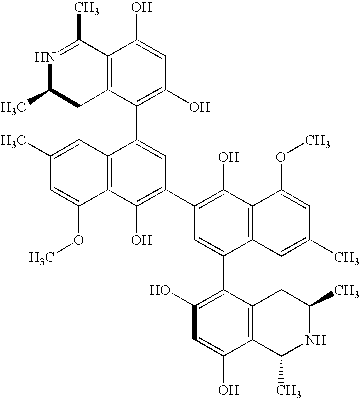 |
| | 5,5′-(1,1′-dihydroxy-8,8′-dimethoxy-6,6′-dimethyl[2,2′-binaphthalene]- |
| | 4,4′-diyl)[3,4,-dihydro-8-methoxy-1,3-dimethyl-6-isoquinolinediol],[1,2,3,4-tetrahydro-1,3-dimethyl-6,8-isoquinolinediol] |
| D39 | 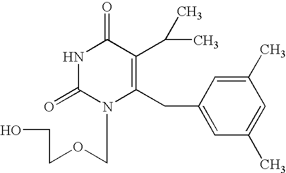 |
| | 6-(3,5-dimethylbenzyl)-1-[(2-hydroxyethoxy)methyl]-5-isopropyluracile |
| D40 | 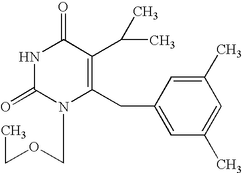 |
| | 6-(3,5-dimethylbenzyl)-1-(ethoxymethyl])-5-isopropyluracile |
| D41 | 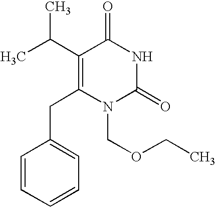 |
| | 2,4(1H,3H)-pyrimidinedione,1-(ethoxymethyl)-5-(1-methylethyl)-6-(phenylmethyl)- |
| D42 | 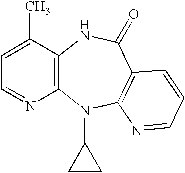 |
| | N11-cyclopropyl-4-methyl-5,11-dihydro-6H-dipyrido [3,2-b:2′,3′-e]-[1,4]diazepin-6-one |
| D43 | 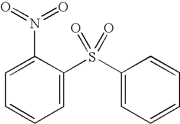 |
| | 2-nitrophenyl phenyl sulfone |
| D44 | 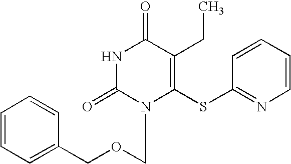 |
| | 1-benzyloxymethyl-5-ethyl-6-(2-pyridylthio)uracil |
| D45 | 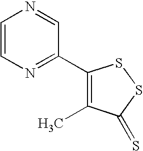 |
| | 4-methyl-5-(pyrazinyl)-3H-1,2-dithiole-3-thione |
| D46 | 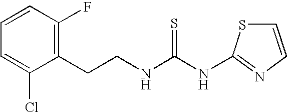 |
| | N-[2-(2-chloro-6-fluorophenethyl)]-N′-(2-thiazolyl)thiourea |
| D47 | 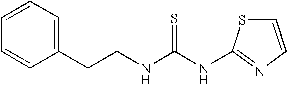 |
| | N-(2-phenethyl)-N′-(2-thiazolyl)thiourea |
| D48 | 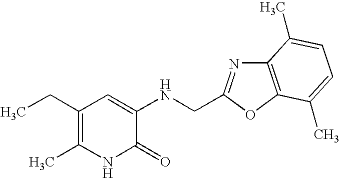 |
| | 3-[(4,7-dimethyl-2-benzoxazolylmethyl)amino]-5-ethyl-6-methylpyridin-2(1H)-one |
| D49 | 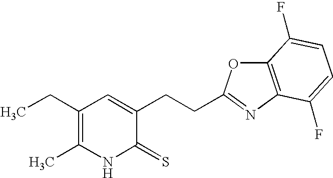 |
| | 3-[2-(4,7difluorobenzoxazol-2-yl)ethyl]-5-ethyl-6-methylpyridin-2(1H)-thione |
| D50 | 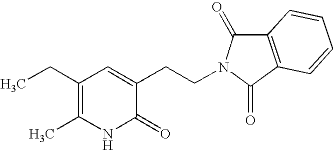 |
| | 3-ethyl-6-methyl-3-[(2-phthalimido)ethyl]-2-pyridinone |
| D51 | 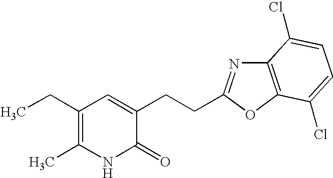 |
| | 3-[(4,7-dichlorobenzoxazolylmethyl)amino]-5-ethyl-6-methylpyridin-2(1H)-one |
| D52 | 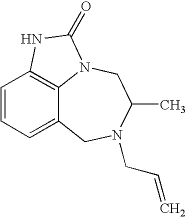 |
| | (+/−)-4,5,6,7-tetrahydro-5-methyl-6-(2-propenyl)-imidazo-[4,5,1-jk][1,4]-benzodiazepin-2(1H)-one |
| D53 | 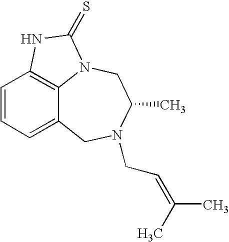 |
| | (+)-S-4,5,6,7-tetrahydro-5-methyl-6-(3-methyl-2-butenyl)-imidazo-[4,5,1-jk][1,4]-benzodiazepin-2(1H)-thione |
| D54 | 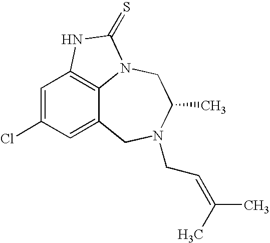 |
| | (+)-S-4,5,6,7-tetrahydro-9-chloro-5-methyl-6-(3-methyl-2-butenyl)- |
| | imidazo[4,5,1-jk][1,4]-benzodiazepin-2(1H)-thione |
| D55 | 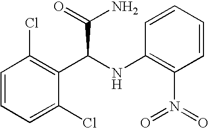 |
| | (−)-2,6-dichloro-.alpha.-[(2-nitrophenyl)amino]benzamide |
| D56 | 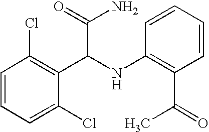 |
| | (+−)-2,6-dichloro-.alpha.-[(2-acetylphenyl)amino]benzamide |
| D57 | 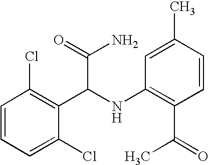 |
| | (+−)-2,6-dichloro-.alpha.-[(2-acetyl-5-methylphenyl)amino]benzamide |
| D58 | 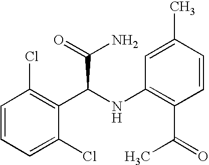 |
| | (−)-2,6-dichloro-.alpha.-[(2-acetyl-5-methylphenyl)amino]benzamide |
| D59 | 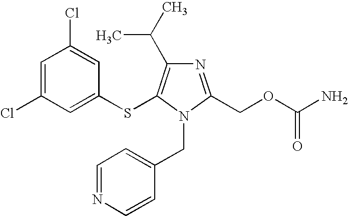 |
| | 5-(3,5-dichlorophenyl)thio-4-isopropyl-1-(4-pyridyl)methyl-1H-imidazol-2-ylmethylcarbamate |
| D60 | 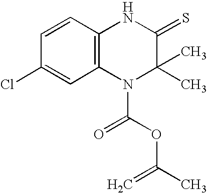 |
| | 6-chloro-3,3-dimethyl-4-(isopropenyloxycarbonyl)-3,4-dihydroquinoxalin-2(1H)-thione |
| D61 | 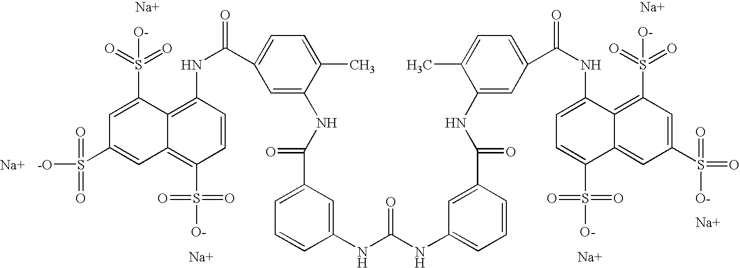 |
| | 8,8′-[carbonylbis[imino-3,1-phenylenecarbonylimino(4-methyl-3,1-phenylene)carbonyl-imino]] |
| | bis-1,3,5-naphthalenetrisulfonic acid hexasodium salt |
| D62 | 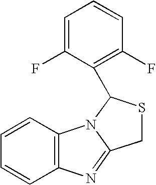 |
| | (R,S)-1-(2,6-difluorophenyl)-1H,3H-thiazolo[3,4-a]benzimidazole |
| D63 | 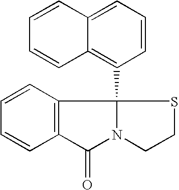 |
| | (+)-(R)-9b-(1-naphthyl)-2,3-dihydrothiazolo[2,3-a] isoindol-5(9bH)-one |
| D64 | 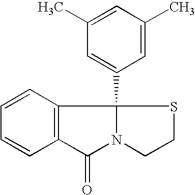 |
| | (+)-(R)-9b-(3,5-dimethylphenyl)-2,3-dihydrothiazolo[2,3-a] isoindol-5(9bH)-one |
| D65 | 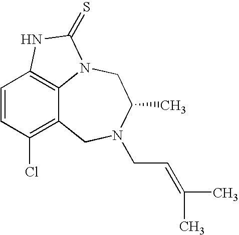 |
| | (+)-(S)-4,5,6,7-tetrahydro-8-chloro-5-methyl-6-(3-methyl-2-butenyl)- |
| | imidazo[4,5,1-jk][1,4]benzodiazepine-2-(1H)-thione |
| D66 | 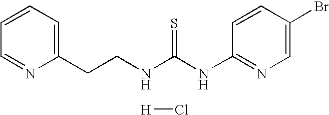 |
| | N-[2-(2-pyridylethyl)-N′-[2-(5-bromopyridyl)] thiourea, hydrochloride |
| D67 | 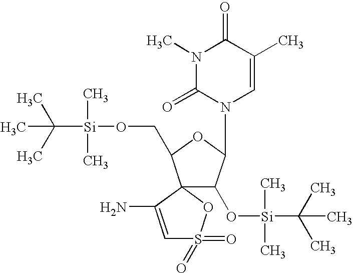 |
| | thymidine, 3-methyl, [2′,5′-bis-O-(tert-butyldimethylsilyl)-.beta.-D-ribofuranosyl]- |
| | 3′-spiro-5-(4-amino-1,2-oxathiole-2,2-dioxide |
| D68 | 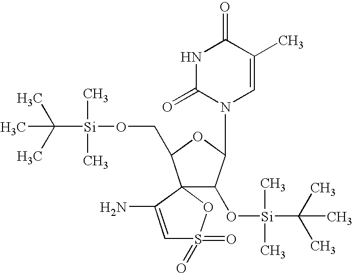 |
| | [1-[2′,5′-bis-O-(tert-butyldimethylsilyl)-.beta.-D-ribofuranosyl]thymine]- |
| | (R)(ribo)-3′-spiro-5-(4-amino-1,2-oxathiole-2,2-dioxide). |
| D69 | 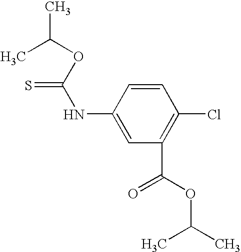 |
| | 4-chloro-3-(isopropoxycarbonyl)phenylcarbamothioic acid, O-isopropyl ester |
| D70 | 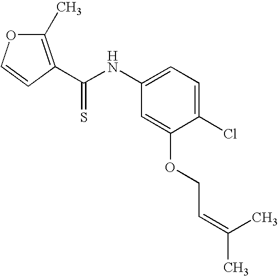 |
| | N-[4-chloro-3-(3-methyl-2-butenyloxy)phenyl]-2-methyl-3-furancarbothioamide |
| D71 | 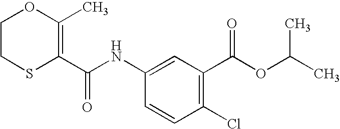 |
| | benzoic acid, 2-chloro-5[(2-methyl-5,6-dihydro-1,4-oxathiin-3-yl) carbonylamino]isopropyl ester |

In yet another alternative of this embodiment, the reverse transcriptase inhibitor is an acyclic nucleoside phosphate analog. By way of example, suitable acyclic nucleoside phosphate analogs for use in the current invention are shown in Table E.

TABLE E

| Com- | |
|---|---|
| pound | |
| No. | Compound |
| E1 | 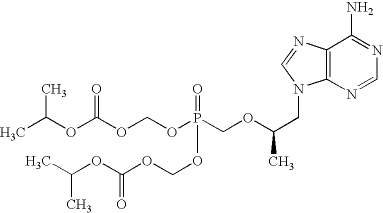 |
| | 9-[(R)-2-[[bis[[(isopropoxycarbonyl)oxy]methoxy]phosphinyl] |
| | methoxy]propyl]adenine |
| E2 | 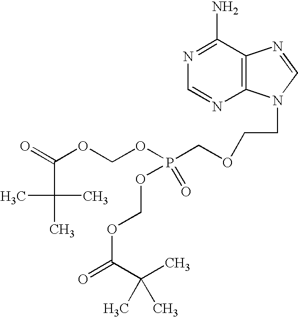 |
| | Bis(pivaloyloxymethyl)-9-(2-phosphonylmethoxyethyl)adenine |
| E3 | 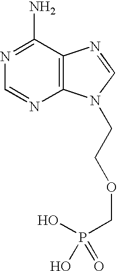 |
| | 9-(2-phosphonylmethoxyethyl)adenine |
| E4 | 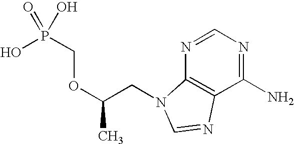 |
| | (R)-9-(2-phosphonylmethoxypropyl)adenine |
| E5 | 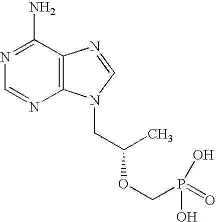 |
| | (S)-9--(2-phosphonylmethoxypropyl)adenine |
| E6 | 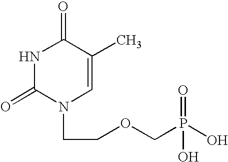 |
| | 2-phosphonylmethoxyethyl-thymine |
| E7 | 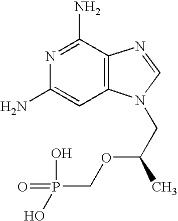 |
| | (R)-9-(2-phosphonylmethoxypropyl)-2,6-diaminopurine |
| E8 | 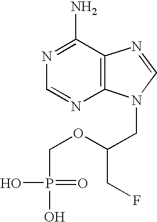 |
| | 9-[(2RS)-(3-fluoro-2-phosphonylmethoxypropyl)]adenine |
| E9 | 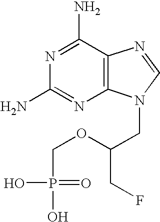 |
| | 9-[(2RS)-3-fluoro-2-phosphonylmethoxypropyl]-2,6- |
| | diaminopurine |
| E10 | 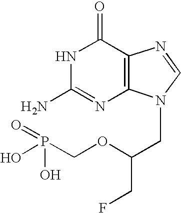 |
| | (RS)-9-[(2RS)-3-fluoro-2-phosphonylmethoxypropyl]-guanine |
| E11 | 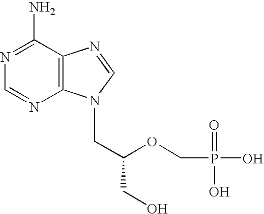 |
| | 9-[(2S)-3-hydroxy-2-phosphonylmethoxylpropyl]adenine |
| E12 | 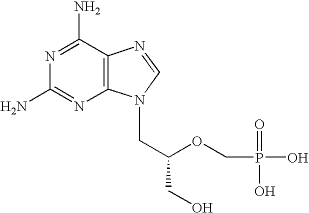 |
| | 9-[(2S)-3-hydroxy-2-phosphonylmethoxylpropyl]-2,6- |
| | diaminopurine |
| E13 | 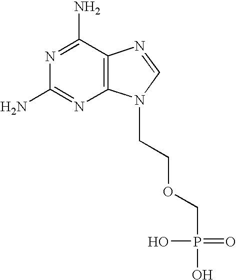 |
| | 9-(2-phosphonylmethoxyethyl)-2,6-diaminopurine |
| E14 | 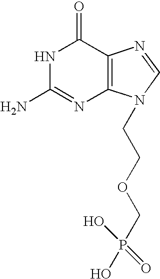 |
| | 9-(2-phosphonylmethoxyethyl)guanine |
| E15 | 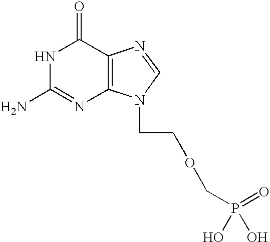 |
| | Phosphonic acid, [[2-(2-amino-9H-purin-9-yl)ethoxy]methyl]- |
| E16 | 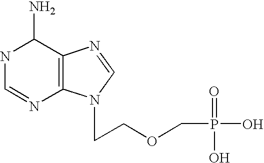 |
| | 2-phosphonylmethoxyethyl-6-oxopurine |

In another embodiment, the viral replication inhibitor is a purine nucleoside phosphorylase (PNP) inhibitor. The enzyme PNP is predominantly present in T cells and is necessary for DNA synthesis in these cells. Inhibition of this enzyme, accordingly, blocks DNA synthesis and thereby prevents T-cell proliferation. Examples of suitable PNP inhibitors are listed in Table F.

TABLE F

| Compound | |
|---|---|
| No. | Compound |
| F1 | 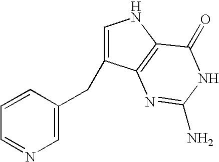 |
| | 4H-pyrrolo(3,2-d)pyrimidin-4-one,1,5- |
| | dihydro-2-amino-1-(3-pyridinylmethyl) |
| F2 | 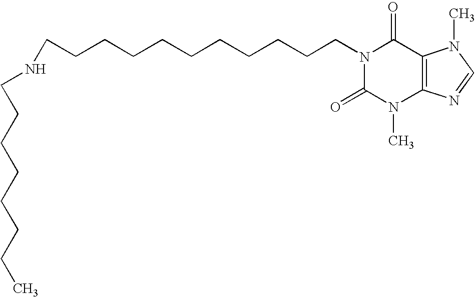 |
| | 1-(11-octylamino-10-hydroxyundecyl)-3,7-dimethylxanthine |

In yet another embodiment, the viral replication inhibitor is a polyamine biosynthesis inhibitor. Generally speaking, the biosynthesis of polyamines is involved in the control of many biological processes such as cell growth, gene transcription and translation. As such, inhibitors of polyamine biosynthesis substantially inhibit HIV replication by reducing the subject's cell growth and proliferation. By way of example, suitable inhibitors of polyamine biosynthesis for use in the present invention are shown in Table G.

TABLE G

| Com- | |
|---|---|
| pound | |
| No. | Compound |
| G1 | 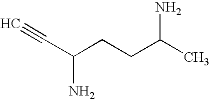 |
| | 6-heptyne-2,5-diamine |
| G2 | 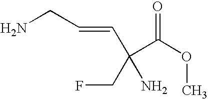 |
| | Methyl 2-fluoromethyl-2,5-diamino-3-pentenoate |
| G3 | 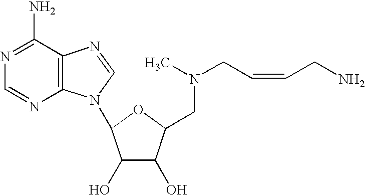 |
| | 5′-[[(Z)-4-amino-2-butenyl]methylamino]-5′-deoxyadenosine |
| G4 | 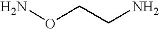 |
| | 1-aminooxyethylamine |

In yet another embodiment, the viral replication inhibitor is an antisense therapy agent. These agents are typically unmodified or modified antisense oligonucleotides directed against various HIV RNA sequences that have been shown to inhibit viral replication, both in a sequence-specific and in a non-sequence specific manner. Because of their complementary, the agent binds to the HIV nucleic acid and thereby prevents its transcription. Of course the particular antisense oligonucleotides employed will vary considerably depending upon its intended target within the HIV genome and one skilled in the art can readily design appropriate antisense oligonucleotides for use in the present invention.

A further aspect of the invention encompasses anti-human immunodeficiency virus agents that inhibit or prevent assembly of the virus after its replication. Generally speaking, viral assembly inhibitors inhibit or prevent viral RNA processing, glycosylation, or capsid formation. In one embodiment, the inhibitor of viral assembly is a viral RNA process inhibitor. Any agent capable of blocking HIV RNA processing may be employed. By way of example, one such target is the RNA binding protein Rev. Rev is essential for HIV replication, since it allows the nuclear export of unspliced and partially spliced viral mRNAs that encode the HIV structural proteins. Inhibition of Rev with an agent such as fleephilone (e.g. shown in Table H), accordingly, inhibits HIV replication by blocking RNA processing. Examples of suitable viral RNA process inhibitors are shown in Table H.

TABLE H

| Compound | |
|---|---|
| No. | Compound |
| H1 | 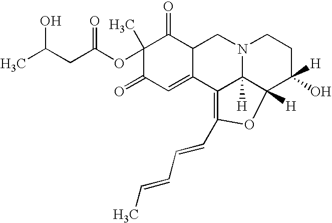 |
| | Fleephilone |
| H2 | 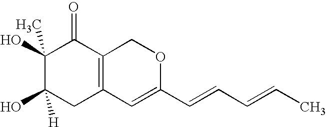 |
| | Harziphilone |

In another embodiment, the inhibitor of viral assembly is a glycosylation inhibitor. Certain HIV viral proteins undergo glycosylation, a step that is necessary for not only replication of the virus, but also its assembly after replication. Any agent capable of blocking HIV glycosylation may be employed. By way of example, one such agent is castanospermine. Castanospermine is a naturally occurring alkaloid and inhibitor of HIV glucosidase-I. Several analogs of castanospermine have been developed and are contemplated for use in the present invention. Other glycosylation inhibitors suitable for use in the present invention are shown in Table I.

TABLE I

| Compound | |
|---|---|
| No. | Compound |
| I1 | 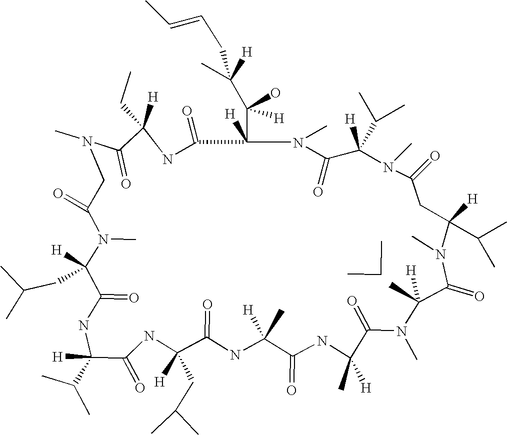 |
| | Cyclosporin A |
| I2 | 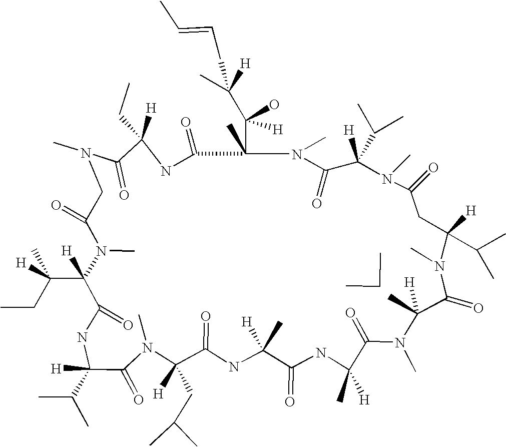 |
| | [Me-Ile-4]Cyclosporin A |
| I3 | Acemannan |
| I4 | 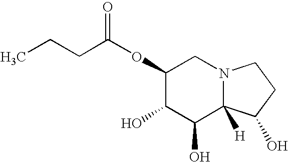 |
| | Butanoic acid, (1S,6S,7S,8R,8aR)-octahydro- |
| | 1,7,8-trihydroxy-6-indolizinyl ester |
| I5 | 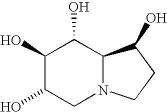 |
| | (1S,6S,7R,8R,8aR)-1,6,7,8-tetrahydroxyindolizidine |
| I6 | 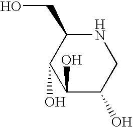 |
| | 1,5-Dideoxy-1,5-imino-D-glucitol |

In yet another embodiment, the viral assembly inhibitor is a zinc finger inhibitor. The inner core of HIV is called the nucleocapsid. It is held together by a complex array of proteins commonly known as “zinc fingers.” By inhibiting the formation of these protein arrays, zinc finger inhibitors prevent the virus from properly assembling its nucleocapsid. Any zinc finger inhibitor that is capable of disrupting zinc finger protein arrays of the HIV nucleocapsid may be utilized in the present invention. For example, suitable agents for use as zinc finger inhibitors in the present invention are shown in Table J.

TABLE J

| Compound | |
|---|---|
| No. | Compound |
| J1 | 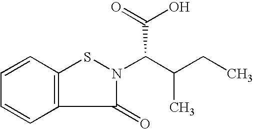 |
| | 3-methyl-2(S)-(1-oxo(3-thiaisoindolin- |
| | 2yl)pentanoic acid |
| J2 | 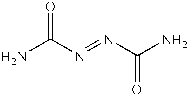 |
| | 1,1′-azobisformamide |
| J3 | 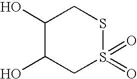 |
| | 1,2-dithiane-4,5-diol,1,1-dioxide,cis |

In yet another embodiment, the viral assembly inhibitor is a protease inhibitor. Protease inhibitors block the protease enzyme. Generally speaking, when new HIV particles break off from an infected cell, protease enzyme is employed to cut long protein strands into the parts required to assemble a mature virus. By inhibiting the protease enzyme, the necessary smaller-sized viral proteins cannot be made, and therefore, proper viral assembly cannot occur. As a result, the virus is prevented from spreading from cell to cell. Any agent capable of inhibiting the HIV protease enzyme may be employed in the present invention. By way of example, suitable protease inhibitors are listed in Table K.

TABLE K

| Compound | |
|---|---|
| No. | Compound |
| K1 | 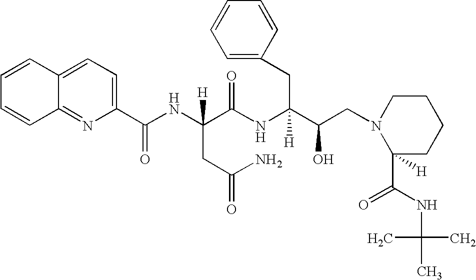 |
| | N-tert-butyl-1-[2(R)-hydroxy-3(S)-[[N-(2-quinolylcarbonyl)-L-asparaginy |
| | amino]-4-phenylbutyl]-2(S)piperidinecarboxamide |
| K2 | 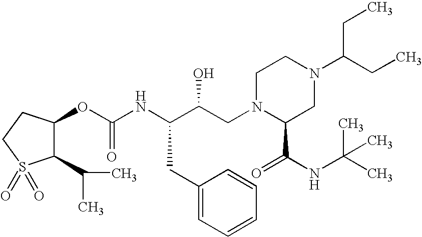 |
| | Carbamic acid, [3-[4-(1-ethylpropyl)-2(S)-[[(1,1-dimethylethyl)amino]carbon |
| | piperazinyl]-2(R)-hydroxy-1(S)-(phenylmethyl)propyl]-, tetrahydro-2(R)- |
| | (1-methylethyl)-1,1-dioxido-3(R)-thienyl ester |
| K3 | 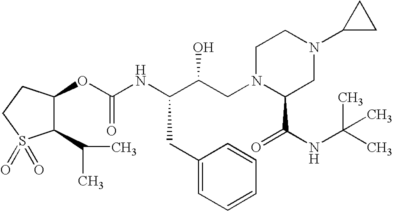 |
| | Carbamic acid, [3-[4-cyclopropyl)-2(S)-[[(1,1-dimethylethyl)amino]carbonyl] |
| | piperazinyl]-2(R)-hydroxy-1(S)-(phenylmethyl)propyl]-, |
| | tetrahydro-2(R)-(1-methylethyl)-1,1-dioxido-3(R)-thienyl ester |
| K4 | 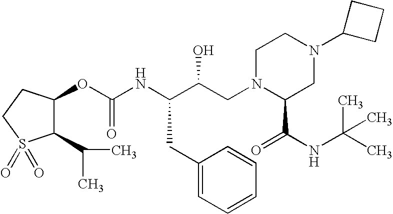 |
| | Carbamic acid, [3-[4-cyclobutyl)-2(S)-[[(1,1-dimethylethyl)amino]carbonyl] |
| | 1-piperazinyl]-2(R)-hydroxy-1(S)-(phenylmethyl)propyl]-, |
| | tetrahydro-2(R)-(1-methylethyl)-1,1-dioxido-3(R)-thienyl ester |
| K5 | 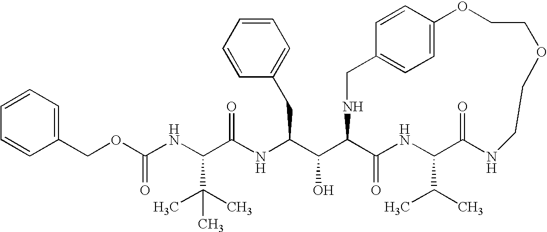 |
| | 1′S,2′S,2′S,9S,12R)-12-[2″-[[N-[(benzyloxy)carbonyl]tert-leucinyl]amino]-1′- |
| | hydroxy-3′-phenylprop-1′-yl]-9-(1-methylethyl)-7,10,13-triaza-1,4-dioxo-8,1 |
| | dioxo[14]paracyclophane |
| K6 | 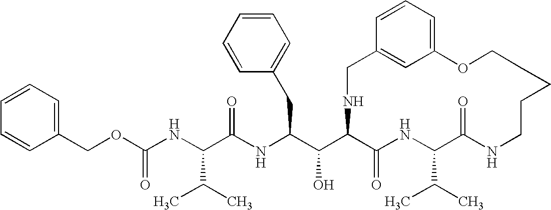 |
| | (1′S,2′S,8S,11R)-11-[2″-[[N-[(benzyloxy)carbonyl]valyl]aminol-1′-hydroxy- |
| | 3′-phenylprop-1′-yl]-8-(1-methylethyl)-6,9,12-triaza-1-oxa-7,10-dioxo[13] |
| | metacyclophane |
| K7 | 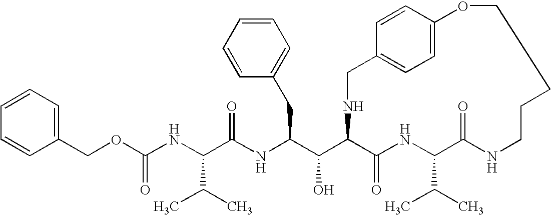 |
| | (1′S,2′S,2″S,9S,11R)-11-[2″-[[N-[(benzyloxy)carbonyl]valyl]amino]-1′ |
| | hydroxy-3′-phenylprop-1′-yl]-8-(1-methylethyl)-6,9,12-triaza-1-oxa-7, |
| | dioxo[13]paracyclophane |
| K8 | 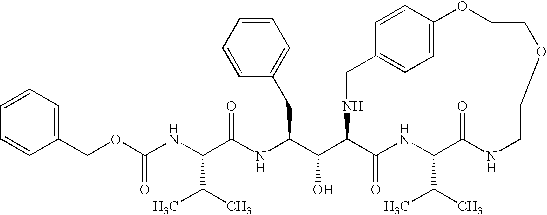 |
| | (1′S,2′S,2″S,9S,12R)-12-[2″-[[N-[(benzyloxy)carbonyl]valyl]amino]-1′- |
| | hydroxy-3′-phenylprop-1′-yl]-9-(1-methylethyl)-7,10,13-triaza-1,4-diaza- |
| | 8,11-dioxo[14]paracyclophane |
| K9 | 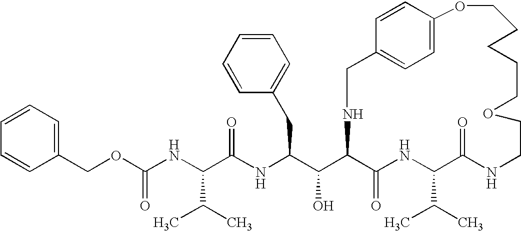 |
| | (1′S,2′S,2″S,15R)-15-[2″-[[N-[(benzyloxy)carbonyl]valyl]amino]-1′- |
| | hydroxy-3′-phenylprop-1′-yl]-12-(1-methylethyl)-10,13,16-triaza-1,4,7- |
| | trioxo-11,14-d ioxo[17]paracyclophane |
| K10 | 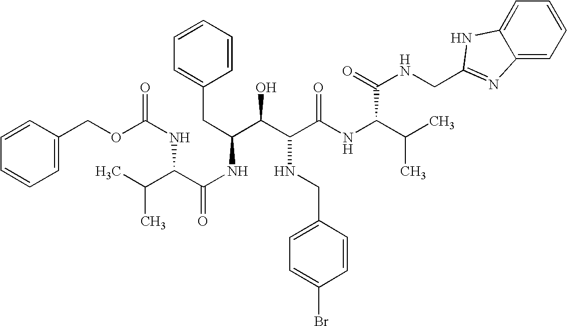 |
| | [1(S),4(S)]-2,4,5-trideoxy-4-[[3-methyl-1-oxo-2-[[(phenylmethoxy)carbonyl] |
| | amino]butyl]amino]-N-[12-methyl-1-[(2-benzimidazolyl)Imethyl]amino] |
| | carbonyl]propyl]-5-phenyl-2-[(4-bromophenyl)methylamino-L-lyxonamide |
| K11 | 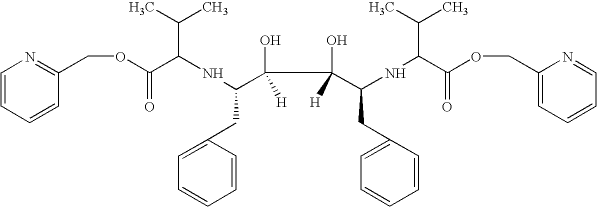 |
| | 2,5-(S,S)-Bis(2-pyridylmethoxyvalyl)1,6-diphenyl-3,4-(S,S)-dihydroxyhexan |
| K12 | 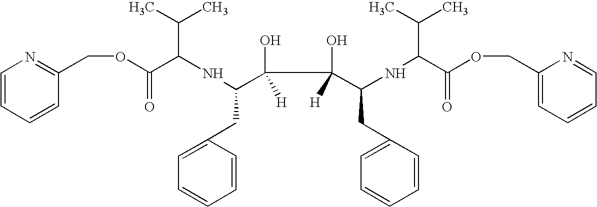 |
| | 1,2,5,6-tetradeoxy-2,5-bis[[3-methyl-2-[[methyl(2-pyridinylmethyl) |
| | amino]carbonyl]amino]-1-oxobutylamino]-1,6-diphenyl-L-altritol |
| K13 | 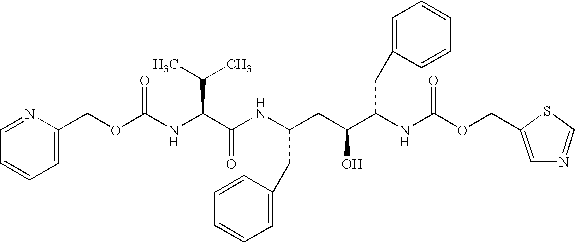 |
| | 10-hydroxy-5-(1-methylethyl)-1-(2-pyridinyl)-3,6-dioxo-8,11- |
| | bis(phenylmethyl)-2-oxa-4,7,12-triazatridecan-13-oic acid, |
| | 5-thiazolylmethyl ester |
| K14 | 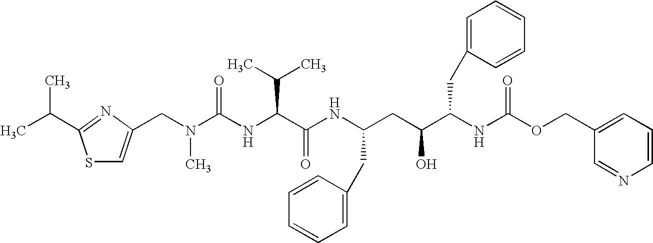 |
| | 10-hydroxy-1-[2-(1-methylethyl)-4-thiazolyl]-5-(1-methylethyl)-3,6-dioxo-8, |
| | 11-bis(phenylmethyl)-2,4,7,12-tetraazatridecan-13-oic acid, 3-pyridinyl |
| | ester |
| K15 | 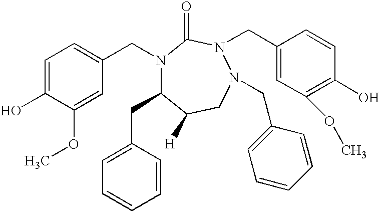 |
| | (5R,6R)-2,4-bis(4-hydroxy-3-methoxybenzyl)-1,5- |
| | dibenzyl-6-hydroxy-3-oxo-1,2,4-triazacycloheptane |
| K16 | 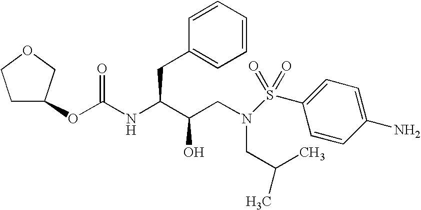 |
| | Carbamic acid, (3-(((4-aminophenyl)sulfonyl)(2-methylpropyl) |
| | amino)-2-hydroxy-1-(phenylmethyl)propyl)-,tetrahydro-3-furanyl ester |
| K17 | 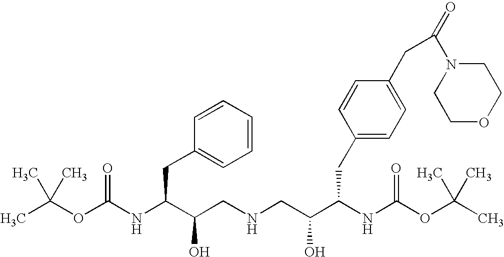 |
| | [1S-[1R*,2S*(2S*,3R*)]]-[3-[[3-[[(1,1-dimethylethoxy)-carbonyl] |
| | amino]-2-hydroxy-4-[4-[2-(4-morpholinyl)-2-oxoethoxy]phenyl] |
| | butyl]amino]-2-hydroxy-1-(phenylmethyl)propyl]carbamic acid, |
| | 1,1-dimethylethyl-ester |
| K18 | 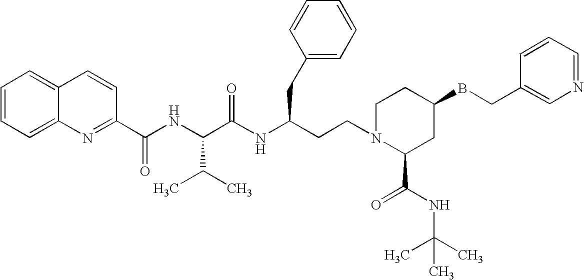 |
| | N-(3-(2(S)-(N-(tert-butyl)carbamonyl)-4(R)-(3-pyridylmethylthio) |
| | piperidyl)-1(S)-benzylpropyl)-3-methyl-2(S)-(2-quinolylcarbonylamino) |
| | butanamide |
| K19 | 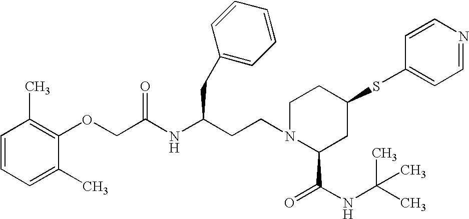 |
| | N-(3-(2(S)-(N-(tert-butyl)carbamoyl)-4(R)-(4-pyridylthio) |
| | piperidyl)-1(S)-benzylpropyl)-2-(2,6-dimethylphenoxy) |
| | ethanamide |
| K20 | 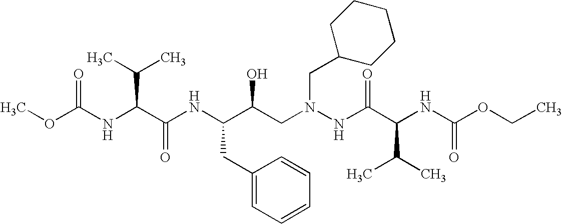 |
| | 1-cyclohexyl-2-[[N-(ethoxycarbonyl)-L-valinyl]amino]-4(S)-hydroxy |
| | 5(S)-[[N-(methoxycarbonyl)-L-valinyl]amino]-6-phenyl-2-azahexan |
| K21 | 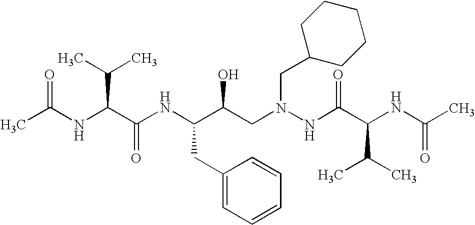 |
| | 1-cyclohexyl-5(S)-2,5-bis[[2-N-(methylcarbonyl)-L-valinyl] |
| | amino]-4(R)-hydroxy-6-phenyl-2-azahexane |
| K22 | 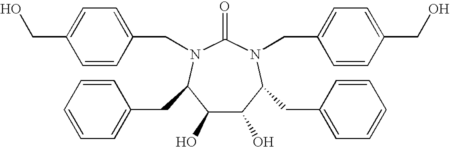 |
| | [4R-(4.alpha.,5.alpha.,6.beta.,7.beta.,7.beta.)]-hexahydro- |
| | 5,6-dihydroxy-1,3-bis[(4-hydroxymethyl)phenyl]methyl]-4,7- |
| | bis(phenylmethyl)-2H-1,3-diazepin-2-one) |
| K23 | 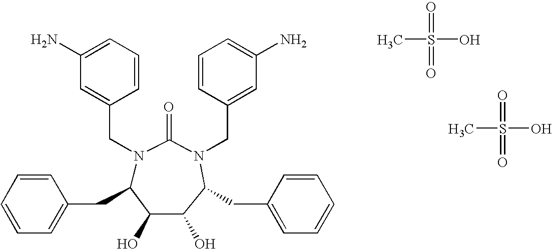 |
| | [4R-(4.alpha.,5.alpha.,6.beta.,7.beta.)]-hexahydro-5,6-dihydroxy-1,3-bis |
| | [(3-aminophenyl)methyl]-4,7-bis(phenylmethyl)-2H-1,3-diazepin-2-one] |
| K24 | 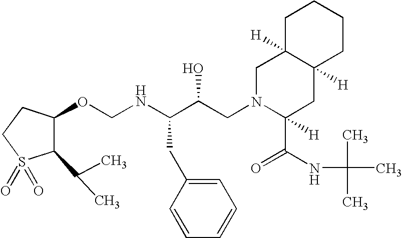 |
| | 2-[3-[3-(R)-[[(2-cis-isopropyl-1,1-dioxotetrahydrothienyloxy) |
| | carbonyl]amino]-4-phenyl-2(R)-hydroxybutyl]]-N-(11- |
| | dimethylethyl)decahydro-3-isoquinolinecarboxamide |
| K25 | 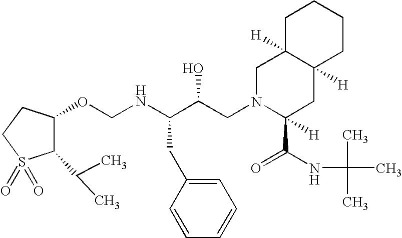 |
| | 2-[3-[3-(S)-[[(2-cis-isopropyl-1,1-dioxotetrahydrothienyloxy) |
| | carbonyl]amino]-4-phenyl-2(R)-hydroxybutyl]]-N-(1,1- |
| | dimethylethyl)decahydro-3-isoquinolinecarboxamide |
| K26 | 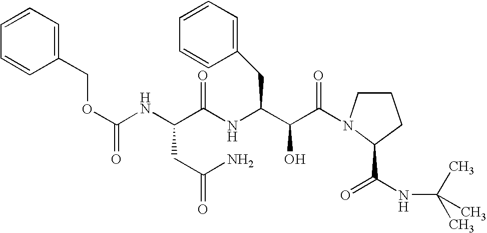 |
| | N\hat{ }2-[(phenylmethoxy)carbonyl]-L-asparaginyl- |
| | (2S,3S)-2-hydroxy-4-phenyl-3-aminobutanoyl- |
| | N-(1,1-dimethylethyl)-L-prolinamide |
| K27 | 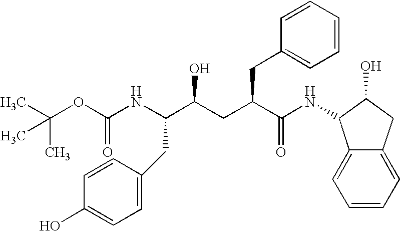 |
| | N-(2(R)-hydroxy-1(S)-indanyl)-5(S)-[(tert-butyloxycarbonyl) |
| | amino]-4(S)-hydroxy-6-(4-hydroxyphenyl)-2-(R)-(phenylmethyl) |
| | hexanamide |
| K28 | 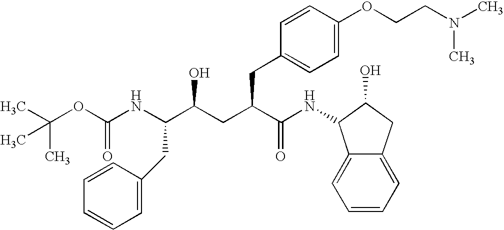 |
| | N-(2(R)-hydroxy-1(S)-indanyl)-5(S)-[(tert-butyloxycarbonyl) |
| | amino]-4(S)-hydroxy-2(R)-[[4-(2-dimethylamino)ethoxy]phenyl] |
| | methyl]-6-(phenyl)hexanamide |
| K29 | 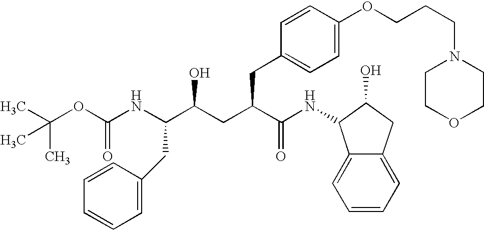 |
| | N-(2(R)-hydroxy-1(S)-indanyl)-5(S)-[(tert-bulyloxycarbonyl) |
| | amino]-4(S)-hydroxy-2(R)-[[4-[3-(4-morpholinyl)propoxy]phenyl] |
| | methyl]-6-phenyl-hexanamide |
| K30 | 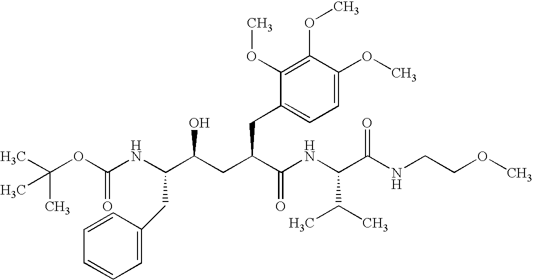 |
| | N-((1S)-1-[N-(2-methoxyethyl)carbamoyl]-2-methylpropyl)(4S, |
| | 5S,2R)-5-[(tert-butoxy)carbonylamino]-4-hydroxy-6-phenyl-2- |
| | [(2,3,4-trimethyoxyphenyl)methyl]hexanamide |
| K31 | 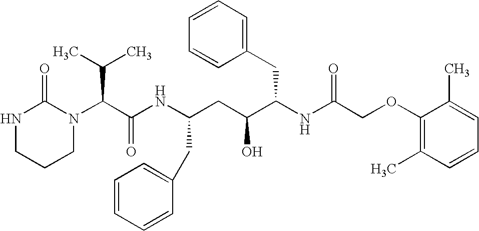 |
| | 1(2H)-pyrimidineacetamide,N-[(1S,3S,4S)-4-[[(2,6-dimethylphenoxy) |
| | acetyl]amino]-3-hydroxy-5-phenyl-1-(phenylmethyl)pentyl]tetrahydro- |
| | a-(1-methylethyl)-2-oxo-,(aS)- |
| K32 | 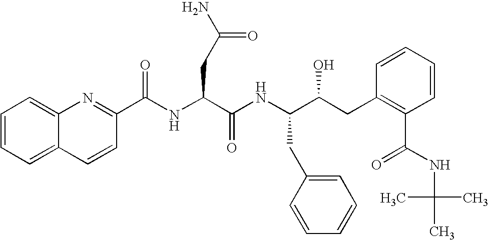 |
| | N\hat{ }1-[3-[2-[[(1,1-dimethylethyl)amino]carbonyl]phenyl]-2-hydroxy- |
| | 1-(phenylmethyl)propyl]-2-[(2-quinolinylcarbonyl)amino] |
| | butanediamide |
| K33 | 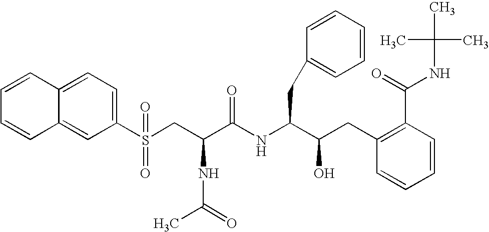 |
| | N-[3-[2-[[(1,1-dimethylethyl)amino]carbonyl]phenyl]-2-hydroxy-1- |
| | (phenylmethyl)propyl]-2(D)-(acetylamino)-3-(2-naphthalenylsulfon |
| | propanamide |
| K34 | 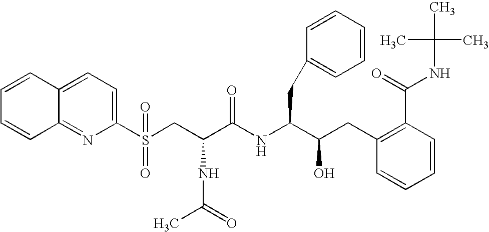 |
| | N-[3-[2-[[(1,1-dimethylethyl)amino]carbonyl]phenyl]-2(R)-hydroxy-1(S)- |
| | (phenylmethyl)propyl]-2(R)-(acetylamino)-3-(1-quinolinylsulfonyl) |
| | propanamide |
| K35 | 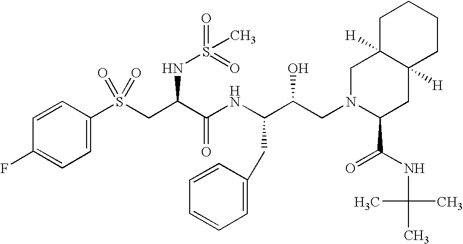 |
| | N-2-[2′(S)-hydroxy-3′(S)-phenylmethyl-4′-aza-5′-oxo-6′(S)-methylsulfonylamido- |
| | (4-fluorophenylsulfonyl)-heptyl]-(4aS,8aS)-decahydroisoquinoline-3(S)-N-t- |
| | butylcarboxamide |
| K36 | 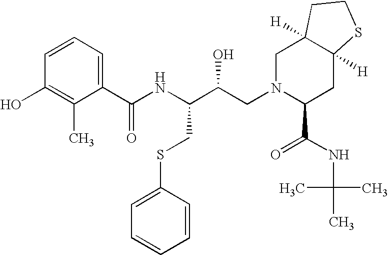 |
| | N-(1,1-dimethylethyl)-5-[2-hydroxy-3-[(3-hydroxy-2-methylbenzoyl)amino]- |
| | [phenyl(thiomethyl]propyl]octahydro-thieno[3,2-c]pyridine-6-carboxamide |
| K37 | 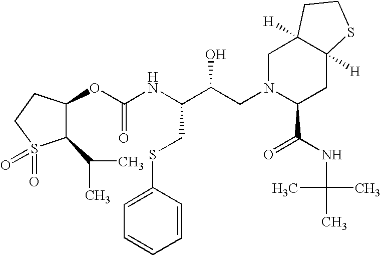 |
| | 5-[3(R)-[[(2(R)-cis-isopropyl-1,1-dioxotetrahydrothienyl-3(R)-oxy)carbon |
| | amino]-4-(phenylthio)-2(R)-hydroxybutyl]-N-(1,1-dimethylethyl) |
| | octahydrothieno[3,2-c]pyridine-6(R)-carboxamide |
| K38 | Loprnavir & Ritonavir |
| K39 | 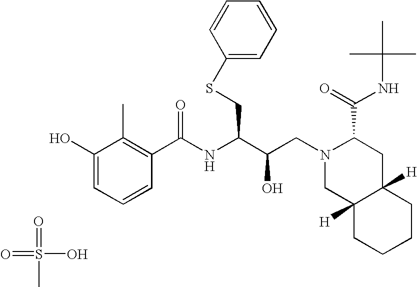 |
| | [(3S-(3R*,4aR*,8aR*,2′S*,3′S*)]-2-[2′-hydroxy-3′-phenylthiomethyl- |
| | 4′-aza-5′-oxo-5′-(2″-methyl-3″-hydroxy-phenyl)pentyl]- |
| | decahydroisoquinoline-3-N-t-butylcarboxamidemethanesulfonic ac |
| K40 | 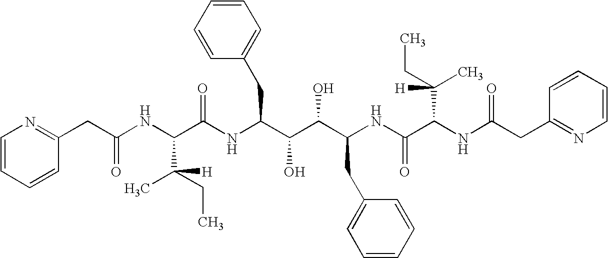 |
| K41 | 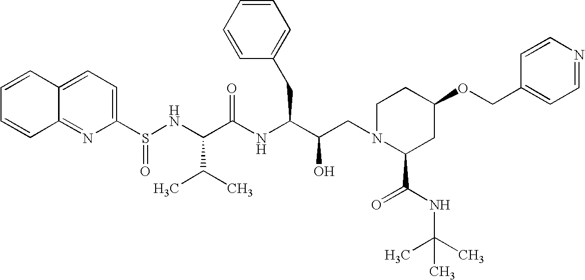 |
| | N-[1(S)-[[[3-[2(S)-[[1,1-dimethylethyl)amino]carbonyl]-4(R)-[(4-pyridinylmethyl) |
| | oxy]-1-piperidinyl]-2(R)-hydroxy-1(S)-(phenylmethyl)propyl]amino]carbonyl]-2- |
| | methylpropyl]-2-quinolinecarboxamide |
| K42 | 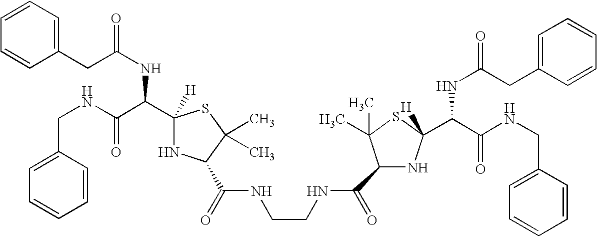 |
| | [2R-[2.alpha.(R*),4.beta.]]-4,4′-[1,2-ethanediylbis(aminocarbonyl) |
| | bis[N-benzyl-5, 5-dimethyl-.alpha.[(phenylacetyl)amino]-2- |
| | thiazolidineacetamide] |
| K43 | 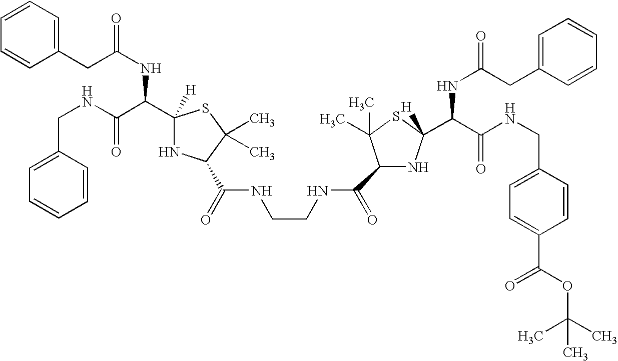 |
| | 2-thiazolidineacetamide,4-[[[2-[[[5,5-dimethyl-2-[2-oxo-1-[(phenylacetyl)amin- |
| | 2-[(phenylmethyl)amino]ethyl]-4-thiazolidinyl]carbonyl]amino]ethyl]amino] |
| | carbonyl]-5,5-dimethyl-.alpha.-[(phenylacetyl)amino]-N-[4(tert-butoxycarbon |
| | phenyl]methyl)-,[2R-[2.alpha.(R*),4.beta.[2R*(R*),4S*]]] |
| K44 | 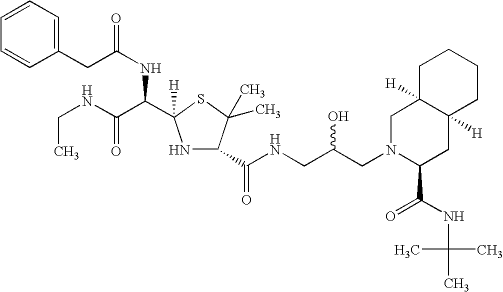 |
| | 4-[[[3-[3-[[1,1-dimethylethyl)amino]carbonyl]decahydro-2-isoquinolinyl]-2 |
| | hydroxypropyl]amino]carbonyl]-5,5-dimethyl-.alpha.-[(phenylacetyl)amino |
| | N-ethyl-2-thiazolidineacetamide,[2R-[2.alpha. |
| K45 | 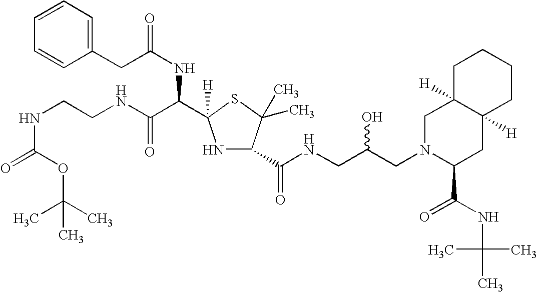 |
| | 4-[[[3-[3-[[(1,1-dimethylethyl)amino]carbonyl]decahydro-2-isoquinolinyl]-2 |
| | hydroxypropyl]amino]carbonyl]-5,5-dimethyl-.alpha.-[(phenylacetyl)amino |
| | N-[2-[[(1,1-dimethylethoxy)carbonyl]amino]ethyl]-2- |
| K46 | 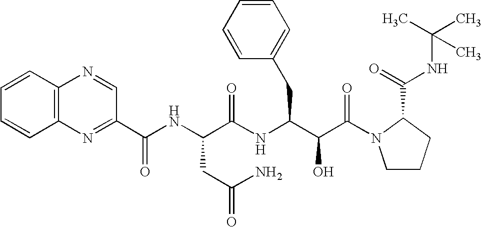 |
| | (2S,3S)-3-[N-(quinoxaline-2-carbonyl)-L-asparaginyl]amino- |
| | 2-hydroxy-4-phenylbutanoyl-L-proline, tert-butylamide |
| K47 | 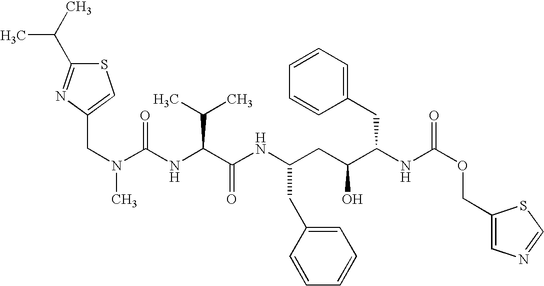 |
| | 2,4,7,12-tetraazatridecan-13-oic |
| | acid,10-hydroxy-2-methyl-5-(1-methylethyl)-1-[2-(1-methylethyl)- |
| | thiazolyl]-3,6-dioxo-8,11-bis(phenylmethyl)-,5-thiazolylmethyl |
| | ester,[5S-(5R*,8R*,10R*,11 R*)]- |
| K48 | 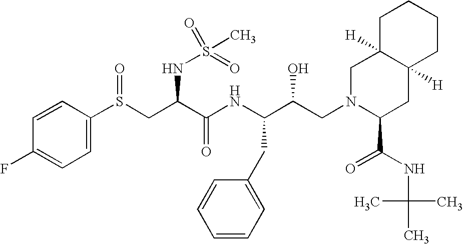 |
| | N-2-[2′(S)-hydroxy-3′(S)-phenylmethyl-4′-aza-5′-oxo-6′(S)-methylsulfonylamio |
| | 7′-(4-fluorophenylsulfinyl)-heptyl]-(4aS,8aS)-decahydroisoquinoline-3(S)- |
| | N-t-butylcarboxamide |
| K49 | 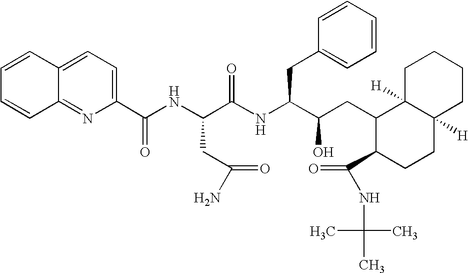 |
| | butanediamide,N1-[(1S,2R)-3-[(3S,4aS,8aS)-3-[[(1,1-dimethylethyl)amino |
| | carbonyl]octahydro-2(1H)-isoquinolinyl]-2-hydroxy-1-(phenylmethyl)propy |
| | 2-[(2-quinolinylcarbonyl)amino]-,(2S)- |
| K50 | 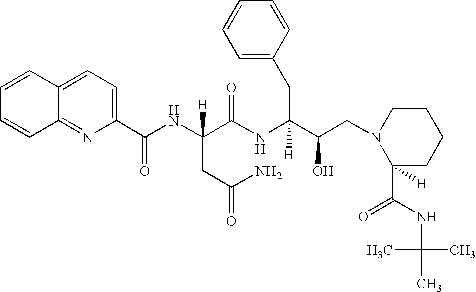 |
| | N-tert-butyl-1-[2(R)-hydroxy-3(S)-[[N-(2-quinolylcarbonyl)-L-asparagin |
| | amino]-4-phenylbutyl]-2(S)piperidinecarboxamide |
| K51 | 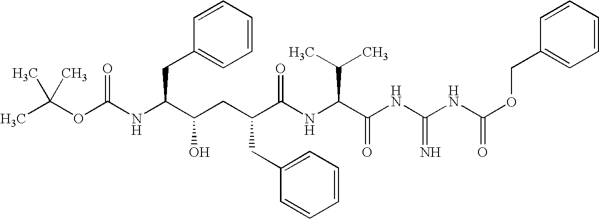 |
| | N-[(1S)-1-(N-(Imino[(phenylmethoxy)carbonylamino]methyl)carbomoyl)- |
| | methylpropyl](4S,5S,2R)-5-[(tert-butoxy)carbonylamino]-4-hydroxy-6- |
| | phenyl-2-benzylhexanamide |
| K52 | 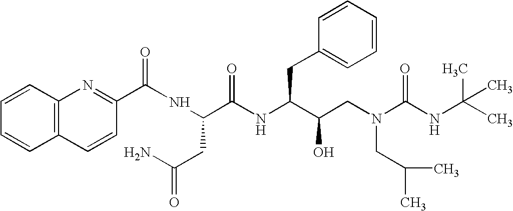 |
| | N-tert-butyl-N′-isobutyl-N′-[2(R)-hydroxy-4-phenyl-3(S)-[4-amino-1,4-diox |
| | 2(S)-(2-quinolinylcarboxamido)butylamino]butyl]urea |
| K53 | 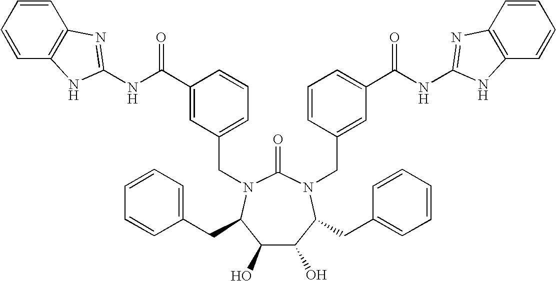 |
| | [4R-(4.alpha.,5.alpha.,6.beta.,7.beta.)]-3,3′-[[tetrahydro-5,6-dihydroxy-2-oxo-4 |
| | bis(phenylmethyl)-1H-1,3-diazepine-1,3(2H)-diyl]-bis(methylene)]bis[N-1H- |
| | benzimidazol-2-ylbenzamide] |
| K54 | 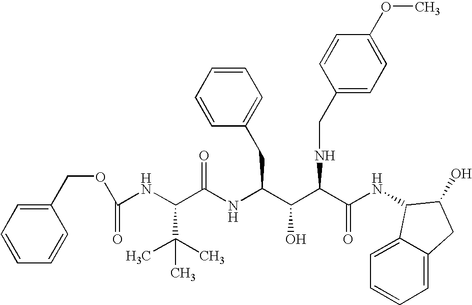 |
| | (2R,3S,4S,1′S,2′R)-4-[[[N-[(benzyloxy)carbonyl]-L-tert-leucyl]amino]-3-hydrox |
| | 2-[(4-methoxybenzyl)amino]-5-phenylpentan(2′-hydroxy-1′-indanyl)amide |
| K55 | 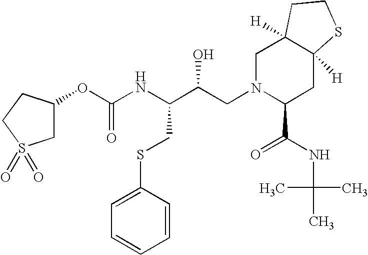 |
| | 5-[3(R)-[[(1,1-dioxotetrahydrothienyl-3(S)-oxy)carbonyl]amino]-4-(phenylthio)-2(R) |
| | hydroxybutyl]-N-(1,1-dimethylethyl)octahydrothieno[3,2-c]pyridine-6(R)-carboxam |
| K56 | 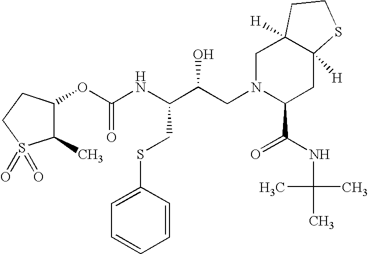 |
| | 5-[3(R)-[[(2(R)-cis-methyl-1,1-dioxotetrahydrothienyl-3(S)-oxy)carbonyl]amin |
| | 4-(phenylthio)-2(R)-hydroxybutyl]-N-(1,1-dimethylethyl)octahydrothieno[3,2-o |
| | pyridine-6(R)-carboxamide |
| K57 | 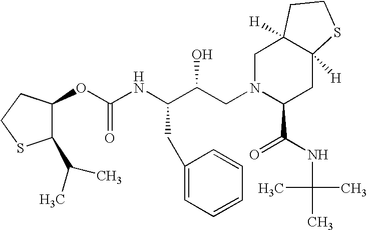 |
| | 5-[3(R)-[[(2(R)-cis-isopropyl-tetrahydrothienyl-3(R)-oxy)carbonyl]amino]-4-phenyl-2(R |
| | hydroxybutyl]-N-(1,1-dimethylethyl)octahydrothieno[3,2-c]pyridine-6(R)-carboxamide |
| K58 | 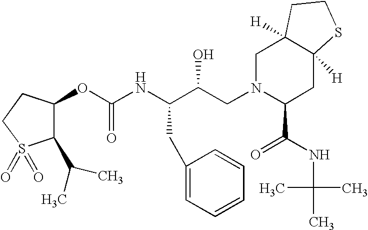 |
| | 5-[3(R)-[[(2(R)-cis-isopropyl-1,1-dioxotetrahydrothienyl-3(R)-oxy)carbonyl]amino]-4 |
| | phenyl-2(R)-hydroxybutyl]-N-(1,1-dimethylethyl)octahydrothieno[3,2-c]pyridine-6(R) |
| | carboxamide |
| K59 | 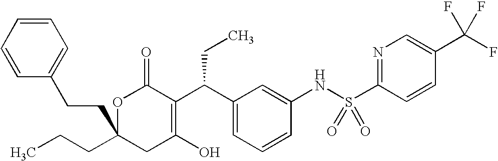 |
| | (6R)-3-((1R)-1-[3-(([5-(trifluoromethyl)(2-pyridyl)]sulfonyl)amino)phenyl]propy |
| | 4-hydroxy-6-(2-phenylethyl)-6-propyl-5,6-dihydro-2H-pyran-2-one |
| K60 | 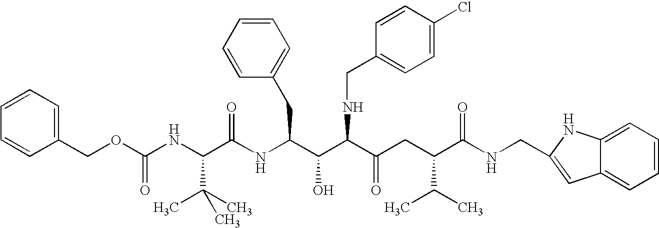 |
| | (2R,3S,4S)-N-[2-(4-chlorobenzylamino)-4-[[N-[(benzyloxy)carbonyl]tert- |
| | leucine]amino]-3-hydroxy-5-phenylpentanoyl]valine(2-benzimidazolyl) |
| | methylamide |
| K61 | 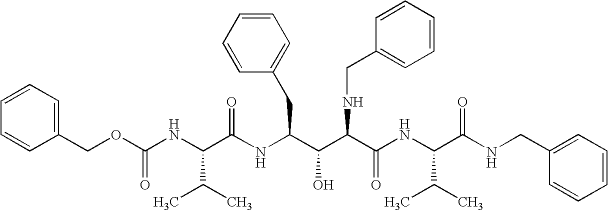 |
| | (2R,3S,4S)-N-[2-(benzylamino)-4-[[N-[(benzyloxy)carbonyl]valyl]amino |
| | 3-hydroxy-5-phenylpentanoyl]valine benzylamide |
| K62 | 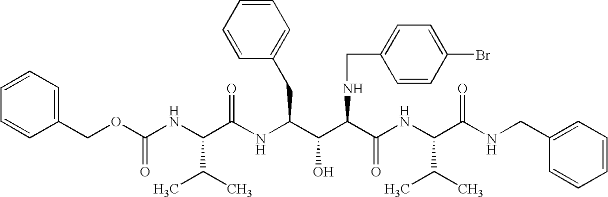 |
| | (2R,3S,4S)-N-[2-[(4-bromophenyl )methylaminol-4-[[N-[(benzyloxy) |
| | carbonyl]valyl]amino]-3-hydroxy-5-phenylpentanoyl]valinebenzylami |
| K63 | 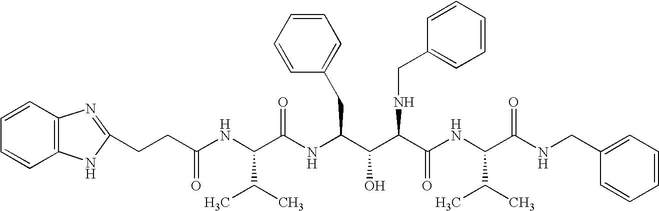 |
| | (2R,3S,4S)-N-[2-(benzylamino)-4-[[N-[(2-benzimidazolyl)propanoyl]valyl]amin |
| | 3-hydroxy-5-phenylpentanoy]valine benzylamide |
| K64 | 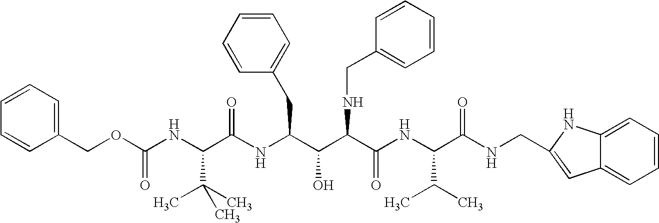 |
| | (2R,3S,4S)-N-[2-(benzylamino)-4-[[N-[(benzyloxy)carbonyl]valyl]amino]- |
| | 3-hydroxy-5-phenylpentanoyl]valine(2-benzimidazolyl)methylamide |
| K65 | 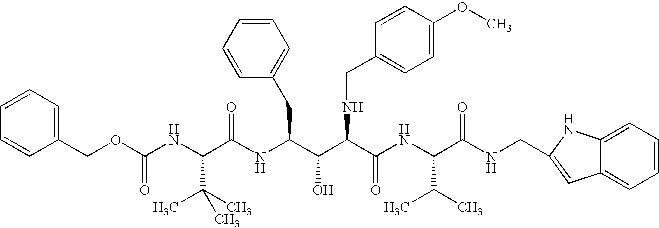 |
| | (2R,3S,4S)-N-[2-[(4-methoxybenzylamino]-4-[[N-[(benzyloxy)carbonyl] |
| | valyl]amino]-3-hydroxy-5-phenylpentanoyl]valine(2-benzimidazolyl) |
| | methylamide |
| K66 | 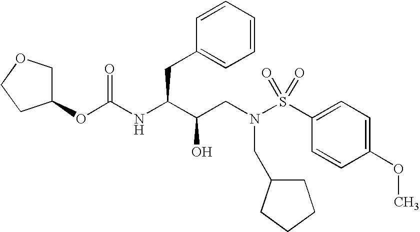 |
| | carbamic acid, [3-[[(4-methoxyphenyl)sulfonyl](cyclopentylmethyl)amino |
| | 2-hydroxy-1-(phenylmethyl)propyl]-tetrahydro-3-furanylester |
| K67 | 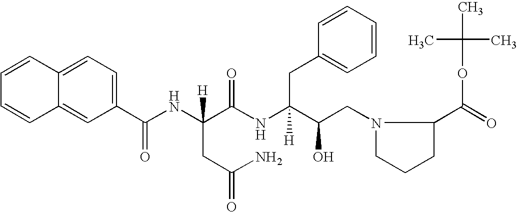 |
| | (2-naphthalcarbonyl)Asn[decarbonylphe-hydroxyethyl]ProtOtertbu |
| K68 | 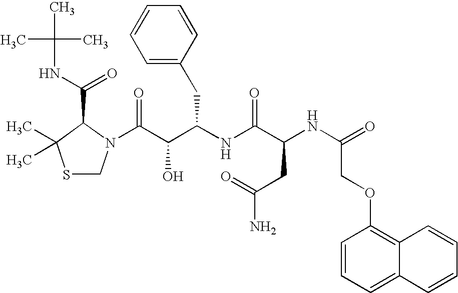 |
| | N\hat{ }1-[3-[4-[[(1,1-dimethylethyl)amino]carbonyl]-5,5-dimethyl-3-thiazolidinyl]- |
| | hydroxy-3-oxo-1-(phenylmethyl)propyl]-2-[[(1-naphthalenyloxy)acetyl]amino |
| | butanediamide,[4R-[3[1S*,(S*).2S*]],4R*]] |
| K69 | 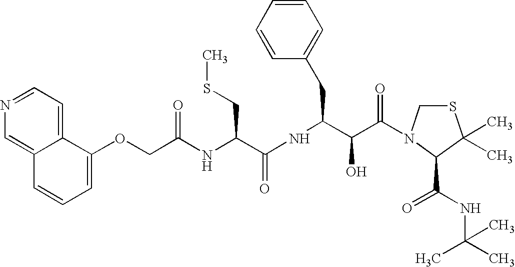 |
| | N-(1,1-dimethylethyl)-3-[2-hydroxy-3-[[2-[[(5-isoquinolinyloxy)acetyl]amino]- |
| | methylthio)-1-oxopropyl]amino]-1-oxo-4-phenylbutyl]-5,5-dimethyl]-4- |
| | thiazolidinecarboxamide,[4R-[3[2S*,3S*(R*)],4R*]] |
| K70 | 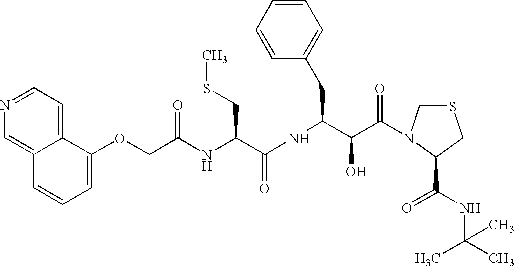 |
| | N-(1,1-dimethylethyl)-3-[2-hydroxy-3-[[2-[[(5-isoquinolinyloxy)acetyl]amino]-3- |
| | methylthio)-1-oxopropyl]amino]-1-oxo-4-phenylbutyl]-4-thiazolidinecarboxamie |
| | [4R-[3[2S*,3S*(R*)],4R*]] |
| K71 | 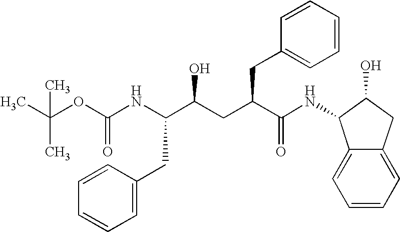 |
| | N-[2(R)-hydroxy-1(S)-indanyl]-5(S)-[[(1,1-dimethylethoxy)carbony |
| | amino]-4(S)-hydroxy-6-phenyl-2(R)-benzylhexanamide |
| K72 | 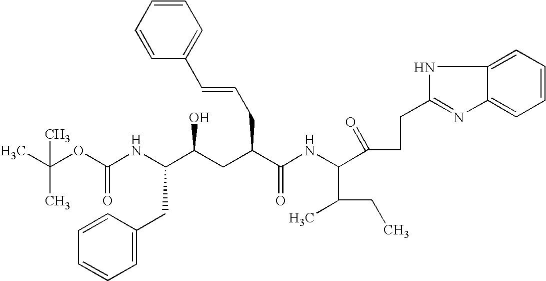 |
| | 6-phenyl-5-(N-t-butyloxycarbonylamino)-4-hydroxy-2-(3-phenylprop-2-ene)- |
| | [(2-(aminomethyl)benzimidazole)-isoleucyl]-hexanone |
| K73 | 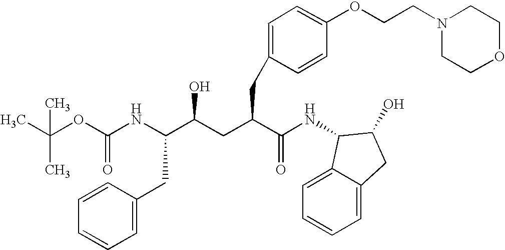 |
| | N-(2(R)-hydroxy-1(S)-indanyl)-5(S)-[(tert-butyloxycarbonyl)amino]-4(S)- |
| | hydroxy-6-phenyl-2(R)-[[4-[2-(4-morpholinyl)ethoxy]phenyl]methyl]hexanami |
| K74 | 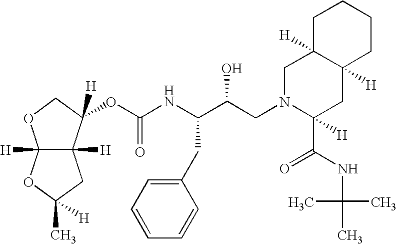 |
| | (3R,3aS,5S,6aR)-N-tert-butyl-2-[2′-hydroxy-4′-phenyl-3′-[[[(3′″-hexahydrofur |
| | [2,3-b]furanyl)oxy]carbonyl]amino]butyl]decahydroisoquinoline-3-carboxami |
| K75 | 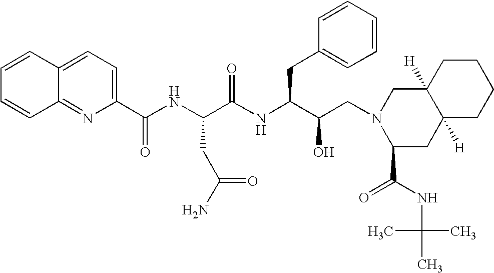 |
| | N1-[(1S,2R)-3-((3S,4aS,8aS)-3-[[(1,1-dimethylethyl)amino]carbonyl]octahydro- |
| | 2(1H)-isoquinolinyl]-2-hydroxy-1-(phenylmethyl)propyl]-2-[(2-quinolinylcarbonyl)amino],-(2S |

In yet another embodiment, the viral assembly inhibitor is a TAT inhibitor. TAT, short for transactivator of transcription, is a small HIV protein essential for both viral replication and the progression of HIV disease. Among its several postulated functions, TAT is known to bind to newly forming HIV transcripts to bring about dramatic changes in the entire process of HIV gene expression. By way of example, the binding of TAT to a newly forming HIV transcript may increase the transcription rate and the production of viral mRNA by a factor of many thousands, perhaps hundreds of thousands depending upon the particular transcript. By inhibiting the formation of these TAT/transcript complexes, the transcription of new HIV particles is significantly reduced. Any agent capable of inhibiting TAT may be utilized in the present invention. By way of example, suitable TAT inhibitors are listed in Table L.

TABLE L

| Compound | |
|---|---|
| No. | Compound |
| L1 | N2-acetyl-D-arginyl-D-arginyl-D-arginyl-D-arginyl-D-arginyl-D- |
| | arginyl-D-arginyl-D-arginyl-D-argininamide, nonaacetate |
| L2 | N-6-aminohexylglycine-N-guanidopropylglycine-N- |
| | guanidopropylglycine-N-benzylglycine-N-guanidopropylglycine-D- |
| | lysyl-D-lysyl-D-arginyl-D-prolylamide |
| L3 | 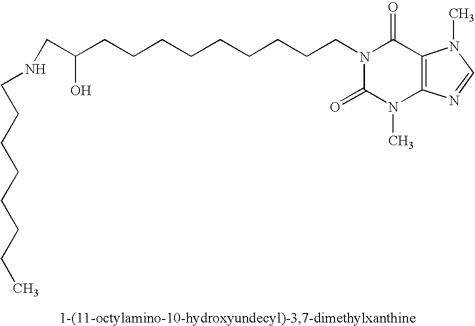 |
| L4 | 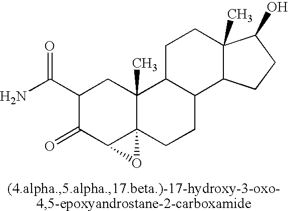 |
| L5 | 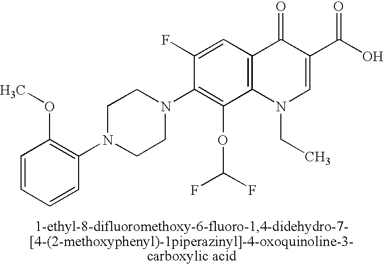 |
| L6 | 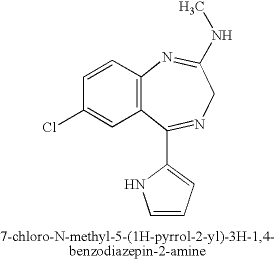 |
| L7 | 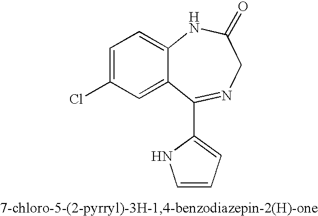 |
| L8 | 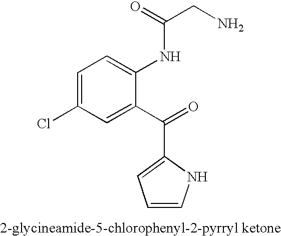 |

Another aspect of the invention encompasses anti-human immunodeficiency virus agents that are integrase inhibitors. Integrase is the HIV enzyme that catalyzes the integration of viral nucleic acid into the subject's own genetic material. These integrated viral genes in turn begin to churn out viral proteins and the new viral RNA needed for the assembly of large numbers of new viral particles. Inhibition of integrase, therefore, substantially slows or prevents HIV replication. Generally speaking, any agent capable of inhibiting HIV integrase may be employed in the present invention. For example, suitable integrase inhibitors are listed in Table M.

TABLE M

| Compound | |
|---|---|
| No. | Compound |
| M1 | 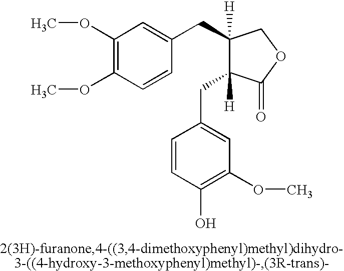 |
| M2 | 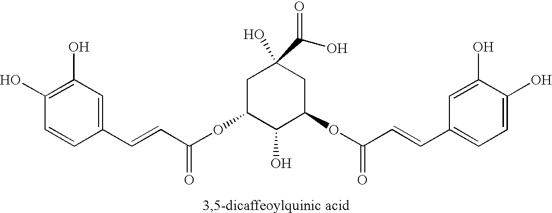 |
| M3 | 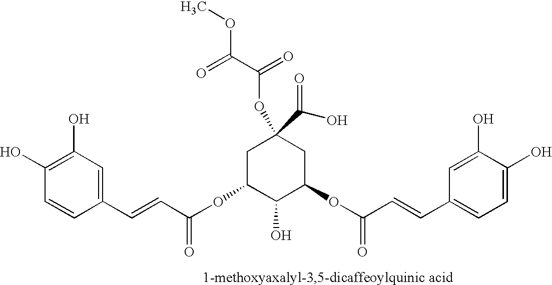 |
| M4 | 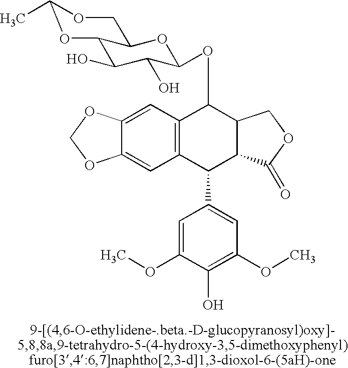 |
| M5 | Hydroxocobalamin |
| M6 | 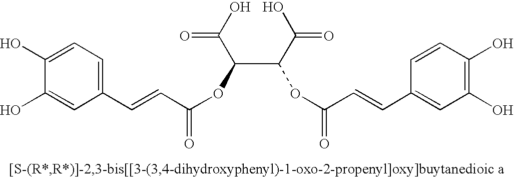 |

Another aspect of the invention encompasses anti-human immunodeficiency virus agents that are human immune enhancing agents. Typically, human immune enhancing agents allow the body to slow the progression of HIV by substantially increasing the immune response of the subject. In one embodiment, the human immune enhancing agent is an antioxidant. In general terms, antioxidants aide in eliminating free radicals that are byproducts of a number of reactions that normally occur in the body. If left unchecked, these free radicals not only compromise cell membrane integrity, but also mediate several disease states including cancer and neurological disorders. Typically, HIV infection results in higher levels of free radical formation in the subject. The administration of antioxidants, therefore, is believed to enhance the response of the subject against the virus by aiding in free radical elimination. Suitable agents for use as antioxidants are shown in Table N.

TABLE N

| Compound | |
|---|---|
| No. | Compound |
| N1 | 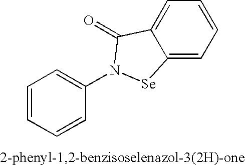 |
| N2 | 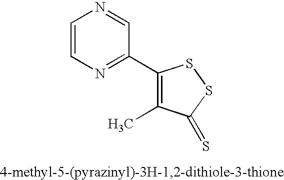 |

In another embodiment, the human immune enhancing agent is an interferon. Interferons are members of a family of glycoproteins, classified as cytokines. Interferon, like several other cytokines, prevent viral replication as well as stimulate other aspects of the subject's own immune system to fight HIV infection. By way of example, one mechanism by which these agents stimulate a subject's immune system is that they bind to specific receptors on cell surfaces, and thereby initiate a cascade of events, including induction of specific proteins. These proteins in turn, stimulate antiviral, antiproliferative, and other actions that mediate immune response. Any interferon that is effective in substantially preventing or inhibiting HIV infection may be employed. By way of example, suitable interferons for use in the present invention are shown in Table O.

TABLE O

| Compound | |
|---|---|
| No. | Compound |
| O1 | 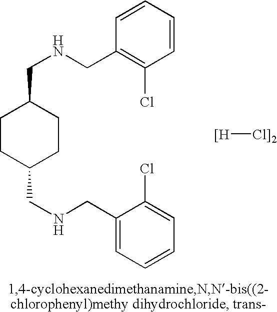 |
| O2 | 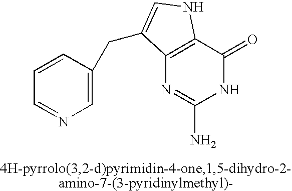 |
| O3 | 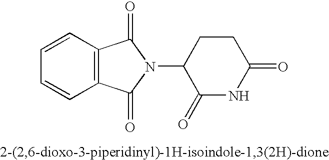 |

Another aspect of the invention encompasses anti-human immunodeficiency virus agents that are natural products. Any natural product that is effective in substantially preventing or inhibiting HIV infection may be employed. By way of example, suitable natural products are shown in Table P.

TABLE P

| Compound | |
|---|---|
| No. | Compound |
| P1 | Acemannan |
| P2 | 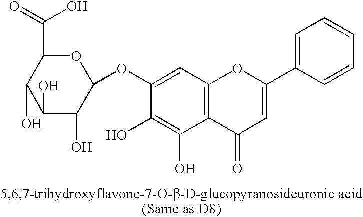 |
| P3 | 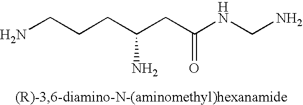 |
| P4 | 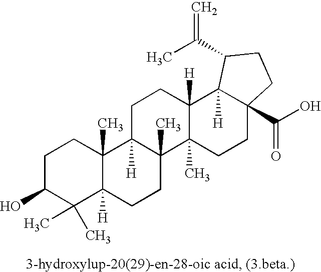 |
| P5 | 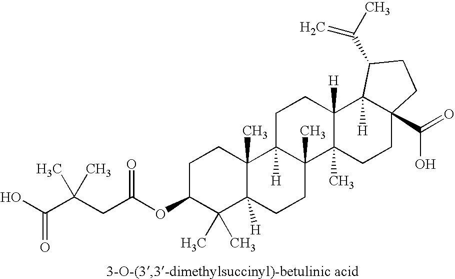 |
| P6 | 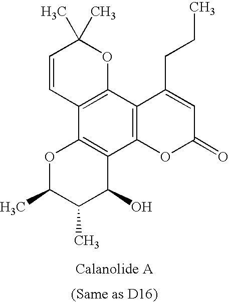 |
| P7 | 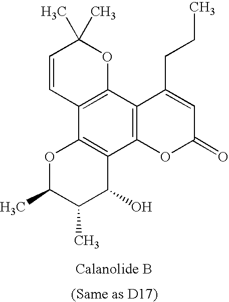 |
| P8 | 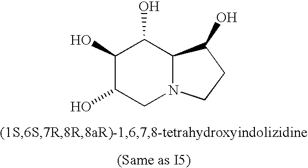 |
| P9 | Conocurvone |
| P10 | Cyanovirin-N |
| P11 | 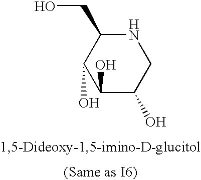 |
| P12 | 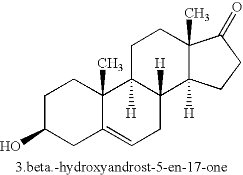 |
| P13 | 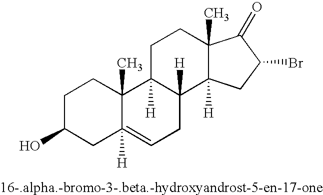 |
| P14 | 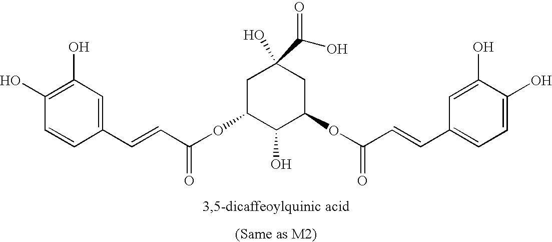 |
| P15 | 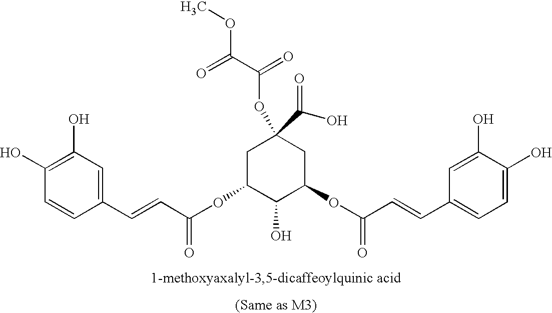 |
| P16 | 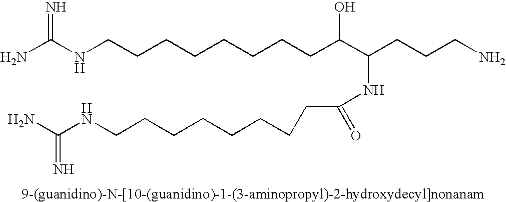 |
| P17 | 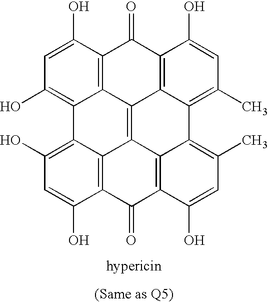 |
| P18 | 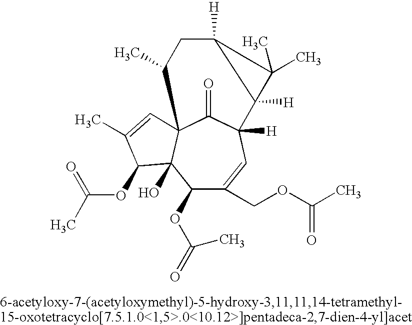 |
| P19 | 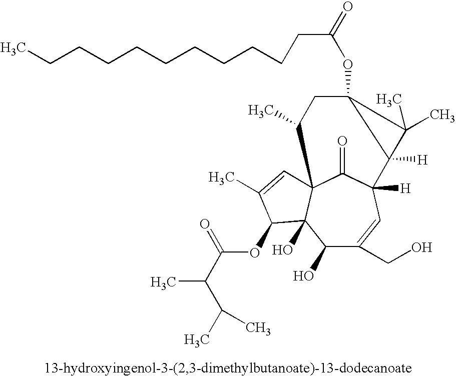 |
| P20 | 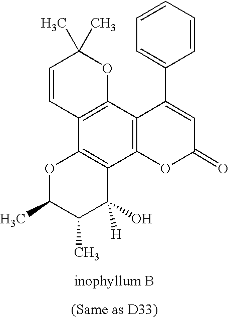 |
| P21 | 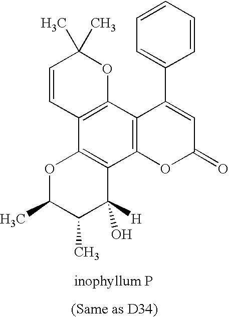 |
| P22 | 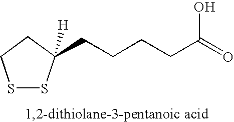 |
| P23 | 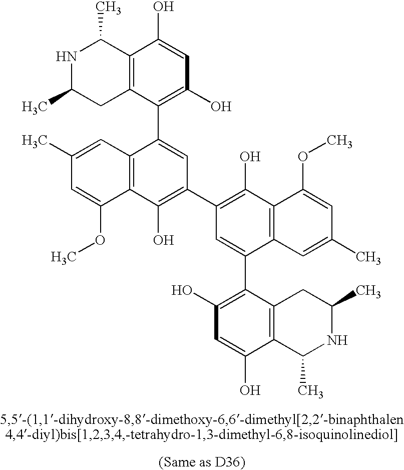 |
| P24 | 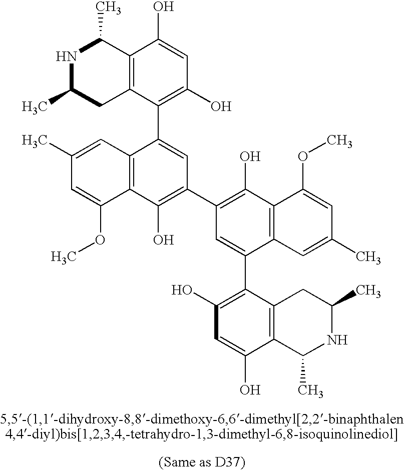 |
| P25 | 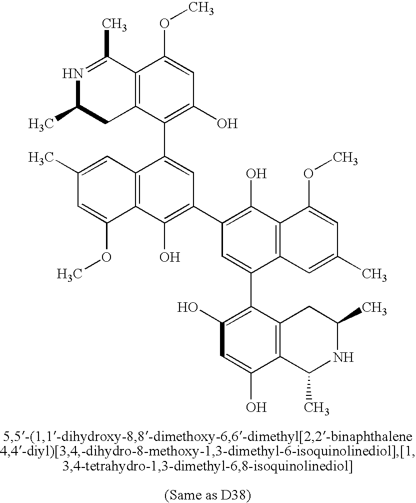 |
| P26 | 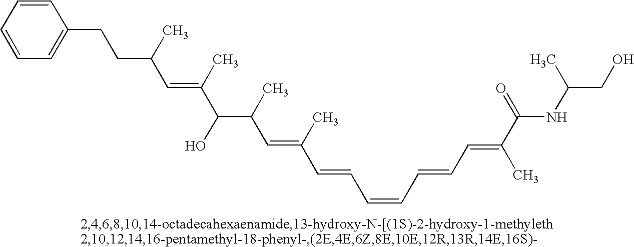 |
| P27 | 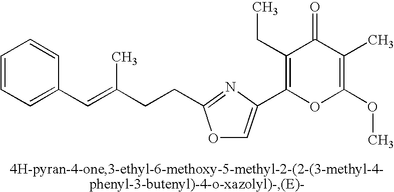 |
| P28 | 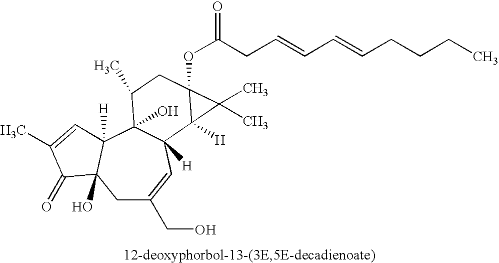 |
| P29 | 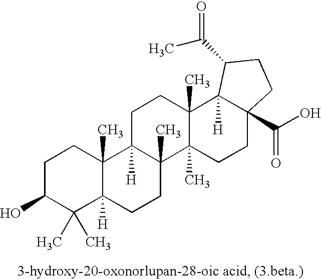 |
| P30 | 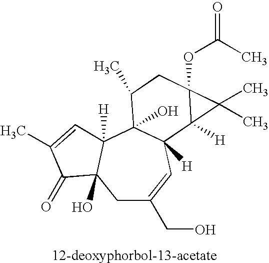 |
| P31 | 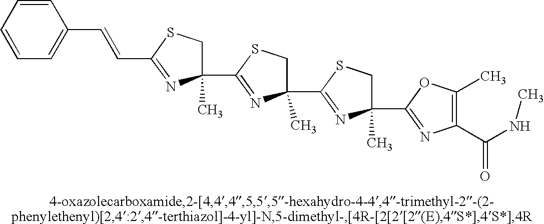 |

Another aspect of the invention encompasses anti-human immunodeficiency virus agents that are antimitotic agents. Antimitotic agents typically inhibit or prevent mitosis or nuclear division of the subject's cell. Generally speaking, these agents slow viral replication and concomitantly, viral growth, by preventing division of a subject's cells infected with HIV.

In one embodiment, the antimitotic agent is podophyllotoxin. Podophyllotoxin selectively arrests mitosis in the metaphase stage of infected cutaneous cells, causing necrosis of the infected cells. The podophyllotoxin may be obtained from a number of sources. For example, in one embodiment, the podophyllotoxin may be obtained from a number of commercially available sources sold under tradenames such as podofilox (brand name “Condylox®” supplied by Oclassen Pharmaceuticals, Inc.), which is a glucoside extract synthesized chemically or purified from the plant families Coniferae and Berberidaceae. In yet another embodiment, the podophyllotoxin may be obtained from podphyllum resin (brand name “Pod-Ben-25” or “Podofin®”), which is a powdered mixture of resins removed from Podophyllum peltatum (more commonly known as the mayapple or American mandrake), a pereninial plant in the Berberidaceae family and found in the woodlands in Canada and the Eastern United States. In another embodiment, the antimitotic agents are oxygenated esters of 4-idodophenylamino benzhydroxamic acid or derivatives thereof as disclosed in WO/002062 13, which is hereby incorporated by reference in its entirety. These agents inhibit MAP kinase, which is an enzyme essential for cellular proliferation. Inhibition of this enzyme completely arrests mitogenesis.

A further aspect of the invention encompasses anti-human immunodeficiency virus agents that are virucidal agents. Virucidal agents are competitive inhibitors of viral DNA polymerase. By way of example, in one embodiment, the virucidal agent is cidofovir. Cidofovir, (S)-1-(3-Hydroxy-2-phosphonylmethoxypropyl) cytosine (HPMPC), is an acyclic nucleoside phosphonate with broad-spectrum activity against a wide variety of DNA viruses, including HIV. The mechanism of action of Cidofovir is based upon the interaction of its active intracellular metabolite, the diphosphorylated HPMPC derivative HPMPCpp, with the viral DNA polymerase. HPMPCpp has been shown to block DNA synthesis by DNA chain termination following incorporation of two consecutive HPMPC moledules at the 3′-end of the DNA chain. Cidofovir can be obtained from commercial sources. In addition, other compounds suitable for use as virucidal agents in the present invention are shown in Table Q.

TABLE Q

| Compound | |
|---|---|
| No. | Compound |
| Q1 | 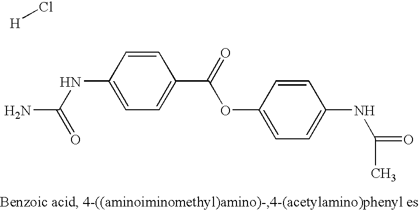 |
| Q2 | Cyanovirin-N |
| Q3 | 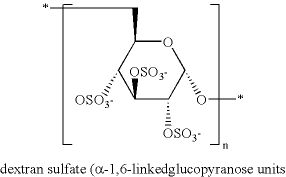 |
| Q4 | 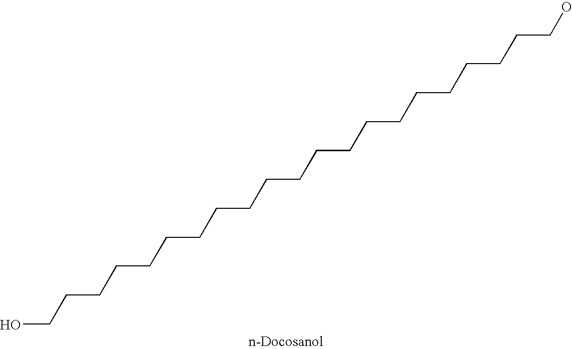 |
| Q5 | 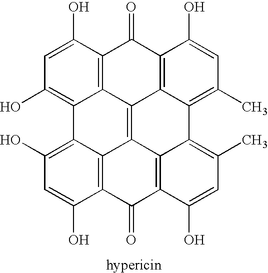 |
| Q6 | 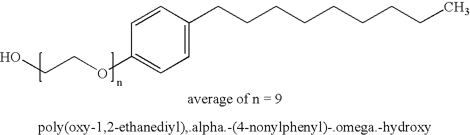 |
| Q7 | 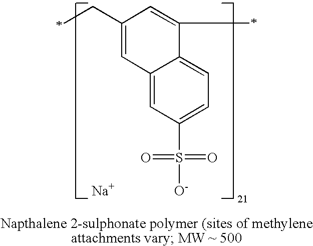 |
| Q8 | 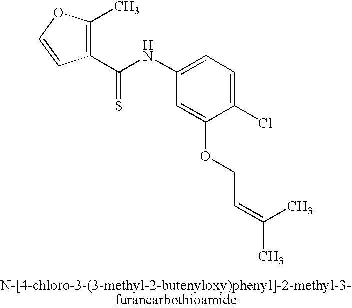 |

In yet a further aspect of the invention, the anti-human immunodeficiency virus agent is an antineoplastic agent. These agents reduce cell proliferation and thus arrest the growth of new cells or tissue, which may be benign or malignant. Although historically employed as a chemotherapeutic agent, antineoplastic agents may be effective against HIV. In one embodiment, the antineoplastic agent is 5-fluorouracil. 5-Fluorouracil (Efudex®, Adrucil®, Fluoroplex®) interferes with DNA synthesis by blocking the methylation of deoxyuridylic acid and inhibits thymidylate syntheses, which subsequently reduces cell proliferation. In another embodiment, the antineoplastic agent is an oxygenated ester of 4-iodophenylamino benzhydroxamic acid. These compounds are further described in WO/0206213, which is hereby incorporated by reference in its entirety. In yet another alternative of this embodiment, the antineoplastic agent is bleomycin (brand name “Blenoxane®). In addition, other compounds suitable for use as antineoplastic agents in the present invention are shown in Table R.

TABLE R

| Com- | |
|---|---|
| pound | |
| No. | Compound |
| R1 | 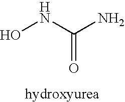 |
| R2 | 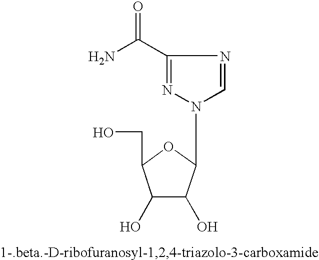 |
| R3 | 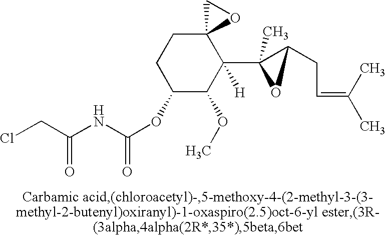 |

Of course, it will be apparent to those skilled in the art that it is possible, and perhaps desirable, to combine various classes of anti-human immunodeficiency virus agents for use in the present invention. Accordingly, it is contemplated that any class of anti-human immunodeficiency virus agent may be combined with one or more other classes to create a composition optimized for treating subjects having various stages of HIV progression. By way of example, one such composition may include an integrase inhibitor, a reverse transcriptase inhibitor, and a protease inhibitor. By way of further example, the composition may include a reverse transcriptase inhibitor, a protease inhibitor, and an interferon. A skilled artisan can readily design compositions having combinations of different classes of anti-human immunodeficiency virus agents so as to optimize treatment for a particular subject.

Generally speaking, the pharmacokinetics of the particular agent to be administered will dictate the most preferred method of administration and dosing regiment. The anti-human immunodeficiency virus agent can be administered as a pharmaceutical composition with or without a carrier. The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution), alcohols, gelatin, talc, viscous paraffin, fatty acid esters, hydroxymethylcellulose, polyvinyl pyrolidone, calcium carbonate, carbohydrates (such as lactose, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17.sup.th Ed., Mack Pub. Co., Easton, Pa.). Such preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active compounds. Typical preservatives can include, potassium sorbate, sodium metabisulfite, methyl paraben, propyl paraben, thimerosal, etc. The compositions can also be combined where desired with other active substances, e.g., enzyme inhibitors, to reduce metabolic degradation.

Moreover, the anti-human immunodeficiency virus agent can be a liquid solution, suspension, emulsion, tablet, pill, capsule, sustained release formulation, or powder. The method of administration can dictate how the composition will be formulated. For example, the composition can be formulated as a suppository, with traditional binders and carriers such as triglycerides. Oral formulation can include standard carriers such as pharmaceutical grades of mannitol, lactose, starch, magnesium stearate, sodium saccharine, cellulose, or magnesium carbonate.

In another embodiment, the anti-human immunodeficiency virus agent can be administered intravenously, parenterally, intramuscular, subcutaneously, orally, nasally, topically, by inhalation, by implant, by injection, or by suppository. For enteral or mucosal application (including via oral and nasal mucosa), particularly suitable are tablets, liquids, drops, suppositories or capsules. A syrup, elixir or the like can be used wherein a sweetened vehicle is employed. Liposomes, microspheres, and microcapsules are available and can be used. Pulmonary administration can be accomplished, for example, using any of various delivery devices known in the art such as an inhaler. See. e.g. S. P. Newman (1984) in Aerosols and the Lung, Clarke and Davis (eds.), Butterworths, London, England, pp. 197-224; PCT Publication No. WO 92/16192; PCT Publication No. WO 91/08760. For parenteral application, particularly suitable are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like.

The actual effective amounts of compound or drug can and will vary according to the specific composition being utilized, the mode of administration and the age, weight and condition of the subject. In general, as used herein, an effective amount of the anti-human immunodeficiency virus agent is an amount that achieves the desired degree of HIV treatment or prevention.

By way of example, in one embodiment, when the anti-human immunodeficiency virus agent is the nucleoside reverse transcriptase inhibitor Zidovudine administered to a human subject with HIV infection, it is typical that the amount used is approximately 0.5 to about 500 milligrams twice a day and more typically about 300 milligrams twice a day. In one alternative of this embodiment, when the nucleoside reverse transcriptase inhibitor Zalcitabine is administered to a human subject with HIV infection, it is typical that the amount used is approximately 0.2 to about 1.0 milligrams twice a day and even more commonly, about 0.75 milligrams three times a day. Table 1A below provides a comparison of some commonly employed nucleoside reverse transcriptase inhibitors.

TABLE 1A
Comparison of Nucleoside Reverse Transcriptase Inhibitors

| | Dosage | Preferred Dosing Method | Food Effect |
|---|---|---|---|
| Zidovudine | 300 | Tablets/ | None |
| | milligrams | Capsules | |
| | twice a day | | |
| Didanosine | >60 kg: 200 | Tablets/ | Take 1 hour |
| | milligrams | Capsules or oral | before or 1 hour |
| | twice a day | solution | after |
| | (tabs) or 250 | | meal |
| | milligrams | | |
| | twice a day | | |
| | (powder) | | |
| | <60 kg: 125 | | |
| | milligrams | | |
| | twice a day | | |
| | (tabs) or 167 | | |
| | milligrams | | |
| | twice a day | | |
| | (powder) | | |
| Zalcitabine | 0.75 | Tablets/ | None |
| | milligrams | Capsules | |
| | three times a | | |
| | day | | |
| Stavudine | >60 kg: 40 | Tablets/ | None |
| | milligrams | Capsules | |
| | twice a day | | |
| | <60 kg: 30 | | |
| | milligrams | | |
| | twice a day | | |
| Lamivudine | 150 | Tablets/ | None |
| | milligrams | Capsules | |
| | twice a day | | |
| Abacavir | 300 | Tablets/ | None |
| | milligrams | Capsules | |
| | twice a day | | |
| Tenofovir | 300 | Tablets/ | Should be |
| | milligrams | Capsules | taken with a |
| | everyday | | meal |

By way of further example, in one embodiment, when the anti-human immunodeficiency virus agent is the non-nucleoside reverse transcriptase inhibitor Delavirdine (Rescriptor®) administered to a human subject with HIV infection, it is typical that the amount used is approximately 0.5 to about 750 milligrams by mouth three times a day, and even more typically about 400 milligrams by mouth three times a day. In one alternative of this embodiment, when the non-nucleoside reverse transcriptase inhibitor Efavirenz (Sustiva®) is administered to a human subject with HIV infection, it is typical that the amount used is approximately 0.5 to about 1000 milligrams by mouth everyday at bedtime and even more commonly, about 600 milligrams by mouth everyday at bedtime. Table 1B below provides a comparison of commonly employed non-nucleoside reverse transcriptase inhibitors.

TABLE 1B
Comparison of Non-Nucleoside Reverse Transcriptase Inhibitors

| | Dosage | Preferred Dosing Method | Food Effect |
|---|---|---|---|
| Nevirapine | 200 | Tablets/ | None |
| | milligrams by | Capsules | |
| | mouth | | |
| | everyday × 14 | | |
| | days, then 200 | | |
| | milligrams by | | |
| | mouth twice a | | |
| | day | | |
| Delavirdine | 400 | Tablets/ | None |
| | milligrams by | Capsules | |
| | mouth three | | |
| | times a day | | |
| Efavirenz | 600 | Tablets/ | Avoid taking |
| | milligrams by | Capsules | after high fat |
| | mouth | | meals |
| | everyday at | | |
| | bedtime | | |

By way of yet further example, in one embodiment, when the anti-human immunodeficiency virus agent is the protease inhibitor Saquinavir (Fortovase®) administered to a human subject with HIV infection, it is typical that the amount used is approximately 0.5 to about 2000 milligrams twice a day and even more typically, about 1200 milligrams twice a day. In one alternative of this embodiment, when the protease inhibitor Nelfinavir (Viracept®) is administered to a human subject with HIV infection, it is typical that the amount used is 0.5 to about 2000 milligrams twice a day and even more typically, about 1200 milligrams twice a day. Table 1C below provides a comparison of commonly utilized protease inhibitors.

TABLE 1C
Comparison of Protease Inhibitors

| | | Preferred | |
|---|---|---|---|
| | Dosage | Dosing Method | Food Effect |
| Indinavir | 800 | Tablets/ | Take 1 hour |
| | milligrams | Capsules | before or 2 |
| | every 8 hours | | hour after |
| | | | meal |
| Ritonavir | 600 | Tablets/ | Take with |
| | milligrams | Capsules or oral | food if |
| | twice a day | solution | possible |
| Saquinavir | Not | Tablets/Capsules | None |
| (Invirase ®) | recommended | | |
| | as single | | |
| | PI | | |
| Saquinavir | 1200 | Tablets/Capsules | Take with |
| (Fortovase ®) | milligrams | | large meal |
| | three times | | |
| | a day | | |
| Amprenavir | 1200 | Tablets/ | Avoid taking |
| | milligrams | Capsules or | after high fat |
| | twice a day | Oral Solution | meals |
| | (caps) | | |
| | 1400 | | |
| | milligrams | | |
| | twice a day | | |
| | (oral solution) | | |
| Nelfinavir | 1250 | Tablets/ | Take with |
| | milligrams | Capusles | food if |
| | twice a day or | | possible |
| | 750 | | |
| | milligrams | | |
| | three times a | | |
| | day | | |
| Lopinavir + Ritonavir | 3 caps or 0.5 | Tablets/ | Take with |
| | milliliter | Capsules or | large meal |
| | twice daily | Oral Solution | |

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

### INDICATIONS TO BE TREATED OR PREVENTED

In one aspect of the invention, the composition is used to treat human immunodeficiency virus (HIV) during all stages of disease progression. The progression of HIV infection can be broken down to four primary stages. During the acute retroviral syndrome phase, which last one to two weeks, subjects infected with the virus experience nonspecific flu-like symptoms such as fever, headache, skin rash, tender lymph nodes, and a vague feeling of discomfort. Following the acute retroviral syndrome phase, infected subjects enter a prolonged asymptomatic phase. Subjects remain in good health during this period, with levels of CD4 T-cells ranging from low to normal. This phase can last for ten years or more. The third phase of HIV infection, the symptomatic phase, is characterized by rapidly falling levels of CD4 T-cells and opportunistic infections that are not life threatening. The symptomatic phase of HIV infection may last from a few months to several years. Advanced AIDS is the final phase of the disease. Death due to severe life threatening opportunistic infections and cancers usually occurs within one to two years.

The timing of the administration of the cyclooxygenase-2 selective inhibitor in relation to the administration of the anti-human immunodeficiency virus agent may also vary from subject to subject and depend upon the stage of disease being treated. In one embodiment of the invention, the cyclooxygenase-2 selective inhibitor and anti-human immunodeficiency virus agent may be administered substantially simultaneously, meaning that both agents may be administered to the subject at approximately the same time. For example, the cyclooxygenase-2 selective inhibitor or pharmaceutically acceptable salt or prodrug thereof is administered during a continuous period beginning on the same day as the beginning of the anti-human immunodeficiency virus agent and extending to a period after the end of the anti-human immunodeficiency virus agent. Alternatively, the cyclooxygenase-2 selective inhibitor and anti-human immunodeficiency virus agent may be administered sequentially, meaning that they are administered at separate times during separate treatments. In one embodiment, for example, the cyclooxygenase-2 selective inhibitor or a pharmaceutically acceptable salt or prodrug thereof is administered during a continuous period beginning prior to administration of the anti-human immunodeficiency virus agent and ending after administration of the anti-human immunodeficiency virus agent. Of course, it is also possible that the cyclooxygenase-2 selective inhibitor may be administered either more or less frequently than the anti-human immunodeficiency virus agent. One skilled in the art can readily design suitable treatment regiments for a particular subject depending on the particular stage of HIV infection being treated. Moreover, it will be apparent to those skilled in the art that it is possible, and perhaps desirable, to combine various times and methods of administration in the practice of the present invention.

A further aspect of the invention provides compositions to treat acquired immunodeficiency syndrome related disorders. As detailed above, as the HIV infection progresses from the first phase to the third phase, T-cell numbers rapidly decline and overall immune response in the subject is significantly compromised. Because of this diminished immune response, the subject is often plagued by a number of disorders that are effectively combated by the immune system of subjects not infected with HIV. These acquired immunodeficiency syndrome related disorders include one or more of the following conditions: skin rash, fever, muscle and joint aches, swelling of the lymph glands, seizures, hepatitis, diarrhea, shingles, herpes simplex infection, thrush, Kaposi's sarcoma, pneumocystis carinii pneumonia, cryptococcal meningitis, toxoplasmosis, mycobacterium avium complex, cytomegalovirus infection, and lymphoma. Accordingly, in addition to a cyclooxygenase-2 selective inhibitor and an anti-human immunodeficiency virus agent, it is contemplated that compositions of the invention may also include any other agent that helps ameliorate the opportunistic infections associated with HIV. Of course, the particular agent employed to combat the opportunistic infection will vary considerably and depend upon the disorder being treated and its stage of progression.

By way of example, when the acquired immunodeficiency syndrome related disorder is pneumocystis carinii pneumonia, the additional agent may include an antibiotic agent. In one embodiment, the antibiotic agent is a combination of trimethoprim (TMP) and sulfamethoxazole (SMX). In yet another embodiment, the antibiotic agent is Atovaquone. In still another embodiment, the antibiotic agent is Dapsone.

By way of further example, when the acquired immunodeficiency syndrome related disorder is toxoplasmosis, the additional agent may include an antiprotozoal agent. In one embodiment, the antiprotozoal agent is a combination of pyrimethamine (Daraprim®) and sulfadiazine. In yet a further embodiment, the antiprotozoal agent is a combination of pyrimethamine (Daraprim®) and clindamycin. In still a further embodiment, the antiprotozoal agent is a combination of pyrimethamine (Daraprim®), sulfadiazine, and Leucovorin.

By way of yet further example, when the acquired immunodeficiency syndrome related disorder is Kaposi's sarcoma or any other type of neoplasia or cancer, the additional agent may include an anti-neoplastic agent. In one embodiment, the antineoplastic agent is an antimetabolite including folate antagonists (e.g. methotrexate), pyrimidine antagonists (e.g. cytarabine, floxuridine, fludarabine, fluorouracil, and gemcitabine), purine antagonists (e.g. cladribine, mercaptopurine, thioguanine), and adenosine deaminase inhibitors (e.g. pentostatin). In an alternative embodiment, the antineoplastic agent is an alkylating agent such as chlorambucil, cyclophosphamide, busulfan, ifosfamide, melphalan, and thiotepa. In yet another embodiment, the antineoplastic agent is an akylator agent such as cisplatin, carboplatin, procarbazine, dacarbazine, and altretamine. In still another embodiment, the antineoplastic agent is an anti-tumor antibiotic such as bleomycin, dactinomycin, and mitomycin. In yet a further embodiment, the antineoplastic agent is an immunological agent such as interferon. In another embodiment, the antineoplastic agent is a plant alkaloid including vinca alkaloids (e.g. vinblastine vincristine and vinorelbine), epipodophyllotoxins (e.g. etoposide and teniposide), taxanes (e.g. docetaxel and paclitaxel), and camptothecins (e.g. topotecan and irinotecan). Of course those skilled in the art will appreciate that the particular antineoplastic agents to be administered with the composition of the invention will vary considerably depending on the type of neoplasia disorder being treated and its stage of progression.

By way of yet further example, when the acquired immunodeficiency syndrome related disorder is cryptococcal meningitis, the additional agent may include an antifungal agent. In one embodiment, the antifungal agent is fluconazole. In yet another embodiment, the antifungal agent is a combination of amphotericin B and flucytosine. In still another embodiment, the antifungal agent is slucytosine.

By way of still further example, when the acquired immunodeficiency syndrome related disorder is an immune mediated response such as skin rash, fever, muscle and joint aches, or swelling of the lymph glands, the additional agent may include an anti-inflammatory agent. In one embodiment, the anti-inflammatory agent is a non-steroidal anti-inflammatory agent. Suitable non-steroidal anti-inflammatory agents include naproxen sodium, diclofenac, suilindace, oxaprozin, diflunisal, aspirin, piroxicam, indomethocin, etodolac, ibuprofen, fenoprofen, ketoprofen, mefenamic acid, nabumetone, tolmetin sodium, and ketorolac tromethamine. In an alternative of this embodiment, the non-steroidal anti-inflammatory agent is acetaminophen. In another embodiment, the anti-inflammatory agent is a steroid.

### EXAMPLES

### Example 1

### Antiviral Therapy in Human Subjects

A human study can be performed according to any of the standard protocols. For example, a study can be conducted as described in, e.g., Kakuda et al., Antimicrobial Agents and Chemotherapy, Vol. 45, No.1, pp.236-242, January 2001. Prior to the initiation of a clinical study involving human subjects, the study should be approved by the appropriate Human Subjects Committee. Subjects are informed about the study and should give written consent prior to participation. The subjects selected for the study include HIV-infected persons (age from about 18 years to about 60 years) with particular characteristics selected for each study. For the purposes of the present study, plasma HIV RNA levels of≧5000 copies/ml and CD4 T lymphocyte counts of≧100 cells/μl are used to select appropriate subjects. Exclusion criteria will depend on a particular study. By way of example, exclusion criteria may include active opportunistic infections that would require interruption of antiretroviral therapy and known history of nonadherence with medication or scheduled physician and clinic visits. Furthermore, after enrollment, individuals missing scheduled clinic visits and not rescheduling within 1 week or in<85% adherence with their assigned regimen as assessed by medication counts or interview should be discontinued from the study.

The study can be designed as a randomized, open-label study comparing the standard antiretroviral therapy and a combination of a standard antiretroviral therapy and a Cox-2 inhibitor. Randomization can be performed by using a permuted block approach with assignments contained in sealed, opaque envelopes sequentially numbered. A standard therapy includes but is not limited to a combination of two nucleoside analogs plus one of the following: 1) the protease inhibitors indinavir or nelfinavir; 2) double-protease combinations of ritonavir plus saquinavir, indinavir or lopinavir; or 3) the non-nucleoside analog efavirenz. Nucleoside analogs include but are not restricted to zidovudine (AZT, ZDV, Retrovir®), Didanosine (ddl, Videx®, Videx EC®), Stavudine (d4T, Zerit®), Zalcitabine (ddC, Hivid®), Lamivudine (3TC, Epivir®), Abacavir (ABC, Ziagen®), and Tenofovir (Viread®). As an example, a standard antiretroviral therapy may consist of AZT, ddC and indinavir. Cox-2 inhibitors tested in combination with the antiretroviral therapy may include any of the Cox-2 inhibitors of the present invention. By way of example, Cox-2 inhibitors include celecoxib, rofecoxib, and valdecoxib.

An exemplary study can be designed to compare the effects of a combination of AZT, ddC and indinavir and a combination of AZT, ddC, indinavir and celecoxib. It should be noted that these particular drug combinations are only listed as examples, and that a number of other drug combinations disclosed herein may be tested. Furthermore, it should be noted that the dosages used will depend on multiple factors, such as a particular drug, the age of the patient, the presence of other conditions, etc. A skilled artisan can readily determine drug-dosing requirements for a particular study.

The initial phase of the study may be designed to last about 6 months, with long-term follow-up studies designed separately, if the initial phases of treatment appear to be successful.

All participants are initially treated with lamivudine (e.g. 150 mg twice daily) and indinavir (800 mg every 8 hours) for the first two weeks. Zidovudine may be started at a dose of 100 mg twice daily for the first week and then increased to 200 mg twice daily for the second week to minimize gastrointestinal side effects. At week 2, patients are randomized to either standard therapy consisting of zidovudine (300 mg twice daily), lamivudine (150 mg twice daily) and indinavir (800 mg every 8 hours) or the same in combination with celecoxib. The amount of celecoxib is preferably between about 1 to about 20 mg/day kg. A skilled artisan conducting the study can determine the appropriate amount of celecoxib for the subjects involved in the study.

Patients are not allowed to eat 1 hour before or 2 hours after ingestion of their medications since food has been shown to affect absorption of these drugs. Laboratory evaluations are performed prior to treatment and at clinical visits at weeks 2, 4, 8, 12, 16, 20, and 24. Blood samples are preferably obtained between 2 and 5 hours following drug administration. This time frame is chosen to avoid the absorption phase and obtain post-absorption concentrations within an optimal window, as assessed by p-optimality criteria, as previously described for AZT (see, e.g., Noormohamed et al., Antimicrob. Agents Chemother., 39:2792-2797, 1995). A clinical assessment and measurement of hematologic parameters and clinical chemistries are performed with every clinical visit. Urinalysis and cholesterol and triglyceride analyses are performed, e.g., every 4 weeks. Adverse reactions are graded and managed according to the approach developed by the AIDS Clinical Trials Group (Division of AIDS, 1996, Division of AIDS table for grading severity of adult adverse experiences, Division of AIDS, NIH, Rockville, Md.). CD4 lymphocytes and plasma HIV RNA are measured at baseline and every 4 weeks during the study. Plasma HIV RNA can be measured by using, e.g., Roche Amplicor Ultrasensitive Assay.

All of the above-mentioned parameters of clinical assessment are used to determine the efficacy of administering a combination of standard antiretroviral therapy and a Cox-2 inhibitor compared to the administration of the same antiretroviral therapy alone.

Statistical analyses can be performed using standard methods. For example, assessment of adherence data can be analyzed using ANOVA. Baseline patient characteristics can be evaluated with the Mann-Whitney U test. A sufficient sample size can be readily determined by a skilled artisan conducting the study. For all statistical analyses, a P value of<0.05 is considered significant.

### Example 2

### Antiviral Therapy in Rhesus Macaques

The following describes a study that can be performed in rhesus macaques. Such study can be performed as described in, e.g., Uberla et al., Proc. Natl. Acad. Sci USA, Vol. 92, pp.8210-8214, August 1995. Other study designs known in the art may also be used.

Numerous drugs approved for antiretroviral therapy of the human immunodeficiency virus type 1 (HIV-1) inhibit the viral reverse transcriptase (RT). Infection of macaques with simian immunodeficiency virus (SIV) closely mimics HIV-1 infection in humans and SIV-infected macaques develop a disease similar to the acquired immunodeficiency syndrome (AIDS), thus allowing for the study of antiviral drugs in these animals. However, the infection of macaques with SIV has some limitations. Reverse transcriptases of HIV-1 and SIV are approximately 60% homologous and differ in their susceptibility to non-nucleoside RT inhibitors. Furthermore, the development of resistance to antiretroviral drugs is likely to result from different mutations in HIV-1 and SIV reverse transcriptases. Uberla et al. have developed a recombinant SIV/HIV-1 hybrid virus that is well suited for study in monkeys (Uberla et al., Proc. Natl. Acad. Sci. USA, Vol. 92, pp. 8210-8214, August 1995). This virus, named RT-SHIV is a SIV strain (SIVmac239), whose RT was replaced with HIV-1 RT from the HxB2 clone. The virus was well characterized, and the studies showed that this chimeric virus was replication-competent in rhesus monkey peripheral blood mononuclear cells (PBMC). SDS/polyacrylamide analysis revealed that RT-SHIV only differed from SIV in the size of RT subunits. Furthermore, in vitro tests showed that the HIV-1 specific RT inhibitor Nevirapine could inhibit the RT activity and replication of RT-SHIV.

Rhesus macaques that are used in the study are housed and fed according to standard protocols. Handling of the monkeys and collection of specimens may be performed according to institutional guidelines where the monkeys are housed.

A number of macaques are infected with the same dose of RT-SHIV intravenously, whereas several macaques are left uninfected to be used as negative controls. Monkeys that are infected are started with the therapy following infection. The timing of the therapy initiation can be determined by a skilled artisan based on a particular study.

Half of the infected monkeys receive a standard antiretroviral therapy, whereas the other half receive the same standard antiretroviral therapy and a Cox-2 inhibitor. The non-limiting combinations of antiretroviral agents and Cox-2 inhibitors are described in Example 1. However, it should be noted that any of the antiretroviral agents and Cox-2 inhibitors of the present invention may be used in the study. Furthermore, dosages of drugs can be adjusted by a skilled artisan conducting the study in order to obtain maximal therapeutic results. For example, drugs to be tested can be titrated in macaques prior to the initiation of the study in order to determine effective dosages of said drugs.

The drugs are preferably given by intravenous route. The duration of the study can vary and can be determined by one of ordinary skill in the art. By way of example, the treatment can be administered every 8 hours for 15 days.

Sera are collected at regular intervals, and the p27 antigen and anti-SIV antibodies are determined as described previously (see, e.g., Lundgren et al., J. Acquired Immune Defic. Syndr., 4:489-498, 1991, and Thorstensson et al., J. Acquired Immune Defic. Syndr., 4:374-379, 1991). The p27 antigen can be determined using, e.g. an antigen capture assay (such as the one commercially available from Coulter). The CD4/CD8 ratio in the PBMC of treated macaques can be determined by fluorescence-activated cell-sorting (FACS) analyses by using labeled anti-CD4 and anti-CD8 antibodies according to standard protocols.

The obtained data can be used to determine the efficacy of the combination therapy comprising a standard antiretroviral regimen and a Cox-2 inhibitor.

1. A composition comprising an anti-human immunodeficiency virus agent and a cyclooxygenase-2 selective inhibitor or a pharmaceutically acceptable salt, ester, isomer or prodrug thereof.

2. The composition of claim 1 wherein the cyclooxygenase-2 selective inhibitor comprises a chromene compound.

3. The composition of claim 2 wherein the chromene compound is a benzopyran or substituted benzopyran analog.

4. The composition of claim 3 wherein the benzopyran or substituted benzopyran analog is selected from the group consisting of benzothiopyrans, dihydroquinolines and dihydronaphthalenes.

5. The composition of claim 1 wherein the cyclooxygenase-2 selective inhibitor comprises a tricyclic compound.

6. The composition of claim 5 wherein the tricyclic compound comprises a benzenesulfonamide or methylsulfonylbenzene.

7. The composition of claim 1 wherein the cyclooxygenase-2 selective inhibitor comprises a phenyl acetic acid derivative.

8. The composition of claim 1 wherein the cyclooxygenase-2 selective inhibitor comprises: 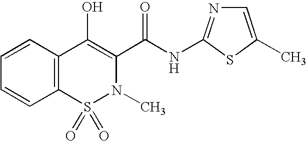
or pharmaceutically acceptable salt, ester, isomer or prodrug thereof.

9. The composition of claim 1 wherein the cyclooxygenase-2 selective inhibitor comprises: 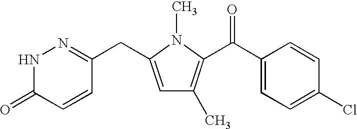
or a pharmaceutically acceptable salt, ester, isomer or prodrug thereof.

10. The composition of claim 1 wherein the cyclooxygenase-2 selective inhibitor comprises a compound of the formula or a pharmaceutically acceptable salt or prodrug thereof: 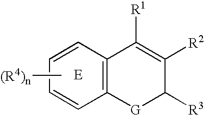
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; and
R⁴ together with the carbon atoms to which it is attached and the remainder of ring E forms a naphthyl radical.

11. The composition of claim 10, wherein:
n is an integer which is 0, 1, 2, 3 or 4;
G is O, S or NR^{b};
R¹ is H;
R^{b} is alkyl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

12. The composition of claim 10, wherein:
n is an integer which is 0, 1, 2, 3 or 4;
G is oxygen or sulfur;
R¹ is H;
R² is carboxyl, lower alkyl, lower aralkyl or lower alkoxycarbonyl;
R³ is lower haloalkyl, lower cycloalkyl or phenyl; and
each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or
wherein R⁴ together with the carbon atoms to which it is attached and the remainder of ring E forms a naphthyl radical.

13. The composition of claim 10, wherein:
R² is carboxyl;
R³ is lower haloalkyl; and
each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

14. The composition of claim 10, wherein:
n is an integer which is 0, 1, 2, 3 or 4;
R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and
each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

15. The composition of claim 10 wherein the cyclooxygenase-2 selective inhibitor comprises a compound of the formula or a pharmaceutically acceptable salt or prodrug thereof: 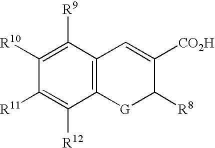
G is oxygen or sulfur;
R⁸ is trifluoromethyl or pentafluoroethyl;
R⁹ is H, chloro, or fluoro;
R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;
R¹¹ is H, methyl, ethyl, isopropyl, tert-butyl, chloro, methoxy, diethylamino, or phenyl; and
R¹² is H, chloro, bromo, fluoro, methyl, ethyl, tert-butyl, methoxy, or phenyl.

16. The composition of claim 10 wherein the cyclooxygenase-2 selective inhibitor, pharmaceutically acceptable salt, isomer or prodrug thereof is selected from the group consisting of:
6-chloro-2-trifluoromethyl-2H-1-benzopyran-3-carboxylic acid;
6-chloro-7-methyl-2-trifluoromethyl-2H-1-benzopyran-3-carboxylic acid;
8-(1-methylethyl)-2-trifluoromethyl-2H-1-benzopyran-3-carboxylic acid;
6-chloro-7-(1,1-dimethylethyl)-2-trifluoromethyl-2H-1-benzopyran-3-carboxylic acid;
6-chloro-8-(1-methylethyl)-2-trifluoromethyl-2H-1-benzopyran-3-carboxylic acid;
2-trifluoromethyl-3H-naphthopyran-3-carboxylic acid;
7-(1,1-dimethylethyl)-2-trifluoromethyl-2H-1-benzopyran-3-carboxylic acid;
6-bromo-2-trifluoromethyl-2H-1-benzopyran-3-carboxylic acid;
8-chloro-2-trifluoromethyl-2H-1-benzopyran-3-carboxylic acid;
6-trifluoromethoxy-2-trifluoromethyl-2H-1-benzopyran-3-carboxylic acid;
5,7-dichloro-2-trifluoromethyl-2H-1-benzopyran-3-carboxylic acid;
8-phenyl-2-trifluoromethyl-2H-1-benzopyran-3-carboxylic acid;
7,8-dimethyl-2-trifluoromethyl-2H-1-benzopyran-3-carboxylic acid;
6,8-bis(dimethylethyl)-2-trifluoromethyl-2H-1-benzopyran-3-carboxylic acid;
7-(1-methylethyl)-2-trifluoromethyl-2H-1-benzopyran-3-carboxylic acid;
7-phenyl-2-trifluoromethyl-2H-1-benzopyran-3-carboxylic acid;
6-chloro-7-ethyl-2-trifluoromethyl-2H-1-benzopyran-3-carboxylic acid;
6-chloro-8-ethyl-2-trifluoromethyl-2H-1-benzopyran-3-carboxylic acid;
6-chloro-7-phenyl-2-trifluoromethyl-2H-1-benzopyran-3-carboxylic acid;
6,7-dichloro-2-trifluoromethyl-2H-1-benzopyran-3-carboxylic acid;
6,8-dichloro-2-trifluoromethyl-2H-1-benzopyran-3-carboxylic acid;
2-trifluoromethyl-3H-naptho[2,1-b]pyran-3-carboxylic acid;
6-chloro-8-methyl-2-trifluoromethyl-2H-1-benzopyran-3-carboxylic acid;
8-chloro-6-methyl-2-trifluoromethyl-2H-1-benzopyran-3-carboxylic acid;
8-chloro-6-methoxy-2-trifluoromethyl-2H-1-benzopyran-3-carboxylic acid;
6-bromo-8-chloro-2-trifluoromethyl-2H-1-benzopyran-3-carboxylic acid;
8-bromo-6-fluoro-2-trifluoromethyl-2H-1-benzopyran-3-carboxylic acid;
8-bromo-6-methyl-2-trifluoromethyl-2H-1-benzopyran-3-carboxylic acid;
8-bromo-5-fluoro-2-trifluoromethyl-2H-1-benzopyran-3-carboxylic acid;
6-chloro-8-fluoro-2-trifluoromethyl-2H-1-benzopyran-3-carboxylic acid;
6-bromo-8-methoxy-2-trifluoromethyl-2H-1-benzopyran-3-carboxylic acid;
6-[[(phenylmethyl)amino]sulfonyl]-2-trifluoromethyl-2H-1-benzopyran-3-carboxylic acid;
6-[(dimethylamino)sulfonyl]-2-trifluoromethyl-2H-1-benzopyran-3-carboxylic acid;
6-[(methylamino)sulfonyl]-2-trifluoromethyl-2H-1-benzopyran-3-carboxylic acid;
6-[(4-morpholino)sulfonyl]-2-trifluoromethyl-2H-1-benzopyran-3-carboxylic acid;
6-[(1,1-dimethylethyl)aminosulfonyl]-2-trifluoromethyl-2H-1-benzopyran-3-carboxylic acid;
6-[(2-methylpropyl)aminosulfonyl]-2-trifluoromethyl-2H-1-benzopyran-3-carboxylic acid;
6-methylsulfonyl-2-trifluoromethyl-2H-1-benzopyran-3-carboxylic acid;
8-chloro-6-[[(phenylmethyl)amino]sulfonyl]-2-trifluoromethyl-2H-1-benzopyran-3-carboxylic acid;
6-phenylacetyl-2-trifluoromethyl-2H-1-benzopyran-3-carboxylic acid;
6,8-dibromo-2-trifluoromethyl-2H-1-benzopyran-3-carboxylic acid;
8-chloro-5,6-dimethyl-2-trifluoromethyl-2H-1-benzopyran-3-carboxylic acid;
6,8-dichloro-(S)-2-trifluoromethyl-2H-1-benzopyran-3-carboxylic acid;
6-benzylsulfonyl-2-trifluoromethyl-2H-1-benzopyran-3-carboxylic acid;
6-[[N-(2-furylmethyl)amino]sulfonyl]-2-trifluoromethyl-2H-1-benzopyran-3-carboxylic acid;
6-[[N-(2-phenylethyl)amino]sulfonyl]-2-trifluoromethyl-2H-1-benzopyran-3-carboxylic acid;
6-iodo-2-trifluoromethyl-2H-1-benzopyran-3-carboxylic acid;
7-(1,1-dimethylethyl)-2-pentafluoroethyl-2H-1-benzopyran-3-carboxylic acid; and
6-chloro-2-trifluoromethyl-2H-1-benzothiopyran-3-carboxylic acid.

17. The composition of claim 10 wherein the cyclooxygenase-2 selective inhibitor, pharmaceutically acceptable salt or prodrug thereof is selected from the group consisting of formulas: 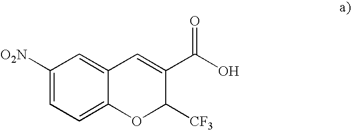
6-Nitro-2-trifluoromethyl-2H-1-benzopyran-3-carboxylic acid; 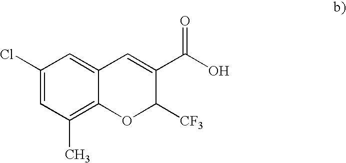
6-Chloro-8-methyl-2-trifluoromethyl -2H-1-benzopyran-3-carboxylic acid; 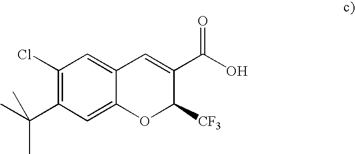
((S)-6-Chloro-7-(1,1-dimethylethyl)-2-(trifluo romethyl-2H-1-benzopyran-3-carboxylic acid; 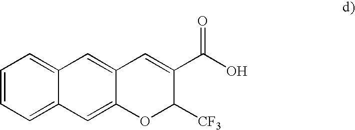
2-Trifluoromethyl-2H-naphtho[2,3-b]pyran-3-carboxylic acid; 10 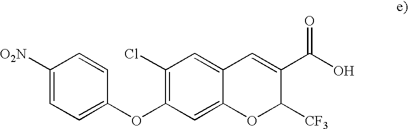
6-Chloro-7-(4-nitrophenoxy)-2-(trifluoromethyl)-2H-1-benzopyran-3-carboxylic acid; 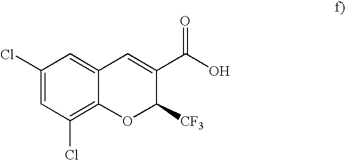
((S)-6,8-Dichloro-2-(trifluoromethyl)-2H-1-benzopyran-3-carboxylic acid; 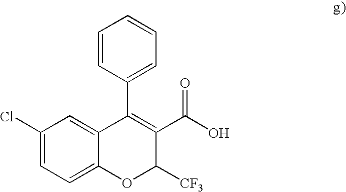
6-Chloro-2-(trifluoromethyl)-4-phenyl-2H-1-benzopyran-3-carboxylic acid; 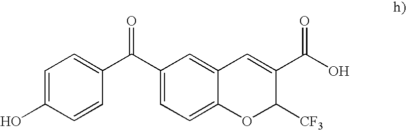
6-(4-Hydroxybenzoyl)-2-(trifluoromethyl) -2H-1-benzopyran-3-carboxylic acid; 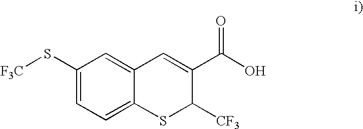
2-(Trifluoromethyl)-6-[(trifluoromethyl)thio]-2H-1-benzothiopyran-3-carboxylic acid; 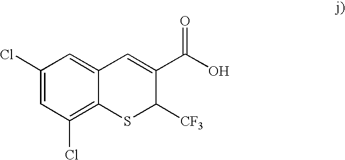
6,8-Dichloro-2-trifluoromethyl-2H-1-benzothiopyran-3-carboxylic acid; 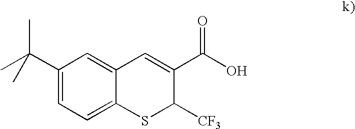
6-(1,1-Dimethylethyl)-2-(trifluoromethyl) -2H-1-benzothiopyran-3-carboxylic acid; 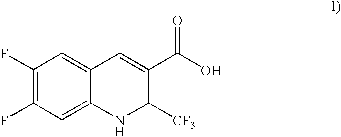
6,7-Difluoro-1,2-dihydro-2-(trifluoro methyl)-3-quinolinecarboxylic acid; 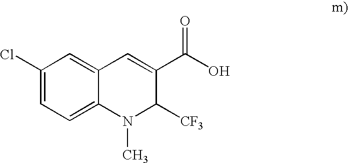
6-Chloro-1,2-dihydro-1-methyl-2-(trifluoro methyl)-3-quinolinecarboxylic acid; 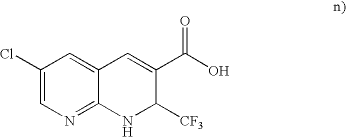
6-Chloro-2-(trifluoromethyl)-1,2-dihydro [1,8]naphthyridine-3-carboxylic acid; 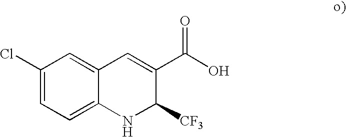
((S)-6-Chloro-1,2-dihydro-2-(trifluoro methyl)-3-quinolinecarboxylic acid;
and any combination thereof.

18. The composition of claim 1 wherein the cyclooxygenase inhibitor comprises a compound of the formula or a pharmaceutically acceptable salt or prodrug thereof 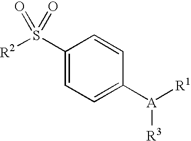
wherein:
A is selected from the group consisting of partially unsaturated or unsaturated heterocyclyl and partially unsaturated or unsaturated carbocyclic rings;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl or amino; and
R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

19. The composition of claim 1 wherein the cyclooxygenase-2 selective inhibitor pharmaceutically acceptable salt or prodrug thereof is selected from the group consisting of: 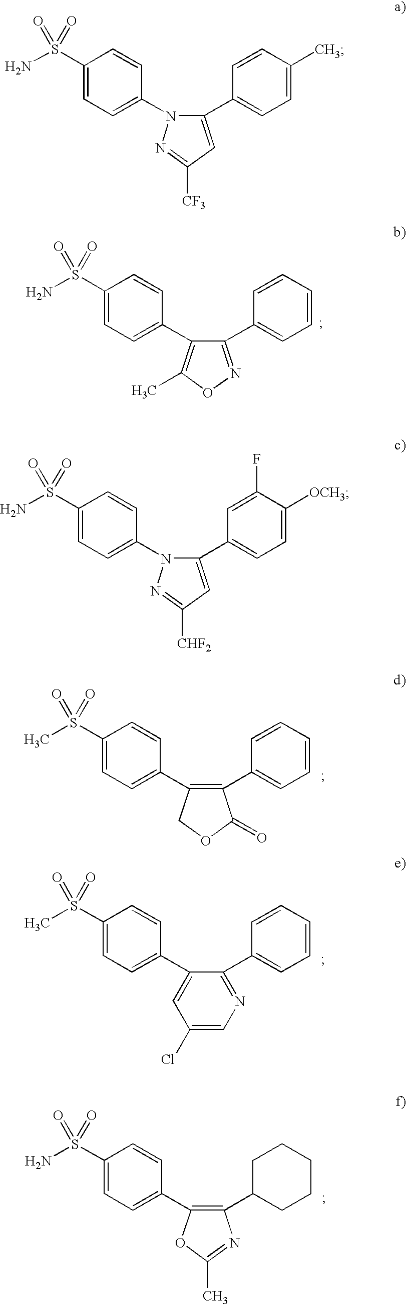
and any combination thereof

20. The composition of claim 1 wherein the cyclooxygenase-2 selective inhibitor or a pharmaceutically acceptable salt or prodrug thereof is selected from the group consisting of: 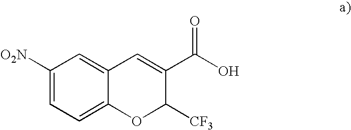
6-Nitro-2-trifluoromethyl-2H-1-benzopyran-3-carboxylic acid; 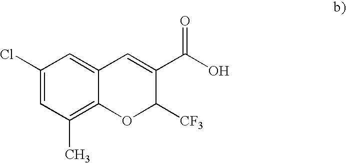
6-Chloro-8-methyl-2-trifluoromethyl -2H-1-benzopyran-3-carboxylic acid; 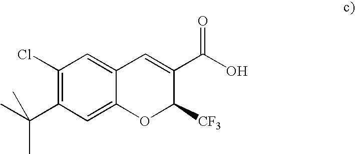
((S)-6-Chloro-7-(1,1-dimethylethyl)-2-(trifluo romethyl-2H-1-benzopyran-3-carboxylic acid; 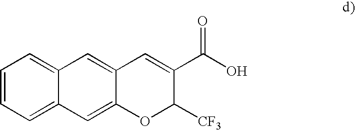
2-Trifluoromethyl-2H-naphtho[2,3-b]pyran-3-carboxylic acid; 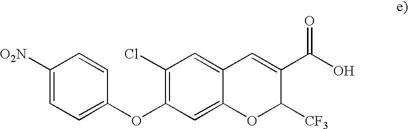
6-Chloro-7-(4-nitrophenoxy)-2-(trifluoromethyl)-2H-1-benzopyran-3-carboxylic acid; 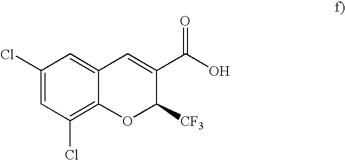
((S)-6,8-Dichloro-2-(trifluoromethyl)-2H-1-benzopyran-3-carboxylic acid; 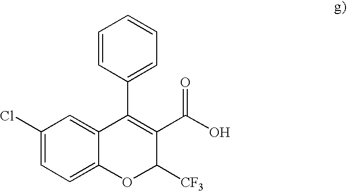
6-Chloro-2-(trifluoromethyl)-4-phenyl-2H-1-benzopyran-3-carboxylic acid; 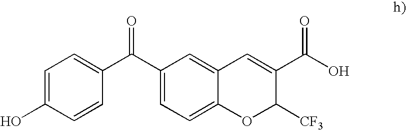
6-(4-Hydroxybenzoyl)-2-(trifluoromethyl) -2H-1-benzopyran-3-carboxylic acid; 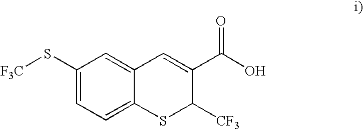
2-(Trifluoromethyl)-6-[(trifluoromethyl)thio]-2H-1-benzothiopyran-3-carboxylic acid; 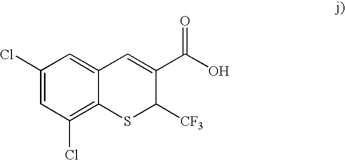
6,8-Dichloro-2-trifluoromethyl-2H-1-benzothiopyran-3-carboxylic acid; 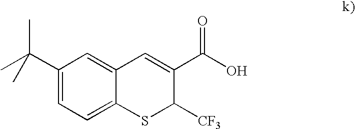
6-(1,1-Dimethylethyl)-2-(trifluoromethyl) -2H-1-benzothiopyran-3-carboxylic acid; 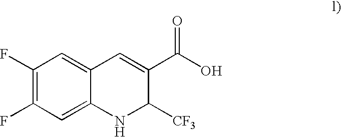
6,7-Difluoro-1,2-dihydro-2-(trifluoro methyl)-3-quinolinecarboxylic acid; 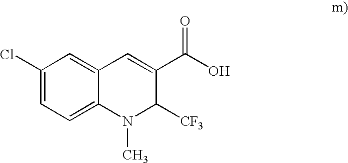
6-Chloro-1,2-dihydro-1-methyl-2-(trifluoro methyl)-3-quinolinecarboxylic acid; 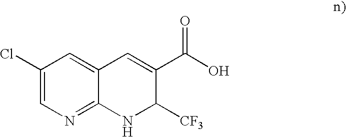
6-Chloro-2-(trifluoromethyl)-1,2-dihydro [1,8]naphthyridine-3-carboxylic acid; 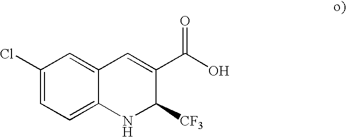
((S)-6-Chloro-1,2-dihydro-2-(trifluoro methyl)-3-quinolinecarboxylic acid; 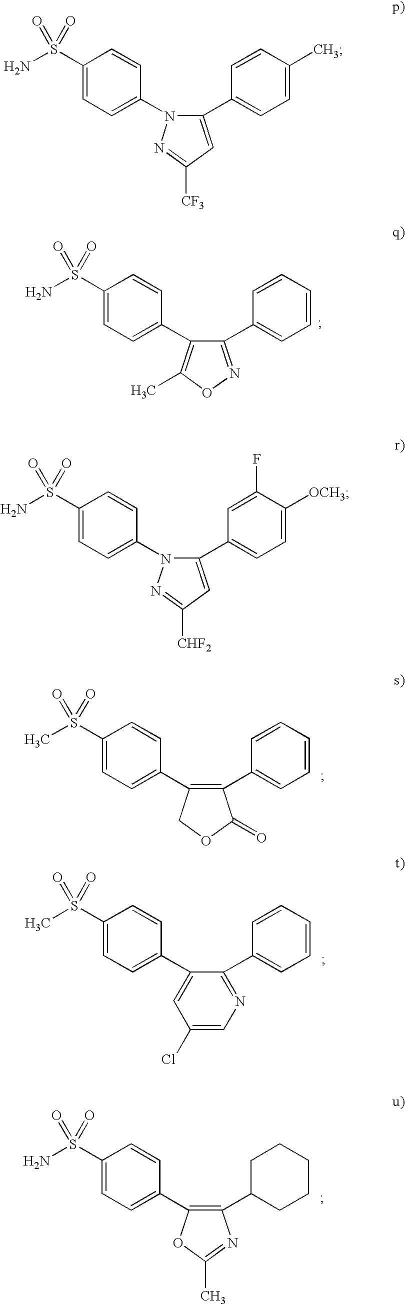 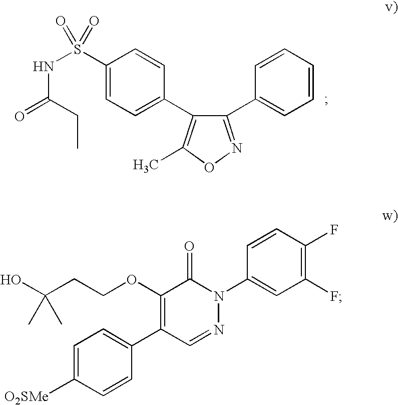
and any combination thereof.

21. The composition of claim 1 wherein the cyclooxygenase-2 selective inhibitor comprises: 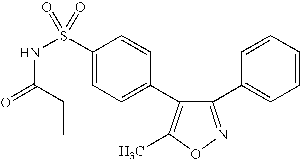
or a pharmaceutically acceptable salt, ester, isomer or prodrug thereof.

22. The composition of claim 1 wherein the cyclooxygenase-2 selective inhibitor comprises: 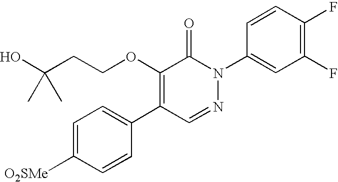
or a pharmaceutically acceptable salt, ester, isomer or prodrug thereof.

23. The composition of claim 1 wherein the cyclooxygenase-2 selective inhibitor comprises 4-[4-(methyl)-sulfonyl)phenyl]-3-phenyl-2(5H)-furanone, or a pharmaceutically acceptable salt, ester, isomer or prodrug thereof.

24. The composition of claim 1 wherein the cyclooxygenase-2 selective inhibitor comprises 4-(5-methyl-3-phenyl-4-isoxazolyl), or a pharmaceutically acceptable salt, ester, isomer or prodrug thereof.

25. The composition of claim 1 wherein the cyclooxygenase-2 selective inhibitor comprises 2-(6-methylpyrid-3-yl)-3-(4-methylsulfonylphenyl)-5-chloropyridine, or a pharmaceutically acceptable salt, ester, isomer or prodrug thereof.

26. The composition of claim 1 wherein the cyclooxygenase-2 selective inhibitor comprises 4-[5-(4-methylphenyl)-3-(trifluoromethyl)-1H-pyrazol-1-yl], or a pharmaceutically acceptable salt, ester, isomer or prodrug thereof.

27. The composition of claim 1 wherein the cyclooxygenase-2 selective inhibitor comprises N-[[4-(5-methyl-3-phenyl-4-isoxazolyl)phenyl]sulfonyl], or a pharmaceutically acceptable salt, ester, isomer or prodrug thereof.

28. The composition of claim 1 wherein the cyclooxygenase-2 selective inhibitor comprises 4-[5-(3-fluoro-4-methoxyphenyl)-3-difluoromethyl)-1H-pyrazol-1-yl]benzenesulfonamide, or a pharmaceutically acceptable salt, ester, isomer or prodrug thereof.

29. The composition of claim 1 wherein the cyclooxygenase-2 selective inhibitor comprises (S)-6,8-dichloro-2-(trifluoromethyl)-2H-1-benzopyran-3-carboxylic acid, or a pharmaceutically acceptable salt, ester, isomer or prodrug thereof.

30. The composition of claim 1 wherein the cyclooxygenase-2 selective inhibitor comprises 2-(3,4-difluorophenyl)-4-(3-hydroxy-3-methylbutoxy)-5-[4-(methylsulfonyl)phenyl]-3(2H)-pyridzainone, or a pharmaceutically acceptable salt, ester, isomer or prodrug thereof.

31. The composition of claim 1 wherein the cyclooxygenase-2 selective inhibitor comprises a compound of the formula or an isomer, a pharmaceutically acceptable salt, ester, or prodrug thereof 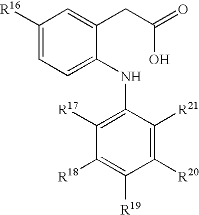
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

32. The composition of claim 31 wherein:
R¹⁶ is ethyl;
R¹⁷ and R¹⁹ are chloro;
R¹⁸ and R²⁰ are hydrogen; and
and R²¹ is methyl.

33. The composition of claim 1 wherein the cyclooxygenase-2 selective inhibitor comprises a compound of the formula or an isomer, a pharmaceutically acceptable salt, an ester, or a prodrug thereof: 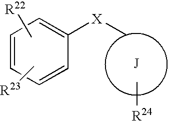
wherein:
X is O or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄.

34. The composition of claim 1 wherein the cyclooxygenase-2 selective inhibitor comprises a compound of the formula or an isomer, a pharmaceutically acceptable salt, ester, or prodrug thereof 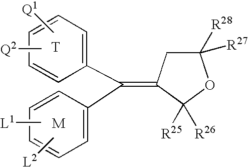
wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,
Q¹ and Q² are methylenedioxy; or
L¹ and L² are methylenedioxy; and
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²⁵ and R²⁶ are O; or,
R²⁷ and R²⁸ are O; or,
R²⁵, R²⁶, together with the carbon atom to which they are attached, form a saturated hydrocarbon ring having from 3 to 7 carbon atoms; or, R²⁷, R²⁸, together with the carbon atom to which they are attached, form a saturated hydrocarbon ring having from 3 to 7 carbon atom.

35. The composition of claim 1 wherein the cyclooxygenase-2 selective inhibitor, pharmaceutically acceptable salt, isomer, or prodrug thereof is selected from the group consisting of:
3-[(3-Chloro-phenyl)-(4-methanesulfonyl-phenyl)-methylene]-dihydro-furan-2-one;
8-acetyl-3-(4-fluorophenyl)-2-(4-methylsulfonyl)phenyl-imidazo(1,2-a);
5,5-dimethyl-4-(4-methylsulfonyl)phenyl-3-phenyl-2-(5H)-furanone;
5-(4-fluorophenyl)-1-[4-(methylsulfonyl)phenyl]-3-(trifluoromethyl)pyrazole;
4-(4-fluorophenyl)-5-[4-(methylsulfonyl)phenyl]-1-phenyl-3-(trifluoromethyl)pyrazole;
4-(5-(4-chlorophenyl)-3-(4-methoxyphenyl)-1H-pyrazol-1-yl)benzenesulfonamide;
4-(3,5 -bis(4-methylphenyl)-1H-pyrazol-1-yl)benzenesulfonamide;
4-(5-(4-chlorophenyl)-3-phenyl-1H-pyrazol-1-yl)benzenesulfonamide;
4-(3,5-bis(4-methoxyphenyl)-1H-pyrazol-1-yl)benzenesulfonamide;
4-(5-(4-chlorophenyl)-3-(4-methylphenyl)-1H-pyrazol-1-yl)benzenesulfonamide;
4-(5-(4-chlorophenyl)-3-(4-nitrophenyl)-1H-pyrazol-1-yl)benzenesulfonamide;
4-(5-(4-chlorophenyl)-3-(5-chloro-2-thienyl)-1H-pyrazol-1-yl)benzenesulfonamide;
4-(4-chloro-3,5-diphenyl-1H-pyrazol-1-yl)benzenesulfonamide;
4-[5-(4-chlorophenyl)-3-(trifluoromethyl)-1H-pyrazol-1-yl]benzenesulfonamide;
4-[5-phenyl-3-(trifluoromethyl)-1H-pyrazol-1-yl]benzenesulfonamide;
4-[5-(4-fluorophenyl)-3-(trifluoromethyl)-1H-pyrazol-1-yl]benzenesulfonamide;
4-[5-(4-methoxyphenyl)-3-(trifluoromethyl)-1H-pyrazol-1-yl]benzenesulfonamide;
4-[5-(4-chlorophenyl)-3-(difluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[4-chloro-5-(4-chlorophenyl)-3-(trifluoromethyl)-1H-pyrazol-1-yl]benzenesulfonamide;
4-[3-(difluoromethyl)-5-(4-methylphenyl)-1H-pyrazol-1-yl]benzenesulfonamide;
4-[3-(difluoromethyl)-5-phenyl-1H-pyrazol-1-yl]benzenesulfonamide;
4-[3-(difluoromethyl)-5-(4-methoxyphenyl)-1H-pyrazol-1-yl]benzenesulfonamide;
4-[3-cyano-5-(4-fluorophenyl)-1H-pyrazol-1-yl]benzenesulfonamide;
4-[3-(difluoromethyl)-5-(3-fluoro-4-methoxyphenyl)-1H-pyrazol-1-yl]benzenesulfonamide;
4-[5-(3-fluoro-4-methoxyphenyl)-3-(trifluoromethyl)-1H-pyrazol-1-yl]benzenesulfonamide;
4-[4-chloro-5-phenyl-1H-pyrazol-1-yl]benzenesulfonamide;
4-[5-(4-chlorophenyl)-3-(hydroxymethyl)-1H-pyrazol-1-yl]benzenesulfonamide;
4-[5-(4-(N,N-dimethylamino)phenyl)-3-(trifluoromethyl)-1H-pyrazol-1-yl]benzenesulfonamide;
5-(4-fluorophenyl)-6-[4-(methylsulfonyl)phenyl]spiro[2.4]hept-5-ene;
4-[6-(4-fluorophenyl)spiro[2.4]hept-5-en-5-yl]benzenesulfonamide;
6-(4-fluorophenyl)-7-[4-(methylsulfonyl)phenyl]spiro[3.4]oct-6-ene;
5-(3-chloro-4-methoxyphenyl)-6-[4-(methylsulfonyl)phenyl]spiro[2.4]hept-5-ene;
4-[6-(3-chloro-4-methoxyphenyl)spiro[2.4]hept-5-en-5-yl]benzenesulfonamide;
5-(3,5-dichloro-4-methoxyphenyl)-6-[4-(methylsulfonyl)phenyl]spiro[2.4]hept-5-ene;
5-(3-chloro-4-fluorophenyl)-6-[4-(methylsulfonyl)phenyl]spiro[2.4]hept-5-ene;
4-[6-(3,4-dichlorophenyl)spiro[2.4]hept-5-en-5-yl]benzenesulfonamide;
2-(3-chloro-4-fluorophenyl)-4-(4-fluorophenyl)-5-(4-methylsulfonylphenyl)thiazole;
2-(2-chlorophenyl)-4-(4-fluorophenyl)-5-(4-methylsulfonylphenyl)thiazole;
5-(4-fluorophenyl)-4-(4-methylsulfonylphenyl)-2-methylthiazole;
4(4-fluorophenyl)-5-(4-methylsulfonylphenyl)-2-trifluoromethylthiazole;
4(4-fluorophenyl)-5-(4-methylsulfonylphenyl)-2-(2-thienyl)thiazole;
4(4-fluorophenyl)-5-(4-methylsulfonylphenyl)-2-benzylaminothiazole;
4-(4-fluorophenyl)-5-(4-methylsulfonylphenyl)-2-(1-propylamino)thiazole;
2-[(3,5-dichlorophenoxy)methyl)-4-(4-fluorophenyl)-5-[4-(methylsulfonyl)phenyl]thiazole;
5-(4-fluorophenyl)-4-(4-methylsulfonylphenyl)-2-trifluoromethylthiazole;
1-methylsulfonyl-4-[1,1-dimethyl-4-(4-fluorophenyl)cyclopenta-2,4-dien-3-yl]benzene
4-[4-(4-fluorophenyl)-1,1-dimethylcyclopenta-2,4-dien-3-yl]benzenesulfonamide;
5-(4-fluorophenyl)-6-[4-(methylsulfonyl)phenyl]spiro[2.4]hepta-4,6-diene;
4-[6-(4-fluorophenyl)spiro[2.4]hepta-4,6-dien-5-yl]benzenesulfonamide;
6-(4-fluorophenyl)-2-methoxy-5-[4-(methylsulfonyl)phenyl]-pyridine-3-carbonitrile;
2-bromo-6-(4-fluorophenyl)-5-[4-(methylsulfonyl)phenyl]-pyridine-3-carbonitrile;
6-(4-fluorophenyl)-5-[4-(methylsulfonyl)phenyl]-2-phenyl-pyridine-3-carbonitrile;
4-[2-(4-methylpyridin-2-yl)-4-(trifluoromethyl)-1H-imidazol-1-yl]benzenesulfonamide;
4-[2-(5-methylpyridin-3-yl)-4-(trifluoromethyl)-1H-imidazol-1-yl]benzenesulfonamide;
4-[2-(2-methylpyridin-3-yl)-4-(trifluoromethyl)-1H-imidazol-1-yl]benzenesulfonamide;
3-[1-[4-(methylsulfonyl)phenyl]-4-(trifluoromethyl)-1H-imidazol-2-yl]pyridine;
2-[1-[4-(methylsulfonyl)phenyl-4-(trifluoromethyl)-1H-imidazol-2-yl]pyridine;
2-methyl-4-[1-[4-(methylsulfonyl)phenyl-4-(trifluoromethyl)-1H-imidazol-2-yl]pyridine;
2-methyl-6-[1-[4-(methylsulfonyl)phenyl-4-(trifluoromethyl)-1H-imidazol-2-yl]pyridine;
4-[2-(6-methylpyridin-3-yl)-4-(trifluoromethyl)-1H-imidazol-1-yl]benzenesulfonamide;
2-(3,4-difluorophenyl)-1-[4-(methylsulfonyl)phenyl]-4-(trifluoromethyl)-1H-imidazole;
4-[2-(4-methylphenyl)-4-(trifluoromethyl)-1H-imidazol-1-yl]benzenesulfonamide;
2-(4-chlorophenyl)-1-[4-(methylsulfonyl)phenyl]-4-methyl-1H-imidazole;
2-(4-chlorophenyl)-1-[4-(methylsulfonyl)phenyl]-4-phenyl-1H-imidazole;
2-(4-chlorophenyl)-4-(4-fluorophenyl)-1-[4-(methylsulfonyl)phenyl]-1H-imidazole;
2-(3-fluoro-4-methoxyphenyl)-1-[4-(methylsulfonyl)phenyl-4-(trifluoromethyl)-1H-imidazole;
1-[4-(methylsulfonyl)phenyl]-2-phenyl-4-trifluoromethyl-1H-imidazole;
2-(4-methylphenyl)-1-[4-(methylsulfonyl)phenyl]-4-trifluoromethyl-1H-imidazole;
4-[2-(3-chloro-4-methylphenyl)-4-(trifluoromethyl)-1H-imidazol-1-yl]benzenesulfonamide;
2-(3-fluoro-5-methylphenyl)-1-[4-(methylsulfonyl)phenyl]-4-(trifluoromethyl)-1H-imidazole;
4-[2-(3-fluoro-5-methylphenyl)-4-(trifluoromethyl)-1H-imidazol-1-yl]benzenesulfonamide;
2-(3-methylphenyl)-1-[4-(methylsulfonyl)phenyl]-4-trifluoromethyl-1H-imidazole;
4-[2-(3-methylphenyl)-4-trifluoromethyl-1H-imidazol-1-yl]benzenesulfonamide;
1-[4-(methylsulfonyl)phenyl]-2-(3-chlorophenyl)-4-trifluoromethyl-1H-imidazole;
4-[2-(3-chlorophenyl)-4-trifluoromethyl-1H-imidazol-1-yl]benzenesulfonamide;
4-[2-phenyl-4-trifluoromethyl-1H-imidazol-1-yl]benzenesulfonamide;
4-[2-(4-methoxy-3-chlorophenyl)-4-trifluoromethyl-1H-imidazol-1-yl]benzenesulfonamide;
1-allyl-4-(4-fluorophenyl)-3-[4-(methylsulfonyl)phenyl]-5-(trifluoromethyl)-1H-pyrazole;
4-[1-ethyl-4-(4-fluorophenyl)-5-(trifluoromethyl)-1H-pyrazol-3-yl]benzenesulfonamide;
N-phenyl-[4-(4-fluorophenyl)-3-[4-(methylsulfonyl)phenyl]-5-(trifluoromethyl)-1H-pyrazol-1-yl]acetamide;
ethyl -[4-(4-fluorophenyl)-3-[4-(methylsulfonyl)phenyl]-5-(trifluoromethyl)-1H-pyrazol-1-yl]acetate;
4(4-fluorophenyl)-3-[4-(methylsulfonyl)phenyl]-1-(2-phenylethyl)-1H-pyrazole;
4(4-fluorophenyl)-3-[4-(methylsulfonyl)phenyl]-1-(2-phenylethyl)-5-(trifluoromethyl)pyrazole;
1-ethyl-4-(4-fluorophenyl)-3-[4-(methylsulfonyl)phenyl]-5-(trifluoromethyl)-1H-pyrazole;
5-(4-fluorophenyl)-4-(4-methylsulfonylphenyl)-2-trifluoromethyl-1H-imidazole;
4-[4-(methylsulfonyl)phenyl]-5-(2-thiophenyl)-2-(trifluoromethyl)-1H-imidazole;
5-(4-fluorophenyl)-2-methoxy-4-[4-(methylsulfonyl)phenyl]-6-(trifluoromethyl)pyridine;
2-ethoxy-5-(4-fluorophenyl)-4-[4-(methylsulfonyl)phenyl]-6-(trifluoromethyl)pyridine;
5-(4-fluorophenyl)-4-[4-(methylsulfonyl)phenyl]-2-(2-propynyloxy)-6-(trifluoromethyl)pyridine;
2-bromo-5-(4-fluorophenyl)-4-[4-(methylsulfonyl)phenyl]-6-(trifluoromethyl)pyridine;
4-[2-(3-chloro-4-methoxyphenyl)-4,5-difluorophenyl]benzenesulfonamide;
1-(4-fluorophenyl)-2-[4-(methylsulfonyl)phenyl]benzene;
5-difluoromethyl-4-(4-methylsulfonylphenyl)-3-phenylisoxazole;
4-[3-ethyl-5-phenylisoxazol-4-yl]benzenesulfonamide;
4-[5-difluoromethyl-3-phenylisoxazol-4-yl]benzenesulfonamide;
4-[5-hydroxymethyl-3-phenylisoxazol-4-yl]benzenesulfonamide;
4-[5-methyl-3-phenyl-isoxazol-4-yl]benzenesulfonamide;
1-[2-(4-fluorophenyl)cyclopenten-1-yl]-4-(methylsulfonyl)benzene;
1-[2-(4-fluoro-2-methylphenyl)cyclopenten-1-yl]-4-(methylsulfonyl)benzene;
1-[2-(4-chlorophenyl)cyclopenten-1-yl]-4-(methylsulfonyl)benzene;
1-[2-(2,4-dichlorophenyl)cyclopenten-1-yl]-4-(methylsulfonyl)benzene;
1-[2-(4-trifluoromethylphenyl)cyclopenten-1-yl]-4-(methylsulfonyl)benzene;
1-[2-(4-methylthiophenyl)cyclopenten-1-yl]-4-(methylsulfonyl)benzene;
1-[2-(4-fluorophenyl)-4,4-dimethylcyclopenten-1-yl]-4-(methylsulfonyl)benzene;
4-[2-(4-fluorophenyl)-4,4-dimethylcyclopenten-1-yl]benzenesulfonamide;
1-[2-(4-chlorophenyl)-4,4-dimethylcyclopenten-1-yl]-4-(methylsulfonyl)benzene;
4-[2-(4-chlorophenyl)-4,4-dimethylcyclopenten-1-yl]benzenesulfonamide;
4-[2-(4-fluorophenyl)cyclopenten-1-yl]benzenesulfonamide;
4-[2-(4-chlorophenyl)cyclopenten-1-yl]benzenesulfonamide;
1-[2-(4-methoxyphenyl)cyclopenten-1-yl]-4-(methylsulfonyl)benzene;
1-[2-(2,3-difluorophenyl)cyclopenten-1-yl]-4-(methylsulfonyl)benzene;
4-[2-(3-fluoro-4-methoxyphenyl)cyclopenten-1-yl]benzenesulfonamide;
1-[2-(3-chloro-4-methoxyphenyl)cyclopenten-1-yl]-4-(methylsulfonyl)benzene;
4-[2-(3-chloro-4-fluorophenyl)cyclopenten-1-yl]benzenesulfonamide;
4-[2-(2-methylpyridin-5-yl)cyclopenten-1-yl]benzenesulfonamide;
ethyl 2-[4-(4-fluorophenyl)-5-[4-(methylsulfonyl) phenyl]oxazol-2-yl]-2-benzylacetate;
2-[4-(4-fluorophenyl)-5-[4-(methylsulfonyl)phenyl]oxazol-2-yl]acetic acid;
2-(tert-butyl)-4-(4-fluorophenyl)-5-[4-(methylsulfonyl)phenyl]oxazole;
4-(4-fluorophenyl)-5-[4-(methylsulfonyl)phenyl]-2-phenyloxazole;
4(4-fluorophenyl)-2-methyl-5-[4-(methylsulfonyl)phenyl]oxazole;
4-[5-(3-fluoro-4-methoxyphenyl)-2-trifluoromethyl-4-oxazolyl]benzenesulfonamide;
6-chloro-7-(1,1-dimethylethyl)-2-trifluoromethyl-2H-1-benzopyran-3-carboxylic acid;
6-chloro-8-methyl-2-trifluoromethyl-2H-1-benzopyran-3-carboxylic acid;
5,5-dimethyl-3-(3-fluorophenyl)-4-methylsulfonyl-2(5H)-furanone;
6-chloro-2-trifluoromethyl-2H-1-benzothiopyran-3-carboxylic acid;
4-[5-(4-chlorophenyl)-3-(trifluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[5-(3-fluoro-4-methoxyphenyl)-3-(difluoromethyl)-1H-pyrazol-1-yl]benzenesulfonamide;
3-[1-[4-(methylsulfonyl)phenyl]-4-trifluoromethyl-1H-imidazol-2-yl]pyridine;
2-methyl-5-[1-[4-(methylsulfonyl)phenyl]-4-trifluoromethyl-1H-imidazol-2-yl]pyridine;
4-[2-(5-methylpyridin-3-yl)-4-(trifluoromethyl)-1H-imidazol1-yl]benzenesulfonamide;
4-[5-methyl-3-phenylisoxazol-4-yl]benzenesulfonamide;
4-[5-hydroxymethyl-3-phenylisoxazol-4-yl]benzenesulfonamide;
[2-trifluoromethyl-5-(3,4-difluorophenyl)-4-oxazolyl]benzenesulfonamide;
4-[2-methyl-4-phenyl-5-oxazolyl]benzenesulfonamide;
4-[5-(2-fluoro-4-methoxyphenyl)-2-trifluoromethyl-4-oxazolyl]benzenesulfonamide;
[2-(2-chloro-6-fluoro-phenylamino)-5-methyl-phenyl]-acetic acid;
N-(4-Nitro-2-phenoxy-phenyl)-methanesulfonamide or nimesulide;
N-[6-(2,4-difluoro-phenoxy)-1-oxo-indan-5-yl]-methanesulfonamide;
N-[6-(2,4-Difluoro-phenylsulfanyl)-1-oxo-1H-inden-5-yl]-methanesulfonamide, soldium salt;
N-[5-(4-fluoro-phenylsulfanyl)-thiophen-2-yl]-methanesulfonamide;
3-(3,4-Difluoro-phenoxy)-4-(4-methanesulfonyl-phenyl)-5-methyl-5-(2,2,2-trifluoroethyl)-5H-furan-2-one;
(5Z)-2-amino-5-[[3,5-bis(1,1-dimethylethyl)-4-hydroxyphenyl]methylene]-4(5H)-thiazolone;
N-[3-(formylamino)-4-oxo-6-phenoxy-4H-1-benzopyran-7-yl]-methanesulfonamide;
(6aR, 10aR)-3-(1,1-dimethylheptyl)-6a,7,10,10a-tetrahydro-1-hydroxy-6,6-dimethyl-6H-dibenzo[b,d]pyran-9-carboxylic acid;
4-[[3,5-bis(1,1-dimethylethyl)-4-hydroxyphenyl]methylene]dihydro-2-methyl-2H-1,2-oxazin-3(4H)-one;
6-dioxo-9H-purin-8-yl-cinnamic acid;
4-[4-(methyl)-sulfonyl)phenyl]-3-phenyl-2(5H)-furanone;
4(5-methyl-3-phenyl-4-isoxazolyl);
2-(6-methylpyrid-3-yl)-3-(4-methylsulfonylphenyl)-5-chloropyridine;
4-[5-(4-methylphenyl)-3-(trifluoromethyl)-1H-pyrazol-1-yl];
N-[[4-(5-methyl-3-phenyl-4-isoxazolyl)phenyl]sulfonyl];
4-[5-(3-fluoro-4-methoxyphenyl)-3-difluoromethyl)-1H-pyrazol-1-yl]benzenesulfonamide;
(S)-6,8-dichloro-2-(trifluoromethyl)-2H-1-benzopyran-3-carboxylic acid;
2-(3,4-difluorophenyl)-4-(3-hydroxy-3-methylbutoxy)-5-[4-(methylsulfonyl)phenyl]-3(2H)-pyridzainone;
2-trifluoromethyl-3H-naptho[2,1-b]pyran-3-carboxylic acid;
6-chloro-7-(1,1-dimethylethyl)-2-trifluoromethyl-2H-1-benzopyran-3-carboxylic acid;
and
[2-(2,4-dichloro-6-ethyl-3,5-dimethyl-phenylamino)-5-propyl-phenyl]-acetic acid.

36. The composition of claim 1 wherein the cyclooxygenase-2 selective inhibitor, pharmaceutically acceptable salt, isomer, or prodrug thereof acts as an immunostimulant.

37. The composition of claim 1 wherein the anti-human immunodeficiency virus agent substantially inhibits HIV infection in the human.

38. The composition of claim 37 wherein the anti-human immunodeficiency virus agent inhibits HIV infection by substantially inhibiting the HIV virus.

39. The composition of claim 37 wherein the anti-human immunodeficiency virus agent substantially inhibits HIV infection by causing the human to substantially inhibit the HIV infection.

40. The composition of claim 1 wherein the anti-human immunodeficiency virus agent is selected from the group consisting of a viral cellular entry inhibitor, a viral replication inhibitor, a viral assembly inhibitor, an integrase inhibitor, and a human immune enhancing agent.

41. The composition of claim 40 wherein the viral replication inhibitor is selected from the group consisting of a nucleoside analog, a non-nucleoside reverse transcriptase inhibitor, an acyclic nucleoside phosphonate analog, a zinc finger inhibitor, a viral gene expression inhibitor, a polyamine biosynthesis inhibitor, and a genetic or anti-sense therapy agent.

42. The composition of claim 41 wherein the nucleoside analog is selected from the group consisting of:
(−)-cis-2-amino-1,9-dihydro-9-[4-hydroxymethyl)-2-cyclopenten-1-yl)-6H-purin-6-one;
2,6-diamino-2′,3′-dideoxypurine-9-ribofuranoside;
9-(2-azido-2,3-dideoxy-b-D-erythro-pentofuranosyl)adenine;
1(2′-fluoro-2′,3′-dideoxy-B-D-erythro-pentofuranosyl)thymine;
9-(2-azido-2,3-dideoxy-b-D-threo-pentofuranosyl)adenine;
3-(3-oxo-1-propenyl)-3′-azido-3′-deoxythymidine;
3-azido-2′,3′-dideoxy-5-chlorocytidine;
3′-azido-3′deoxy-6-azathymidine;
2′,3′-dideoxy-3′-fluoro-4-thiothymidine;
2′,3′-dideoxy-3′-fluoro-5-chlorocytidine;
9-(3′-fluoro-2′,3′-dideoxy-B-D-erythropentafuranosyl)adenine;
3′-fluoro-2′,3′-dideoxycytidine;
2,6-diaminopurine-3′-fluoro-2′,3′-dideoxyriboside;
3′-fluoro-2′,3′-dideoxyguanosine;
3′-fluoro-2′,3′-dideoxyuridine;
1-[2′,3′-dideoxy-3′-C-(hydroxymethyl)-.beta.-D-erythro-pentofuranosyl]cytosine;
3′-azido-2′,3′-dideoxy-5-trifluoromethyluridine;
3′-azido-2′,3′-dideoxy-5-[(cyanomethyl)oxy]uridine;
3′-azido-2′,3′-dideoxy-5-fluorocytidine;
3′-azido-2′,3′-dideoxy-5-methylcytidine;
3′-azido-2′,3′-dideoxy-5-aminouridine;
3′-azido-2′,3′-dideoxy-5-methyaminouridine;
3′-azido-2′,3′-dideoxy-5-dimethylaminouridine;
3′-azido-2′,3′-dideoxy-5-hydroxyuridine;
3′-azido-2′,3′-dideoxy-5-thiocyanatouridine;
9-(3′-azido-2′,3′-dideoxy-B-D-erythropentafuranosyl)adenine;
3′-azido-2′,3′-dideoxycytidine;
3′-azido-2′,3′-dideoxyguanosine;
3′-azido-2′,3′-dideoxy-N4-5-dimethylcytidine;
3′-azido-2′,3′-dideoxy-N4-OH-5-methylcytidine;
4′-azido-3′-deoxythymidine;
4′-azido-5-chloro-2′-deoxyuridine;
4′-azido-2′-deoxyadenosine;
4′-azido-2′-deoxycytidine;
4′-azido-2′-deoxyguanosine;
4′-azido-2′-deoxyinosine;
4′-azido-2′-deoxyuridine;
1(4-azido-2-deoxy-.beta.-D-erythro-pentofuranosyl)-5-methyl-2,4-dioxopyrimidine;
4′-cyanothymidine;
5-fluoro-2′,3′-dideoxycytidine;
3′-azido-3′-deoxythymidine-5′-(butylmethoxyvalinyl)phosphate;
6-chloro-9-(2,3-dideoxy-.beta.-D-glyceropentofuranosyl)-9H-purine;
2′,3′-dideoxy-3′-fluoro-5-chlorouridine;
butanedioic acid, compd. with (1S-cis)-4-[2-amino-6-(cyclopropylamino)-9H-purine-9-yl]-2-cyclopentene-1-methanol (1:1);
5′-alkylglycoside carbonate of 3′-azido-3′-deoxythymidine;
3′-azido-2′,3′-dideoxy-5-bromouridine;
3′-azido-5-chloro-2′,3′-dideoxyuridine;
3′-azido-2′,3′-dideoxy-5-ethyluridine;
3′-azido-2′,3′-dideoxy-5-fluorouridine;
3′-azido-2′,3′-dideoxy-5-iodouridine;
2,5′-anhydro-3′-azido-3′-deoxythymidine;
1-(2,3-dideoxy-3-azido-a-L-threo-pentofuranosyl)thymine;
5′-[(1,4-dihydro1methyl-3-pyridinylcarbonyl)oxy]-3′-azido-2′3′-deoxythymidine;
3′-azido-3′-deoxythymidilyl-(5′,5′)-2′,3′-dideoxy-5′-adenylic acid, 2-cyanoethyl ester;
3-azido-3′-deoxythymidilyl-(5′,5′)-2′,3′-dideoxy-5′-adenylic acid;
3′-azido-3′-deoxythymidilyl-(5′,5′)-2′,3′-dideoxy-5′inosinic acid;
O,O′-bis(3′-azido-3′-deoxythymidin-5′-yl)methylphosphonate;
2,5′-anhydro-3′-azido-2′,3′-dideoxyuridine;
2,4(1H,3H)-pyrimidinedione,5-(3-azido-2,3-dideoxy-.beta.-D-erythro-pentofuranosyl);
(1S,4R)-4-[2-amino-6-(cyclopropylamino)-9H-purin-9-yl]-2-cyclopentene-1-methanol & .beta.-L-(−)-2′,3′-dideoxy-3′-thiacytidine & 3′-azido-3′-deoxythymidine;
(±)-9-[(1.beta.-2.alpha.-3.beta.)-2,3-bis(hydroxymethyl)-1-cyclobutyl]adenine;
9-[1.beta.-2.alpha.-3.beta.]-2,3-bis(hydroxymethyl)-1-cyclobutyl]guanine;
9-(2,3-dideoxy-.beta.-D-ribofuranosyl)-6-(methylthio)purine;
2,3-dideoxydidehydroadenosine;
3′-azido-3′-deoxythymidine;
2′,3′-dideoxydidehydrocytidine;
2,6-diaminopurine-2′,3′-dideoxydidehydrorboside;
b-D-2′,3′-didehydro-2′,3′-dideoxy-5-fluorocytidine;
2′,3′-didehydro-2′,3′-dideoxyguanosine;
2′,3′-dideoxyadenosine;
2′,3′-dideoxyguanosine;
3′-deoxythymidine;
2′,3′-dideoxyinosine;
6-dimethylaminopurine-2′,3′-dideoxyriboside;
(−)-2′-deoxy-3′-oxa-4′-thiocytidine;
(+)-2′-deoxy-3′-oxa-4′-thiocytidine;
(−)-2′-deoxy-3′-oxa-4′-thio-5-fluorocytidine;
(+)-2′-deoxy-3′-oxa-4′-thio-5-fluorocytidine;
(−)-(2R,4R)-9-[2-(hydroxymethyl)-1,3-dioxolan-4-yl]guanine;
(+)-(2S,4R)-1-[2-(hydroxymethyl)-1,3-dioxolan-4-yl]-5-fluorocytosine;
2′,3′-dideoxy-3′-fluoro-5-bromouridine;
3′-fluoro-2′,3′-dideoxy-5-iodouridine;
3′-fluoro-3′-deoxythymidine;
(−)-(2R,5S)-5-fluoro1-[2-(hydroxymethyl)-1,3-oxathiolan-5-yl]cytosine;
(+)-(2R,5R)-5-fluoro-1-[2-(hydroxymethyl)-1,3-oxathiolan-5-yl]cytosine;
.beta.-L-2′,3′-didehydro-2′,3′-dideoxyadenosine;
2′,3′-dideoxy-2′,3′-didehydro-.beta.-L-5-5-fluorocytidine;
.beta.-L-2′,3′-didehydro-2′,3′-dideoxyinosine;
.beta.-L-2′,3′-didehydro-2′,3′-dideoxyguanosine;
2(1H)-pyrimidinone, 4-amino-5-fluoro-1-[(2S,5R)-tetrahydro-5-(hydroxymethyl)-2-furanyl];
cis-1-[2′-hydroxymethyl-5′-(1,3-oxathiolanyl)]cytosine;
9-(2″-fluoro-2′,3′-dideoxy-B-D-threopentafuranosyl)adenine;
5-methyl-3′-azido-2′,3′-dideoxyisocytidine;
N-ethyl-2′,3′-dideoxyadenosine;
6-methyl-2′,3′-dideoxyadenosine;
1.beta.-D-ribofuranosyl-1,2,4-triazolo-3-carboxamide;
1-(2′,3′-dideoxy-2′-fluoro-.beta.-D-threo-pentofuranosyl)cytosine;
thymidine, 2′,3′-didehydro-,3′-deoxy;
(1S,4R)-4-[2-amino-6-(cyclopropylamino)-9H-purin-9-yl]-2-cyclopentene-1-methanol & .beta.-L-(−)-2′,3′-dideoxy-3′-thiacytidine & 3′-azido-3′-deoxythymidine;
3′-azido-2′,3′-dideoxyuridine;
2′,3′-dideoxycytidine;
9-[(R)-2-[[bis[[(isopropoxycarbonyl)oxy]methoxy]phosphinyl]methoxy]propyl]adenine;
or a prodrug thereof.

43. The composition of claim 41 wherein the non-nucleoside reverse transcriptase inhibitor is selected from the group consisting of:
6-chloro-3-(phenylthio)-2-indolecarboxamide;
1-[(5-methanesulfonamidoindol-2-yl)carbonyl]-4-[N-ethyl-N-[3-[(1,1-dimethylethyl)amino]-2-pyridinyl]amino]piperidine;
methyl-3′,3″-dichloro-4′,4″-dimethoxy-5′,5″-bis(methoxycarbonyl)-6,6-diphenylhexenoate;
Methyl-3-bromo-5-(1-(5-bromo-4-methoxy-3-(methoxycarbonyl)phenyl)hept-1-enyl)-2-methoxybenzoate;
5-(3,5-dichlorophenyl)thio-4-isopropyl-1-(4-pyridyl)methyl-1H-imidazol-2-ylmethylcarbamate;
1-[(5-methoxyindol-2-yl)carbonyl]-4-[3-(ethylamino)-2-pyridyl]piperazine;
Aurintricarboxylic acid;
5,6,7-trihydroxyflavone-7-O-b-D-glucopyranosideuronic acid;
1-[(6-cyano-2-indolyl)carbonyl]-4-[3-(isopropylamino)-2-pyridinyl]piperazine;
1-[3-(ethylamino)-2-pyridinyl]-4-[(5-hydroxy-2-indolyl)carbonyl]piperazine;
1-[(6-formyl-2-indolyl)carbonyl]-4-[3-(isopropylamino)-2-pyridinyl]piperazine;
1-[[5-(methylsulfonyloxy)-2-indolyl]carbonyl]-4-[3-(isopropylamino)-2-pyridinyl]piperazine;
1-[5-[[N-(methyl)methylsulfonylamino]-2-indolyl]carbonyl]-4-[3-(isopropylamino)-2-pyridinyl]piperazine;
1(indolyl-2-carbonyl)-4-[3-[(1-methylethyl)amino]pyridyl]piperazine;
bis(2-nitrophenyl)sulfone;
Calanolide A;
Calanolide B;
5-(3,5-dichlorophenyl)thio-4-isopropyl-1-(4-pyridyl)methyl-1H-imidazol-2-ylmethylcarbamate;
6-benzyl-5-methyl-2-(cyclohexyloxy)pyrimidin-2-one;
1-(5-methanesulphonamido)-1H-indol-2-yl-carbonyl)-4-[3-(isopropylamino)-2-pyridinyl]piperazine;
6-chloro-4-(cyclopropylethynyl)-3,4-dihydro-4-(trifluoromethyl)-,(4S)2(1H)-quinazolinone;
6-benzyl-1-(ethoxymethyl)-5-ethyluracil;
5-ethyl-1-ethoxymethyl-6-(3,5-dimethylbenzyl)uracil;
5-ethyl1(ethoxymethyl)-6-(phenylselenenyl)uracil;
1-[(2-hydroxyethoxy)methyl]-6-(phenylthio)thymine;
1-[(ethoxy)methyl]-6-phenylthio)-5-ethyluracil;
(−)-6-chloro-4-cyclopropylethynyl-4-trifluoromethyl-1,4-dihydro-2H-3,1-benzoxazin-one;
1(ethoxymethyl)-5-(1-methylethyl)-6-(phenylmethyl)-2,4(1H,3H)-pyrimidinedione;
Phosphonoformic acid trisodium salt;
(S)-7-methoxy-3,4-dihydro-2-[(methylthio)methyl]-3-thioxo-2(1H)-quinoxalinecarboxylic acid, isopropyl ester;
1[(2-hydroxyethoxy)methyl]-6-(3-methylphenyl)thio)thymine; 1-[(2-hydroxyethoxy)methyl]-6-(phenylthio)-2-thiothymine;
inophyllum B;
inophyllum P;
5-chloro-3-(phenylsulfonyl)indole-2-carboxamide;
5,5′-(1,1′-dihydroxy-8,8′-dimethoxy-6,6′-dimethyl[2,2′-binaphthalene]-4,4′-diyl)bis[1,2,3,4,-tetrahydro-1,3-dimethyl-6,8-isoquinolinediol];
5,5′-(1,1′-dihydroxy-8,8′-dimethoxy-6,6′-dimethyl[2,2′-binaphthalene]-4,4′-diyl)bis[1,2,3,4,-tetrahydro-1,3-dimethyl-6,8-isoquinolinediol];
5,5′(1,1′-dihydroxy-8,8′-dimethoxy-6,6′-dimethyl[2,2′-binaphthalene]-4,4′-diyl)[3,4,dihydro-8-methoxy-1,3-dimethyl-6-isoquinolinediol],[1,2,3,4-tetrahydro-1,3-dimethyl-6,8-isoquinolinediol];
6-(3,5-dimethylbenzyl)-1-[(2-hydroxyethoxy)methyl]-5-isopropyluracile;
6-(3,5-dimethylbenzyl)-1-(ethoxymethyl])-5-isopropyluracile;
1-(ethoxymethyl)-5-(1-methylethyl)-6-(phenylmethyl)-2,4(1H,3H)-pyrimidinedione;
N11-cyclopropyl-4-methyl-5,11-dihydro-6H-dipyrido[3,2-b:2′,3′-e]-[1,4]diazepin-6-one;
2-nitrophenyl phenyl sulfone;
1-benzyloxymethyl-5-ethyl-6-(2-pyridylthio)uracil;
4methyl-5-(pyrazinyl)-3H-1,2-dithiole-3-thione;
N-[2-(2-chloro-6-fluorophenethyl)]-N′-(2-thiazolyl)thiourea;
N-(2-phenethyl)-N′-(2-thiazolyl)thiourea;
3-[(4,7-dimethyl-2-benzoxazolylmethyl)amino]-5-ethyl-6-methylpyridin-2(1H)-one;
3-[2-(4,7difluorobenzoxazol-2-yl)ethyl]-5-ethyl-6-methylpyridin-2(1H)-thione;
3-ethyl-6-methyl-3-[(2-phthalimido)ethyl]-2-pyridinone;
3-[(4,7-dichlorobenzoxazolylmethyl)amino]-5-ethyl-6-methylpyridin-2(1H)-one;
(±)-4,5,6,7-tetrahydro-5-methyl-6-(2-propenyl)-imidazo-[4,5,1-jk][1,4]-benzodiazepin-2(1H)-one;
(+)-S-4,5,6,7-tetrahydro-5-methyl-6-(3-methyl-2-butenyl)-imidazo-[4,5,1-jk][1,4]-benzodiazepin-2(1H)-thione;
(+)-S-4,5,6,7-tetrahydro-9-chloro-5-methyl-6-(3-methyl-2-butenyl)-imidazo[4,5,1-jk][1,4]-benzodiazepin-2(1H)-thione;
(−)-2,6-dichloro-.alpha.-[(2-nitrophenyl)amino]benzamide;
(±)-2,6-dichloro-.alpha.-[(2-acetylphenyl)amino]benzamide;
(±)-2,6-dichloro-.alpha.-[(2-acetyl-5-methylphenyl)amino]benzamide;
(−)-2,6-dichloro-.alpha.-[(2-acetyl-5-methylphenyl)amino]benzamide;
5-(3,5-dichlorophenyl)thio-4-isopropyl-1-(4-pyridyl)methyl-1H-imidazol-2-ylmethylcarbamate;
6-chloro-3,3-dimethyl-4-(isopropenyloxycarbonyl)-3,4-dihydroquinoxalin-2(1H)-thione;
8,8′-[carbonylbis[imino-3,1-phenylenecarbonylimino(4-methyl-3,1-phenylene)carbonyl-imino]]bis-1,3,5-naphthalenetrisulfonic acid hexasodium salt;
(R,S)-1-(2,6-difluorophenyl)-1H,3H-thiazolo[3,4-a]benzimidazole;
(+)-(R)-9b-(1-naphthyl)-2,3-dihydrothiazolo[2,3-a]isoindol-5(9bH)-one;
(+)-(R)-9b-(3,5-dimethylphenyl)-2,3-dihydrothiazolo[2,3-a]isoindol-5(9bH)-one;
(+)-(S)-4,5,6,7-tetrahydro-8-chloro-5-methyl-6-(3-methyl-2-butenyl)imidazo[4,5,1-jk][1,4]benzodiazepine-2-(1H)-thione;
N-[2-(2-pyridylethyl)-N′-[2-(5-bromopyridyl)]thiourea, hydrochloride;
thymidine, 3-methyl, [2′,5′bis-O-(tert-butyldimethylsilyl)-.beta.-D-ribofuranosyl]-3′-spiro-5-(4-amino-1,2-oxathiole-2,2-dioxide;
[1-[2′,5′-bis-O-(tert-butyldimethylsilyl)-.beta.-D-ribofuranosyl]thymine]-(R)(ribo)-3′-spiro-5-(4-amino-1,2-oxathiole-2,2-dioxide);
4-chloro-3-(isopropoxycarbonyl)phenylcarbamothioic acid, O-isopropyl ester;
N-[4-chloro-3-(3-methyl-2-butenyloxy)phenyl]-2-methyl-3-furancarbothioamide;
2-chloro-5[(2-methyl-5,6-dihydro-1,4-oxathiin-3-yl) carbonylamino]isopropyl ester benzoic acid; or a prodrug thereof.

44. The composition of claim 40 wherein the viral assembly inhibitor is selected from the group consisting of a protease inhibitor, a viral packaging inhibitor, a glycosylation inhibitor, and a viral RNA processing inhibitor.

45. The composition of claim 44 wherein the protease inhibitor is selected from the group consisting of:
N-tert-butyl-1-[2(R)-hydroxy-3(S)-[[N-(2-quinolylcarbonyl)-L-asparaginyl]amino]-4-phenylbutyl]-2(S)piperidinecarboxamide;
Carbamic acid, [3-[4-(1-ethylpropyl)-2(S)-[[(1,1-dimethylethyl)amino]carbonyl]-1-piperazinyl]-2(R)-hydroxy-1(S)-(phenylmethyl)propyl]-, tetrahydro-2(R)-(1-methylethyl)-1,1-dioxido-3(R)-thienyl ester;
Carbamic acid, [3-[4-cyclopropyl)-2(S)-[[(1,1-dimethylethyl)amino]carbonyl]-1-piperazinyl]-2(R)-hydroxy-1(S)-(phenylmethyl)propyl]-, tetrahydro-2(R)-(1-methylethyl)-1,1-dioxido-3(R)-thienyl ester;
Carbamic acid, [3-[4-cyclobutyl)-2(S)-[[(1,1-dimethylethyl)amino]carbonyl]-1-piperazinyl]-2(R)-hydroxy-1(S)-(phenylmethyl)propyl]-, tetrahydro-2(R)-(1-methylethyl)-1,1-dioxido-3(R)-thienyl ester;
1′S,2′S,2′S,9S,12R)-12-[2″-[[N-[(benzyloxy)carbonyl]tert-leucinyl]amino]-1′- hydroxy-3′-phenylprop-1′-yl]-9-(1-methylethyl)-7,10,13-triaza-1,4-dioxo-8,11-dioxo[14]paracyclophane;
(1′S,2′S,8S,11R)-11-[2″-[[N-[(benzyloxy)carbonyl]valyl]amino]-1′-hydroxy-3′-phenylprop-1′-yl]-8-(1-methylethyl)-6,9,12-triaza-1-oxa-7,10-dioxo[13]metacyclophane;
(1′S,2′S,2″S,9S,11R)-11-[2″-[[N-[(benzyloxy)carbonyl]valyl]amino]-1′-hydroxy-3′-phenylprop-1′-yl]-8-(1-methylethyl)-6,9,12-triaza-1-oxa-7,10-dioxo[13]paracyclophane;
(1′S,2′S,2″S,9S,12R)-12-[2″-[[N-[(benzyloxy)carbonyl]valyl]amino]-1′-hydroxy-3′-phenylprop-1′-yl]-9-(1-methylethyl)-7,10,13-triaza-1,4-diaza-8,11-dioxo[14]paracyclophane;
(1′S,2′S,2″S,15R)-15-[2″-[[N-[(benzyloxy)carbonyl]valyl]amino]-1′-hydroxy-3′-phenylprop-1′-yl]-12-(1-methylethyl)-10,13,16-triaza-1,4,7-trioxo-11,14-dioxo[17]paracyclophane;
[1(S),4(S)]-2,4,5-trideoxy-4-[[3-methyl-1-oxo-2-[[(phenylmethoxy)carbonyl]amino]butyl]amino]-N-[[2-methyl-1-[(2-benzimidazolyl)lmethyl]amino]carbonyl]propyl]-5-phenyl-2-[(4-bromophenyl)methylamino-L-lyxonamide;
2,5-(S,S)-Bis(2-pyridylmethoxyvalyl)1,6-diphenyl-3,4-(S,S)-dihydroxyhexane;
1,2,5,6-tetradeoxy-2,5-bis[[3-methyl-2-[[methyl(2-pyridinylmethyl)amino]carbonyl]amino]-1-oxobutylamino]-1,6-diphenyl-L-altritol;
10-hydroxy-5-(1-methylethyl)1(2-pyridinyl)-3,6-dioxo-8,11-bis(phenylmethyl)-2-oxa-4,7,12-triazatridecan-13-oic acid, 5-thiazolylmethyl ester;
10-hydroxy-1-[2-(1-methylethyl)-4-thiazolyl]-5-(1-methylethyl)-3,6-dioxo-8,11-bis(phenylmethyl)-2,4,7,12-tetraazatridecan-13-oic acid, 3-pyridinyl ester;
(5R,6R)-2,4-bis(4-hydroxy-3-methoxybenzyl)-1,5-dibenzyl-6-hydroxy-3-oxo-1,2,4-triazacycloheptane;
Carbamic acid, (3-(((4-aminophenyl)sulfonyl)(2-methylpropyl)amino)-2-hydroxy-1-(phenylmethyl)propyl)-,tetrahydro-3-furanyl ester;
[1S-[1R*,2S*(2S*,3R*)]]-[3-[[3-[[(1,1-dimethylethoxy)-carbonyl]amino]-2-hydroxy-4-[4-[2-(4-morpholinyl)-2-oxoethoxy]phenyl]butyl]amino]-2-hydroxy-1-(phenylmethyl)propyl]carbamic acid, 1,1-dimethylethyl-ester;
N-(3-(2(S)-(N-(tert-butyl)carbamonyl)-4(R)-(3-pyridylmethylthio)piperidyl)-1(S)-benzylpropyl)-3-methyl-2(S)-(2-quinolylcarbonylamino)butanamide;.
N-(3-(2(S)-(N-(tert-butyl)carbamoyl)-4(R)-(4-pyridylthio)piperidyl)-1(S)-benzylpropyl)-2-(2,6-dimethylphenoxy)ethanamide;
1cyclohexyl-2-[[N-(ethoxycarbonyl)-L-valinyl]amino]-4(S)-hydroxy-5(S)-[[N-(methoxycarbonyl)-L-valinyl]amino]-6-phenyl-2-azahexane;
1cyclohexyl-5(S)-2,5-bis[[2-N-(methylcarbonyl)-L-valinyl]amino]-4(R)-hydroxy-6-phenyl-2-azahexane;
[4R-(4.alpha.,5.alpha.,6.beta.,7.beta.,7.beta.)]-hexahydro-5,6-dihydroxy-1,3-bis[(4-hydroxymethyl)phenyl]methyl]-4,7-bis(phenylmethyl)-2H-1,3-diazepin-2-one);
[4R-(4.alpha.,5.alpha.,6.beta.,7.beta.)]-hexahydro-5,6-dihydroxy-1,3-bis[(3-aminophenyl)methyl]-4,7-bis(phenylmethyl)-2H-1,3-diazepin-2-one];
2-[3-[3-(R)-[[(2-cis-isopropyl-1,1-dioxotetrahydrothienyloxy)carbonyl]amino]-4-phenyl-2(R)-hydroxybutyl]]-N-(1,1-dimethylethyl)decahydro-3-isoquinolinecarboxamide;
2-[3-[3-(S)-[[(2-cis-isopropyl-1,1-dioxotetrahydrothienyloxy)carbonyl]amino]-4-phenyl-2(R)-hydroxybutyl]]-N-(1,1-dimethylethyl)decahydro-3-isoquinolinecarboxamide;
N\hat{ }2-[(phenylmethoxy)carbonyl]-L-asparaginyl-(2S,3S)-2-hydroxy-4-phenyl-3-aminobutanoyl-N-(1,-dimethylethyl)-L-prolinamide;
N-(2(R)-hydroxy-1(S)-indanyl)-5(S)-[(tert-butyloxycarbonyl)amino]-4(S)-hydroxy-6-(4-hydroxyphenyl)-2-(R)-(phenylmethyl)hexanamide;
N-(2(R)-hydroxy-1(S)-indanyl)-5(S)-[(tert-butyloxycarbonyl)amino]-4(S)-hydroxy-2(R)-[[4-(2-dimethylamino)ethoxy]phenyl]methyl]-6-(phenyl)hexanamide;
N-(2(R)-hydroxy-1(S)-indanyl)-5(S)-[(tert-butyloxycarbonyl)amino]-4(S)-hydroxy-2(R)-[[4-[3-(4-morpholinyl)propoxy]phenyl]methyl]-6-phenyl-hexanamide;
N-((1S)-1-[N-(2-methoxyethyl)carbamoyl]-2-methylpropyl)(4S,5S,2R)-5-[(tertbutoxy)carbonylamino]-4-hydroxy-6-phenyl-2-[(2,3,4-trimethyoxyphenyl)methyl]hexanamide;
N-[(1S,3S,4S)-4-[[(2,6-dimethylphenoxy)acetyl]amino]-3-hydroxy-5-phenyl1-(phenylmethyl)pentyl]tetrahydro-a-(1-methylethyl)-2-oxo-,(aS)-1(2H)-pyrimidineacetamide;
N\hat{ }1-[3-[2-[[(1,1-dimethylethyl)amino]carbonyl]phenyl]-2-hydroxy-1-(phenylmethyl)propyl]-2-[(2-quinolinylcarbonyl)amino]butanediamide;
N-[3-[2-[[(1,1-dimethylethyl)amino]carbonyl]phenyl]-2-hydroxy-1-(phenylmethyl)propyl]-2(D)-(acetylamino)-3-(2-naphthalenylsulfonyl)propanamide;
N-[3-[2-[[(1,1-dimethylethyl)amino]carbonyl]phenyl]-2(R)-hydroxy-1(S)-(phenylmethyl)propyl]-2(R)-(acetylamino)-3-(1-quinolinylsulfonyl) propanamide;
N-2-[2′(S)-hydroxy-3′(S)-phenylmethyl-4′-aza-5′-oxo-6′(S)-methylsulfonylamido-7′-(4-fluorophenylsulfonyl)-heptyl]-(4aS,8aS)-decahydroisoquinoline-3(S)-N-t-butylcarboxamide;
N-(1,1-dimethylethyl)-5-[2-hydroxy-3-[(3-hydroxy-2-methylbenzoyl)amino]-3-[phenyl(thiomethyl]propyl]octahydro-thieno[3,2-c]pyridine-6-carboxamide;
5-[3(R)-[[(2(R)-cis-isopropyl-1,1-dioxotetrahydrothienyl-3(R)-oxy)carbonyl]amino]-4-(phenylthio)-2(R)-hydroxybutyl]-N-(1,1-dimethylethyl)octahydrothieno[3,2-c]pyridine-6(R)-carboxamide;
Lopinavir & Ritonavir;
[(3S-(3R*,4aR*,8aR*,2S*,3′S*)]-2-[2′-hydroxy-3′-phenylthiomethyl-4′-aza-5′-oxo-5′-(2″-methyl-3″-hydroxy-phenyl)pentyl]-decahydroisoquinoline-3-N-t-butylcarboxamidemethanesulfonic acid);
N-[1(S)-[[[3-[2(S)-[[1,1-dimethylethyl)amino]carbonyl]-4(R)-[(4-pyridinylmethyl)oxy]-1-piperidinyl]-2(R)-hydroxy-1(S)-(phenylmethyl)propyl]amino]carbonyl]-2-methylpropyl]-2-quinolinecarboxamide;
[2R-[2.alpha.(R*),4.beta.]]-4,4′-[1,2-ethanediylbis(aminocarbonyl)bis[N-benzyl-5,5-dimethyl-.alpha.[(phenylacetyl)amino]-2-thiazolidineacetamide];
4-[[[2-[[[5,5-dimethyl-2-[2-oxo-1-[(phenylacetyl)amino]-2-[(phenylmethyl)amino]ethyl]-4-thiazolidinyl]carbonyl]amino]ethyl]amino]carbonyl]-5,5-dimethyl-.alpha.-[(phenylacetyl)amino]-N-[4(tertbutoxycarbonyl)phenyl]methyl)-,[2R-[2.alpha.(R*),4.beta.[2R*(R*),4S*]]]-2-thiazolidineacetamide;
4-[[[3-[3-[[(1,1-dimethylethyl)amino]carbonyl]decahydro-2-isoquinolinyl]-2-hydroxypropyl]amino]carbonyl]-5,5-dimethyl-.alpha.-[(phenylacetyl)amino]-N-ethyl-2-thiazolidineacetamide,[2R-[2.alpha.]];
4-[[[3-[3-[[(1,1-dimethylethyl)amino]carbonyl]decahydro-2-isoquinolinyl]-2-hydroxypropyl]amino]carbonyl]-5,5-dimethyl-.alpha.-[(phenylacetyl)amino]-N-[2- [[(1,1-dimethylethoxy)carbonyl]amino]ethyl]-2-;
(2S,3S)-3-[N-(quinoxaline-2-carbonyl)-L-asparaginyl]amino-2-hydroxy-4-phenylbutanoyl-L-proline, tert-butylamide;
[5S-(5R*,8R*,10R*,11R*)]-2,4,7,12-tetraazatridecan-13-oic acid, 10-hydroxy-2-methyl-5-(1-methylethyl)-1-[2-(1-methylethyl)-4-thiazolyl]-3,6-dioxo-8,11-bis(phenylmethyl)-5-thiazolylmethyl ester;
N-2-[2′(S)-hydroxy-3′(S)-phenylmethyl-4′-aza-5′-oxo-6′(S)-methylsulfonylamido-7′-(4-fluorophenylsulfinyl)-heptyl]-(4aS,8aS)-decahydroisoquinoline-3(S)-N-t-butylcarboxamide;
N1-[(1S,2R)-3-[(3S,4aS,8aS)-3-[[(1,1-dimethylethyl)amino]carbonyl]octahydro-2(1H)-isoquinolinyl]-2-hydroxy-1-(phenylmethyl)propyl]-2-[(2-quinolinylcarbonyl)amino]-(2S)-butanediamide;
N-tert-butyl-1-[2(R)-hydroxy-3(S)-[[N-(2-quinolylcarbonyl)-L-asparaginyl]amino]-4-phenylbutyl]-2(S)piperidinecarboxamide;
N-[(1S)-1-(N-(lmino[(phenylmethoxy)carbonylamino]methyl)carbomoyl)-2-methylpropyl](4S,5S,2R)-5-[(tert-butoxy)carbonylamino]-4-hydroxy-6-phenyl-2-benzylhexanamide;
N-tert-butyl-N′-isobutyl-N′-[2(R)-hydroxy-4-phenyl-3(S)-[4-amino-1,4-dioxo-2(S)-(2-quinolinylcarboxamido)butylamino]butyl]urea;
[4R-(4.alpha.,5.alpha.,6.beta.,7.beta.)]-3,3′-[[tetrahydro-5,6-dihydroxy-2-oxo-4,7-bis(phenylmethyl)-1H-1,3-diazepine-1,3(2H)-diyl]-bis(methylene)]bis[N-1H-benzimidazol-2-ylbenzamide];
(2R,3S,4S,1′S,2′R)-4-[[[N-[(benzyloxy)carbonyl]-L-tert-leucyl]amino]-3-hydroxy-2-[(4-methoxybenzyl)amino]-5-phenylpentan(2′-hydroxy-1′-indanyl)amide;
5-[3(R)-[[(1,1-dioxotetrahydrothienyl-3(S)-oxy)carbonyl]amino]-4-(phenylthio)-2(R)-hydroxybutyl]-N-(1,1-dimethylethyl)octahydrothieno[3,2-c]pyridine-6(R)-carboxamide;
5-[3(R)-[[(2(R)-cis-methyl-1,1-dioxotetrahydrothienyl-3(S)-oxy)carbonyl]amino]-4-(phenylthio)-2(R)-hydroxybutyl]-N-(1,1-dimethylethyl)octahydrothieno[3,2-c]pyridine-6(R)-carboxamide;
5-[3(R)-[[(2(R)-cis-isopropyl-tetrahydrothienyl-3(R)-oxy)carbonyl]amino]-4-phenyl-2(R)-hydroxybutyl]-N-(1,1-dimethylethyl)octahydrothieno[3,2-c]pyridine-6(R)-carboxamide;
5-[3(R)-[[(2(R)-cis-isopropyl-1,1-dioxotetrahydrothienyl-3(R)-oxy)carbonyl]amino]-4-phenyl-2(R)-hydroxybutyl]-N-(1,1-dimethylethyl)octahydrothieno[3,2-c]pyridine-6(R)-carboxamide;
(6R)-3-((1R)-1-[3-(([5-(trifluoromethyl)(2-pyridyl)]sulfonyl)amino)phenyl]propyl)-4-hydroxy-6-(2-phenylethyl)-6-propyl-5,6-dihydro-2H-pyran-2-one;
(2R,3S,4S)-N-[2-(4-chlorobenzylamino)-4-[[N-[(benzyloxy)carbonyl]tert-leucine]amino]-3-hydroxy-5-phenylpentanoyl]valine(2-benzimidazolyl)methylamide;
(2R,3S,4S)-N-[2-(benzylamino)-4-[[N-[(benzyloxy)carbonyl]valyl]amino]-3-hydroxy-5-phenylpentanoyl]valine benzylamide;
(2R,3S,4S)-N-[2-[(4-bromophenyl)methylamino]-4-[[N-[(benzyloxy)carbonyl]valyl]amino]-3-hydroxy-5-phenylpentanoyl]valinebenzylamide;
(2R,3S,4S)-N-[2-(benzylamino)-4-[[N-[(2-benzimidazolyl)propanoyl]valyl]amino]-3-hydroxy-5-phenylpentanoy]valine benzylamide;
(2R,3S,4S)-N-[2-(benzylamino)-4-[[N-[(benzyloxy)carbonyl]valyl]amino]-3-hydroxy-5-phenylpentanoyl]valine(2-benzimidazolyl)methylamide;
(2R,3S,4S)-N-[2-[(4-methoxybenzylamino]-4-[[N-[(benzyloxy)carbonyl]valyl]amino]-3-hydroxy-5-phenylpentanoyl]valine(2-benzimidazolyl)methylamide;
[3-[[(4-methoxyphenyl)sulfonyl](cyclopentylmethyl)amino]-2-hydroxy-1-(phenylmethyl)propyl]-tetrahydro-3-furanylester carbamic acid;
(2-naphthalcarbonyl)Asn[decarbonylphe-hydroxyethyl]ProtOtertbutyl;
N\hat{ }1-[3-[4-[[(1,1-dimethylethyl)amino]carbonyl]-5,5-dimethyl-3-thiazolidinyl]-2-hydroxy-3-oxo-1-(phenylmethyl)propyl]-2-[[(1-naphthalenyloxy)acetyl]amino]butanediamide,[4R-[3 [1S*,(S*).2S*]],4R*]];
N-(1,1-dimethylethyl)-3-[2-hydroxy-3-[[2-[[(5-isoquinolinyloxy)acetyl]amino]-3-methylthio)-1-oxopropyl]amino]-1-oxo-4-phenylbutyl]-5,5-dimethyl]-4-thiazolidinecarboxamide,[4R-[3[2S*,3S*(R*)],4R*]];
N-(1,1-dimethylethyl)-3-[2-hydroxy-3-[[2-[[(5-isoquinolinyloxy)acetyl]amino]-3-methylthio)-1-oxopropyl]amino]-1-oxo-4-phenylbutyl]-4-thiazolidinecarboxamide,[4R-[3[2S*,3S*(R*)],4R*]];
N-[2(R)-hydroxy-1(S)-indanyl]-5(S)-[[(1,1-dimethylethoxy)carbonyl]amino]-4(S)-hydroxy-6-phenyl-2(R)-benzylhexanamide;
6-phenyl-5-(N-t-butyloxycarbonylamino)-4-hydroxy-2-(3-phenylprop-2-ene)-1-[(2-(aminomethyl)benzimidazole)-isoleucyl]-hexanone;
N-(2(R)-hydroxy-1(S)-indanyl)-5(S)-[(tert-butyloxycarbonyl)amino]-4(S)-hydroxy-6-phenyl-2(R)-[[4-[2-(4-morpholinyl)ethoxy]phenyl]methyl]hexanamide;
(3R,3aS,5S,6aR)-N-tert-butyl-2-[2′-hydroxy-4′-phenyl-3′-[[[(3′″-hexahydrofuro[2,3-b]furanyl)oxy]carbonyl]amino]butyl]decahydroisoquinoline-3-carboxamide;
N1-[(1S,2R)-3-[(3S,4aS,8aS)-3-[[(1,1-dimethylethyl)amino]carbonyl]octahydro-2(1H)-isoquinolinyl]-2-hydroxy-1-(phenylmethyl)propyl]-2-[(2-quinolinylcarbonyl) amino]
or a prodrug thereof.

46. The composition of claim 40 wherein the viral cellular entry inhibitor is selected from the group consisting of a virion receptor binding antagonist, a virion co-receptor binding antagonist, a viral fusion inhibitor, and a viral uncoating inhibitor.

47. The composition of claim 46 wherein the receptor binding antagonist or co-receptor binding antagonist is a CD4 receptor antagonist.

48. The composition of claim 41 wherein the human immune enhancing agent is selected from the group consisting of an antimetabolite, an antineoplastic agent, an immune modulator, a cytokine, a therapeutic vaccine or antibody, an antioxidant, a hormone, and a vitamin.

49. The composition of claim 40 wherein the integrase inhibitor is selected from the group consisting of:
4-((3,4-dimethoxyphenyl)methyl)dihydro-3-((4-hydroxy-3-methoxyphenyl)methyl)-(3R-trans)-2(3H)-furanone;
3,5-dicaffeoylquinic acid;
1-methoxyaxalyl-3,5-dicaffeoylquinic acid;
9-[(4,6-O-ethylidene-.beta.-D-glucopyranosyl)oxy]-5,8,8a,9-tetrahydro-5-(4-hydroxy-3,5-dimethoxyphenyl)furo[3′,4′:6,7]naphtho[2,3-d]1,3-dioxol-6-(5aH)-one;
Hydroxocobalamin;
[S-(R*,R*)]-2,3-bis[[3-(3,4-dihydroxyphenyl)-1-oxo-2-propenyl]oxy]butanedioic acid.

50. The composition of claim 1 wherein the cyclooxygenase-2 selective inhibitor is celecoxib.

51. The composition of claim 1 wherein the anti-human immunodeficiency virus agent is selected from a natural product.

52. The composition of claim 51 wherein the natural product is selected from the group consisting of:
(R)-3,6-diamino-N-(aminomethyl)hexanamide;
3-hydroxylup-20(29)-en-28-oic acid, (3.beta.);
3-O-(3′,3′-dimethylsuccinyl)-betulinic acid;
Conocurvone;
Cyanovirin-N;
3.beta.-hydroxyandrost-5-en-17-one;
16-.alpha.-bromo-3-.beta.-hydroxyandrost-5-en-17-one;
9-(guanidino)-N-[10-(guanidino)-1-(3-aminopropyl)-2-hydroxydecyl]nonanamide;
6-acetyloxy-7-(acetyloxymethyl)-5-hydroxy-3,11,11,14-tetramethyl-15-oxotetracyclo[7.5.1.0<1,5>.0<10,12>]pentadeca-2,7-dien-4-yl] acetate;
13-hydroxyingenol-3-(2,3-dimethylbutanoate)-13-dodecanoate;
1,2-dithiolane-3-pentanoic acid;
2,4,6,8,10,14-octadecahexaenamide,13-hydroxy-N-[(1S)-2-hydroxy-1-methylethyl]-2,10,12,14,16-pentamethyl-18-phenyl-,(2E,4E,6Z,8E, 10E,12R,13R,14E,16S);
4H-pyran-4-one,3-ethyl-6-methoxy-5-methyl-2-(2-(3-methyl-4-phenyl-3-butenyl)-4-o-xazolyl)-,(E);
12-deoxyphorbol-13-(3E,5E-decadienoate);
3-hydroxy-20-oxonorlupan-28-oic acid, (3.beta.);
12-deoxyphorbol-13-acetate;
4oxazolecarboxamide,2-[4,4′,4″,5,5′,5″-hexahydro-4,4′,4″-trimethyl-2″-(2-phenylethenyl)[2,4′:2′,4″-terthiazol]-4-yl]-N,5-dimethyl-,[4R-[2[2′[2″(E),4″S*],4′S*],4R*]];
Acemannan;
5,6,7-trihydroxyflavone-7-O-b-D-glucopyranosideuronic acid;
Calanolide A;
Calanolide B;
(1S,6S,7R,8R,8aR)-1,6,7,8-tetrahydroxyindolizidine;
1,5-Dideoxy-1,5-imino-D-glucitol;
3,5-dicaffeoylquinic acid;
1-methoxyaxalyl-3,5-dicaffeoylquinic acid;
hypericin;
inophyllum B;
inophyllum P;
5,5′-(1,1′-dihydroxy-8,8′-dimethoxy-6,6′-dimethyl[2,2′-binaphthalene]-4,4′-diyl)bis[1,2,3,4,-tetrahydro-1,3-dimethyl-6,8-isoquinolinediol];
5,5′-(1,1′-dihydroxy-8,8′-dimethoxy-6,6′-dimethyl[2,2′-binaphthalene]-4,4′-diyl)bis[1,2,3,4,-tetrahydro-1,3-dimethyl-6,8-isoquinolinediol]; and
5,5′-(1,1′-dihydroxy-8,8′-dimethoxy-6,6′-dimethyl[2,2′-binaphthalene]-4,4′-diyl)[3,4,-dihydro-8-methoxy-1,3-dimethyl-6-isoquinolinediol],[1,2,3,4-tetrahydro-1,3-dimethyl-6,8-isoquinolinediol].

53. The composition of claim 37 wherein the anti-human immunodeficiency virus agent substantially inhibits viral replication, colonization, or assembly in the human.

54. The composition of claim 38 wherein the anti-human immunodeficiency virus agent substantially inhibits mitosis in cells of the human.

55. The composition of claim 38 wherein the anti-human immunodeficiency virus agent substantially increases the immune response of the human.

56. The composition of claim 38 wherein the anti-human immunodeficiency virus agent substantially reduces cellular replication in the human.

57. The composition of claim 56 wherein the agent is a virucidal agent.

58. The composition of claim 57 wherein the virucidal agent is selected from the group consisting of cidofovir, formaldehyde, and glutaral or a prodrug thereof.

59. The composition of claim 57 wherein the virucidal agent is a T-cell proliferation inhibitor.

60. A method for the treatment or prevention of human immunodeficiency viral infection in a human, the method comprising administering to the human a cyclooxygenase-2 selective inhibitor or a pharmaceutically acceptable salt, isomer or prodrug thereof and anti-human immunodeficiency virus agent.

61. The method of claim 60 wherein the cyclooxygenase-2 selective inhibitor comprises a chromene compound.

62. The method of claim 61 wherein the chromene compound is a benzopyran or substituted benzopyran analog.

63. The method of claim 62 wherein the benzopyran or substituted benzopyran analog is selected from the group consisting of benzothiopyrans, dihydroquinolines and dihydronaphthalenes.

64. The method of claim 60 wherein the cyclooxygenase-2 selective inhibitor comprises a tricyclic compound.

65. The method of claim 64 wherein the tricyclic compound comprises a benzenesulfonamide or methylsulfonylbenzene.

66. The method of claim 60 wherein the cyclooxygenase-2 selective inhibitor comprises a phenyl acetic acid derivative.

67. The method of claim 60 wherein the cyclooxygenase-2 selective inhibitor comprises: 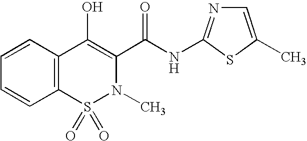
or pharmaceutically acceptable salt, ester, isomer or prodrug thereof.

68. The method of claim 60 wherein the cyclooxygenase-2 selective inhibitor comprises: 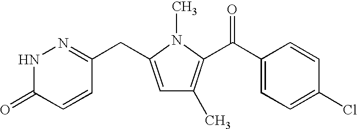
or a pharmaceutically acceptable salt, ester, isomer or prodrug thereof.

69. The method of claim 60 wherein the cyclooxygenase-2 selective inhibitor comprises a compound of the formula or a pharmaceutically acceptable salt or prodrug thereof: 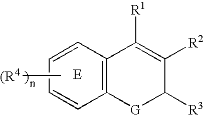
wherein:
n is an integer which is 0, 1, 2, 3 or 4;
G is O, S or NR^{a};
R¹ is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;
wherein R⁴ together with the carbon atoms to which it is attached and the remainder of ring E forms a naphthyl radical.

70. The method of claim 69 wherein:
n is an integer which is 0, 1, 2, 3 or 4;
G is O, S or NR^{b};
R¹ is H;
R^{b} is alkyl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

71. The method of claim 69 wherein:
n is an integer which is 0, 1, 2, 3 or 4;
G is oxygen or sulfur;
R¹ is H;
R² is carboxyl, lower alkyl, lower aralkyl or lower alkoxycarbonyl;
R³ is lower haloalkyl, lower cycloalkyl or phenyl; and
each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or wherein R⁴ together with the carbon atoms to which it is attached and the remainder of ring E forms a naphthyl radical.

72. The method of claim 69 wherein:
R² is carboxyl;
R³ is lower haloalkyl; and
each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

73. The method of claim 69 wherein:
n is an integer which is 0, 1, 2, 3 or 4;
R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and
each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

74. The method of claim 69 wherein the cyclooxygenase-2 selective inhibitor comprises a compound of the formula or a pharmaceutically acceptable salt or prodrug thereof: 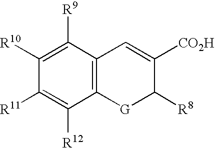
wherein:
G is oxygen or sulfur;
R⁸ is trifluoromethyl or pentafluoroethyl;
R⁹ is H, chloro, or fluoro;
R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;
R¹¹ is H, methyl, ethyl, isopropyl, tert-butyl, chloro, methoxy, diethylamino, or phenyl; and
R¹² is H, chloro, bromo, fluoro, methyl, ethyl, tert-butyl, methoxy, or phenyl.

75. The method of claim 69 wherein the cyclooxygenase-2 selective inhibitor, pharmaceutically acceptable salt, isomer or prodrug thereof is selected from the group consisting of:
6-chloro-2-trifluoromethyl-2H-1-benzopyran-3-carboxylic acid;
6-chloro-7-methyl-2-trifluoromethyl-2H-1-benzopyran-3-carboxylic acid;
8-(1-methylethyl)-2-trifluoromethyl-2H-1-benzopyran-3-carboxylic acid;
6-chloro-7-(1,1-dimethylethyl)-2-trifluoromethyl-2H-1-benzopyran-3-carboxylic acid;
6-chloro-8-(1-methylethyl)-2-trifluoromethyl-2H-1-benzopyran-3-carboxylic acid;
2-trifluoromethyl-3H-naphthopyran-3-carboxylic acid;
7-(1,1-dimethylethyl)-2-trifluoromethyl-2H-1-benzopyran-3-carboxylic acid;
6-bromo-2-trifluoromethyl-2H-1-benzopyran-3-carboxylic acid;
8-chloro-2-trifluoromethyl-2H-1-benzopyran-3-carboxylic acid;
6-trifluoromethoxy-2-trifluoromethyl-2H-1-benzopyran-3-carboxylic acid;
5,7-dichloro-2-trifluoromethyl-2H-1-benzopyran-3-carboxylic acid;
8-phenyl-2-trifluoromethyl-2H-1-benzopyran-3-carboxylic acid;
7,8-dimethyl-2-trifluoromethyl-2H-1-benzopyran-3-carboxylic acid;
6,8-bis(dimethylethyl)-2-trifluoromethyl-2H-1-benzopyran-3-carboxylic acid;
7-(1-methylethyl)-2-trifluoromethyl-2H-1-benzopyran-3-carboxylic acid;
7-phenyl-2-trifluoromethyl-2H1benzopyran-3-carboxylic acid;
6-chloro-7-ethyl-2-trifluoromethyl-2H-1-benzopyran-3-carboxylic acid;
6-chloro-8-ethyl-2-trifluoromethyl-2H-1-benzopyran-3-carboxylic acid;
6-chloro-7-phenyl-2-trifluoromethyl-2H-1-benzopyran-3-carboxylic acid;
6,7-dichloro-2-trifluoromethyl-2H-1-benzopyran-3-carboxylic acid;
6,8-dichloro-2-trifluoromethyl-2H-1-benzopyran-3-carboxylic acid;
2-trifluoromethyl-3H-naptho[2,1-b]pyran-3-carboxylic acid;
6-chloro-8-methyl-2-trifluoromethyl-2H-1-benzopyran-3-carboxylic acid;
8-chloro-6-methyl-2-trifluoromethyl-2H1benzopyran-3-carboxylic acid;
8-chloro-6-methoxy-2-trifluoromethyl-2H1benzopyran-3-carboxylic acid;
6-bromo-8-chloro-2-trifluoromethyl-2H-1-benzopyran-3-carboxylic acid;
8-bromo-6-fluoro-2-trifluoromethyl-2H-1-benzopyran-3-carboxylic acid;
8-bromo-6-methyl-2-trifluoromethyl-2H1benzopyran-3-carboxylic acid;
8-bromo-5-fluoro-2-trifluoromethyl-2H-1-benzopyran-3-carboxylic acid;
6-chloro-8-fluoro-2-trifluoromethyl-2H-1-benzopyran-3-carboxylic acid;
6-bromo-8-methoxy-2-trifluoromethyl-2H-1-benzopyran-3-carboxylic acid;
6-[[(phenylmethyl)amino]sulfonyl]-2-trifluoromethyl-2H-1-benzopyran-3-carboxylic acid;
6-[(dimethylamino)sulfonyl]-2-trifluoromethyl-2H-1-benzopyran-3-carboxylic acid;
6-[(methylamino)sulfonyl]-2-trifluoromethyl-2H1benzopyran-3-carboxylic acid;
6-[(4-morpholino)sulfonyl]-2-trifluoromethyl-2H1benzopyran-3-carboxylic acid;
6-[(1,1-dimethylethyl)aminosulfonyl]-2-trifluoromethyl-2H-1-benzopyran-3-carboxylic acid;
6-[(2-methylpropyl)aminosulfonyl]-2-trifluoromethyl-2H-1-benzopyran-3-carboxylic acid;
6-methylsulfonyl-2-trifluoromethyl-2H-1-benzopyran-3-carboxylic acid;
8-chloro-6-[[(phenylmethyl)amino]sulfonyl]-2-trifluoromethyl-2H1benzopyran-3-carboxylic acid;
6-phenylacetyl-2-trifluoromethyl-2H-1-benzopyran-3-carboxylic acid;
6,8-dibromo-2-trifluoromethyl-2H-1-benzopyran-3-carboxylic acid;
8-chloro-5,6-dimethyl-2-trifluoromethyl-2H-1-benzopyran-3-carboxylic acid;
6,8-dichloro-(S)-2-trifluoromethyl-2H-1-benzopyran-3-carboxylic acid;
6-benzylsulfonyl-2-trifluoromethyl-2H-1-benzopyran-3-carboxylic acid;
6-[[N-(2-furylmethyl)amino]sulfonyl]-2-trifluoromethyl-2H-1-benzopyran-3-carboxylic acid;
6-[[N-(2-phenylethyl)amino]sulfonyl]-2-trifluoromethyl-2H-1-benzopyran-3-carboxylic acid;
6-iodo-2-trifluoromethyl-2H1benzopyran-3-carboxylic acid;
7-(1,1-dimethylethyl)-2-pentafluoroethyl-2H-1-benzopyran-3-carboxylic acid; and
6-chloro-2-trifluoromethyl-2H-1-benzothiopyran-3-carboxylic acid.

76. The method of claim 69 wherein the cyclooxygenase-2 selective inhibitor, pharmaceutically acceptable salt or prodrug thereof is selected from the group consisting of formulas: 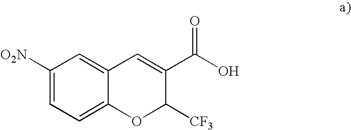
6-Nitro-2-trifluoromethyl-2H-1-benzopyran-3-carboxylic acid; 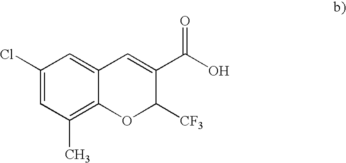
6-Chloro-8-methyl-2-trifluoromethyl -2H-1-benzopyran-3-carboxylic acid; 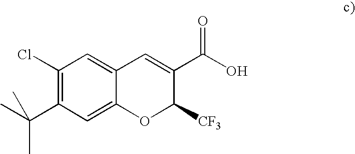
((S)-6-Chloro-7-(1,1-dimethylethyl)-2-(trifluo romethyl-2H-1-benzopyran-3-carboxylic acid; 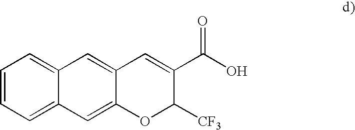
2-Trifluoromethyl-2H-naphtho[2,3-b]pyran-3-carboxylic acid; 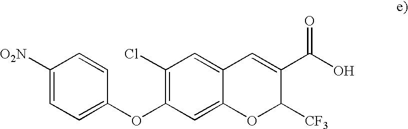
6-Chloro-7-(4-nitrophenoxy)-2-(trifluoromethyl)-2H-1-benzopyran-3-carboxylic acid; 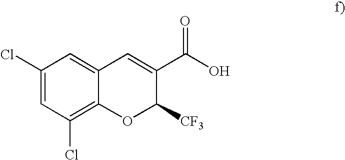
((S)-6, 8-Dichloro-2-(trifluoromethyl)-2H-1-benzopyran-3-carboxylic acid; 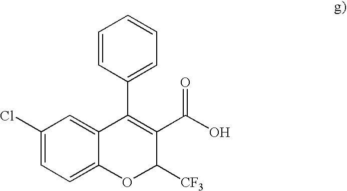
6-Chloro-2-(trifluoromethyl)-4-phenyl-2H-1-benzopyran-3-carboxylic acid; 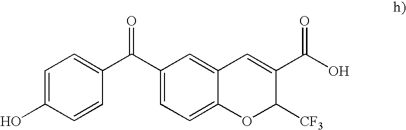
6-(4-Hydroxybenzoyl)-2-(trifluoromethyl) -2H-1-benzopyran-3-carboxylic acid; 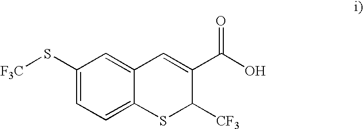
2-(Trifluoromethyl)-6-[(trifluoromethyl)thio]-2H-1-benzothiopyran-3-carboxylic acid; 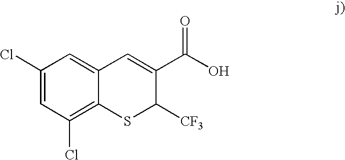
6,8-Dichloro-2-trifluoromethyl-2H-1-benzothiopyran-3-carboxylic acid; 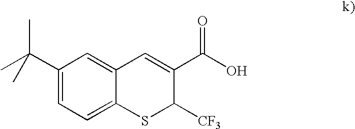
6-(1,1-Dimethylethyl)-2-(trifluoromethyl) -2H-1-benzothiopyran-3-carboxylic acid; 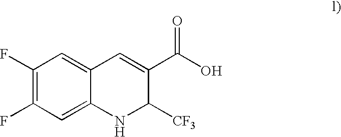
6,7-Difluoro-1,2-dihydro-2-(trifluoro methyl)-3-quinolinecarboxylic acid; 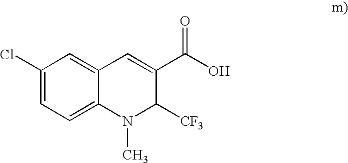
6-Chloro-1,2-dihydro-1-methyl-2-(trifluoro methyl)-3-quinolinecarboxylic acid; 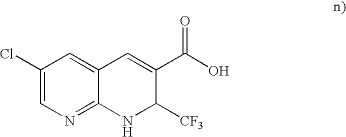
6-Chloro-2-(trifluoromethyl)-1,2-dihydro [1,8]naphthyridine-3-carboxylic acid; 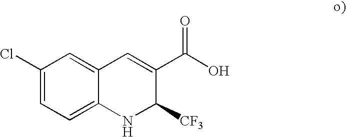
((S)-6-Chloro-1,2-dihydro-2-(trifluoro methyl)-3-quinolinecarboxylic acid;
and any combination thereof.

77. The method of claim 60 wherein the cyclooxygenase-2 selective inhibitor comprises a composition of the formula or a pharmaceutically acceptable salt or prodrug thereof: 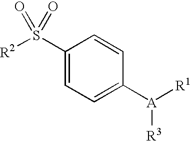
wherein:
A is selected from the group consisting of partially unsaturated or unsaturated heterocyclyl and partially unsaturated or unsaturated carbocyclic rings;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl or amino; and
R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

78. The method of claim 60 wherein the cyclooxygenase-2 selective inhibitor pharmaceutically acceptable salt or prodrug thereof is selected from the group consisting of: 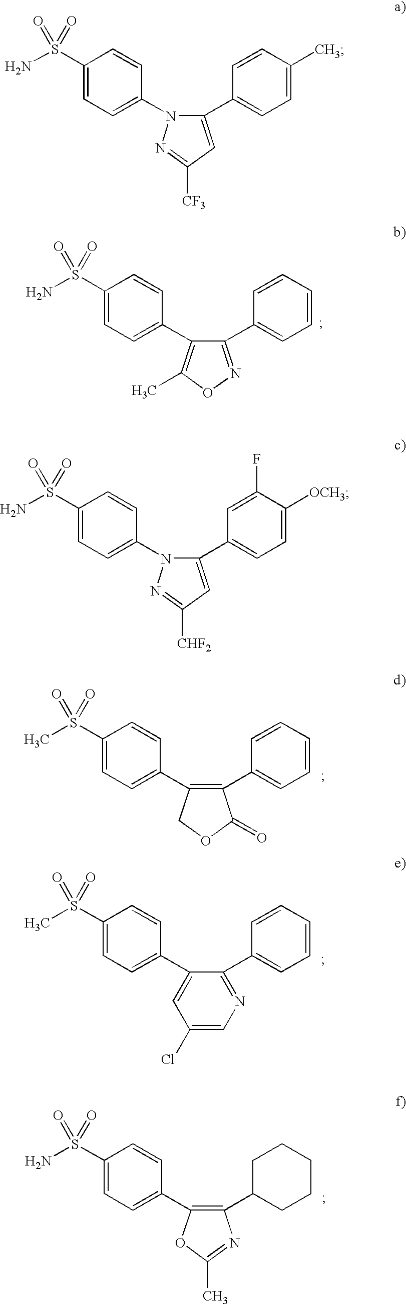
and any combination thereof.

79. The method of claim 60 wherein the cyclooxygenase-2 selective inhibitor or a pharmaceutically acceptable salt or prodrug thereof is selected from the group consisting of: 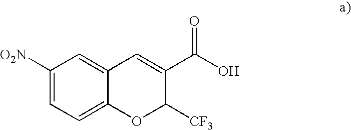
6-Nitro-2-trifluoromethyl-2H-1-benzopyran-3-carboxylic acid; 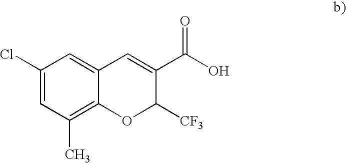
6-Chloro-8-methyl-2-trifluoromethyl -2H-1-benzopyran-3-carboxylic acid; 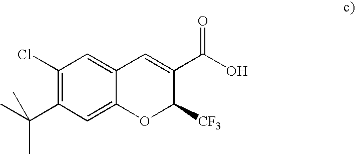
((S)-6-Chloro-7-(1,1-dimethylethyl)-2-(trifluo romethyl-2H-1-benzopyran-3-carboxylic acid; 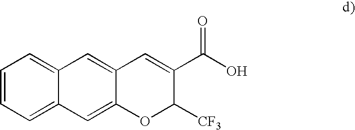
2-Trifluoromethyl-2H-naphtho[2,3-b]pyran-3-carboxylic acid; 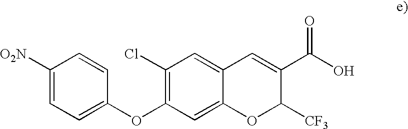
6-Chloro-7-(4-nitrophenoxy)-2-(trifluoromethyl)-2H-1-benzopyran-3-carboxylic acid; 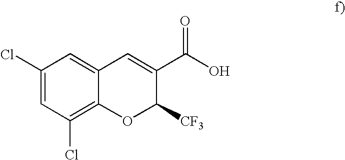
((S)-6,8-Dichloro-2-(trifluoromethyl)-2H-1-benzopyran-3-carboxylic acid; 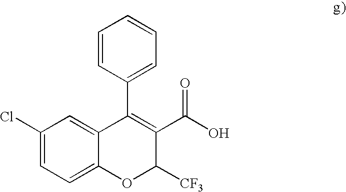
6-Chloro-2-(trifluoromethyl)-4-phenyl-2H-1-benzopyran-3-carboxylic acid; 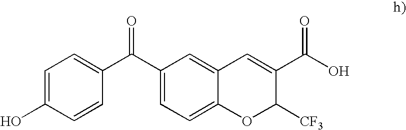
6-(4-Hydroxybenzoyl)-2-(trifluoromethyl) -2H-1-benzopyran-3-carboxylic acid; 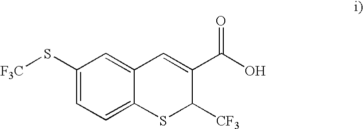
2-(Trifluoromethyl)-6-[(trifluoromethyl)thio]-2H-1-benzothiopyran-3-carboxylic acid; 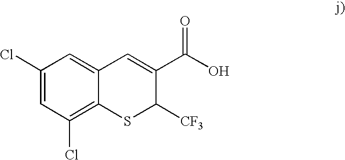
6,8-Dichloro-2-trifluoromethyl-2H-1-benzothiopyran-3-carboxylic acid; 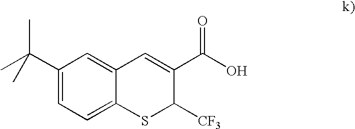
6-(1,1-Dimethylethyl)-2-(trifluoromethyl) -2H-1-benzothiopyran-3-carboxylic acid; 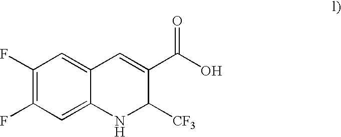
6,7-Difluoro-1,2-dihydro-2-(trifluoro methyl)-3-quinolinecarboxylic acid; 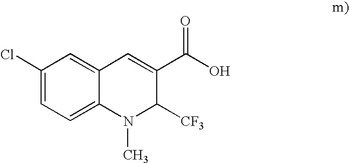
6-Chloro-1,2-dihydro-1-methyl-2-(trifluoro methyl)-3-quinolinecarboxylic acid; 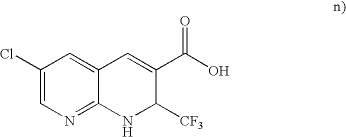
6-Chloro-2-(trifluoromethyl)-1,2-dihydro [1,8]naphthyridine-3-carboxylic acid; 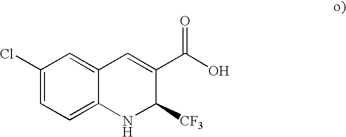
((S)-6-Chloro-1,2-dihydro-2-(trifluoro methyl)-3-quinolinecarboxylic acid; 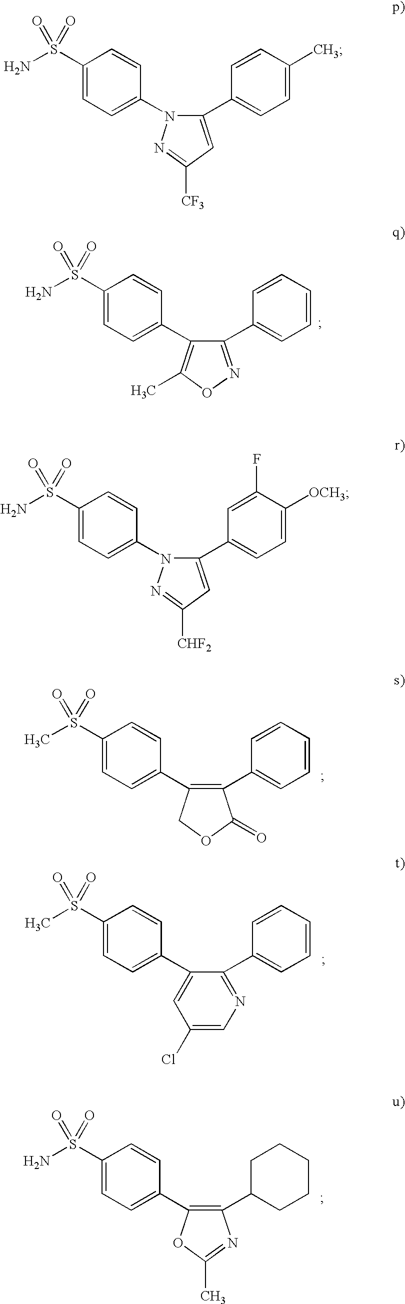 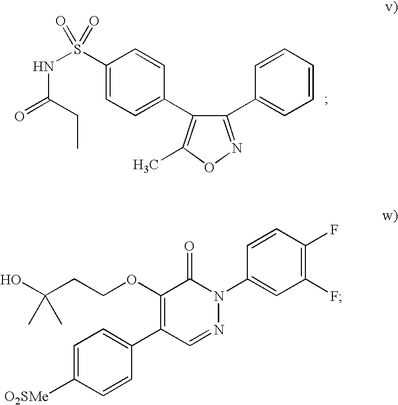
and any combination thereof.

80. The method of claim 60 wherein the cyclooxygenase-2 selective inhibitor comprises: 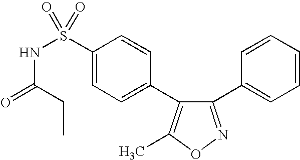
or a pharmaceutically acceptable salt, ester, isomer or prodrug thereof.

81. The method of claim 60 wherein the cyclooxygenase-2 selective inhibitor comprises: 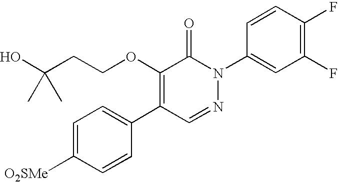
or a pharmaceutically acceptable salt, ester, isomer or prodrug thereof.

82. The method of claim 60 wherein the cyclooxygenase-2 selective inhibitor comprises 4-[4-(methyl)-sulfonyl)phenyl]-3-phenyl-2(5H)-furanone, or a pharmaceutically acceptable salt, ester, isomer or prodrug thereof.

83. The method of claim 60 wherein the cyclooxygenase-2 selective inhibitor comprises 4-(5-methyl-3-phenyl-4-isoxazolyl), or a pharmaceutically acceptable salt, ester, isomer or prodrug thereof.

84. The method of claim 60 wherein the cyclooxygenase-2 selective inhibitor comprises 2-(6-methylpyrid-3-yl)-3-(4-methylsulfonylphenyl)-5-chloropyridine, or a pharmaceutically acceptable salt, ester, isomer or prodrug thereof.

85. The method of claim 60 wherein the cyclooxygenase-2 selective inhibitor comprises 4-[5-(4-methylphenyl)-3-(trifluoromethyl)-1H-pyrazol-1-yl], or a pharmaceutically acceptable salt, ester, isomer or prodrug thereof.

86. The method of claim 60 wherein the cyclooxygenase-2 selective inhibitor comprises N-[[4-(5-methyl-3-phenyl-4-isoxazolyl)phenyl]sulfonyl], or a pharmaceutically acceptable salt, ester, isomer or prodrug thereof.

87. The method of claim 60 wherein the cyclooxygenase-2 selective inhibitor comprises 4-[5-(3-fluoro-4-methoxyphenyl)-3-difluoromethyl)-1H-pyrazol-1-yl]benzenesulfonamide, or a pharmaceutically acceptable salt, ester, isomer or prodrug thereof.

88. The method of claim 60 wherein the cyclooxygenase-2 selective inhibitor comprises (S)-6,8-dichloro-2-(trifluoromethyl)-2H1benzopyran-3-carboxylic acid, or a pharmaceutically acceptable salt, ester, isomer or prodrug thereof.

89. The method of claim 60 wherein the cyclooxygenase-2 selective inhibitor comprises 2-(3,4-difluorophenyl)-4-(3-hydroxy-3-methylbutoxy)-5-[4-(methylsulfonyl)phenyl]-3(2H)-pyridzainone, or a pharmaceutically acceptable salt, ester, isomer or prodrug thereof.

90. The method of claim 60 wherein the cyclooxygenase-2 selective inhibitor comprises a compound of the formula or a pharmaceutically acceptable salt or prodrug thereof: 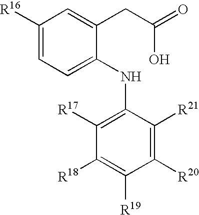
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

91. The method of claim 90 wherein:
R¹⁶ is ethyl;
R¹⁷ and R¹⁹ are chloro;
R¹⁸ and R²⁰ are hydrogen; and
and R²¹ is methyl.

92. The method of claim 60 wherein the cyclooxygenase-2 selective inhibitor comprises a compound of the formula or a pharmaceutically acceptable salt or prodrug thereof: 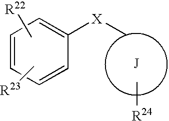
wherein:
X is O or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄.

93. The method of claim 60 wherein the cyclooxygenase-2 selective inhibitor comprises a compound of the formula or a pharmaceutically acceptable salt or prodrug thereof 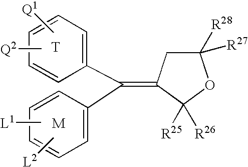
wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q² , L¹ or L² are independently hydrogen, halogen, lower alkyl having from 1 to 6 carbon atoms, trifluoromethyl, or lower methoxy having from 1 to 6 carbon atoms; and at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,
Q¹ and Q² are methylenedioxy; or
L¹ and L² are methylenedioxy; and
R²⁵, R², R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²⁵ and R²⁶ are O; or,
R²⁷ and R²⁸ are O; or,
R²⁵, R²⁶, together with the carbon atom to which they are attached, form a saturated hydrocarbon ring having from 3 to 7 carbon atoms; or,
R²⁷, R²⁸, together with the carbon atom to which they are attached, form a saturated hydrocarbon ring having from 3 to 7 carbon atoms.

94. The method of claim 60 wherein the cyclooxygenase-2 selective inhibitor pharmaceutically acceptable salt, isomer, or prodrug thereof is selected from the group consisting of:
3-[(3-Chloro-phenyl)-(4-methanesulfonyl-phenyl)-methylene]-dihydro-furan-2-one;
8-acetyl-3-(4-fluorophenyl)-2-(4-methylsulfonyl)phenyl-imidazo(1,2-a);
5,5-dimethyl-4-(4-methylsulfonyl)phenyl-3-phenyl-2-(5H)-furanone;
5-(4-fluorophenyl)-1-[4-(methylsulfonyl)phenyl]-3-(trifluoromethyl)pyrazole;
4(4-fluorophenyl)-5-[4-(methyl sulfonyl)phenyl]1phenyl-3-(trifluoromethyl)pyrazole;
4(5-(4-chlorophenyl)-3-(4-methoxyphenyl)-1H-pyrazol-1-yl)benzenesulfonamide;
4(3,5-bis(4-methylphenyl)-1H-pyrazol-1-yl)benzenesulfonamide;
4(5-(4-chlorophenyl)-3-phenyl-1H-pyrazol-1-yl)benzenesulfonamide;
4(3,5-bis(4-methoxyphenyl)-1H-pyrazol-1-yl)benzenesulfonamide;
4(5-(4-chlorophenyl)-3-(4-methylphenyl)-1H-pyrazol-1-yl)benzenesulfonamide;
4(5-(4-chlorophenyl)-3-(4-nitrophenyl)-1H-pyrazol-1-yl)benzenesulfonamide;
4(5-(4-chlorophenyl)-3-(5-chloro-2-thienyl)-1H-pyrazol-1-yl)benzenesulfonamide;
4(4-chloro-3,5-diphenyl-1H-pyrazol-1-yl)benzenesulfonamide;
4-[5-(4-chlorophenyl)-3-(trifluoromethyl)-1H-pyrazol-1-yl]benzenesulfonamide;
4-[5-phenyl-3-(trifluoromethyl)-1H-pyrazol-1-yl]benzenesulfonamide;
4-[5-(4-fluorophenyl)-3-(trifluoromethyl)-1H-pyrazol-1-yl]benzenesulfonamide;
4-[5-(4-methoxyphenyl)-3-(trifluoromethyl)-1H-pyrazol-1-yl]benzenesulfonamide;
4-[5-(4-chlorophenyl)-3-(difluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[4-chloro-5-(4-chlorophenyl)-3-(trifluoromethyl)-1H-pyrazol-1-yl]benzenesulfonamide;
4-[3-(difluoromethyl)-5-(4-methylphenyl)-1H-pyrazol-1-yl]benzenesulfonamide;
4-[3-(difluoromethyl)-5-phenyl-1H-pyrazol-1-yl]benzenesulfonamide;
4-[3-(difluoromethyl)-5-(4-methoxyphenyl)-1H-pyrazol-1-yl]benzenesulfonamide;
4-[3-cyano-5-(4-fluorophenyl)-1H-pyrazol-1-yl]benzenesulfonamide;
4-[3-(difluoromethyl)-5-(3-fluoro-4-methoxyphenyl)-1H-pyrazol-1-yl]benzenesulfonamide;
4-[5-(3-fluoro-4-methoxyphenyl)-3-(trifluoromethyl)-1H-pyrazol-1-yl]benzenesulfonamide;
4-[4-chloro-5-phenyl-1H-pyrazol-1-yl]benzenesulfonamide;
4-[5-(4-chlorophenyl)-3-(hydroxymethyl)-1H-pyrazol-1-yl]benzenesulfonamide;
4-[5-(4-(N,N-dimethylamino)phenyl)-3-(trifluoromethyl)-1H-pyrazol-1-yl]benzenesulfonamide;
5-(4-fluorophenyl)-6-[4-(methylsulfonyl)phenyl]spiro[2.4]hept-5-ene;
4-[6-(4-fluorophenyl)spiro[2.4]hept-5-en-5-yl]benzenesulfonamide;
6-(4-fluorophenyl)-7-[4-(methylsulfonyl)phenyl]spiro[3.4]oct-6-ene;
5-(3-chloro-4-methoxyphenyl)-6-[4-(methylsulfonyl)phenyl]spiro[2.4]hept-5-ene;
4-[6-(3-chloro-4-methoxyphenyl)spiro[2.4]hept-5-en-5-yl]benzenesulfonamide;
5-(3,5-dichloro-4-methoxyphenyl)-6-[4-(methylsulfonyl)phenyl]spiro[2.4]hept-5-ene;
5-(3-chloro-4-fluorophenyl)-6-[4-(methylsulfonyl)phenyl]spiro[2.4]hept-5-ene;
4-[6-(3,4-dichlorophenyl)spiro[2.4]hept-5-en-5-yl]benzenesulfonamide;
2-(3-chloro-4-fluorophenyl)-4-(4-fluorophenyl)-5-(4-methylsulfonylphenyl)thiazole;
2-(2-chlorophenyl)-4-(4-fluorophenyl)-5-(4-methylsulfonylphenyl)thiazole;
5-(4-fluorophenyl)-4-(4-methylsulfonylphenyl)-2-methylthiazole;
4(4-fluorophenyl)-5-(4-methylsulfonylphenyl)-2-trifluoromethylthiazole;
4(4-fluorophenyl)-5-(4-methylsulfonylphenyl)-2-(2-thienyl)thiazole;
4(4-fluorophenyl)-5-(4-methylsulfonylphenyl)-2-benzylaminothiazole;
4-(4-fluorophenyl)-5-(4-methylsulfonylphenyl)-2-(-propylamino)thiazole;
2-[(3,5-dichlorophenoxy)methyl)-4-(4-fluorophenyl)-5-[4-(methylsulfonyl)phenyl]thiazole;
5-(4-fluorophenyl)-4-(4-methylsulfonylphenyl)-2-trifluoromethylthiazole;
1-methylsulfonyl-4-[1,1-dimethyl-4-(4-fluorophenyl)cyclopenta-2,4-dien-3-yl]benzene;
4-[4-(4-fluorophenyl)-1,1-dimethylcyclopenta-2,4-dien-3-yl]benzenesulfonamide;
5-(4-fluorophenyl)-6-[4-(methylsulfonyl)phenyl]spiro[2.4]hepta-4,6-diene;
4-[6-(4-fluorophenyl)spiro[2.4]hepta-4,6-dien-5-yl]benzenesulfonamide;
6-(4-fluorophenyl)-2-methoxy-5-[4-(methylsulfonyl)phenyl]-pyridine-3-carbonitrile;
2-bromo-6-(4-fluorophenyl)-5-[4-(methylsulfonyl)phenyl]-pyridine-3-carbonitrile;
6-(4-fluorophenyl)-5-[4-(methylsulfonyl)phenyl]-2-phenyl-pyridine-3-carbonitrile;
4-[2-(4-methylpyridin-2-yl)-4-(trifluoromethyl)-1H-imidazol-1-yl]benzenesulfonamide;
4-[2-(5-methylpyridin-3-yl)-4-(trifluoromethyl)-1H-imidazol-1-yl]benzenesulfonamide;
4-[2-(2methylpyridin-3-yl)-4-(trifluoromethyl)-1H-imidazol-1-yl]benzenesulfonamide;
3-[1-[4-(methylsulfonyl)phenyl]-4-(trifluoromethyl)-1H-imidazol-2-yl]pyridine;
2-[1-[4-(methylsulfonyl)phenyl-4-(trifluoromethyl)-1H-imidazol-2-yl]pyridine;
2-methyl-4-[1-[4-(methylsulfonyl)phenyl-4-(trifluoromethyl)-1H-imidazol-2-yl]pyridine;
2-methyl-6-[1-[4-(methylsulfonyl)phenyl-4-(trifluoromethyl)-1H-imidazol-2-yl]pyridine;
4-[2-(6-methylpyridin-3-yl)-4-(trifluoromethyl)-1H-imidazol1-yl]benzenesulfonamide;
2-(3,4-difluorophenyl)-1-[4-(methylsulfonyl)phenyl]-4-(trifluoromethyl)-1H-imidazole;
4-[2-(4-methylphenyl)-4-(trifluoromethyl)-1H-imidazol-1-yl]benzenesulfonamide;
2-(4-chlorophenyl)-1-[4-(methylsulfonyl)phenyl]-4-methyl-1H-imidazole;
2-(4-chlorophenyl)-1-[4-(methylsulfonyl)phenyl]-4-phenyl-1H-imidazole;
2-(4-chlorophenyl)-4-(4-fluorophenyl)-1-[4-(methylsulfonyl)phenyl]-1H-imidazole;
2-(3-fluoro-4-methoxyphenyl)-1-[4-(methylsulfonyl)phenyl-4-(trifluoromethyl)-1H-imidazole;
1-[4-(methylsulfonyl)phenyl]-2-phenyl-4-trifluoromethyl-1H-imidazole;
2-(4-methylphenyl)-1-[4-(methylsulfonyl)phenyl]-4-trifluoromethyl-1H-imidazole;
4-[2-(3-chloro-4-methylphenyl)-4-(trifluoromethyl)-1H-imidazol-1-yl]benzenesulfonamide;
2-(3-fluoro-5-methylphenyl)-1-[4-(methylsulfonyl)phenyl]-4-(trifluoromethyl)-1H-imidazole;
4-[2-(3-fluoro-5-methylphenyl)-4-(trifluoromethyl)-1H-imidazol-1-yl]benzenesulfonamide;
2-(3-methylphenyl)-1-[4-(methylsulfonyl)phenyl]-4-trifluoromethyl-1H-imidazole;
4-[2-(3-methylphenyl)-4-trifluoromethyl-1H-imidazol-1-yl]benzenesulfonamide;
1-[4-(methylsulfonyl)phenyl]-2-(3-chlorophenyl)-4-trifluoromethyl-1H-imidazole;
4-[2-(3-chlorophenyl)-4-trifluoromethyl-1H-imidazol-1-yl]benzenesulfonamide;
4-[2-phenyl-4-trifluoromethyl-1H-imidazol-1-yl]benzenesulfonamide;
4-[2-(4-methoxy-3-chlorophenyl)-4-trifluoromethyl-1H-imidazol-1-yl]benzenesulfonamide;
1-allyl-4-(4-fluorophenyl)-3-[4-(methylsulfonyl)phenyl]-5-(trifluoromethyl)-1H-pyrazole;
4-[1-ethyl-4-(4-fluorophenyl)-5-(trifluoromethyl)-1H-pyrazol-3-yl]benzenesulfonamide;
N-phenyl-[4-(4-fluorophenyl)-3-[4-(methylsulfonyl)phenyl]-5-(trifluoromethyl)-1H-pyrazol-1-yl]acetamide;
ethyl [4-(4-fluorophenyl)-3-[4-(methylsulfonyl)phenyl]-5-(trifluoromethyl)-1H-pyrazol-1-yl]acetate;
4(4-fluorophenyl)-3-[4-(methylsulfonyl)phenyl]-1-(2-phenylethyl)-1H-pyrazole;
4(4-fluorophenyl)-3-[4-(methylsulfonyl)phenyl]-1-(2-phenylethyl)-5-(trifluoromethyl)pyrazole;
1ethyl-4-(4-fluorophenyl)-3-[4-(methylsulfonyl)phenyl]-5-(trifluoromethyl)-1H-pyrazole;
5-(4-fluorophenyl)-4-(4-methylsulfonylphenyl)-2-trifluoromethyl-1H-imidazole;
4-[4-(methylsulfonyl)phenyl]-5-(2-thiophenyl)-2-(trifluoromethyl)-1H-imidazole;
5-(4-fluorophenyl)-2-methoxy-4-[4-(methylsulfonyl)phenyl]-6-(trifluoromethyl)pyridine;
2-ethoxy-5-(4-fluorophenyl)-4-[4-(methylsulfonyl)phenyl]-6-(trifluoromethyl)pyridine;
5-(4-fluorophenyl)-4-[4-(methylsulfonyl)phenyl]-2-(2-propynyloxy)-6-(trifluoromethyl)pyridine;
2-bromo-5-(4-fluorophenyl)-4-[4-(methylsulfonyl)phenyl]-6-(trifluoromethyl)pyridine;
4-[2-(3-chloro-4-methoxyphenyl)-4,5-difluorophenyl]benzenesulfonamide;
1-(4-fluorophenyl)-2-[4-(methylsulfonyl)phenyl]benzene;
5-difluoromethyl-4-(4-methylsulfonylphenyl)-3-phenylisoxazole;
4-[3-ethyl-5-phenylisoxazol-4-yl]benzenesulfonamide;
4-[5-difluoromethyl-3-phenylisoxazol-4-yl]benzenesulfonamide;
4-[5-hydroxymethyl-3-phenylisoxazol-4-yl]benzenesulfonamide;
4-[5-methyl-3-phenyl-isoxazol-4-yl]benzenesulfonamide;
1-[2-(4-fluorophenyl)cyclopenten-1-yl]-4-(methylsulfonyl)benzene;
1-[2-(4-fluoro-2-methylphenyl)cyclopenten-1-yl]-4-(methylsulfonyl)benzene;
1-[2-(4-chlorophenyl)cyclopenten-1-yl]-4-(methylsulfonyl)benzene;
1-[2-(2,4-dichlorophenyl)cyclopenten-1-yl]-4-(methylsulfonyl)benzene;
1-[2-(4-trifluoromethylphenyl)cyclopenten-1-yl]-4-(methylsulfonyl)benzene;
1-[2-(4-methylthiophenyl)cyclopenten-1-yl]-4-(methylsulfonyl)benzene;
1-[2-(4-fluorophenyl)-4,4-dimethylcyclopenten-1-yl]-4-(methylsulfonyl)benzene;
4-[2-(4-fluorophenyl)-4,4-dimethylcyclopenten-1-yl]benzenesulfonamide;
1-[2-(4-chlorophenyl)-4,4-dimethylcyclopenten-1-yl]-4-(methylsulfonyl)benzene;
4-[2-(4-chlorophenyl)-4,4-dimethylcyclopenten-1-yl]benzenesulfonamide;
4-[2-(4-fluorophenyl)cyclopenten-1-yl]benzenesulfonamide;
4-[2-(4-chlorophenyl)cyclopenten-1-yl]benzenesulfonamide;
1-[2-(4-methoxyphenyl)cyclopenten-1-yl]-4-(methylsulfonyl)benzene;
1-[2-(2,3-difluorophenyl)cyclopenten-1-yl]-4-(methylsulfonyl)benzene;
4-[2-(3-fluoro-4-methoxyphenyl)cyclopenten-1-yl]benzenesulfonamide;
1-[2-(3-chloro-4-methoxyphenyl)cyclopenten-1-yl]-4-(methylsulfonyl)benzene;
4-[2-(3-chloro-4-fluorophenyl)cyclopenten-1-yl]benzenesulfonamide;
4-[2-(2-methylpyridin-5-yl)cyclopenten-1-yl]benzenesulfonamide;
ethyl 2-[4-(4-fluorophenyl)-5-[4-(methylsulfonyl) phenyl]oxazol-2-yl]-2-benzyl-acetate;
2-[4-(4-fluorophenyl)-5-[4-(methylsulfonyl)phenyl]oxazol-2-yl]acetic acid;
2-(tert-butyl)-4-(4-fluorophenyl)-5-[4-(methylsulfonyl)phenyl]oxazole;
4-(4-fluorophenyl)-5-[4-(methylsulfonyl)phenyl]-2-phenyloxazole;
4-(4-fluorophenyl)-2-methyl-5-[4-(methylsulfonyl)phenyl]oxazole;
4-[5-(3-fluoro-4-methoxyphenyl)-2-trifluoromethyl-4-oxazolyl]benzenesulfonamide;
6-chloro-7-(1,1-dimethylethyl)-2-trifluoromethyl-2H-1-benzopyran-3-carboxylic acid;
6-chloro-8-methyl-2-trifluoromethyl-2H-1-benzopyran-3-carboxylic acid;
5,5-dimethyl-3-(3-fluorophenyl)-4-methylsulfonyl-2(5H)-furanone;
6-chloro-2-trifluoromethyl-2H-1benzothiopyran-3-carboxylic acid;
4-[5-(4-chlorophenyl)-3-(trifluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[5-(3-fluoro-4-methoxyphenyl)-3-(difluoromethyl)-1H-pyrazol-1-yl]benzenesulfonamide;
3-[1-[4-(methylsulfonyl)phenyl]-4-trifluoromethyl-1H-imidazol-2-yl]pyridine;
2-methyl-5-[1-[4-(methylsulfonyl)phenyl]-4-trifluoromethyl-1H-imidazol-2-yl]pyridine;
4-[2-(5 -methylpyridin-3-yl)-4-(trifluoromethyl)-1H-imidazol-1-yl]benzenesulfonamide;
4-[5-methyl-3-phenylisoxazol-4-yl]benzenesulfonamide;
4-[5-hydroxymethyl-3-phenylisoxazol-4-yl]benzenesulfonamide;
[2-trifluoromethyl-5-(3,4-difluorophenyl)-4-oxazolyl]benzenesulfonamide;
4-[2-methyl-4-phenyl-5-oxazolyl]benzenesulfonamide;
4-[5-(2-fluoro-4-methoxyphenyl)-2-trifluoromethyl-4-oxazolyl]benzenesulfonamide;
[2-(2-chloro-6-fluoro-phenylamino)-5-methyl-phenyl]-acetic acid;
N-(4-Nitro-2-phenoxy-phenyl)-methanesulfonamide or nimesulide;
N-[6-(2,4-difluoro-phenoxy)-1-oxo-indan-5-yl]-methanesulfonamide;
N-[6-(2,4-Difluoro-phenylsulfanyl)-1-oxo-1H-inden-5-yl]-methanesulfonamide, soldium salt;
N-[5-(4-fluoro-phenylsulfanyl)-thiophen-2-yl]-methanesulfonamide;
3-(3,4-Difluoro-phenoxy)-4-(4-methanesulfonyl-phenyl)-5-methyl-5-(2,2,2-trifluoro-ethyl)-5H-furan-2-one;
(5Z)-2-amino-5-[[3,5-bis(1,1-dimethylethyl)-4-hydroxyphenyl]methylene]-4(5H)-thiazolone;
N-[3-(formylamino)-4-oxo-6-phenoxy-4H-1-benzopyran-7-yl]-methanesulfonamide;
(6aR,10aR)-3-(1,1-dimethylheptyl)-6a,7,10,10a-tetrahydro-1-hydroxy-6,6-dimethyl-6H-dibenzo[b,d]pyran-9-carboxylic acid;
4-[[3,5-bis(1,1-dimethylethyl)-4-hydroxyphenyl]methylene]dihydro-2-methyl-2H-1,2-oxazin-3(4H)-one;
6-dioxo-9H-purin-8-yl-cinnamic acid;
4-[4-(methyl)-sulfonyl)phenyl]-3-phenyl-2(5H)-furanone;
4-(5-methyl-3-phenyl-4-isoxazolyl);
2-(6-methylpyrid-3-yl)-3-(4-metbylsulfonylphenyl)-5-chloropyridine;
4-[5-(4-methylphenyl)-3-(trifluoromethyl)-1H-pyrazol-1-yl];
N-[[4-(5-methyl-3-phenyl-4-isoxazolyl)phenyl]sulfonyl];
4-[5-(3-fluoro-4-methoxyphenyl)-3-difluoromethyl)-1H-pyrazol-1-yl]benzenesulfonamide;
(S)-6,8-dichloro-2-(trifluoromethyl)-2H-1-benzopyran-3-carboxylic acid;
2-(3,4-difluorophenyl)-4-(3-hydroxy-3-methylbutoxy)-5-[4-(methylsulfonyl)phenyl]-3(2H)-pyridzainone;
2-trifluoromethyl-3H-naptho[2,1-b]pyran-3-carboxylic acid;
6-chloro-7-(1,1-dimethylethyl)-2-trifluoromethyl-2H-1-benzopyran-3-carboxylic acid;
and
[2-(2,4-dichloro-6-ethyl-3,5-dimethyl-phenylamino)-5-propyl-phenyl]-acetic acid.

95. The method of claim 60 wherein the cyclooxygenase-2 selective inhibitor, pharmaceutically acceptable salt, isomer, or prodrug thereof acts as an immunostimulant.

96. The method of claim 60 wherein the anti-human immunodeficiency virus agent substantially inhibits HIV infection in the human.

97. The method of claim 96 wherein the anti-human immunodeficiency virus agent inhibits HIV infection by substantially inhibiting the HIV virus.

98. The method of claim 96 wherein the anti-human immunodeficiency virus agent substantially inhibits HIV infection by causing the human to substantially inhibit the HIV infection.

99. The method of claim 96 wherein the anti-human immunodeficiency virus agent is selected from the group consisting of a viral cellular entry inhibitor, a viral replication inhibitor, a viral assembly inhibitor, an integrase inhibitor, and a human immune enhancing agent.

100. The method of claim 99 wherein the viral replication inhibitor is selected from the group consisting of a nucleoside analog, a non-nucleoside reverse transcriptase inhibitor, a acyclic nucleoside phosphonate analog, a zinc finger inhibitor, a viral gene expression inhibitor, a polyamine biosynthesis inhibitor, and a genetic or anti-sense therapy agent.

101. The method of claim 100 wherein the nucleoside analog is selected from the group consisting of:
(−)-cis-2-amino-1,9-dihydro-9-[4-hydroxymethyl)-2-cyclopenten-1-yl)-6H-purin-6-one;
2,6-diamino-2′,3′-dideoxypurine-9-ribofuranoside;
9-(2-azido-2,3-dideoxy-b-D-erythro-pentofuranosyl)adenine;
1-(2′-fluoro-2′,3′-dideoxy-B-D-erythro-pentofuranosyl)thymine;
9-(2-azido-2,3-dideoxy-b-D-threo-pentofuranosyl)adenine;
3-(3-oxo-1-propenyl)-3′-azido-3′-deoxythymidine;
3-azido-2′,3′-dideoxy-5-chlorocytidine;
3′-azido-3′-deoxy-6-azathymidine;
2′,3′-dideoxy-3′-fluoro-4-thiothymidine;
2′,3′-dideoxy-3′-fluoro-5-chlorocytidine;
9-(3′-fluoro-2′,3′-dideoxy-B-D-erythropentafuranosyl)adenine;
3′-fluoro-2′,3′-dideoxycytidine;
2,6-diaminopurine-3′-fluoro-2′,3′-dideoxyriboside;
3′-fluoro-2′,3′-dideoxyguanosine;
3′-fluoro-2′,3′-dideoxyuridine;
1-[2′,3′-dideoxy-3′-C-(hydroxymethyl)-.beta.-D-erythro-pentofuranosyl]cytosine;
3′-azido-2′,3′-dideoxy-5-trifluoromethyluridine;
3′-azido-2′,3′-dideoxy-5-[(cyanomethyl)oxy]uridine;
3′-azido-2′,3′-dideoxy-5-fluorocytidine;
3′-azido-2′,3′-dideoxy-5-methylcytidine;
3′-azido-2′,3′-dideoxy-5-aminouridine;
3′-azido-2′,3′-dideoxy-5-methyaminouridine;
3′-azido-2′,3′-dideoxy-5-dimethylaminouridine;
3′-azido-2′,3′-dideoxy-5-hydroxyuridine;
3′-azido-2′,3′-dideoxy-5-thiocyanatouridine;
9-(3′-azido-2′,3′-dideoxy-B-D-erythropentafuranosyl)adenine;
3′-azido-2′,3′-dideoxycytidine;
3′-azido-2′,3′-dideoxyguanosine;
3′-azido-2′,3′-dideoxy-N4-5-dimethylcytidine;
3′-azido-2′,3′-dideoxy-N4-OH-5-methylcytidine;
4′-azido-3′-deoxythymidine;
4′-azido-5-chloro-2′-deoxyuridine;
4′-azido-2′-deoxyadenosine;
4′-azido-2′-deoxycytidine;
4′-azido-2′-deoxyguanosine;
4′-azido-2′-deoxyinosine;
4-azido-2′-deoxyuridine;
1-(4-azido-2-deoxy-.beta.-D-erythro-pentofuranosyl)-5-methyl-2,4-dioxopyrimidine;
4′-cyanothymidine;
5-fluoro-2′,3′-dideoxycytidine;
3′-azido-3′-deoxythymidine-5′-(butylmethoxyvalinyl)phosphate;
6-chloro-9-(2,3-dideoxy-.beta.-D-glyceropentofuranosyl)-9H-purine;
2′,3′-dideoxy-3′-fluoro-5-chlorouridine;
butanedioic acid, compd. with (1S-cis)-4-[2-amino-6-(cyclopropylamino)-9H-purine-9-yl]-2-cyclopentene-1-methanol (1:1);
5′-alkylglycoside carbonate of 3′-azido-3′-deoxythymidine;
3′-azido-2′,3′-dideoxy-5-bromouridine;
3′-azido-5-chloro-2′,3′-dideoxyuridine;
3′-azido-2′,3′-dideoxy-5-ethyluridine;
3′-azido-2′,3′-dideoxy-5-fluorouridine;
3′-azido-2′,3′-dideoxy-5-iodouridine;
2,5′-anhydro-3′-azido-3′-deoxythymidine;
1-(2,3-dideoxy-3-azido-a-L-threo-pentofuranosyl)thymine;
5′-[(1,4-dihydro-1-methyl-3-pyridinylcarbonyl)oxy]-3′-azido-2′3′-deoxythymidine;
3′-azido-3′-deoxythymidilyl-(5′,5′-2′,3′-dideoxy-5′-adenylic acid, 2-cyanoethyl ester;
3-azido-3′-deoxythymidilyl-(5′,5′)-2′,3′-dideoxy-5′-adenylic acid;
3′-azido-3′-deoxythymidilyl-(5′,5′)-2′,3′-dideoxy-5′inosinic acid;
O,O′-bis(3′-azido-3′-deoxythymidin-5′-yl)methylphosphonate;
2,5′-anhydro-3′-azido-2′,3′-dideoxyuridine;
2,4(1H,3H)-pyrimidinedione,5-(3-azido-2,3-dideoxy-.beta.-D-erythro-pentofuranosyl);
(1S,4R)-4-[2-amino-6-(cyclopropylamino)-9H-purin-9-yl]-2-cyclopentene-1-methanol & .beta.-L-(−)-2′,3′-dideoxy-3′-thiacytidine & 3′-azido-3′-deoxythymidine;
(±)-9-[(1.beta.-2.alpha.-3.beta.)-2,3-bis(hydroxymethyl)-1-cyclobutyl]adenine;
9-[1.beta.-2.alpha.-3.beta.]-2,3-bis(hydroxymethyl)-1-cyclobutyl]guanine;
9-(2,3-dideoxy-.beta.-D-ribofuranosyl)-6-(methylthio)purine;
2,3-dideoxydidehydroadenosine;
3′-azido-3′-deoxythymidine;
2′,3′-dideoxydidehydrocytidine;
2,6-diaminopurine-2′,3′-dideoxydidehydrorboside;
b-D-2′,3′-didehydro-2′,3′-dideoxy-5-fluorocytidine;
2′,3′-didehydro-2′,3′-dideoxyguanosine;
2′,3′-dideoxyadenosine;
2′,3′-dideoxyguanosine;
3′-deoxythymidine;
2′,3′-dideoxyinosine;
6-dimethylaminopurine-2′,3′-dideoxyriboside;
(−)-2′-deoxy-3′-oxa-4′-thiocytidine;
(+)-2′-deoxy-3′-oxa-4′-thiocytidine;
(−)-2′-deoxy-3′-oxa-4′-thio-5-fluorocytidine;
(+)-2′-deoxy-3′-oxa-4′-thio-5 -fluorocytidine;
(−)-(2R,4R)-9-[2-(hydroxymethyl)-1,3-dioxolan-4-yl]guanine;
(+)-(2S,4R)-1-[2-(hydroxymethyl)-1,3-dioxolan-4-yl]-5-fluorocytosine;
2′,3′-dideoxy-3′-fluoro-5-bromouridine;
3′-fluoro-2′,3′-dideoxy-5-iodouridine;
3′-fluoro-3′-deoxythymidine;
(−)-(2R,5S)-5-fluoro-1-[2-(hydroxymethyl)-1,3-oxathiolan-5-yl]cytosine;
(+)-(2R,5R)-5-fluoro-1-[2-(hydroxymethyl)-1,3-oxathiolan-5-yl]cytosine;
.beta.-L-2′,3′-didehydro-2′,3′-dideoxyadenosine;
2′,3′-dideoxy-2′,3′-didehydro-.beta.-L-5-5-fluorocytidine;
.beta.-L-2′,3′-didehydro-2′,3′-dideoxyinosine;
.beta.-L-2′,3′-didehydro-2′,3′-dideoxyguanosine;
2(1H)-pyrimidinone, 4-amino-5-fluoro-1-[(2S,5R)-tetrahydro-5-(hydroxymethyl)-2-furanyl];
cis-1-[2′-hydroxymethyl-5′-(1,3-oxathiolanyl)]cytosine;
9-(2″-fluoro-2′,3′-dideoxy-B-D-threopentafuranosyl)adenine;
5-methyl-3′-azido-2′3′-dideoxyisocytidine;
N-ethyl-2′,3′-dideoxyadenosine;
6-methyl-2′,3′-dideoxyadenosine;
1.beta.-D-ribofuranosyl-1,2,4-triazolo-3-carboxamide;
1-(2′,3′-dideoxy-2′-fluoro-.beta.-D-threo-pentofuranosyl)cytosine;
thymidine, 2′,3′-didehydro-,3′-deoxy;
(1S,4R)-4-[2-amino-6-(cyclopropylamino)-9H-purin-9-yl]-2-cyclopentene-1-methanol & .beta.-L-(−)-2′,3′-dideoxy-3′-thiacytidine & 3′-azido-3′-deoxythymidine;
3′-azido-2′,3′-dideoxyuridine;
2′,3′-dideoxycytidine;
9-[(R)-2-[[bis[[(isopropoxycarbonyl)oxy]methoxy]phosphinyl]methoxy]propyl]adenine;
or a prodrug thereof.

102. The method of claim 100 wherein the non-nucleoside reverse transcriptase inhibitor is selected from the group consisting of:
(−)-cis-2-amino-1,9-dihydro-9-[4-hydroxymethyl)-2-cyclopenten-1-yl)-6H-purin-6-one;
2,6-diamino-2′,3′-dideoxypurine-9-ribofuranoside;
9-(2-azido-2,3-dideoxy-b-D-erythro-pentofuranosyl)adenine;
1-(2′-fluoro-2′,3′-dideoxy-B-D-erythro-pentofuranosyl)thymine;
9-(2-azido-2,3-dideoxy-b-D-threo-pentofuranosyl)adenine;
3-(3-oxo-1-propenyl)-3′-azido-3′-deoxythymidine;
3-azido-2′,3′-dideoxy-5-chlorocytidine;
3′-azido-3′deoxy-6-azathymidine;
2′,3′-dideoxy-3′-fluoro-4-thiothymidine;
2′,3′-dideoxy-3′-fluoro-5-chlorocytidine;
9-(3′-fluoro-2′,3′-dideoxy-B-D-erythropentafuranosyl)adenine;
3′-fluoro-2′,3′-dideoxycytidine;
2,6-diaminopurine-3′-fluoro-2′,3′-dideoxyriboside;
3′-fluoro-2′,3′-dideoxyguanosine;
3′-fluoro-2′3′-dideoxyuridine;
1-[2′,3′-dideoxy-3′-C-(hydroxymethyl)-.beta.-D-erythro-pentofuranosyl]cytosine;
3′-azido-2′,3′-dideoxy-5-trifluoromethyluridine;
3′-azido2′,3′-dideoxy-5-[(cyanomethyl)oxy]uridine;
3′-azido-2′,3′-dideoxy-5-fluorocytidine;
3′-azido-2′,3′-dideoxy-5-methylcytidine;
3′-azido-2′,3′-dideoxy-5-aminouridine;
3′-azido-2′,3′-dideoxy-5-methyaminouridine;
3′-azido-2′,3′-dideoxy-5-dimethylaminouridine;
3′-azido-2′,3′-dideoxy-5-hydroxyuridine;
3′-azido-2′,3′-dideoxy-5-thiocyanatouridine;
9-(3′-azido-2′,3′-dideoxy-B-D-erythropentafuranosyl)adenine;
3′-azido-2′,3′-dideoxycytidine;
3′-azido-2′,3′-dideoxyguanosine;
3′-azido-2′,3′-dideoxy-N4-5-dimethylcytidine;
3′-azido-2′,3′-dideoxy-N4-OH-5-methylcytidine;
4′-azido-3′-deoxythymidine;
4′-azido-5-chloro-2′-deoxyuridine;
4′-azido-2′-deoxyadenosine;
4′-azido-2′-deoxycytidine;
4′-azido-2′-deoxyguanosine;
4′-azido-2′-deoxyinosine;
4′-azido-2′-deoxyuridine;
1(4-azido-2-deoxy-.beta.-D-erythro-pentofuranosyl)-5-methyl-2,4-dioxopyrimidine;
4′-cyanothymidine;
5-fluoro-2′,3′-dideoxycytidine;
3′-azido-3′-deoxythymidine-5′-(butylmethoxyvalinyl)phosphate;
6-chloro-9-(2,3-dideoxy-.beta.-D-glyceropentofuranosyl)-9H-purine;
2′,3′-dideoxy-3′-fluoro-5-chlorouridine;
butanedioic acid, compd. with (1S-cis)-4-[2-amino-6-(cyclopropylamino)-9H-purine-9-yl]-2-cyclopentene-1-methanol (1:1);
5′-alkylglycoside carbonate of 3′-azido-3′-deoxythymidine;
3′-azido-2′,3′-dideoxy-5-bromouridine;
3′-azido-5-chloro-2′,3′-dideoxyuridine;
3′-azido-2′,3′-dideoxy-5-ethyluridine;
3′-azido-2′,3′-dideoxy-5-fluorouridine;
3′-azido-2′,3′-dideoxy-5-iodouridine;
2,5′-anhydro-3′-azido-3′-deoxythymidine;
1-(2,3-dideoxy-3-azido-a-L-threo-pentofuranosyl)thymine;
5′-[(1,4-dihydro-1-methyl-3-pyridinylcarbonyl)oxy]-3′-azido-2′3′-deoxythymidine;
3′-azido-3′-deoxythymidilyl-(5′,5′)-2′,3′-dideoxy-5′-adenylic acid, 2-cyanoethyl ester;
3-azido-3′-deoxythymidilyl-(5′,5′)-2′,3′-dideoxy-5′-adenylic acid;
3′-azido-3′-deoxythymidilyl-(5′,5′)-2′,3′-dideoxy-5′inosinic acid;
O,O′-bis(3′-azido-3′-deoxythymidin-5′-yl)methylphosphonate;
2,5′-anhydro-3′-azido-2′,3′-dideoxyuridine;
2,4(1H,3H)-pyrimidinedione,5-(3-azido-2,3-dideoxy-.beta.-D-erythro-pentofuranosyl);
(1S,4R)-4-[2-amino-6-(cyclopropylamino)-9H-purin-9-yl]-2-cyclopentene-1-methanol & .beta.-L-(−)-2′,3′-dideoxy-3 ′-thiacytidine & 3′-azido-3′-deoxythymidine;
(±)-9-[(1.beta.-2.alpha.-3.beta.)-2,3-bis(hydroxymethyl)-1-cyclobutyl]adenine;
9-[1.beta.-2.alpha.-3.beta.]-2,3-bis(hydroxymethyl)-1-cyclobutyl]guanine;
9-(2,3-dideoxy-.beta.-D-ribofuranosyl)-6-(methylthio)purine;
2,3-dideoxydidehydroadenosine;
3′-azido-3′-deoxythymidine;
2′,3′-dideoxydidehydrocytidine;
2,6-diaminopurine-2′,3′-dideoxydidehydrorboside;
b-D-2′,3′-didehydro-2′,3′-dideoxy-5-fluorocytidine;
2′,3′-didehydro-2′,3′-dideoxyguanosine;
2′,3′-dideoxyadenosine;
2′,3′-dideoxyguanosine;
3′-deoxythymidine;
2′,3′-dideoxyinosine;
6-dimethylaminopurine-2′,3′-dideoxyriboside;
(−)-2′-deoxy-3′-oxa-4′-thiocytidine;
(+)-2′-deoxy-3′-oxa-4′-thiocytidine;
(−)-2′-deoxy-3′-oxa-4′-thio-5-fluorocytidine;
(+)-2′-deoxy-3′-oxa-4′-thio-5-fluorocytidine;
(−)-(2R,4R)-9-[2-(hydroxymethyl)-1,3-dioxolan-4-yl]guanine;
(+)-(2S,4R)-1-[2-(hydroxymethyl)-1,3-dioxolan-4-yl]-5-fluorocytosine;
2′,3′-dideoxy-3′-fluoro-5-bromouridine;
3′-fluoro-2′,3′-dideoxy-5-iodouridine;
3′-fluoro-3′-deoxythymidine;
(−)-(2R,5S)-5-fluoro-1-[2-(hydroxymethyl)-1,3-oxathiolan-5-yl]cytosine;
(+)-(2R,5R)-5-fluoro-1-[2-(hydroxymethyl)-1,3-oxathiolan-5-yl]cytosine;
.beta.-L-2′,3′-didehydro-2′,3′-dideoxyadenosine;
2′,3′-dideoxy-2′,3′-didehydro-.beta.-L-5-5-fluorocytidine;
.beta.-L-2′,3′-didehydro-2′,3′-dideoxyinosine;
.beta.-L-2′,3′-didehydro-2′,3′-dideoxyguanosine;
2(1H)-pyrimidinone, 4-amino-5-fluoro-1-[(2S,5R)-tetrahydro-5-(hydroxymethyl)-2-furanyl];
cis-1-[2′-hydroxymethyl-5′-(1,3-oxathiolanyl)]cytosine;
9-(2″-fluoro-2′,3′-dideoxy-B-D-threopentafuranosyl)adenine;
5-methyl-3′-azido-2′3′-dideoxyisocytidine;
N-ethyl-2′,3′-dideoxyadenosine;
6-methyl-2′,3′-dideoxyadenosine;
1.beta.-D-ribofuranosyl-1,2,4-triazolo-3-carboxamide;
1(2′,3′-dideoxy-2′-fluoro-.beta.-D-threo-pentofuranosyl)cytosine;
thymidine, 2′,3′-didehydro-,3′-deoxy;
(1S,4R)-4-[2-amino-6-(cyclopropylamino)-9H-purin-9-yl]-2-cyclopentene-1-methanol & .beta.-L-(−)-2′,3′-dideoxy-3′-thiacytidine & 3′-azido-3′-deoxythymidine;
3′-azido-2′,3′-dideoxyuridine;
2′,3′-dideoxycytidine; 9-[(R)-2-[[bis[[(isopropoxycarbonyl)oxy]methoxy]phosphinyl]methoxy]propyl]adenine;
or a prodrug thereof.

103. The method of claim 99 wherein the viral assembly inhibitor is selected from the group consisting of a protease inhibitor, a viral packaging inhibitor, a glycosylation inhibitor, and a viral RNA processing inhibitor.

104. The method of claim 103 wherein the protease inhibitor is selected from the group consisting of:
(−)-cis-2-amino-1,9-dihydro-9-[4-hydroxymethyl)-2-cyclopenten-1-yl)-6H-purin-6-one;
2,6-diamino-2′,3′-dideoxypurine-9-ribofuranoside;
9-(2-azido-2,3-dideoxy-b-D-erythro-pentofuranosyl)adenine;
1(2′-fluoro-2′,3′-dideoxy-B-D-erythro-pentofuranosyl)thymine;
9-(2-azido-2,3-dideoxy-b-D-threo-pentofuranosyl)adenine;
3-(3-oxo-1-propenyl)-3′-azido-3′-deoxythymidine;
3-azido-2′,3′-dideoxy-5-chlorocytidine;
3′-azido-3′-deoxy-6-azathymidine;
2′,3′-dideoxy-3′-fluoro-4-thiothymidine;
2′,3′-dideoxy-3′-fluoro-5-chlorocytidine;
9-(3′-fluoro-2′,3′-dideoxy-B-D-erythropentafuranosyl)adenine;
3′-fluoro-2′,3′-dideoxycytidine;
2,6-diaminopurine-3′-fluoro-2′,3′-dideoxyriboside;
3′-fluoro-2′,3′-dideoxyguanosine;
3′-fluoro-2′,3′-dideoxyuridine;
1-[2′,3′-dideoxy-3′-C-(hydroxymethyl)-.beta.-D-erythro-pentofuranosyl]cytosine;
3′-azido-2′,3′-dideoxy-5-trifluoromethyluridine;
3′-azido-2′,3′-dideoxy-5-[(cyanomethyl)oxy]uridine;
3′-azido-2′,3′-dideoxy-5-fluorocytidine;
3′-azido-2′,3′-dideoxy-5-methylcytidine;
3′-azido-2′,3′-dideoxy-5-aminouridine;
3′-azido-2′,3′dideoxy-5-methyaminouridine;
3′-azido-2′,3′-dideoxy-5-dimethylaminouridine;
3′-azido-2′,3′-dideoxy-5-hydroxyuridine;
3′-azido-2′,3′-dideoxy-5-thiocyanatouridine;
9-(3′-azido-2′,3′-dideoxy-B-D-erythropentafuranosyl)adenine;
3′-azido-2′,3′-dideoxycytidine;
3′-azido-2′,3′-dideoxyguanosine;
3′-azido-2′,3′-dideoxy-N4-5-dimethylcytidine;
3′-azido-2′,3′-dideoxy-N4-OH-5-methylcytidine;
4′-azido-3′-deoxythymidine;
4′-azido-5-chloro-2′-deoxyuridine;
4′-azido-2′-deoxyadenosine;
4′-azido-2′-deoxycytidine;
4′-azido-2′-deoxyguanosine;
4′-azido-2′-deoxyinosine;
4′-azido-2′-deoxyuridine;
1(4-azido-2-deoxy-.beta.-D-erythro-pentofuranosyl)-5-methyl-2,4-dioxopyrimidine;
4′-cyanothymidine;
5-fluoro-2′,3′-dideoxycytidine;
3′-azido-3′-deoxythymidine-5′-(butylmethoxyvalinyl)phosphate;
6-chloro-9-(2,3-dideoxy-.beta.-D-glyceropentofuranosyl)-9H-purine;
2′,3′-dideoxy-3′-fluoro-5-chlorouridine;
butanedioic acid, compd. with (1S-cis)-4-[2-amino-6-(cyclopropylamino)-9H-purine-9-yl]-2-cyclopentene-1-methanol (1:1);
5′-alkylglycoside carbonate of 3′-azido-3′-deoxythymidine;
3′-azido-2′,3′-dideoxy-5-bromouridine;
3′-azido-5-chloro-2′,3′-dideoxyuridine;
3′-azido-2′,3′-dideoxy-5-ethyluridine;
3′-azido-2′,3′-dideoxy-5-fluorouridine;
3′-azido-2′,3′-dideoxy-5-iodouridine;
2,5′-anhydro-3′-azido-3′-deoxythymidine;
1(2,3-dideoxy-3-azido-a-L-threo-pentofuranosyl)thymine;
5′-[(1,4-dihydro-1-methyl-3-pyridinylcarbonyl)oxy]-3′-azido-2′3′-deoxythymidine;
3′-azido-3′-deoxythymidilyl-(5′,5′)-2′,3′-dideoxy-5′-adenylic acid, 2-cyanoethyl ester;
3-azido-3′-deoxythymidilyl-(5′,5′)-2′,3′-dideoxy-5′-adenylic acid;
3′-azido-3′-deoxythymidilyl-(5′,5′)-2′,3′-dideoxy-5′inosinic acid;
O,O′-bis(3′-azido-3′-deoxythymidin-5′-yl)methylphosphonate;
2,5′-anhydro-3′-azido-2′,3′-dideoxyuridine;
2,4(1H,3H)-pyrimidinedione,5-(3-azido-2,3-dideoxy-.beta.-D-erythro-pentofuranosyl);
(1S,4R)-4-[2-amino-6-(cyclopropylamino)-9H-purin-9-yl]-2-cyclopentene-1-methanol & .beta.-L-(−)-2′,3′-dideoxy-3′-thiacytidine & 3′-azido-3′-deoxythymidine;
(±)-9-[(1.beta.-2.alpha.-3.beta.)-2,3-bis(hydroxymethyl)-1-cyclobutyl]adenine;
9-[1.beta.-2.alpha.-3.beta.]-2,3-bis(hydroxymethyl)-1-cyclobutyl]guanine;
9-(2,3-dideoxy-.beta.-D-ribofuranosyl)-6-(methylthio)purine;
2,3-dideoxydidehydroadenosine;
3′-azido-3′-deoxythymidine;
2′,3′-dideoxydidehydrocytidine;
2,6-diaminopurine-2′,3′-dideoxydidehydrorboside;
b-D-2′,3′-didehydro-2′,3′-dideoxy-5-fluorocytidine;
2′,3′-didehydro-2′,3′-dideoxyguanosine;
2′,3′-dideoxyadenosine;
2′,3′-dideoxyguanosine;
3′-deoxythymidine;
2′,3′-dideoxyinosine;
6-dimethylaminopurine-2′,3′-dideoxyriboside;
(−)-2′-deoxy-3′-oxa-4′-thiocytidine;
(±)-2′-deoxy-3′-oxa-4′-thiocytidine;
(−)-2′-deoxy-3′-oxa-4′-thio-5-fluorocytidine;
(+)-2′-deoxy-3′-oxa-4′-thio-5-fluorocytidine;
(−)-(2R,4R)-9-[2-(hydroxymethyl)-1,3-dioxolan-4-yl]guanine;
(+)-(2S,4R)-1-[2-(hydroxymethyl)-1,3-dioxolan-4-yl]-5-fluorocytosine;
2′,3′-dideoxy-3′-fluoro-5-bromouridine;
3′-fluoro-2′,3′-dideoxy-5-iodouridine;
3′-fluoro-3′-deoxythymidine;
(−)-(2R,5S)-5-fluoro-1-[2-(hydroxymethyl)-1,3-oxathiolan-5-yl]cytosine;
(+)-(2R,5R)-5-fluoro-1-[2-(hydroxymethyl)-1,3-oxathiolan-5-yl]cytosine;
.beta.-L-2′,3′-didehydro-2′,3′-dideoxyadenosine;
2′,3′-dideoxy-2′,3′-didehydro-.beta.-L-5-5-fluorocytidine;
.beta.-L-2′,3′-didehydro-2′,3′-dideoxyinosine;
.beta.-L-2′,3′-didehydro-2′,3′-dideoxyguanosine;
2(1H)-pyrimidinone, 4-amino-5-fluoro-1-[(2S,5R)-tetrahydro-5-(hydroxymethyl)-2-furanyl];
cis-1-[2′-hydroxymethyl-5′-(1,3-oxathiolanyl)]cytosine;
9-(2″-fluoro-2′,3′-dideoxy-B-D-threopentafuranosyl)adenine;
5-methyl-3′-azido-2′3′-dideoxyisocytidine;
N-ethyl-2′,3′-dideoxyadenosine;
6-methyl-2′,3′-dideoxyadenosine;
1.beta.-D-ribofuranosyl-1,2,4-triazolo-3-carboxamide;
1-(2′,3′-dideoxy-2′-fluoro-.beta.-D-threo-pentofuranosyl)cytosine;
thymidine, 2′,3′-didehydro-,3′-deoxy;
(1S,4R)-4-[2-amino-6-(cyclopropylamino)-9H-purin-9-yl]-2-cyclopentene-1-methanol & beta.-L-(−)-2′,3′-dideoxy-3′-thiacytidine & 3′-azido-3′-deoxythymidine;
3′-azido-2′,3′-dideoxyuridine;
2′,3′-dideoxycytidine;
9-[(R)-2-[[bis[[(isopropoxycarbonyl)oxy]methoxy]phosphinyl]methoxy]propyl]adenine;
or a prodrug thereof.

105. The method of claim 99 wherein the viral cellular entry inhibitor is selected from the group consisting of a virion receptor binding antagonist, a virion co-receptor binding antagonist, a viral fusion inhibitor, and a viral uncoating inhibitor.

106. The method of claim 105 wherein the receptor binding antagonist or co-receptor binding antagonist is a CD4 receptor antagonist.

107. The method of claim 99 wherein the human immune enhancing agent is selected from the group consisting of an antimetabolite, an antineoplastic agent, an immune modulator, a cytokine, a therapeutic vaccine or antibody, an antioxidant, a hormone, and a vitamin.

108. The method of claim 99 wherein the integrase inhibitor is selected from the group consisting of:
4((3,4-dimethoxyphenyl)methyl)dihydro-3-((4-hydroxy-3-methoxyphenyl)methyl)-,(3R-trans)-2(3H)-furanone;
3,5-dicaffeoylquinic acid;
1methoxyaxalyl-3,5-dicaffeoylquinic acid;
9-[(4,6-O-ethylidene-.beta.-D-glucopyranosyl)oxy]-5,8,8a,9-tetrahydro-5-(4-hydroxy-3,5-dimethoxyphenyl)furo[3′,4′:6,7]naphtho[2,3-d]1,3-dioxol-6-(5aH)-one;
Hydroxocobalamin;
[S-(R*,R*)]-2,3-bis[[3-(3,4-dihydroxyphenyl)-1-oxo-2-propenyl]oxy]butanedioic acid.

109. The method of claim 96 wherein the anti-human immunodeficiency virus agent is selected from a natural product.

110. The method of claim 109 wherein the natural product is selected from the group consisting of:
(R)-3,6-diamino-N-(aminomethyl)hexanamide;
3-hydroxylup-20(29)-en-28-oic acid, (3.beta.);
3-O-(3′,3′-dimethylsuccinyl)-betulinic acid;
Conocurvone;
Cyanovirin-N;
3.beta.-hydroxyandrost-5-en-17-one;
16-.alpha.-bromo-3-.beta.-hydroxyandrost-5-en-17-one;
9-(guanidino)-N-[10-(guanidino)-1-(3-aminopropyl)-2-hydroxydecyl]nonanamide;
6-acetyloxy-7-(acetyloxymethyl)-5-hydroxy-3,11,11,14-tetramethyl-15-oxotetracyclo[7.5.1.0<1,5>.0<10,12>]pentadeca-2,7-dien-4-yl] acetate;
13-hydroxyingenol-3-(2,3-dimethylbutanoate)-13-dodecanoate;
1,2-dithiolane-3-pentanoic acid;
2,4,6,8,10,14-octadecahexaenamide, 13-hydroxy-N-[(1S)-2-hydroxy-1-methylethyl]-2,10,12,14,16-pentamethyl-18-phenyl-,(2E,4E,6Z,8E,10E,12R,13R,14E,16S);
4H-pyran-4-one,3-ethyl-6-methoxy-5-methyl-2-(2-(3-methyl-4-phenyl-3-butenyl)-4-o-xazolyl)-,(E);
12-deoxyphorbol-13-(3E,5E-decadienoate);
3-hydroxy-20-oxonorlupan-28-oic acid, (3.beta.);
12-deoxyphorbol-13-acetate;
4oxazolecarboxamide,2-[4,4′,4″,5,5′,5″-hexahydro-4,4′,4″-trimethyl-2″-(2-phenylethenyl)[2,4′:2′,4″-terthiazol]-4-yl]-N,5-dimethyl-,[4R-[2[2′[2″(E),4″S*],4′S*],4R*]];
Acemannan;
5,6,7-trihydroxyflavone-7-O-b-D-glucopyranosideuronic acid;
Calanolide A;
Calanolide B;
(1S,6S,7R,8R,8aR)-1,6,7,8-tetrahydroxyindolizidine;
1,5-Dideoxy-1,5-imino-D-glucitol;
3,5-dicaffeoylquinic acid;
1-methoxyaxalyl-3,5-dicaffeoylquinic acid;
hypericin;
inophyllum B;
inophyllum P;
5,5′-(1,1′-dihydroxy-8,8′-dimethoxy-6,6′-dimethyl[2,2′-binaphthalene]-4,4′-diyl)bis[1,2,3,4,-tetrahydro-1,3-dimethyl-6,8-isoquinolinediol];
5,5′-(1,1′-dihydroxy-8,8′-dimethoxy-6,6′-dimethyl[2,2′-binaphthalene]-4,4′-diyl)bis[1,2,3,4,-tetrahydro-1,3-dimethyl-6,8-isoquinolinediol]; and
5,5′-(1,1′-dihydroxy-8,8′-dimethoxy-6,6′-dimethyl[2,2′-binaphthalene]-4,4′-diyl)[3,4,-dihydro-8-methoxy-1,3-dimethyl-6-isoquinolinediol],[1,2,3,4-tetrahydro-1,3-dimethyl-6,8-isoquinolinediol].

111. The method of claim 97 wherein the anti-human immunodeficiency virus agent substantially inhibits viral replication, colonization, or assembly in the human.

112. The method of claim 98 wherein the anti-human immunodeficiency virus agent substantially inhibits mitosis in cells of the human.

113. The method of claim 98 wherein the anti-human immunodeficiency virus agent substantially increases the immune response of the human.

114. The method of claim 98 wherein the anti-human immunodeficiency virus agent substantially reduces cellular replication in the human.

115. The method of claim 96 wherein the anti-human immunodeficiency virus agent is a virucidal agent.

116. The method of claim 115 wherein the virucidal agent is selected from the group consisting of cidofovir, formaldehyde, and glutaral or a prodrug thereof.

117. The method of claim 115 wherein the virucidal agent is a T-cell proliferation inhibitor.

118. The method of claim 60 wherein the HIV infection causes acquired immunodeficiency syndrome.

119. The method of claim 60 wherein the human immunodeficiency viral infection causes a reduction in the number of T-cells present in the human.

120. The method of claim 119 wherein the T-cells are CD4+ T-cells.

121. The method of claim 120 wherein the T-cells are helper T-cells.

122. The method of claim 96 wherein the anti-human immunodeficiency virus agent substantially reduces cellular replication in the human.

123. The method of claim 96 wherein the human immunodeficiency viral infection results in the formation of an acquired immunodeficiency syndrome related disorder in the human.

124. The method of claim 123 wherein the acquired immunodeficiency syndrome related disorder is selected from the group consisting of skin rash, fever, muscle and joint aches, swelling of the lymph glands, seizures, hepatitis, diarrhea, shingles, herpes simplex infection, thrush, Kaposi′s sarcoma, pneumocystis carinii pneumonia, cryptococcal meningitis, toxoplasmosis, mycobacterium avium complex, cytomegalovirus infection, and lymphoma.

125. The method of claim 96 wherein the anti-human immunodeficiency virus agent is administered orally.

126. The method of claim 96 wherein the anti-human immunodeficiency virus agent is administered intravenously.

127. The method of claim 60 wherein the cyclooxygenase-2 selective inhibitor is administered during a continuous period prior to administration of the anti-human immunodeficiency virus agent.

128. The method of claim 60 wherein the cyclooxygenase-2 selective inhibitor is administered during a continuous period after the administration of the anti-human immunodeficiency virus agent.

129. The method of claim 60 wherein the cyclooxygenase-2 selective inhibitor is administered during a continuous period simultaneous with administration of the anti-human immunodeficiency virus agent.

130. The method of claim 60 wherein the cyclooxygenase-2 selective inhibitor and the anti-human immunodeficiency virus agent are administered sequentially.

131. The method of claim 60 wherein the cyclooxygenase-2 selective inhibitor and the anti-human immunodeficiency virus agent are administered substantially simultaneously.

132. The method of claim 123 further comprising administering an agent to the human wherein the agent treats or prevents the acquired immunodeficiency syndrome related disorder.

133. The method of claim 132 wherein the agent is ibuprofen when the acquired immunodeficiency syndrome related disorder is fever.

134. The method of claim 132 wherein the agent is an anti-neoplastic agent when the acquired immunodeficiency syndrome related disorder is cancer.

135. The method of claim 132 wherein the agent is an antifungal agent when the acquired immunodeficiency syndrome related disorder is meningitis.

136. The method of claim 132 wherein the agent is an antibiotic agent when the acquired immunodeficiency syndrome related disorder is pneumocystis carinii pneumonia.

137. The method of claim 132 wherein the agent is an antiprotozoal agent when the acquired immunodeficiency syndrome related disorder is toxoplasmosis.

138. The method of claim 132 wherein the agent is an antiviral agent when the acquired immunodeficiency syndrome related disorder is cytomegalovirus.

139. The composition of claim 40 wherein the viral cellular entry inhibitor is enfuvirtide.

140. The composition of claim 40 wherein the viral cellular entry inhibitor is hydroxyurea.

141. The method of claim 96 wherein the anti-human immunodeficiency virus agent is enfuvirtide.

142. The method of claim 96 wherein the anti-human immunodeficiency virus agent is hydroxyurea.
